SEQUENCE LISTING (577 sequences)

SEQ ID NO: 1 - protein, 86 aa, synthetic construct
MDGVSSEANEENDNIERPVRRRHSSILKPPRSPLQDLRGGNETVQESNALRNKKNSRAAAAADTIKVFQTESHMKIVRKSEMEETE

SEQ ID NO: 2 - protein, 80 aa, synthetic construct
MDGVSSEANEENDNIERPVRRRHASILKPPRSPLQDLRGGNETVQESNALRNKKNSRRVAFADTIKVFQTESHMKIVRKS

SEQ ID NO: 3 - DNA, 4518 bp, synthetic construct
  source 1..4518: mol_type = other DNA
atggatggggtgtcttcagaggctaatgaagaaaatgacaatatagagagacctgttagaagacggcattcttcaatattgaaacccccaaggagtcctcttcaggacctcagaggtgggaatgaaacagttcaagagtcaaacgcgttaaggaataagaaaaactctcgtgcagccgccgctgcagatactataaaggtattccagacggagtctcatatgaaaatagtgagaaagtcagaaatggaagaaacagaaggcggttccggcggagggtcggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcattggtagtgggagcaacggcagcagcggatcctga

SEQ ID NO: 4 - DNA, 4500 bp, synthetic construct
  source 1..4500: mol_type = other DNA
atggatggggtgtcttcagaggctaatgaagaaaatgacaatatagagagacctgttagaagacggcatgcctcaatattgaaacccccaaggagtcctcttcaggacctcagaggtgggaatgaaacagttcaagagtcaaacgcgttaaggaataagaaaaactctcgtcgagtcgcctttgcagatactataaaggtattccagacggagtctcatatgaaaatagtgagaaagtcaggcggttccggcggagggtcggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggcattggtagtgggagcaacggcagcagcggatcctga

SEQ ID NO: 5 - protein, 7 aa, synthetic construct
GGSGGGS

SEQ ID NO: 6 - RNA, 20 nt, synthetic construct
  source 1..20: mol_type = other RNA
gcatgggcggcatgaaccgg

SEQ ID NO: 7 - RNA, 20 nt, synthetic construct
  source 1..20: mol_type = other RNA
ggcagcggcgacgacgacca

SEQ ID NO: 8 - RNA, 20 nt, synthetic construct
  source 1..20: mol_type = other RNA
agttgaatacacacaacaca

SEQ ID NO: 9 - RNA, 20 nt, synthetic construct
  source 1..20: mol_type = other RNA
ttctaccattgacctcaaag

SEQ ID NO: 10 - RNA, 20 nt, synthetic construct
  source 1..20: mol_type = other RNA
tggacatttggagcgctctc

SEQ ID NO: 11 - RNA, 20 nt, synthetic construct
  source 1..20: mol_type = other RNA
aacagtccctttcatagagc

SEQ ID NO: 12 - RNA, 20 nt, synthetic construct
  source 1..20: mol_type = other RNA
gcttcaacactgttagttga

SEQ ID NO: 13 - RNA, 20 nt, synthetic construct
  source 1..20: mol_type = other RNA
ttccaatctgctccgcctaa

SEQ ID NO: 14 - RNA, 20 nt, synthetic construct
  source 1..20: mol_type = other RNA
ttcctttaggcggagcagat

SEQ ID NO: 15 - RNA, 20 nt, synthetic construct
  source 1..20: mol_type = other RNA
ctttttgcagaatctgcaag

SEQ ID NO: 16 - RNA, 20 nt, synthetic construct
  source 1..20: mol_type = other RNA
ttcaagcgctttgaggccaa

SEQ ID NO: 17 - RNA, 20 nt, synthetic construct
  source 1..20: mol_type = other RNA
ttcgagcgctttgaggccta

SEQ ID NO: 18 - RNA, 20 nt, synthetic construct
  source 1..20: mol_type = other RNA
ccacctgcagattctacaaa

SEQ ID NO: 19 - RNA, 20 nt, synthetic construct
  source 1..20: mol_type = other RNA
ccttttgtagaatctgcagg

SEQ ID NO: 20 - RNA, 20 nt, synthetic construct
  source 1..20: mol_type = other RNA
ctttctgcactacctggaag

SEQ ID NO: 21 - RNA, 20 nt, synthetic construct
  source 1..20: mol_type = other RNA
agttgaacacacacaacaca

SEQ ID NO: 22 - RNA, 20 nt, synthetic construct
  source 1..20: mol_type = other RNA
tacaagtctgctctgtgtaa

SEQ ID NO: 23 - RNA, 20 nt, synthetic construct
  source 1..20: mol_type = other RNA
ttcctcttgatagagcagtt

SEQ ID NO: 24 - RNA, 20 nt, synthetic construct
  source 1..20: mol_type = other RNA
atggctgcattccacacaca

SEQ ID NO: 25 - RNA, 20 nt, synthetic construct
  source 1..20: mol_type = other RNA
tggatatatggaccgcattg

SEQ ID NO: 26 - RNA, 20 nt, synthetic construct
  source 1..20: mol_type = other RNA
ctgcttgttatgtctgcaag

SEQ ID NO: 27 - RNA, 20 nt, synthetic construct
  source 1..20: mol_type = other RNA
actcttgctgtggcattttc

SEQ ID NO: 28 - RNA, 20 nt, synthetic construct
  source 1..20: mol_type = other RNA
aacctgctctatgaaacgga

SEQ ID NO: 29 - RNA, 20 nt, synthetic construct
  source 1..20: mol_type = other RNA
gaatgttcaactctgagagc

SEQ ID NO: 30 - RNA, 20 nt, synthetic construct
  source 1..20: mol_type = other RNA
cagaaagagtgtctcaaacc

SEQ ID NO: 31 - RNA, 20 nt, synthetic construct
  source 1..20: mol_type = other RNA
aacacttcccttcatacagc

SEQ ID NO: 32 - RNA, 20 nt, synthetic construct
  source 1..20: mol_type = other RNA
gatagctttgaaggtttcgt

SEQ ID NO: 33 - RNA, 20 nt, synthetic construct
  source 1..20: mol_type = other RNA
gtttcaaacctgctgtatga

SEQ ID NO: 34 - RNA, 20 nt, synthetic construct
  source 1..20: mol_type = other RNA
acttgagtacacacatcaca

SEQ ID NO: 35 - RNA, 20 nt, synthetic construct
  source 1..20: mol_type = other RNA
tttgaggacttcgttggaag

SEQ ID NO: 36 - RNA, 20 nt, synthetic construct
  source 1..20: mol_type = other RNA
gcttccaacgaagtcctcaa

SEQ ID NO: 37 - RNA, 20 nt, synthetic construct
  source 1..20: mol_type = other RNA
gacttcattgaggccttcgt

SEQ ID NO: 38 - RNA, 20 nt, synthetic construct
  source 1..20: mol_type = other RNA
ttcagagctgctctgtcaag

SEQ ID NO: 39 - RNA, 20 nt, synthetic construct
  source 1..20: mol_type = other RNA
ttccaacgaaatcttcacag

SEQ ID NO: 40 - RNA, 20 nt, synthetic construct
  source 1..20: mol_type = other RNA
tgcctctattcaactcacag

SEQ ID NO: 41 - RNA, 20 nt, synthetic construct
  source 1..20: mol_type = other RNA
cacctctgtgagttgaatag

SEQ ID NO: 42 - RNA, 20 nt, synthetic construct
  source 1..20: mol_type = other RNA
ctttctggagtatctggatg

SEQ ID NO: 43 - RNA, 20 nt, synthetic construct
  source 1..20: mol_type = other RNA
aacactctttctggagtatc

SEQ ID NO: 44 - RNA, 20 nt, synthetic construct
  source 1..20: mol_type = other RNA
tgtgtactcagctaacagag

SEQ ID NO: 45 - RNA, 20 nt, synthetic construct
  source 1..20: mol_type = other RNA
gttcatctctatgagtcgaa

SEQ ID NO: 46 - RNA, 20 nt, synthetic construct
  source 1..20: mol_type = other RNA
gcacgtttcaaacactcttt

SEQ ID NO: 47 - RNA, 20 nt, synthetic construct
  source 1..20: mol_type = other RNA
ttgaaacgtgctcaaagtaa

SEQ ID NO: 48 - RNA, 20 nt, synthetic construct
  source 1..20: mol_type = other RNA
tttgaaacgtgctcaaagta

SEQ ID NO: 49 - RNA, 20 nt, synthetic construct
  source 1..20: mol_type = other RNA
tcgactcatagagatgaaca

SEQ ID NO: 50 - RNA, 20 nt, synthetic construct
  source 1..20: mol_type = other RNA
acttgaatgcacatatcaca

SEQ ID NO: 51 - RNA, 20 nt, synthetic construct
  source 1..20: mol_type = other RNA
tcttaggcctaaggtgaaaa

SEQ ID NO: 52 - RNA, 20 nt, synthetic construct
  source 1..20: mol_type = other RNA
tgagtacacacatcacaaag

SEQ ID NO: 53 - RNA, 20 nt, synthetic construct
  source 1..20: mol_type = other RNA
gatagttctgaggatttcgt

SEQ ID NO: 54 - RNA, 20 nt, synthetic construct
  source 1..20: mol_type = other RNA
tggatatcttggcctcttag

SEQ ID NO: 55 - RNA, 20 nt, synthetic construct
  source 1..20: mol_type = other RNA
ctgtttgtgaagcctgccag

SEQ ID NO: 56 - RNA, 20 nt, synthetic construct
  source 1..20: mol_type = other RNA
gatatacccgtttcgaacga

SEQ ID NO: 57 - RNA, 20 nt, synthetic construct
  source 1..20: mol_type = other RNA
tgcttctgtttagttctgtg

SEQ ID NO: 58 - RNA, 20 nt, synthetic construct
  source 1..20: mol_type = other RNA
cacagagctgaacattcctt

SEQ ID NO: 59 - RNA, 20 nt, synthetic construct
  source 1..20: mol_type = other RNA
gaattgaaccaccgttttga

SEQ ID NO: 60 - RNA, 20 nt, synthetic construct
  source 1..20: mol_type = other RNA
aggatatttgcctagccttg

SEQ ID NO: 61 - RNA, 20 nt, synthetic construct
  source 1..20: mol_type = other RNA
gatcgctttcaggcctacgt

SEQ ID NO: 62 - RNA, 20 nt, synthetic construct
  source 1..20: mol_type = other RNA
acagagtagaacattccctt

SEQ ID NO: 63 - RNA, 20 nt, synthetic construct
  source 1..20: mol_type = other RNA
gacatccttgaggctttcgt

SEQ ID NO: 64 - RNA, 20 nt, synthetic construct
  source 1..20: mol_type = other RNA
aaactgctccttcaaaacga

SEQ ID NO: 65 - RNA, 20 nt, synthetic construct
  source 1..20: mol_type = other RNA
agcattctcagaaactgctt

SEQ ID NO: 66 - RNA, 20 nt, synthetic construct
  source 1..20: mol_type = other RNA
ggcagctttgaggatttcgt

SEQ ID NO: 67 - RNA, 20 nt, synthetic construct
  source 1..20: mol_type = other RNA
ggttcaacactgtcagttga

SEQ ID NO: 68 - RNA, 20 nt, synthetic construct
  source 1..20: mol_type = other RNA
ttggagcgctttcaggacga

SEQ ID NO: 69 - RNA, 20 nt, synthetic construct
  source 1..20: mol_type = other RNA
gcattctcagaaacttcgtt

SEQ ID NO: 70 - RNA, 20 nt, synthetic construct
  source 1..20: mol_type = other RNA
aacactctttctgcattccc

SEQ ID NO: 71 - RNA, 20 nt, synthetic construct
  source 1..20: mol_type = other RNA
tggatatttggctagctggg

SEQ ID NO: 72 - RNA, 20 nt, synthetic construct
  source 1..20: mol_type = other RNA
aaattgctgcatcaaaagaa

SEQ ID NO: 73 - RNA, 20 nt, synthetic construct
  source 1..20: mol_type = other RNA
gacgttccctttttcaccaa

SEQ ID NO: 74 - RNA, 20 nt, synthetic construct
  source 1..20: mol_type = other RNA
tcaactcatagagatgaaca

SEQ ID NO: 75 - RNA, 20 nt, synthetic construct
  source 1..20: mol_type = other RNA
gttcatctctatgagttgaa

SEQ ID NO: 76 - RNA, 20 nt, synthetic construct
  source 1..20: mol_type = other RNA
cttgacgcctacggtgaaaa

SEQ ID NO: 77 - RNA, 20 nt, synthetic construct
  source 1..20: mol_type = other RNA
gtatatggaagtggacgttt

SEQ ID NO: 78 - RNA, 20 nt, synthetic construct
  source 1..20: mol_type = other RNA
tggacgtttcggacggtttg

SEQ ID NO: 79 - RNA, 20 nt, synthetic construct
  source 1..20: mol_type = other RNA
aacattgcctttcctagagc

SEQ ID NO: 80 - RNA, 20 nt, synthetic construct
  source 1..20: mol_type = other RNA
ctctttctgtgggatccgca

SEQ ID NO: 81 - RNA, 20 nt, synthetic construct
  source 1..20: mol_type = other RNA
gaggtccaaatatccccttg

SEQ ID NO: 82 - RNA, 20 nt, synthetic construct
  source 1..20: mol_type = other RNA
tctgcaagtggacgtttgga

SEQ ID NO: 83 - RNA, 20 nt, synthetic construct
  source 1..20: mol_type = other RNA
tggatattcagacccctttg

SEQ ID NO: 84 - RNA, 20 nt, synthetic construct
  source 1..20: mol_type = other RNA
aaggatcctttacagagagc

SEQ ID NO: 85 - RNA, 20 nt, synthetic construct
  source 1..20: mol_type = other RNA
tctagctttgaggatttcgt

SEQ ID NO: 86 - RNA, 20 nt, synthetic construct
  source 1..20: mol_type = other RNA
gcattctcagaagcttcatt

SEQ ID NO: 87 - RNA, 20 nt, synthetic construct
  source 1..20: mol_type = other RNA
agcattctcagaagcttcat

SEQ ID NO: 88 - RNA, 20 nt, synthetic construct
  source 1..20: mol_type = other RNA
ttggagcgctctcaggacta

SEQ ID NO: 89 - RNA, 20 nt, synthetic construct
  source 1..20: mol_type = other RNA
tctcaggactacggtgaaaa

SEQ ID NO: 90 - RNA, 20 nt, synthetic construct
  source 1..20: mol_type = other RNA
ggttcaacactgttagttga

SEQ ID NO: 91 - RNA, 20 nt, synthetic construct
  source 1..20: mol_type = other RNA
tctcaggaatacggtgataa

SEQ ID NO: 92 - RNA, 20 nt, synthetic construct
  source 1..20: mol_type = other RNA
tctcaggactgcggtgaaaa

SEQ ID NO: 93 - RNA, 20 nt, synthetic construct
  source 1..20: mol_type = other RNA
tgtgtgtgtattcaactcac

SEQ ID NO: 94 - RNA, 20 nt, synthetic construct
  source 1..20: mol_type = other RNA
gggagatttcaagcactttg

SEQ ID NO: 95 - RNA, 20 nt, synthetic construct
  source 1..20: mol_type = other RNA
tatgaggccaatggtacaaa

SEQ ID NO: 96 - RNA, 20 nt, synthetic construct
  source 1..20: mol_type = other RNA
tgaatgcagagatcacaacg

SEQ ID NO: 97 - RNA, 20 nt, synthetic construct
  source 1..20: mol_type = other RNA
cacagagttgaaccttactt

SEQ ID NO: 98 - RNA, 20 nt, synthetic construct
  source 1..20: mol_type = other RNA
gatgtatttgaggccttcgt

SEQ ID NO: 99 - RNA, 20 nt, synthetic construct
  source 1..20: mol_type = other RNA
tttgagtgctttgaagccta

SEQ ID NO: 100 - RNA, 20 nt, synthetic construct
  source 1..20: mol_type = other RNA
agaaatcccgtttactacga

SEQ ID NO: 101 - RNA, 20 nt, synthetic construct
  source 1..20: mol_type = other RNA
tggaggtatcaagcgctttg

SEQ ID NO: 102 - RNA, 20 nt, synthetic construct
  source 1..20: mol_type = other RNA
gaaccttcctttagacagag

SEQ ID NO: 103 - RNA, 20 nt, synthetic construct
  source 1..20: mol_type = other RNA
aagcactttgaggccattgg

SEQ ID NO: 104 - RNA, 20 nt, synthetic construct
  source 1..20: mol_type = other RNA
ttcctttagacagagcggat

SEQ ID NO: 105 - RNA, 20 nt, synthetic construct
  source 1..20: mol_type = other RNA
ttgaggccttcgtagtaaac

SEQ ID NO: 106 - RNA, 20 nt, synthetic construct
  source 1..20: mol_type = other RNA
tttgaggccattggtggaaa

SEQ ID NO: 107 - RNA, 20 nt, synthetic construct
  source 1..20: mol_type = other RNA
ttccaatccgctctgtctaa

SEQ ID NO: 108 - RNA, 20 nt, synthetic construct
  source 1..20: mol_type = other RNA
aatccgctctgtctaaagga

SEQ ID NO: 109 - RNA, 20 nt, synthetic construct
  source 1..20: mol_type = other RNA
cagtttgtaaagtcagcaac

SEQ ID NO: 110 - RNA, 20 nt, synthetic construct
  source 1..20: mol_type = other RNA
tttgtggaattttcaggtgg

SEQ ID NO: 111 - RNA, 20 nt, synthetic construct
  source 1..20: mol_type = other RNA
aacgtctttgaggccttcgt

SEQ ID NO: 112 - RNA, 20 nt, synthetic construct
  source 1..20: mol_type = other RNA
tcactgagaattcttctgtc

SEQ ID NO: 113 - RNA, 20 nt, synthetic construct
  source 1..20: mol_type = other RNA
tttgaggccttcgtagtaaa

SEQ ID NO: 114 - RNA, 20 nt, synthetic construct
  source 1..20: mol_type = other RNA
acagagttgaagcttccttt

SEQ ID NO: 115 - RNA, 20 nt, synthetic construct
  source 1..20: mol_type = other RNA
attgaagcctacggtagaaa

SEQ ID NO: 116 - RNA, 20 nt, synthetic construct
  source 1..20: mol_type = other RNA
ttactacgaaggcctcaaag

SEQ ID NO: 117 - RNA, 20 nt, synthetic construct
  source 1..20: mol_type = other RNA
ttccaacgaagacttcaaag

SEQ ID NO: 118 - RNA, 20 nt, synthetic construct
  source 1..20: mol_type = other RNA
cagtttgtaatgtctgcagc

SEQ ID NO: 119 - RNA, 20 nt, synthetic construct
  source 1..20: mol_type = other RNA
gacctctttgaagtcttcgt

SEQ ID NO: 120 - RNA, 20 nt, synthetic construct
  source 1..20: mol_type = other RNA
gagttgaagcttcctttagg

SEQ ID NO: 121 - RNA, 20 nt, synthetic construct
  source 1..20: mol_type = other RNA
aatctgcactgtctaaagga

SEQ ID NO: 122 - RNA, 20 nt, synthetic construct
  source 1..20: mol_type = other RNA
tcagtaacttctttgggttg

SEQ ID NO: 123 - RNA, 20 nt, synthetic construct
  source 1..20: mol_type = other RNA
cttgtctgtggaatttgcaa

SEQ ID NO: 124 - RNA, 20 nt, synthetic construct
  source 1..20: mol_type = other RNA
acttgtctgtggaatttgca

SEQ ID NO: 125 - RNA, 20 nt, synthetic construct
  source 1..20: mol_type = other RNA
ttgtctgtggaatttgcaag

SEQ ID NO: 126 - RNA, 20 nt, synthetic construct
  source 1..20: mol_type = other RNA
ttcaagcgctttgaagtgaa

SEQ ID NO: 127 - RNA, 20 nt, synthetic construct
  source 1..20: mol_type = other RNA
aaactgctgtgtcaaaagga

SEQ ID NO: 128 - RNA, 20 nt, synthetic construct
  source 1..20: mol_type = other RNA
gcagaaagagtgtttcaaac

SEQ ID NO: 129 - RNA, 20 nt, synthetic construct
  source 1..20: mol_type = other RNA
tgatgtttgcattcagctca

SEQ ID NO: 130 - RNA, 20 nt, synthetic construct
  source 1..20: mol_type = other RNA
agaatctgcaggtggatatg

SEQ ID NO: 131 - RNA, 20 nt, synthetic construct
  source 1..20: mol_type = other RNA
ctttctctagtatctggaag

SEQ ID NO: 132 - RNA, 20 nt, synthetic construct
  source 1..20: mol_type = other RNA
tcagaaagtggatattcgga

SEQ ID NO: 133 - RNA, 20 nt, synthetic construct
  source 1..20: mol_type = other RNA
cattctgtagtatctggaag

SEQ ID NO: 134 - RNA, 20 nt, synthetic construct
  source 1..20: mol_type = other RNA
ttcgagcgctttgagtccta

SEQ ID NO: 135 - RNA, 20 nt, synthetic construct
  source 1..20: mol_type = other RNA
gatggctctgaggatttcgt

SEQ ID NO: 136 - RNA, 20 nt, synthetic construct
  source 1..20: mol_type = other RNA
tctgaggatttcgttggaag

SEQ ID NO: 137 - RNA, 20 nt, synthetic construct
  source 1..20: mol_type = other RNA
tggatattcggatggctctg

SEQ ID NO: 138 - RNA, 20 nt, synthetic construct
  source 1..20: mol_type = other RNA
gatagctctgaagatttcgt

SEQ ID NO: 139 - RNA, 20 nt, synthetic construct
  source 1..20: mol_type = other RNA
caactaacagagttgaacct

SEQ ID NO: 140 - RNA, 20 nt, synthetic construct
  source 1..20: mol_type = other RNA
gatagcttagagggattcgt

SEQ ID NO: 141 - RNA, 20 nt, synthetic construct
  source 1..20: mol_type = other RNA
tttcaggcctatggagagaa

SEQ ID NO: 142 - RNA, 20 nt, synthetic construct
  source 1..20: mol_type = other RNA
aggttcagctctgtgaattg

SEQ ID NO: 143 - RNA, 20 nt, synthetic construct
  source 1..20: mol_type = other RNA
ttagagggattcgttggaaa

SEQ ID NO: 144 - RNA, 20 nt, synthetic construct
  source 1..20: mol_type = other RNA
tagagggattcgttggaaag

SEQ ID NO: 145 - RNA, 20 nt, synthetic construct
  source 1..20: mol_type = other RNA
cacagagctgaacctttgtt

SEQ ID NO: 146 - RNA, 20 nt, synthetic construct
  source 1..20: mol_type = other RNA
tttctgagaatgctcctgtc

SEQ ID NO: 147 - RNA, 20 nt, synthetic construct
  source 1..20: mol_type = other RNA
ggtatttcctttctctccat

SEQ ID NO: 148 - RNA, 20 nt, synthetic construct
  source 1..20: mol_type = other RNA
ctttgaggcctatggttaaa

SEQ ID NO: 149 - RNA, 20 nt, synthetic construct
  source 1..20: mol_type = other RNA
aacagagttgaaccattgct

SEQ ID NO: 150 - RNA, 20 nt, synthetic construct
  source 1..20: mol_type = other RNA
tggatatttcgagctctttg

SEQ ID NO: 151 - RNA, 20 nt, synthetic construct
  source 1..20: mol_type = other RNA
ttcgagctctttgaggccta

SEQ ID NO: 152 - RNA, 20 nt, synthetic construct
  source 1..20: mol_type = other RNA
actccttttgtaggatctgc

SEQ ID NO: 153 - RNA, 20 nt, synthetic construct
  source 1..20: mol_type = other RNA
ccttttgtaggatctgcagg

SEQ ID NO: 154 - RNA, 20 nt, synthetic construct
  source 1..20: mol_type = other RNA
ccacctgcagatcctacaaa

SEQ ID NO: 155 - RNA, 20 nt, synthetic construct
  source 1..20: mol_type = other RNA
gatatttcctttctccccgt

SEQ ID NO: 156 - RNA, 20 nt, synthetic construct
  source 1..20: mol_type = other RNA
tttcaggcctacggggagaa

SEQ ID NO: 157 - RNA, 20 nt, synthetic construct
  source 1..20: mol_type = other RNA
tcaagcgctttcaggcctac

SEQ ID NO: 158 - RNA, 20 nt, synthetic construct
  source 1..20: mol_type = other RNA
caagcgctttcaggcctacg

SEQ ID NO: 159 - RNA, 20 nt, synthetic construct
  source 1..20: mol_type = other RNA
ctttcggcactacctggaag

SEQ ID NO: 160 - RNA, 20 nt, synthetic construct
  source 1..20: mol_type = other RNA
aacactctttcggcactacc

SEQ ID NO: 161 - RNA, 20 nt, synthetic construct
  source 1..20: mol_type = other RNA
caacttgcagattctactca

SEQ ID NO: 162 - RNA, 20 nt, synthetic construct
  source 1..20: mol_type = other RNA
ttctaccattgacctcaacg

SEQ ID NO: 163 - RNA, 20 nt, synthetic construct
  source 1..20: mol_type = other RNA
ttcagccgcgttgaggtcaa

SEQ ID NO: 164 - RNA, 20 nt, synthetic construct
  source 1..20: mol_type = other RNA
gctgcattccacacacacgg

SEQ ID NO: 165 - RNA, 20 nt, synthetic construct
  source 1..20: mol_type = other RNA
tttccaaagaatgcctccaa

SEQ ID NO: 166 - RNA, 20 nt, synthetic construct
  source 1..20: mol_type = other RNA
cttggaaatcctacaagaac

SEQ ID NO: 167 - RNA, 20 nt, synthetic construct
  source 1..20: mol_type = other RNA
gttcttgtaggatttccaag

SEQ ID NO: 168 - RNA, 20 nt, synthetic construct
  source 1..20: mol_type = other RNA
tctctgagaattcttctgtc

SEQ ID NO: 169 - RNA, 20 nt, synthetic construct
  source 1..20: mol_type = other RNA
tggaggcattctttggaaaa

SEQ ID NO: 170 - RNA, 20 nt, synthetic construct
  source 1..20: mol_type = other RNA
ttggaggcattctttggaaa

SEQ ID NO: 171 - RNA, 20 nt, synthetic construct
  source 1..20: mol_type = other RNA
ttttccaaagaatgcctcca

SEQ ID NO: 172 - RNA, 20 nt, synthetic construct
  source 1..20: mol_type = other RNA
tgaacgcacacatcacaatg

SEQ ID NO: 173 - RNA, 20 nt, synthetic construct
  source 1..20: mol_type = other RNA
gagcccttggaggcattctt

SEQ ID NO: 174 - RNA, 20 nt, synthetic construct
  source 1..20: mol_type = other RNA
tgtgaattcaactcacagtg

SEQ ID NO: 175 - RNA, 20 nt, synthetic construct
  source 1..20: mol_type = other RNA
tggatattttgagcccttgg

SEQ ID NO: 176 - RNA, 20 nt, synthetic construct
  source 1..20: mol_type = other RNA
atgcgctataaatatcccct

SEQ ID NO: 177 - RNA, 20 nt, synthetic construct
  source 1..20: mol_type = other RNA
acttgcagctactacaagaa

SEQ ID NO: 178 - RNA, 20 nt, synthetic construct
  source 1..20: mol_type = other RNA
tgtacattcaactcacagag

SEQ ID NO: 179 - RNA, 20 nt, synthetic construct
  source 1..20: mol_type = other RNA
cacaaagtcgtttctgagat

SEQ ID NO: 180 - RNA, 20 nt, synthetic construct
  source 1..20: mol_type = other RNA
cttcttgtagtagctgcaag

SEQ ID NO: 181 - RNA, 20 nt, synthetic construct
  source 1..20: mol_type = other RNA
ttccaaagaaggcctccaat

SEQ ID NO: 182 - RNA, 20 nt, synthetic construct
  source 1..20: mol_type = other RNA
gacctattggaggccttctt

SEQ ID NO: 183 - RNA, 20 nt, synthetic construct
  source 1..20: mol_type = other RNA
tggatatttggacctattgg

SEQ ID NO: 184 - RNA, 20 nt, synthetic construct
  source 1..20: mol_type = other RNA
tgaaaacccgtttccaacga

SEQ ID NO: 185 - RNA, 20 nt, synthetic construct
  source 1..20: mol_type = other RNA
cacttgcagctactacaaga

SEQ ID NO: 186 - RNA, 20 nt, synthetic construct
  source 1..20: mol_type = other RNA
tctgcattcaactcaccgag

SEQ ID NO: 187 - RNA, 20 nt, synthetic construct
  source 1..20: mol_type = other RNA
tttgtatttggacctccttg

SEQ ID NO: 188 - RNA, 20 nt, synthetic construct
  source 1..20: mol_type = other RNA
gaaatggtccaccgtgtgtg

SEQ ID NO: 189 - RNA, 20 nt, synthetic construct
  source 1..20: mol_type = other RNA
ttgcagatccttcagaaaga

SEQ ID NO: 190 - RNA, 20 nt, synthetic construct
  source 1..20: mol_type = other RNA
actctgagaattcttctgtc

SEQ ID NO: 191 - RNA, 20 nt, synthetic construct
  source 1..20: mol_type = other RNA
cttgaagcgtatggtagaaa

SEQ ID NO: 192 - RNA, 20 nt, synthetic construct
  source 1..20: mol_type = other RNA
cttgcagatccttcagaaag

SEQ ID NO: 193 - RNA, 20 nt, synthetic construct
  source 1..20: mol_type = other RNA
ctttctgaaggatctgcaag

SEQ ID NO: 194 - RNA, 20 nt, synthetic construct
  source 1..20: mol_type = other RNA
tttcaggcctatggtagaaa

SEQ ID NO: 195 - RNA, 20 nt, synthetic construct
  source 1..20: mol_type = other RNA
aaactgctgtatccaaagga

SEQ ID NO: 196 - RNA, 20 nt, synthetic construct
  source 1..20: mol_type = other RNA
gaactcctttgggtcttcgt

SEQ ID NO: 197 - RNA, 20 nt, synthetic construct
  source 1..20: mol_type = other RNA
aggcgctctaaatatccgct

SEQ ID NO: 198 - RNA, 20 nt, synthetic construct
  source 1..20: mol_type = other RNA
attgaagcccacagtagaaa

SEQ ID NO: 199 - RNA, 20 nt, synthetic construct
  source 1..20: mol_type = other RNA
tgtgcattcaactcagcgag

SEQ ID NO: 200 - RNA, 20 nt, synthetic construct
  source 1..20: mol_type = other RNA
ttcaggcctatggtagaaaa

SEQ ID NO: 201 - RNA, 20 nt, synthetic construct
  source 1..20: mol_type = other RNA
tgtatactaagagcgctttg

SEQ ID NO: 202 - RNA, 20 nt, synthetic construct
  source 1..20: mol_type = other RNA
ctttgggtcttcgttggaaa

SEQ ID NO: 203 - RNA, 20 nt, synthetic construct
  source 1..20: mol_type = other RNA
cgtttccaacgaagacccaa

SEQ ID NO: 204 - RNA, 20 nt, synthetic construct
  source 1..20: mol_type = other RNA
cttgaagcctatgctagaaa

SEQ ID NO: 205 - RNA, 20 nt, synthetic construct
  source 1..20: mol_type = other RNA
tttgggtcttcgttggaaac

SEQ ID NO: 206 - RNA, 20 nt, synthetic construct
  source 1..20: mol_type = other RNA
tgagagcgctttcaggccta

SEQ ID NO: 207 - RNA, 20 nt, synthetic construct
  source 1..20: mol_type = other RNA
ggtacattgagagcgctttc

SEQ ID NO: 208 - RNA, 20 nt, synthetic construct
  source 1..20: mol_type = other RNA
ttccaaactgctcggtcaag

SEQ ID NO: 209 - RNA, 20 nt, synthetic construct
  source 1..20: mol_type = other RNA
ttcctcttgaccgagcagtt

SEQ ID NO: 210 - RNA, 20 nt, synthetic construct
  source 1..20: mol_type = other RNA
atagcgcattgagcctacgg

SEQ ID NO: 211 - RNA, 20 nt, synthetic construct
  source 1..20: mol_type = other RNA
gatgtttctttttccgccgt

SEQ ID NO: 212 - RNA, 20 nt, synthetic construct
  source 1..20: mol_type = other RNA
gtcttcacataaaaggcaga

SEQ ID NO: 213 - RNA, 20 nt, synthetic construct
  source 1..20: mol_type = other RNA
tggataattggacctcctag

SEQ ID NO: 214 - RNA, 20 nt, synthetic construct
  source 1..20: mol_type = other RNA
tgtgcattcgacgcacagaa

SEQ ID NO: 215 - RNA, 20 nt, synthetic construct
  source 1..20: mol_type = other RNA
ttggaacgccttgaagcgta

SEQ ID NO: 216 - RNA, 20 nt, synthetic construct
  source 1..20: mol_type = other RNA
aggcgttccaaatatccgct

SEQ ID NO: 217 - RNA, 20 nt, synthetic construct
  source 1..20: mol_type = other RNA
aaactgctctgtgaaaaggg

SEQ ID NO: 218 - RNA, 20 nt, synthetic construct
  source 1..20: mol_type = other RNA
ttccaaactgctctctcaag

SEQ ID NO: 219 - RNA, 20 nt, synthetic construct
  source 1..20: mol_type = other RNA
ttcctcttgagagagcagtt

SEQ ID NO: 220 - RNA, 20 nt, synthetic construct
  source 1..20: mol_type = other RNA
tggaggccttctttggaaat

SEQ ID NO: 221 - RNA, 20 nt, synthetic construct
  source 1..20: mol_type = other RNA
gggatatttggacttctttg

SEQ ID NO: 222 - RNA, 20 nt, synthetic construct
  source 1..20: mol_type = other RNA
tctcagaaactactgtgtga

SEQ ID NO: 223 - RNA, 20 nt, synthetic construct
  source 1..20: mol_type = other RNA
gaaatgttccaccgtgtgtg

SEQ ID NO: 224 - RNA, 20 nt, synthetic construct
  source 1..20: mol_type = other RNA
gtggttgtagtatttccaag

SEQ ID NO: 225 - RNA, 20 nt, synthetic construct
  source 1..20: mol_type = other RNA
tggataattggaccgccttg

SEQ ID NO: 226 - RNA, 20 nt, synthetic construct
  source 1..20: mol_type = other RNA
gcacacaaccaaagaagttt

SEQ ID NO: 227 - RNA, 20 nt, synthetic construct
  source 1..20: mol_type = other RNA
cacagagtggaaccttcctt

SEQ ID NO: 228 - RNA, 20 nt, synthetic construct
  source 1..20: mol_type = other RNA
ttcctcttggtagagcagtt

SEQ ID NO: 229 - RNA, 20 nt, synthetic construct
  source 1..20: mol_type = other RNA
acagagtgcaacattcctct

SEQ ID NO: 230 - RNA, 20 nt, synthetic construct
  source 1..20: mol_type = other RNA
cccattgcagattctacaaa

SEQ ID NO: 231 - RNA, 20 nt, synthetic construct
  source 1..20: mol_type = other RNA
ttcccaactgctctaccaag

SEQ ID NO: 232 - RNA, 20 nt, synthetic construct
  source 1..20: mol_type = other RNA
attcctcttggtagagcagt

SEQ ID NO: 233 - RNA, 20 nt, synthetic construct
  source 1..20: mol_type = other RNA
aaggcgctctaatatccgct

SEQ ID NO: 234 - RNA, 20 nt, synthetic construct
  source 1..20: mol_type = other RNA
gcttctgtcttggttttatg

SEQ ID NO: 235 - RNA, 20 nt, synthetic construct
  source 1..20: mol_type = other RNA
gcggatattagagcgccttg

SEQ ID NO: 236 - RNA, 20 nt, synthetic construct
  source 1..20: mol_type = other RNA
gcttggaaatactacaacca

SEQ ID NO: 237 - RNA, 20 nt, synthetic construct
  source 1..20: mol_type = other RNA
tagagcagtttgaaacgccg

SEQ ID NO: 238 - RNA, 20 nt, synthetic construct
  source 1..20: mol_type = other RNA
gttgaatgcagacatcacag

SEQ ID NO: 239 - RNA, 20 nt, synthetic construct
  source 1..20: mol_type = other RNA
aatctgctctctctaaagca

SEQ ID NO: 240 - RNA, 20 nt, synthetic construct
  source 1..20: mol_type = other RNA
ttgcaacgaaggcctcaaag

SEQ ID NO: 241 - RNA, 20 nt, synthetic construct
  source 1..20: mol_type = other RNA
tttgaggccttcgttgcaaa

SEQ ID NO: 242 - RNA, 20 nt, synthetic construct
  source 1..20: mol_type = other RNA
gaccgcattgaggccttcgt

SEQ ID NO: 243 - RNA, 20 nt, synthetic construct
  source 1..20: mol_type = other RNA
tgaggccttcgttgcaaacg

SEQ ID NO: 244 - RNA, 20 nt, synthetic construct
  source 1..20: mol_type = other RNA
agaaaccccgtttgcaacga

SEQ ID NO: 245 - RNA, 20 nt, synthetic construct
  source 1..20: mol_type = other RNA
ctttttgtggagtttgcaag

SEQ ID NO: 246 - RNA, 20 nt, synthetic construct
  source 1..20: mol_type = other RNA
gatttgaaacactcttgctg

SEQ ID NO: 247 - RNA, 20 nt, synthetic construct
  source 1..20: mol_type = other RNA
cttgctgtggcattttcagg

SEQ ID NO: 248 - RNA, 20 nt, synthetic construct
  source 1..20: mol_type = other RNA
ttcaaatctgctctgtgcaa

SEQ ID NO: 249 - RNA, 20 nt, synthetic construct
  source 1..20: mol_type = other RNA
tcaaatctgctctgtgcaaa

SEQ ID NO: 250 - RNA, 20 nt, synthetic construct
  source 1..20: mol_type = other RNA
tggatacatggacctgtttg

SEQ ID NO: 251 - RNA, 20 nt, synthetic construct
  source 1..20: mol_type = other RNA
aaagtctgcaagtggataca

SEQ ID NO: 252 - RNA, 20 nt, synthetic construct
  source 1..20: mol_type = other RNA
cttcttttggaatctgcaag

SEQ ID NO: 253 - RNA, 20 nt, synthetic construct
  source 1..20: mol_type = other RNA
aaccttccgtttcatagagc

SEQ ID NO: 254 - RNA, 20 nt, synthetic construct
  source 1..20: mol_type = other RNA
ttccgtttcatagagcaggt

SEQ ID NO: 255 - RNA, 20 nt, synthetic construct
  source 1..20: mol_type = other RNA
cttgattgcaaacatcacga

SEQ ID NO: 256 - RNA, 20 nt, synthetic construct
  source 1..20: mol_type = other RNA
gatagctgtgaggatttcgt

SEQ ID NO: 257 - RNA, 20 nt, synthetic construct
  source 1..20: mol_type = other RNA
tgagaatcacgtttgtgatg

SEQ ID NO: 258 - RNA, 20 nt, synthetic construct
  source 1..20: mol_type = other RNA
gagaacacacatcacaatca

SEQ ID NO: 259 - RNA, 20 nt, synthetic construct
  source 1..20: mol_type = other RNA
gaaaggttcaactctgttag

SEQ ID NO: 260 - RNA, 20 nt, synthetic construct
  source 1..20: mol_type = other RNA
caactgtcagaattgaacct

SEQ ID NO: 261 - RNA, 20 nt, synthetic construct
  source 1..20: mol_type = other RNA
ttggagcgctttctgaacta

SEQ ID NO: 262 - RNA, 20 nt, synthetic construct
  source 1..20: mol_type = other RNA
ctgcacaactgctctatgtg

SEQ ID NO: 263 - RNA, 20 nt, synthetic construct
  source 1..20: mol_type = other RNA
cttctcgtactatctggcag

SEQ ID NO: 264 - RNA, 20 nt, synthetic construct
  source 1..20: mol_type = other RNA
gcacaactgctctatgtgag

SEQ ID NO: 265 - RNA, 20 nt, synthetic construct
  source 1..20: mol_type = other RNA
tgcacaactgctctatgtga

SEQ ID NO: 266 - RNA, 20 nt, synthetic construct
  source 1..20: mol_type = other RNA
tttccattcaagtcacagag

SEQ ID NO: 267 - RNA, 20 nt, synthetic construct
  source 1..20: mol_type = other RNA
agattctgcatgcggatatt

SEQ ID NO: 268 - RNA, 20 nt, synthetic construct
  source 1..20: mol_type = other RNA
gaagtactgcatgaaacgaa

SEQ ID NO: 269 - RNA, 20 nt, synthetic construct
  source 1..20: mol_type = other RNA
gttccactctgtgacttgaa

SEQ ID NO: 270 - RNA, 20 nt, synthetic construct
  source 1..20: mol_type = other RNA
gatcgctttgaggatttcgt

SEQ ID NO: 271 - RNA, 20 nt, synthetic construct
  source 1..20: mol_type = other RNA
cggatatttggatagctttg

SEQ ID NO: 272 - RNA, 20 nt, synthetic construct
  source 1..20: mol_type = other RNA
ctttttggagtatctggaag

SEQ ID NO: 273 - RNA, 20 nt, synthetic construct
  source 1..20: mol_type = other RNA
agtggacattttgagctcct

SEQ ID NO: 274 - RNA, 20 nt, synthetic construct
  source 1..20: mol_type = other RNA
tggacattttgagctccttg

SEQ ID NO: 275 - RNA, 20 nt, synthetic construct
  source 1..20: mol_type = other RNA
ctttttcagcataggcccca

SEQ ID NO: 276 - RNA, 20 nt, synthetic construct
  source 1..20: mol_type = other RNA
cttggggcctatgctgaaaa

SEQ ID NO: 277 - RNA, 20 nt, synthetic construct
  source 1..20: mol_type = other RNA
tgatgtgtgtcctcaacaaa

SEQ ID NO: 278 - RNA, 20 nt, synthetic construct
  source 1..20: mol_type = other RNA
aacgacatagaagctatctc

SEQ ID NO: 279 - RNA, 20 nt, synthetic construct
  source 1..20: mol_type = other RNA
aggttcaagtccgtttgttg

SEQ ID NO: 280 - RNA, 20 nt, synthetic construct
  source 1..20: mol_type = other RNA
aaagtgctctgtccaaacca

SEQ ID NO: 281 - RNA, 20 nt, synthetic construct
  source 1..20: mol_type = other RNA
tgcttctgtctagtttctgt

SEQ ID NO: 282 - RNA, 20 nt, synthetic construct
  source 1..20: mol_type = other RNA
aaactgctctgtcagtacaa

SEQ ID NO: 283 - RNA, 20 nt, synthetic construct
  source 1..20: mol_type = other RNA
aggatatttggatagctgtg

SEQ ID NO: 284 - RNA, 20 nt, synthetic construct
  source 1..20: mol_type = other RNA
ttcccattcatagagcaggt

SEQ ID NO: 285 - RNA, 20 nt, synthetic construct
  source 1..20: mol_type = other RNA
ggatattcggatagctttga

SEQ ID NO: 286 - RNA, 20 nt, synthetic construct
  source 1..20: mol_type = other RNA
aacagagttgaacctttgtg

SEQ ID NO: 287 - RNA, 20 nt, synthetic construct
  source 1..20: mol_type = other RNA
acctagagagaagcattctc

SEQ ID NO: 288 - RNA, 20 nt, synthetic construct
  source 1..20: mol_type = other RNA
gatagctaggaagatttcct

SEQ ID NO: 289 - RNA, 20 nt, synthetic construct
  source 1..20: mol_type = other RNA
gacactctttctgcactacc

SEQ ID NO: 290 - RNA, 20 nt, synthetic construct
  source 1..20: mol_type = other RNA
ctgttagttgagaacacaca

SEQ ID NO: 291 - RNA, 20 nt, synthetic construct
  source 1..20: mol_type = other RNA
ttgaggatttcgttggacac

SEQ ID NO: 292 - RNA, 20 nt, synthetic construct
  source 1..20: mol_type = other RNA
gtgtccaacgaaatcctcaa

SEQ ID NO: 293 - RNA, 20 nt, synthetic construct
  source 1..20: mol_type = other RNA
tttgaggatttcgttggaca

SEQ ID NO: 294 - RNA, 20 nt, synthetic construct
  source 1..20: mol_type = other RNA
atctagggagaagcattctc

SEQ ID NO: 295 - RNA, 20 nt, synthetic construct
  source 1..20: mol_type = other RNA
gatagctttgcggatttcgt

SEQ ID NO: 296 - RNA, 20 nt, synthetic construct
  source 1..20: mol_type = other RNA
tttcaggcctgtggtgagaa

SEQ ID NO: 297 - RNA, 20 nt, synthetic construct
  source 1..20: mol_type = other RNA
tttaagcgctttcaggcctg

SEQ ID NO: 298 - RNA, 20 nt, synthetic construct
  source 1..20: mol_type = other RNA
tgttcagtcctgtgacttga

SEQ ID NO: 299 - RNA, 20 nt, synthetic construct
  source 1..20: mol_type = other RNA
gatgtttgccttcaagtcac

SEQ ID NO: 300 - RNA, 20 nt, synthetic construct
  source 1..20: mol_type = other RNA
aagtccaaaaaagcacttgc

SEQ ID NO: 301 - RNA, 20 nt, synthetic construct
  source 1..20: mol_type = other RNA
tttgacgccaatcttagaca

SEQ ID NO: 302 - RNA, 20 nt, synthetic construct
  source 1..20: mol_type = other RNA
tgattagttagacccctttg

SEQ ID NO: 303 - RNA, 20 nt, synthetic construct
  source 1..20: mol_type = other RNA
actctgctctctctaaagaa

SEQ ID NO: 304 - RNA, 20 nt, synthetic construct
  source 1..20: mol_type = other RNA
actctttgtaagtctgcagg

SEQ ID NO: 305 - RNA, 20 nt, synthetic construct
  source 1..20: mol_type = other RNA
ctttctgtggagtttgcaag

SEQ ID NO: 306 - RNA, 20 nt, synthetic construct
  source 1..20: mol_type = other RNA
cctttctttagagggagcag

SEQ ID NO: 307 - RNA, 20 nt, synthetic construct
  source 1..20: mol_type = other RNA
cctctgctccctctaaagaa

SEQ ID NO: 308 - RNA, 20 nt, synthetic construct
  source 1..20: mol_type = other RNA
gatatacccgtttcgaagga

SEQ ID NO: 309 - RNA, 20 nt, synthetic construct
  source 1..20: mol_type = other RNA
tttgtggccttccttcgaaa

SEQ ID NO: 310 - RNA, 20 nt, synthetic construct
  source 1..20: mol_type = other RNA
tgaagatatacccgtttcga

SEQ ID NO: 311 - RNA, 20 nt, synthetic construct
  source 1..20: mol_type = other RNA
ttgtggccttccttcgaaac

SEQ ID NO: 312 - RNA, 20 nt, synthetic construct
  source 1..20: mol_type = other RNA
tgtctgtgaatgcttccgtt

SEQ ID NO: 313 - RNA, 20 nt, synthetic construct
  source 1..20: mol_type = other RNA
ttcgaaggaaggccacaaag

SEQ ID NO: 314 - RNA, 20 nt, synthetic construct
  source 1..20: mol_type = other RNA
gagttgaatgcagtcatcac

SEQ ID NO: 315 - RNA, 20 nt, synthetic construct
  source 1..20: mol_type = other RNA
gacctctgtgaagatttcgt

SEQ ID NO: 316 - RNA, 20 nt, synthetic construct
  source 1..20: mol_type = other RNA
agattttccttttccaccac

SEQ ID NO: 317 - RNA, 20 nt, synthetic construct
  source 1..20: mol_type = other RNA
tttgaggcctactgtagtaa

SEQ ID NO: 318 - RNA, 20 nt, synthetic construct
  source 1..20: mol_type = other RNA
tgactgcattcaactcacag

SEQ ID NO: 319 - RNA, 20 nt, synthetic construct
  source 1..20: mol_type = other RNA
gagctgaacattcctttaga

SEQ ID NO: 320 - RNA, 20 nt, synthetic construct
  source 1..20: mol_type = other RNA
ctttctttggattctgcaag

SEQ ID NO: 321 - RNA, 20 nt, synthetic construct
  source 1..20: mol_type = other RNA
ggattctgcaagtggatatg

SEQ ID NO: 322 - RNA, 20 nt, synthetic construct
  source 1..20: mol_type = other RNA
gaggtgaacaatcctgctga

SEQ ID NO: 323 - RNA, 20 nt, synthetic construct
  source 1..20: mol_type = other RNA
ggaaagttcaattcctgaag

SEQ ID NO: 324 - RNA, 20 nt, synthetic construct
  source 1..20: mol_type = other RNA
ttggaaactgcgccatctaa

SEQ ID NO: 325 - RNA, 20 nt, synthetic construct
  source 1..20: mol_type = other RNA
gattctacagaaagtgggtt

SEQ ID NO: 326 - RNA, 20 nt, synthetic construct
  source 1..20: mol_type = other RNA
ggattctgcaagttgatatg

SEQ ID NO: 327 - RNA, 20 nt, synthetic construct
  source 1..20: mol_type = other RNA
cagatatttggacctctttg

SEQ ID NO: 328 - RNA, 20 nt, synthetic construct
  source 1..20: mol_type = other RNA
aatctgctctttctgaagga

SEQ ID NO: 329 - RNA, 20 nt, synthetic construct
  source 1..20: mol_type = other RNA
gtctttgtaaagtctgcaag

SEQ ID NO: 330 - RNA, 20 nt, synthetic construct
  source 1..20: mol_type = other RNA
tgatgtgtgtgttcaactca

SEQ ID NO: 331 - RNA, 20 nt, synthetic construct
  source 1..20: mol_type = other RNA
ctatttgtgcagtttccagt

SEQ ID NO: 332 - RNA, 20 nt, synthetic construct
  source 1..20: mol_type = other RNA
ctttttgtggaatttgcagc

SEQ ID NO: 333 - RNA, 20 nt, synthetic construct
  source 1..20: mol_type = other RNA
ctttttgtggagtttccatg

SEQ ID NO: 334 - RNA, 20 nt, synthetic construct
  source 1..20: mol_type = other RNA
cagctgcaaattccacaaaa

SEQ ID NO: 335 - RNA, 20 nt, synthetic construct
  source 1..20: mol_type = other RNA
aagcgattgaaatctccaac

SEQ ID NO: 336 - RNA, 20 nt, synthetic construct
  source 1..20: mol_type = other RNA
aagcgattgaaatctccaca

SEQ ID NO: 337 - RNA, 20 nt, synthetic construct
  source 1..20: mol_type = other RNA
gatgtttccttttctaccgt

SEQ ID NO: 338 - RNA, 20 nt, synthetic construct
  source 1..20: mol_type = other RNA
ttcaatcgctttgagaccaa

SEQ ID NO: 339 - RNA, 20 nt, synthetic construct
  source 1..20: mol_type = other RNA
tcccagaaaaacgagacaga

SEQ ID NO: 340 - RNA, 20 nt, synthetic construct
  source 1..20: mol_type = other RNA
atggaagtggacttatcgga

SEQ ID NO: 341 - RNA, 20 nt, synthetic construct
  source 1..20: mol_type = other RNA
tttcaggcctaaggtgagaa

SEQ ID NO: 342 - RNA, 20 nt, synthetic construct
  source 1..20: mol_type = other RNA
ctcttaggcctaaggtgaaa

SEQ ID NO: 343 - RNA, 20 nt, synthetic construct
  source 1..20: mol_type = other RNA
ctgttagttgagtacacaca

SEQ ID NO: 344 - RNA, 20 nt, synthetic construct
  source 1..20: mol_type = other RNA
tggacatttcgagcactctt

SEQ ID NO: 345 - RNA, 20 nt, synthetic construct
  source 1..20: mol_type = other RNA
aggttgaactctgtgagttg

SEQ ID NO: 346 - RNA, 20 nt, synthetic construct
  source 1..20: mol_type = other RNA
ggcctcttagaggccttcgt

SEQ ID NO: 347 - RNA, 20 nt, synthetic construct
  source 1..20: mol_type = other RNA
tttgaggccaaaagcagaaa

SEQ ID NO: 348 - RNA, 20 nt, synthetic construct
  source 1..20: mol_type = other RNA
tggagacttcaagcgctttg

SEQ ID NO: 349 - RNA, 20 nt, synthetic construct
  source 1..20: mol_type = other RNA
gcagatttgagacactcttt

SEQ ID NO: 350 - RNA, 20 nt, synthetic construct
  source 1..20: mol_type = other RNA
ttcgaatctgctctgtctaa

SEQ ID NO: 351 - RNA, 20 nt, synthetic construct
  source 1..20: mol_type = other RNA
aaagaggtccgaatatccac

SEQ ID NO: 352 - RNA, 20 nt, synthetic construct
  source 1..20: mol_type = other RNA
ctgtttggaaagtctgcacg

SEQ ID NO: 353 - RNA, 20 nt, synthetic construct
  source 1..20: mol_type = other RNA
ttagatgccttcgttggaaa

SEQ ID NO: 354 - RNA, 20 nt, synthetic construct
  source 1..20: mol_type = other RNA
tagatgccttcgttggaaac

SEQ ID NO: 355 - RNA, 20 nt, synthetic construct
  source 1..20: mol_type = other RNA
gacctcttagatgccttcgt

SEQ ID NO: 356 - RNA, 20 nt, synthetic construct
  source 1..20: mol_type = other RNA
aggtccgaatatccactggc

SEQ ID NO: 357 - RNA, 20 nt, synthetic construct
  source 1..20: mol_type = other RNA
ttccaacgaaggcatctaag

SEQ ID NO: 358 - RNA, 20 nt, synthetic construct
  source 1..20: mol_type = other RNA
gatttgagacactcttttgg

SEQ ID NO: 359 - RNA, 20 nt, synthetic construct
  source 1..20: mol_type = other RNA
ttcgatgccaatggtagaaa

SEQ ID NO: 360 - RNA, 20 nt, synthetic construct
  source 1..20: mol_type = other RNA
ttcaagcgcttcgatgccaa

SEQ ID NO: 361 - RNA, 20 nt, synthetic construct
  source 1..20: mol_type = other RNA
tctttctcagaaactgctct

SEQ ID NO: 362 - RNA, 20 nt, synthetic construct
  source 1..20: mol_type = other RNA
atctttctcagaaactgctc

SEQ ID NO: 363 - RNA, 20 nt, synthetic construct
  source 1..20: mol_type = other RNA
cttgcagactttacaaacac

SEQ ID NO: 364 - RNA, 20 nt, synthetic construct
  source 1..20: mol_type = other RNA
ttccaaactgctctgtcaaa

SEQ ID NO: 365 - RNA, 20 nt, synthetic construct
  source 1..20: mol_type = other RNA
tttccaaactgctctgtcaa

SEQ ID NO: 366 - RNA, 20 nt, synthetic construct
  source 1..20: mol_type = other RNA
ttccctttgacagagcagtt

SEQ ID NO: 367 - RNA, 20 nt, synthetic construct
  source 1..20: mol_type = other RNA
gagggctttgtggtttgtgg

SEQ ID NO: 368 - RNA, 20 nt, synthetic construct
  source 1..20: mol_type = other RNA
ttcaaagcttctctctcgaa

SEQ ID NO: 369 - RNA, 20 nt, synthetic construct
  source 1..20: mol_type = other RNA
cataattcgttttccaccac

SEQ ID NO: 370 - RNA, 20 nt, synthetic construct
  source 1..20: mol_type = other RNA
gaatgcagacatcacgaaga

SEQ ID NO: 371 - RNA, 20 nt, synthetic construct
  source 1..20: mol_type = other RNA
gctattccctttactaccat

SEQ ID NO: 372 - RNA, 20 nt, synthetic construct
  source 1..20: mol_type = other RNA
ctcaaacctgctccatccaa

SEQ ID NO: 373 - RNA, 20 nt, synthetic construct
  source 1..20: mol_type = other RNA
gacctctccgaagatgtctt

SEQ ID NO: 374 - RNA, 20 nt, synthetic construct
  source 1..20: mol_type = other RNA
tctgcattcaactcacagtg

SEQ ID NO: 375 - RNA, 20 nt, synthetic construct
  source 1..20: mol_type = other RNA
ctctttctgtggcatctgca

SEQ ID NO: 376 - RNA, 20 nt, synthetic construct
  source 1..20: mol_type = other RNA
tctttctgtggcatctgcaa

SEQ ID NO: 377 - RNA, 20 nt, synthetic construct
  source 1..20: mol_type = other RNA
cccgtttccaaagacatctt

SEQ ID NO: 378 - RNA, 20 nt, synthetic construct
  source 1..20: mol_type = other RNA
tgaatgcaaacatcacgaag

SEQ ID NO: 379 - RNA, 20 nt, synthetic construct
  source 1..20: mol_type = other RNA
gcttctgttttagttctgtg

SEQ ID NO: 380 - RNA, 20 nt, synthetic construct
  source 1..20: mol_type = other RNA
tccgaagatgtctttggaaa

SEQ ID NO: 381 - RNA, 20 nt, synthetic construct
  source 1..20: mol_type = other RNA
cttgttgtggaatgtgcaag

SEQ ID NO: 382 - RNA, 20 nt, synthetic construct
  source 1..20: mol_type = other RNA
ttccaaagacatcttcggag

SEQ ID NO: 383 - RNA, 20 nt, synthetic construct
  source 1..20: mol_type = other RNA
gtttggaaacactcttgttg

SEQ ID NO: 384 - RNA, 20 nt, synthetic construct
  source 1..20: mol_type = other RNA
ttctaaactgctacatcgca

SEQ ID NO: 385 - RNA, 20 nt, synthetic construct
  source 1..20: mol_type = other RNA
tctgtgtccttcgttcgaaa

SEQ ID NO: 386 - RNA, 20 nt, synthetic construct
  source 1..20: mol_type = other RNA
ttcgaacgaaggacacagag

SEQ ID NO: 387 - RNA, 20 nt, synthetic construct
  source 1..20: mol_type = other RNA
cacagagttgaaccctccta

SEQ ID NO: 388 - RNA, 20 nt, synthetic construct
  source 1..20: mol_type = other RNA
ctgtgtccttcgttcgaaac

SEQ ID NO: 389 - RNA, 20 nt, synthetic construct
  source 1..20: mol_type = other RNA
ttcaacactgctctatccat

SEQ ID NO: 390 - RNA, 20 nt, synthetic construct
  source 1..20: mol_type = other RNA
acactgctctatccatagga

SEQ ID NO: 391 - RNA, 20 nt, synthetic construct
  source 1..20: mol_type = other RNA
aacactgctctatccatagg

SEQ ID NO: 392 - RNA, 20 nt, synthetic construct
  source 1..20: mol_type = other RNA
tagatatttggacctctctg

SEQ ID NO: 393 - RNA, 20 nt, synthetic construct
  source 1..20: mol_type = other RNA
cttttcgtagtgtctacaag

SEQ ID NO: 394 - RNA, 20 nt, synthetic construct
  source 1..20: mol_type = other RNA
tttgaggagtaccgtagtaa

SEQ ID NO: 395 - RNA, 20 nt, synthetic construct
  source 1..20: mol_type = other RNA
tgaaaggaaagttcaactcg

SEQ ID NO: 396 - RNA, 20 nt, synthetic construct
  source 1..20: mol_type = other RNA
gacctctgtgaggaattcgt

SEQ ID NO: 397 - RNA, 20 nt, synthetic construct
  source 1..20: mol_type = other RNA
ggaaacgggagaatcttcac

SEQ ID NO: 398 - RNA, 20 nt, synthetic construct
  source 1..20: mol_type = other RNA
ttccaacgaattcctcacag

SEQ ID NO: 399 - RNA, 20 nt, synthetic construct
  source 1..20: mol_type = other RNA
gtgaggaattcgttggaaac

SEQ ID NO: 400 - RNA, 20 nt, synthetic construct
  source 1..20: mol_type = other RNA
tgtgaggaattcgttggaaa

SEQ ID NO: 401 - RNA, 20 nt, synthetic construct
  source 1..20: mol_type = other RNA
tgcatatttggacctctgtg

SEQ ID NO: 402 - RNA, 20 nt, synthetic construct
  source 1..20: mol_type = other RNA
tggatatttggtcctctctg

SEQ ID NO: 403 - RNA, 20 nt, synthetic construct
  source 1..20: mol_type = other RNA
tcatcacagagaagcttctg

SEQ ID NO: 404 - RNA, 20 nt, synthetic construct
  source 1..20: mol_type = other RNA
ggggataattgcactctttg

SEQ ID NO: 405 - RNA, 20 nt, synthetic construct
  source 1..20: mol_type = other RNA
gtttccaatcactctttctg

SEQ ID NO: 406 - RNA, 20 nt, synthetic construct
  source 1..20: mol_type = other RNA
gattccacagaaagagtgat

SEQ ID NO: 407 - RNA, 20 nt, synthetic construct
  source 1..20: mol_type = other RNA
tggatatttaggcctctctg

SEQ ID NO: 408 - RNA, 20 nt, synthetic construct
  source 1..20: mol_type = other RNA
tctgaggaattcgttggaaa

SEQ ID NO: 409 - RNA, 20 nt, synthetic construct
  source 1..20: mol_type = other RNA
ttccaacgaattcctcagag

SEQ ID NO: 410 - RNA, 20 nt, synthetic construct
  source 1..20: mol_type = other RNA
tttgaggcctaccgtagtaa

SEQ ID NO: 411 - RNA, 20 nt, synthetic construct
  source 1..20: mol_type = other RNA
gtcctctctgagcatttcgt

SEQ ID NO: 412 - RNA, 20 nt, synthetic construct
  source 1..20: mol_type = other RNA
tttcaaacctgctctaccaa

SEQ ID NO: 413 - RNA, 20 nt, synthetic construct
  source 1..20: mol_type = other RNA
ttcaaacctgctctaccaaa

SEQ ID NO: 414 - RNA, 20 nt, synthetic construct
  source 1..20: mol_type = other RNA
aaactgctccttcaaaacgg

SEQ ID NO: 415 - RNA, 20 nt, synthetic construct
  source 1..20: mol_type = other RNA
gctagttttgaggatttcgt

SEQ ID NO: 416 - RNA, 20 nt, synthetic construct
  source 1..20: mol_type = other RNA
ttccctatcatagagcaggt

SEQ ID NO: 417 - RNA, 20 nt, synthetic construct
  source 1..20: mol_type = other RNA
attccaacctgctctatgat

SEQ ID NO: 418 - RNA, 20 nt, synthetic construct
  source 1..20: mol_type = other RNA
tttcaggcctatgttggaaa

SEQ ID NO: 419 - RNA, 20 nt, synthetic construct
  source 1..20: mol_type = other RNA
tgcttctgcctagttgttac

SEQ ID NO: 420 - RNA, 20 nt, synthetic construct
  source 1..20: mol_type = other RNA
tttcaggcctacgttggaaa

SEQ ID NO: 421 - RNA, 20 nt, synthetic construct
  source 1..20: mol_type = other RNA
aggttctactcctttagttg

SEQ ID NO: 422 - RNA, 20 nt, synthetic construct
  source 1..20: mol_type = other RNA
tttgaggatttcgtgggaaa

SEQ ID NO: 423 - RNA, 20 nt, synthetic construct
  source 1..20: mol_type = other RNA
gcatagctttgaggatttcg

SEQ ID NO: 424 - RNA, 20 nt, synthetic construct
  source 1..20: mol_type = other RNA
gagcgctttcaggcctacgt

SEQ ID NO: 425 - RNA, 20 nt, synthetic construct
  source 1..20: mol_type = other RNA
cctagccttgaggatttcgt

SEQ ID NO: 426 - RNA, 20 nt, synthetic construct
  source 1..20: mol_type = other RNA
ccaacgaaatcctcaaggct

SEQ ID NO: 427 - RNA, 20 nt, synthetic construct
  source 1..20: mol_type = other RNA
tttcctttttcaccttaggc

SEQ ID NO: 428 - RNA, 20 nt, synthetic construct
  source 1..20: mol_type = other RNA
gctagctttggggatttcgc

SEQ ID NO: 429 - RNA, 20 nt, synthetic construct
  source 1..20: mol_type = other RNA
tttcagggctaaggtgaaaa

SEQ ID NO: 430 - RNA, 20 nt, synthetic construct
  source 1..20: mol_type = other RNA
agtggatatttggctagctt

SEQ ID NO: 431 - RNA, 20 nt, synthetic construct
  source 1..20: mol_type = other RNA
tttggggatttcgctggaag

SEQ ID NO: 432 - RNA, 20 nt, synthetic construct
  source 1..20: mol_type = other RNA
ccagttccagatactacaaa

SEQ ID NO: 433 - RNA, 20 nt, synthetic construct
  source 1..20: mol_type = other RNA
ccttttgtagtatctggaac

SEQ ID NO: 434 - RNA, 20 nt, synthetic construct
  source 1..20: mol_type = other RNA
ggaatctgcaagtggctatt

SEQ ID NO: 435 - RNA, 20 nt, synthetic construct
  source 1..20: mol_type = other RNA
tggctatttggctagatttg

SEQ ID NO: 436 - RNA, 20 nt, synthetic construct
  source 1..20: mol_type = other RNA
tttcaggcccatgttggaaa

SEQ ID NO: 437 - RNA, 20 nt, synthetic construct
  source 1..20: mol_type = other RNA
ctttcaggcccatgttggaa

SEQ ID NO: 438 - RNA, 20 nt, synthetic construct
  source 1..20: mol_type = other RNA
agtatatttgcctagccttg

SEQ ID NO: 439 - RNA, 20 nt, synthetic construct
  source 1..20: mol_type = other RNA
ttggagcgatttcagggcta

SEQ ID NO: 440 - RNA, 20 nt, synthetic construct
  source 1..20: mol_type = other RNA
ggacttttggagcgatttca

SEQ ID NO: 441 - RNA, 20 nt, synthetic construct
  source 1..20: mol_type = other RNA
ctttcaggcctattttggaa

SEQ ID NO: 442 - RNA, 20 nt, synthetic construct
  source 1..20: mol_type = other RNA
ttaccggcctaaggtgaaaa

SEQ ID NO: 443 - RNA, 20 nt, synthetic construct
  source 1..20: mol_type = other RNA
tggacatttggagcacttac

SEQ ID NO: 444 - RNA, 20 nt, synthetic construct
  source 1..20: mol_type = other RNA
ctttcaggcctatgttggaa

SEQ ID NO: 445 - RNA, 20 nt, synthetic construct
  source 1..20: mol_type = other RNA
ttcaggactgctctatgaaa

SEQ ID NO: 446 - RNA, 20 nt, synthetic construct
  source 1..20: mol_type = other RNA
tttcaggactgctctatgaa

SEQ ID NO: 447 - RNA, 20 nt, synthetic construct
  source 1..20: mol_type = other RNA
tggatatttggctagtttgg

SEQ ID NO: 448 - RNA, 20 nt, synthetic construct
  source 1..20: mol_type = other RNA
gctagtttggaggatttcgt

SEQ ID NO: 449 - RNA, 20 nt, synthetic construct
  source 1..20: mol_type = other RNA
ggacttttggagcgctttca

SEQ ID NO: 450 - RNA, 20 nt, synthetic construct
  source 1..20: mol_type = other RNA
cttgaggctttcgttggaaa

SEQ ID NO: 451 - RNA, 20 nt, synthetic construct
  source 1..20: mol_type = other RNA
tggatattcagacatccttg

SEQ ID NO: 452 - RNA, 20 nt, synthetic construct
  source 1..20: mol_type = other RNA
cgtttccaacgaaagcctca

SEQ ID NO: 453 - RNA, 20 nt, synthetic construct
  source 1..20: mol_type = other RNA
ttcagccgctttgagttcaa

SEQ ID NO: 454 - RNA, 20 nt, synthetic construct
  source 1..20: mol_type = other RNA
gacatctttgaggctttcgt

SEQ ID NO: 455 - RNA, 20 nt, synthetic construct
  source 1..20: mol_type = other RNA
tggatattagggcagctttg

SEQ ID NO: 456 - RNA, 20 nt, synthetic construct
  source 1..20: mol_type = other RNA
gttttcgtggaatctgcaag

SEQ ID NO: 457 - RNA, 20 nt, synthetic construct
  source 1..20: mol_type = other RNA
aggttcaacactgtcagttg

SEQ ID NO: 458 - RNA, 20 nt, synthetic construct
  source 1..20: mol_type = other RNA
tttcaggtctacggtgaaaa

SEQ ID NO: 459 - RNA, 20 nt, synthetic construct
  source 1..20: mol_type = other RNA
ttcaaacctgctctctcaaa

SEQ ID NO: 460 - RNA, 20 nt, synthetic construct
  source 1..20: mol_type = other RNA
tttcaaacctgctctctcaa

SEQ ID NO: 461 - RNA, 20 nt, synthetic construct
  source 1..20: mol_type = other RNA
tttcaggacgacggtgaaaa

SEQ ID NO: 462 - RNA, 20 nt, synthetic construct
  source 1..20: mol_type = other RNA
agcattctcagaaacttcgt

SEQ ID NO: 463 - RNA, 20 nt, synthetic construct
  source 1..20: mol_type = other RNA
tgggtattaggccagcttgg

SEQ ID NO: 464 - RNA, 20 nt, synthetic construct
  source 1..20: mol_type = other RNA
aagtgggtattaggccagct

SEQ ID NO: 465 - RNA, 20 nt, synthetic construct
  source 1..20: mol_type = other RNA
ctttctgcattccctggaag

SEQ ID NO: 466 - RNA, 20 nt, synthetic construct
  source 1..20: mol_type = other RNA
gatttcgttgcaaacgggaa

SEQ ID NO: 467 - RNA, 20 nt, synthetic construct
  source 1..20: mol_type = other RNA
aagctgctctttgcaaagaa

SEQ ID NO: 468 - RNA, 20 nt, synthetic construct
  source 1..20: mol_type = other RNA
ttcccttttatagagcaggt

SEQ ID NO: 469 - RNA, 20 nt, synthetic construct
  source 1..20: mol_type = other RNA
aagtggatatttggctagct

SEQ ID NO: 470 - RNA, 20 nt, synthetic construct
  source 1..20: mol_type = other RNA
tttcaggcctaacgtgaaaa

SEQ ID NO: 471 - RNA, 20 nt, synthetic construct
  source 1..20: mol_type = other RNA
tttcaggcgtatggtgaaaa

SEQ ID NO: 472 - RNA, 20 nt, synthetic construct
  source 1..20: mol_type = other RNA
gctagctgggaggatttcgt

SEQ ID NO: 473 - RNA, 20 nt, synthetic construct
  source 1..20: mol_type = other RNA
gggaggatttcgttggaaac

SEQ ID NO: 474 - RNA, 20 nt, synthetic construct
  source 1..20: mol_type = other RNA
tgggaggatttcgttggaaa

SEQ ID NO: 475 - RNA, 20 nt, synthetic construct
  source 1..20: mol_type = other RNA
gatgtgtttgctcaactaac

SEQ ID NO: 476 - RNA, 20 nt, synthetic construct
  source 1..20: mol_type = other RNA
ggattgaaccatcgttttga

SEQ ID NO: 477 - RNA, 20 nt, synthetic construct
  source 1..20: mol_type = other RNA
gcaatttggaaacacccttt

SEQ ID NO: 478 - RNA, 20 nt, synthetic construct
  source 1..20: mol_type = other RNA
cgttctggagtatctggatg

SEQ ID NO: 479 - RNA, 20 nt, synthetic construct
  source 1..20: mol_type = other RNA
actcttgttgtggaaaatgc

SEQ ID NO: 480 - RNA, 20 nt, synthetic construct
  source 1..20: mol_type = other RNA
cttgttgtggaaaatgcagg

SEQ ID NO: 481 - RNA, 20 nt, synthetic construct
  source 1..20: mol_type = other RNA
ctagcgatttcgttggaaac

SEQ ID NO: 482 - RNA, 20 nt, synthetic construct
  source 1..20: mol_type = other RNA
gatagctctagcgatttcgt

SEQ ID NO: 483 - RNA, 20 nt, synthetic construct
  source 1..20: mol_type = other RNA
tctagcgatttcgttggaaa

SEQ ID NO: 484 - RNA, 20 nt, synthetic construct
  source 1..20: mol_type = other RNA
tgtgtactcggctaacagag

SEQ ID NO: 485 - RNA, 20 nt, synthetic construct
  source 1..20: mol_type = other RNA
cgtttcaaagagcagctttg

SEQ ID NO: 486 - RNA, 20 nt, synthetic construct
  source 1..20: mol_type = other RNA
gcaggtttgaaacgctcttt

SEQ ID NO: 487 - RNA, 20 nt, synthetic construct
  source 1..20: mol_type = other RNA
agagtgtatccaaactgctc

SEQ ID NO: 488 - RNA, 20 nt, synthetic construct
  source 1..20: mol_type = other RNA
ttccttttgccagagcagtt

SEQ ID NO: 489 - RNA, 20 nt, synthetic construct
  source 1..20: mol_type = other RNA
cgtttcagagagcagctctg

SEQ ID NO: 490 - RNA, 20 nt, synthetic construct
  source 1..20: mol_type = other RNA
ggagcgctctgaggtctacg

SEQ ID NO: 491 - RNA, 20 nt, synthetic construct
  source 1..20: mol_type = other RNA
tggagcgctctgaggtctac

SEQ ID NO: 492 - RNA, 20 nt, synthetic construct
  source 1..20: mol_type = other RNA
gtgaactcagctaacagatg

SEQ ID NO: 493 - RNA, 20 nt, synthetic construct
  source 1..20: mol_type = other RNA
aagtggacgtttcggacgtt

SEQ ID NO: 494 - RNA, 20 nt, synthetic construct
  source 1..20: mol_type = other RNA
tggacgtttcggacgtttgg

SEQ ID NO: 495 - RNA, 20 nt, synthetic construct
  source 1..20: mol_type = other RNA
ttcggacgtttggaggccca

SEQ ID NO: 496 - RNA, 20 nt, synthetic construct
  source 1..20: mol_type = other RNA
atggaagtagacgtttcgga

SEQ ID NO: 497 - RNA, 20 nt, synthetic construct
  source 1..20: mol_type = other RNA
ttggagagccttgacaccta

SEQ ID NO: 498 - RNA, 20 nt, synthetic construct
  source 1..20: mol_type = other RNA
ggaatctcagaatcttcttc

SEQ ID NO: 499 - RNA, 20 nt, synthetic construct
  source 1..20: mol_type = other RNA
aagtggatgtttggatagct

SEQ ID NO: 500 - RNA, 20 nt, synthetic construct
  source 1..20: mol_type = other RNA
tggatgtttggatagcttgg

SEQ ID NO: 501 - RNA, 20 nt, synthetic construct
  source 1..20: mol_type = other RNA
agtgagtgcatacgtcataa

SEQ ID NO: 502 - RNA, 20 nt, synthetic construct
  source 1..20: mol_type = other RNA
tttcaaagctgctctctgaa

SEQ ID NO: 503 - RNA, 20 nt, synthetic construct
  source 1..20: mol_type = other RNA
gtgaactcagctaacacacg

SEQ ID NO: 504 - RNA, 20 nt, synthetic construct
  source 1..20: mol_type = other RNA
gagctgaacattcgttatga

SEQ ID NO: 505 - RNA, 20 nt, synthetic construct
  source 1..20: mol_type = other RNA
cttgcagattccaaagaaag

SEQ ID NO: 506 - RNA, 20 nt, synthetic construct
  source 1..20: mol_type = other RNA
agtttgcttccgttcagtta

SEQ ID NO: 507 - RNA, 20 nt, synthetic construct
  source 1..20: mol_type = other RNA
gtttgcttccgttcagttat

SEQ ID NO: 508 - RNA, 20 nt, synthetic construct
  source 1..20: mol_type = other RNA
acacttttggtagaatctgc

SEQ ID NO: 509 - RNA, 20 nt, synthetic construct
  source 1..20: mol_type = other RNA
ggaatctgcaaggggatatg

SEQ ID NO: 510 - RNA, 20 nt, synthetic construct
  source 1..20: mol_type = other RNA
ctctttctttggaatctgca

SEQ ID NO: 511 - RNA, 20 nt, synthetic construct
  source 1..20: mol_type = other RNA
tctttctgtgggatccgcaa

SEQ ID NO: 512 - RNA, 20 nt, synthetic construct
  source 1..20: mol_type = other RNA
ctttctgtgggatccgcaag

SEQ ID NO: 513 - RNA, 20 nt, synthetic construct
  source 1..20: mol_type = other RNA
cacttgcagattctactaca

SEQ ID NO: 514 - RNA, 20 nt, synthetic construct
  source 1..20: mol_type = other RNA
gacctctttgaagatttcac

SEQ ID NO: 515 - RNA, 20 nt, synthetic construct
  source 1..20: mol_type = other RNA
ttcaaacgaaggctacaaag

SEQ ID NO: 516 - RNA, 20 nt, synthetic construct
  source 1..20: mol_type = other RNA
agggaaagttcaactctgtg

SEQ ID NO: 517 - RNA, 20 nt, synthetic construct
  source 1..20: mol_type = other RNA
gaacctgaactctcaaaggc

SEQ ID NO: 518 - RNA, 20 nt, synthetic construct
  source 1..20: mol_type = other RNA
ctttttcgagaatctgcaag

SEQ ID NO: 519 - RNA, 20 nt, synthetic construct
  source 1..20: mol_type = other RNA
tttcgaacctgaactctcaa

SEQ ID NO: 520 - RNA, 20 nt, synthetic construct
  source 1..20: mol_type = other RNA
catatacccgtttcgaacga

SEQ ID NO: 521 - RNA, 20 nt, synthetic construct
  source 1..20: mol_type = other RNA
gcgggcttggaggactgtgt

SEQ ID NO: 522 - RNA, 20 nt, synthetic construct
  source 1..20: mol_type = other RNA
agaatctgtaagtggatacg

SEQ ID NO: 523 - RNA, 20 nt, synthetic construct
  source 1..20: mol_type = other RNA
ttggaaactgctccatcaaa

SEQ ID NO: 524 - RNA, 20 nt, synthetic construct
  source 1..20: mol_type = other RNA
tttcaggccttttccaccac

SEQ ID NO: 525 - RNA, 20 nt, synthetic construct
  source 1..20: mol_type = other RNA
gagctgaacatgccttttga

SEQ ID NO: 526 - RNA, 20 nt, synthetic construct
  source 1..20: mol_type = other RNA
cacgttttgtagaatctgca

SEQ ID NO: 527 - RNA, 20 nt, synthetic construct
  source 1..20: mol_type = other RNA
aagtggatatttggaccact

SEQ ID NO: 528 - RNA, 20 nt, synthetic construct
  source 1..20: mol_type = other RNA
tttctgagagtgctaccgtc

SEQ ID NO: 529 - RNA, 20 nt, synthetic construct
  source 1..20: mol_type = other RNA
tggatatttggaccactggg

SEQ ID NO: 530 - RNA, 20 nt, synthetic construct
  source 1..20: mol_type = other RNA
ttcgaacgaaggccacccag

SEQ ID NO: 531 - RNA, 20 nt, synthetic construct
  source 1..20: mol_type = other RNA
gtgacgatggagtttaactc

SEQ ID NO: 532 - RNA, 20 nt, synthetic construct
  source 1..20: mol_type = other RNA
tgggtggccttcgttcgaaa

SEQ ID NO: 533 - RNA, 20 nt, synthetic construct
  source 1..20: mol_type = other RNA
gggtggccttcgttcgaaac

SEQ ID NO: 534 - RNA, 20 nt, synthetic construct
  source 1..20: mol_type = other RNA
ggatatttggacctctttga

SEQ ID NO: 535 - RNA, 20 nt, synthetic construct
  source 1..20: mol_type = other RNA
gtcaaagctgcgctatcaaa

SEQ ID NO: 536 - RNA, 20 nt, synthetic construct
  source 1..20: mol_type = other RNA
tgtcaaagctgcgctatcaa

SEQ ID NO: 537 - RNA, 20 nt, synthetic construct
  source 1..20: mol_type = other RNA
aaaactgctccatcaaaagg

SEQ ID NO: 538 - RNA, 20 nt, synthetic construct
  source 1..20: mol_type = other RNA
atgtgcaagtggctatttag

SEQ ID NO: 539 - RNA, 20 nt, synthetic construct
  source 1..20: mol_type = other RNA
tgtgcaagtggctatttagc

SEQ ID NO: 540 - RNA, 20 nt, synthetic construct
  source 1..20: mol_type = other RNA
aagtggctatttagcgggct

SEQ ID NO: 541 - RNA, 20 nt, synthetic construct
  source 1..20: mol_type = other RNA
tggctatttagcgggcttgg

SEQ ID NO: 542 - RNA, 20 nt, synthetic construct
  source 1..20: mol_type = other RNA
gagttgaacaatccttctga

SEQ ID NO: 543 - RNA, 20 nt, synthetic construct
  source 1..20: mol_type = other RNA
cagttgaaccctccttttga

SEQ ID NO: 544 - RNA, 20 nt, synthetic construct
  source 1..20: mol_type = other RNA
ttctcagaaacgactttgtg

SEQ ID NO: 545 - RNA, 20 nt, synthetic construct
  source 1..20: mol_type = other RNA
tttgaggcctgtggtagtga

SEQ ID NO: 546 - RNA, 20 nt, synthetic construct
  source 1..20: mol_type = other RNA
cactaccacaggcctcaaag

SEQ ID NO: 547 - RNA, 20 nt, synthetic construct
  source 1..20: mol_type = other RNA
tttgaggcctacggtcgtat

SEQ ID NO: 548 - RNA, 20 nt, synthetic construct
  source 1..20: mol_type = other RNA
aggcctacggtcgtatagga

SEQ ID NO: 549 - RNA, 20 nt, synthetic construct
  source 1..20: mol_type = other RNA
gttccttcctatacgaccgt

SEQ ID NO: 550 - RNA, 20 nt, synthetic construct
  source 1..20: mol_type = other RNA
gttctttccttcactaccac

SEQ ID NO: 551 - RNA, 20 nt, synthetic construct
  source 1..20: mol_type = other RNA
cagaaactactttgtgagga

SEQ ID NO: 552 - RNA, 20 nt, synthetic construct
  source 1..20: mol_type = other RNA
tttgaggcctgtggtagtaa

SEQ ID NO: 553 - RNA, 20 nt, synthetic construct
  source 1..20: mol_type = other RNA
gtcgaagctgcgctatcaaa

SEQ ID NO: 554 - RNA, 20 nt, synthetic construct
  source 1..20: mol_type = other RNA
cgaacacaaacatcacaaag

SEQ ID NO: 555 - RNA, 20 nt, synthetic construct
  source 1..20: mol_type = other RNA
tgtgcaagtggatatttagc

SEQ ID NO: 556 - RNA, 20 nt, synthetic construct
  source 1..20: mol_type = other RNA
tgtcgaagctgcgctatcaa

SEQ ID NO: 557 - RNA, 20 nt, synthetic construct
  source 1..20: mol_type = other RNA
gttctttcctttactaccac

SEQ ID NO: 558 - RNA, 20 nt, synthetic construct
  source 1..20: mol_type = other RNA
tactaccacaggcctcaaag

SEQ ID NO: 559 - RNA, 20 nt, synthetic construct
  source 1..20: mol_type = other RNA
tggatatttagcgggcttgg

SEQ ID NO: 560 - RNA, 20 nt, synthetic construct
  source 1..20: mol_type = other RNA
aggcctacggtagtacagga

SEQ ID NO: 561 - RNA, 20 nt, synthetic construct
  source 1..20: mol_type = other RNA
tttgaggcctacggtagtac

SEQ ID NO: 562 - RNA, 20 nt, synthetic construct
  source 1..20: mol_type = other RNA
gagccctttgcagcctatgg

SEQ ID NO: 563 - RNA, 20 nt, synthetic construct
  source 1..20: mol_type = other RNA
ttggagccctttgcagccta

SEQ ID NO: 564 - RNA, 20 nt, synthetic construct
  source 1..20: mol_type = other RNA
tttccaccataggctgcaaa

SEQ ID NO: 565 - RNA, 20 nt, synthetic construct
  source 1..20: mol_type = other RNA
ttttccaccataggctgcaa

SEQ ID NO: 566 - RNA, 20 nt, synthetic construct
  source 1..20: mol_type = other RNA
ttccaaactgctcaatcaag

SEQ ID NO: 567 - RNA, 20 nt, synthetic construct
  source 1..20: mol_type = other RNA
ttcctcttgattgagcagtt

SEQ ID NO: 568 - RNA, 20 nt, synthetic construct
  source 1..20: mol_type = other RNA
cagcgctttgaggcctgcgg

SEQ ID NO: 569 - RNA, 20 nt, synthetic construct
  source 1..20: mol_type = other RNA
gagcactttgaggcctgttg

SEQ ID NO: 570 - RNA, 20 nt, synthetic construct
  source 1..20: mol_type = other RNA
gatatttccttctccacaac

SEQ ID NO: 571 - RNA, 20 nt, synthetic construct
  source 1..20: mol_type = other RNA
ggttcaaatctgtcagttga

SEQ ID NO: 572 - RNA, 20 nt, synthetic construct
  source 1..20: mol_type = other RNA
tgatgtgtgcactcatctca

SEQ ID NO: 573 - RNA, 20 nt, synthetic construct
  source 1..20: mol_type = other RNA
tggatatttgcagcgctttg

SEQ ID NO: 574 - RNA, 20 nt, synthetic construct
  source 1..20: mol_type = other RNA
ttgcagcgctttgaggcctg

SEQ ID NO: 575 - RNA, 20 nt, synthetic construct
  source 1..20: mol_type = other RNA
ttggagtgctttgaggcata

SEQ ID NO: 576 - RNA, 20 nt, synthetic construct
  source 1..20: mol_type = other RNA
tttgaggcctgttgtggaga

SEQ ID NO: 577 - RNA, 20 nt, synthetic construct
  source 1..20: mol_type = other RNA
aacattccctttgagagagc

(19) United States
(12) Patent Application Publication
(10) Pub. No.: US 2026/0085330 A1
(43) Pub. Date: Mar. 26, 2026

(54) KARYOCREATE (KARYOTYPE CRISPR ENGINEERED ANEUPLOIDY TECHNOLOGY)

(71) Applicant: New York University, New York, NY (US)

(72) Inventors: Teresa DAVOLI, New York, NY (US); Nazario BOSCO, New York, NY (US)

(21) Appl. No.: 19/109,981

(22) PCT Filed: Sep. 8, 2023
(86) PCT No.: PCT/US2023/073784

§ 371 Date: Mar. 7, 2025

Related U.S. Application Data
(60) Provisional application No. 63/375,181, filed on Sep. 9, 2022.

(51) Int. Cl. C12N 15/90; C07K 14/46; C12N 9/22; C12N 15/11

(52) U.S. Cl. CPC C12N 15/907; C07K 14/46; C12N 9/226; C12N 15/11; C07K 2319/00; C12N 2310/20

(57) ABSTRACT

Provided are a fusion protein comprising a mutated kinetochore protein and dCas9. The fusion protein is used in conjunction with guide RNAs target the fusion protein to a location of kinetochore assembly on a centromere such that the fusion protein interferes with chromosome segregation. Use of the fusion protein and the guide RNAs in cells results the cells acquiring an aneuploidy karyotype. Expression vectors that encode the fusion proteins and/or the guide RNAs and their uses in the method of producing an aneuploidy karyotype are also provided.

### CROSS REFERENCE TO RELATED APPLICATION

This application claims priority to U.S. provisional application No. 63/375,181, filed Sep. 9, 2022, the entire disclosure of which is incorporated herein by reference.

### STATEMENT REGARDING FEDERALLY SPONSORED RESEARCH

This invention was made with government support under grant nos. 4R00CA212621-03 and R37CA248631, awarded by the National Institutes of Health. The government has certain rights in the invention.

### SEQUENCE LISTING

The instant application contains a Sequence Listing, which is submitted in .xml format and is hereby incorporated by reference in its entirety. Said .xml file is named “KaryoCreate.xml”, was created on Sep. 1, 2023, and is 519,038 bytes in size.

### RELATED INFORMATION

Aneuploidy, i.e. chromosomal gains or losses, is rare in normal tissues¹⁻³ as it causes cellular stress phenotypes^{4,5}. Despite its detrimental effect, aneuploidy is common in cancer, where specific chromosomes tend to be gained or lost more frequently than others²⁻⁶. We and others have proposed that recurrent patterns of aneuploidy are selected for in cancer to maximize oncogene dosage and minimize tumor-suppressor gene dosage^{4,7}.

A challenge in studying aneuploidy is the lack of straightforward methods to generate cell models with a specific chromosome added or removed. Common methods to induce aneuploidy utilize chemical inhibition of mitotic proteins, e.g. MPS1, resulting in random chromosome missegregation^{8,9}. Microcell-mediated chromosome transfer induces chromosome gains but this method is quite complicated^{10,11}. Centromere inactivation of the Y chromosome can induce its missegregation^{12,13}. Newer strategies to induce chromosome losses involve using CRISPR/Cas9 to eliminate all or part of chromosomes^{5,14,15}. Other recently described methods use non-centromeric repeats to induce specific losses or, more rarely, gains of chromosomes 1 and 9^{16,17}.

Human centromeres contain repetitive α-satellite DNA hierarchically organized in megabase-long arrays called higher-order repeats (HOR), a subset of which bind CENPA, a histone H3 variant critical to kinetochore function¹⁸⁻²¹. In humans, HORs are generally specific to individual chromosomes: 15 autosomes and the 2 sex chromosomes have unique centromeric arrays¹⁹ and the rest can be grouped in two families based on centromere similarity (chromosomes 1, 5, 19 and chromosomes 13, 14, 21, 22). CENPA-bound centromeric sequences direct the kinetochore assembly which enables microtubule binding to mitotic chromosomes²². The KMN network (KNL1/MIS12 complex/NDC80 complex) is important in modulating kinetochore-microtubule attachments²³. In mitosis, each sister kinetochore must be attached to opposite spindle poles to allow their equal and correct segregation²⁴. Properly attached chromatids experience an inter-kinetochore mechanical tension required to satisfy the spindle assembly checkpoint (SAC) and allow progression into anaphase^{24,25}. SAC activation triggers the activity of Aurora B kinase, which destabilizes kinetochore-microtubule attachments by phosphorylating different targets including NDC80 and KNL1^{26,27}. Aurora B activity is counteracted by the action of PP1 phosphatase, recruited to the kinetochores through KNL1²⁸. The balance between kinase and phosphatase activities determines the fate of the kinetochore-microtubule attachment and the timing of the metaphase-to-anaphase transition. In view of these complexities and the lack of previously methods to induce specific chromosome gains and to produce aneuploidy, there is an ongoing need to provide alternatives to the existing methods. The disclosure is pertinent to this need.

### BRIEF SUMMARY

Aneuploidy, the presence of chromosome gains or losses, is a hallmark of cancer and congenital syndromes, such as Down Syndrome. The present disclosure provides compositions and methods for producing aneuploidy. The disclosure provides an approach to generating aneuploidy that is referred to herein as KaryoCreate (Karyotype CRISPR Engineered Aneuploidy Technology). KaryoCreate comprises a CRISPR/Cas9-based technology that uses gRNAs targeting chromosome-specific human centromeric repeats to direct a mutant KNL1/dCas9 construct that interferes with normal mitotic functions, generating chromosome-specific aneuploidy. Using this method, the disclosure demonstrated production of cell models of highly recurrent aneuploidies in human gastro-intestinal cancers and presents data supporting tumor-associated phenotypes occurring after chromosome 18q loss in colorectal cells. The disclosure thus includes a system that enables generation of chromosome-specific aneuploidies by co-expression of a single guide (sg) RNA targeting chromosome-specific CENPA-binding α-satellite repeats together with dCas9 fused to a mutant form of KNL1.

The disclosure includes unique and highly specific sgRNAs for 21 out of 24 human chromosomes. Further, 15 chromosomes out of 24 were validated by imaging and 10 out of 24 were validated by KaryoCreate. The disclosure may be adaptable for use with the remaining human chromosomes, and for use with cells from non-human animals. Expression of the sgRNAs with KNL1Mut-dCas9 leads to missegregation and induction of gains or losses of the targeted chromosome in cellular progeny with an average efficiency of 8% and 12% for gains and losses, respectively (up to 20%), tested and validated across 10 chromosomes. Using KaryoCreate in colon epithelial cells, we show that chromosome 18q loss, a frequent occurrence in gastrointestinal cancers, promotes resistance to TGFβ, likely due to synergistic hemizygous deletion of multiple genes. Thus, the disclosure provides a new technology to create and study chromosome missegregation and aneuploidy in the context of cancer and other conditions that are correlated with the presence of aneuploidy. In one non-limiting embodiment, engineered chromosome 18q loss using a described system promotes tumor-associated phenotypes in colon-derived cells.

### DESCRIPTION OF FIGURES

FIGS. 1A-IF. Prediction and validation of chromosome-specific sgRNAs targeting human α-satellite centromeric sequences. (A) Schematic representation of the computational prediction of chromosome-specific centromeric sgRNAs based on specificity score and predicted efficiency. (B) Idiogram of human karyotype reporting the number of sgRNAs predicted with specificity ≥99% and validated by imaging for each chromosome. (C) Left: Proliferation assay of centromeric sgRNAs in hCECs expressing Cas9 or empty vector (EV). sgRNAα-β refers to a sgRNA specific for chromosome a where β is the sgRNA serial number. Percentage of live cells relative to EV determined 7 days after transduction by cell counting. Mean and S.D. (standard deviation) are from triplicates; p-values are from Wilcoxon test comparing each condition to NC (*=p<0.05); conditions with significant p-values are in red. Imaging validation is also indicated (see (D).) Right: Western blot showing Cas9 expression. (D) Top: Imaging validation of centromere targeting in hCEC clones (containing 3 copies of chr7 or chr13) expressing 3×mScarlet-dCas9 and the indicated sgRNAs. Representative images of interphase are shown (percentages of cells displaying the expected number of foci are in Table S1). Scale bars: 5 μM. Bottom: Low-pass WGS confirming specific aneuploidies in the two clones. (E) Imaging of hCECs (trisomic for chr7) expressing sgRNA7-1 or sgRNA18-4 showing colocalization of 3×mScarlet-dCas9 foci (red) and chromosome 7 or 18 centromeric FISH probes (green); FISH protocol was used after PFA fixation. Colocalization is quantified at right (mean and S.D. from triplicates). (F) Validation of additional sgRNAs as in (D).

FIGS. 2A-2H. KNL1^{Mut}-dCas9 targeted to centromeres induces modest mitotic delay and chromosome missegregation. (A) Left: Maps of KNL1^{RVSF/AAAA}-dCas9 and dCas9-KNL1^{RVSF/AAAA} constructs. Right: Western blot showing the expression of the indicated constructs in hCECs. (B) Top: Time-lapse imaging of hCECs expressing H2B-GFP, KNL1^{Mut}-dCas9, and the indicated sgRNA. Cells were analyzed for time spent in mitosis and for lagging chromosomes (quantified in C and D), and representative images are shown. Bottom: Analysis performed in H2B-GFP hCECs co-expressing 3×mScarlet-KNL1^{Mut}-dCas9 and sgChr7-1, indicating specific chromosome missegregation. (C) Quantification of mitotic duration (time spent between metaphase and anaphase onset) of cells in (B) (mean and S.D. from triplicates; ≥25 dividing cells analyzed per condition). (D) Quantification as in (C) reporting % of mitoses showing lagging chromosomes. (E) Immunofluorescence (IF) analysis of mitotic HCT116 cells expressing KNL1^{Mut}-dCas9 and sgChr7-1 or sgChr18-4 or sgNC stained as indicated. White arrows point to misaligned chromosomes. (F) Quantification of chromosome congression defects in (E) (mean and S.D. from triplicates). (G) Analysis of micronuclei in hCECs expressing KNL1^{Mut}-dCas9 and sgChr7-1, sgChr18-4, or sgNC. The percentage of cells with micronuclei relative to EV was determined 7 days after transduction (mean and S.D. from triplicates; ≥50 cells per condition). (H) Representative images and quantification of chr-18-containing micronuclei in cells treated as in (G), from triplicate experiments.

FIGS. 3A-3G. KNL1^{Mut}-dCas9 is recruited to human centromeres and allows induction of chromosome-specific gains and losses. (A) KaryoCreate conceptualization: Chromosome specificity of human α-satellite centromeric sequences makes it possible to induce missegregation of a specific chromosome while leaving the others unaffected. (B) Western blot showing the expression of KaryoCreate constructs in hCECs, either through transient transfection with a constitutive promoter (pHAGE-CMV) or through infection with a doxycycline (Doxy)-inducible promoter (pIND20). (C) KaryoCreate experimental plan with transient KNL1^{Mut}-dCas9 expression and (transient or constitutive) sgRNA expression; cells are harvested after 7-9 days for validation by FISH and can then be plated to create single-cell clones. (D) Representative FISH images using probes specific for chr7 or chr18 on hCECs showing gains and losses after KaryoCreate with the indicated sgRNAs. (E) Quantification of the experiment shown in (D) for chr7 (top) or chr18 (bottom); see also Table S2 for automated image quantification. Mean and S.D. from triplicates. Gain and loss are the first and second bars in each set of two bars, from left to right, respectively. (F) Representative metaphase spreads from hCECs treated as in (D) and analyzed by FISH using probes specific for chr7 and chr18 as indicated. (G) Quantification of FISH signals from (F) (mean and S.D. from triplicates). Gain and loss are the first and second bars in each set of two bars, from left to right, respectively.

FIG. 4. KaryoCreate induces both arm-level and chromosome-level gains and losses across different human chromosomes. Heatmap depicting arm-level copy numbers inferred from scRNA-seq analysis in KaryoCreate experiments using the indicated sgRNAs. scRNA-seq was used to quantify the presence of chromosome- or arm-level gains or losses using a modified version of CopyKat (see Methods). Rows represent individual cells, columns represent chromosomes, gains in and losses as indicated. ‘Higher expression of KNL1^{Mut}-dCas9’ indicates that the cells were transduced with a larger amount of the construct (as in FIG. 8D). See also Table S3 for quantification of arm- and chromosome-level events.

FIGS. 5A-5G. Loss of 18q in colon cancer cells promotes resistance to TGFβ signaling. (A) Frequency of copy number alteration in colorectal cancer (TCGA) indicated as percentage of patients with gain or loss for each chromosome. (B) Kaplan-Meier survival analysis for colorectal cancer patients (TCGA) displaying or not displaying 18q loss (N=number). (C) Top: Shallow WGS analysis of single-cell-derived clones obtained by KaryoCreate using sgNC or sgChr18-4 performed on diploid hCECs to identify arm-level gains and losses. Each row represents a single clone. Bottom: Plots of copy number alterations from WGS of two representative clones treated with sgChr18-4. (D) Bulk RNA-seq showing differential expression analysis between clone 14 (18q loss) and clone 13 (diploid) using DESeq2 and GSEA (performed using the Hallmark gene sets); the top 7 pathways depleted in clone 14 are shown, including TGFβ signaling as the top depleted one. (E) Effects of TGFβ (20 ng/ml) on clone 13 and 14 growth monitored for 9 days. Cells were counted every 3 days in quadruplicates. p-value is from Wilcoxon test comparing the difference in cell number between treated and untreated clone 14 cultures versus the same difference calculated for clone 13 cultures. (F) Top 10 predicted tumor-suppressor genes (TSG) on 18q and their genomic locations. TSG were predicted based on the correlation between DNA and RNA levels, survival analysis, and TUSON-based q-value for the prediction of TSGs⁴ (see Methods). (G) Western blot analysis for SMAD2, SMAD4, and GAPDH (as control) in clones 13 and 14. Quantification of SMAD2/SMAD4 levels after normalization against GAPDH.

FIGS. 6A-6E (related to FIGS. 1A-1F). Prediction and validation of chromosome-specific sgRNAs targeting human α-satellite centromeric sequences. (A) Left: Proliferation assay on RPEs p21/Rb shRNA expressing Cas9 or empty vector (EV) transduced with lentiviral vectors expressing the indicated sgRNAs. The same number of cells were plated in 6-well plates and the percentage of live cells relative to EV was determined 7 days after transduction. Mean and S.D. from triplicates, p-values from Wilcoxon test (*=p<0.05). Imaging validation is also indicated in red. Right: Western blot showing Cas9 expression. (B) Left: Imaging of hCECs (47, +7) expressing 3×mScarlet-dCas9 and sgChr7-1 in the polyclonal population and in a derived clone (clone 8) with high 3×mScarlet-dCas9 expression. As compared to the polyclonal population, clone 8 contains a higher percentage of cells showing the expected foci. Average frequency of cells displaying foci is shown for the polyclonal and clonal populations (>100 cells counted; in triplicates). Right: Western blot analysis of the expression level of 3×mScarlet-dCas9 in the polyclonal population and clone 8. The percentage of cells showing foci was 45% in the hCEC polyclonal population transduced with 3×mScarlet-dCas9 and increased to 72% in clone 8. (C) Imaging of hCECs expressing 3×mScarlet-dCas9 and the indicated sgRNAs. Representative images of interphase cells are shown; the percentage of cells displaying foci is shown in Table S1. See also FIG. 1F. (D) Imaging of RPEs p21/Rb shRNA expressing 3×mScarlet-dCas9 fusion and the indicated sgRNAs. Representative images of interphase cells are shown. (E) Top: Correlation between the intensity of the signal of the 3×mScarlet-dCas9 foci (measured with ImageJ/Fiji) and the sgRNA activity score (Doench et al., 2016, 2014) of cells treated as in (C). Bottom: Correlation between the intensity of the signal of the 3×mScarlet-dCas9 foci and the number of predicted sgRNA binding sites on the specific centromere (based on the T2T genome assembly) of cells treated as in (C). Pearson correlation coefficients and corresponding p-values are shown.

FIGS. 7A-7F (related to FIGS. 2A-2H). Analysis of KNL1^{Mut}-dCas9 and other fusion proteins targeted to centromeres. (A) Maps of the dCas9, KNL1^{RVSF/AAAA}-dCas9, KNL1^{S24A:S60A}-dCas9, NDC80-CH1-dCas9, and NDC80-CH2-dCas9 constructs. The predicted function of each construct is indicated on the right. See text for details. (B) Western blot showing the expression of the indicated constructs in hCECs. (C) Western blot showing the expression of the indicated constructs, in which different mutated segments of KNL1 or NDC80 are fused to the N- or C-terminus of dCas9; see also (A). L: linker with amino acid sequence GGSGGGS (SEQ ID NO: 5). (D) Imaging of hCECs (47, +7) expressing 3×mScarlet-KNL1^{Mut}-dCas9 and transduced with sgChr7-1 or sgChr18-4. (E) Proliferation rate of hCECs transduced with KNL1^{Mut}-dCas9 and with the indicated sgRNAs. Mean and S.D. from triplicates are shown for each time point. (F) FISH imaging and quantification of micronuclei containing chromosome 7 or 13 in hCECs treated with KNL1^{Mut}-dCas9 and the indicated sgRNA (as in FIG. 2G); quantification of micronuclei counts is shown below. Experiments were performed in duplicates, and for each replicate, at least 100 cells were scored.

FIGS. 8A-8H (related to FIGS. 3A-3G). Analysis of KNL1^{Mut}-dCas9 and other fusion proteins targeted to centromeres for the induction of chromosome-specific gains and losses. (A) KaryoCreate experiment in hCECs comparing the efficiency of different methods for delivering KNL1^{Mut}-dCas9, as quantified by FISH. Methods: (1) transfection of pHAGE-KNL1^{Mut}-dCas9, whose expression of KNL1^{Mut}-dCas9 is driven by the CMV promoter; (2) lentiviral-mediated transduction with pIND20-KNL1^{Mut}-dCas9, whereby the vector is integrated in the genome of the target cells and expression of KNL1^{Mut}-dCas9 is driven by doxycycline treatment (1 μg/ml); (3) lentiviral-mediated transduction with pHAGE-DD-KNL1^{Mut}-dCas9, whereby expression of KNL1^{Mut}-dCas9 is driven by treatment with shield-1 to stabilize the protein. All cells were transduced with sgChr7-1, and FISH quantification of chr7 gains/losses is shown (mean and S.D. from triplicates). Gain and loss are the first and second bars in each set of two bars, from left to right, respectively. (B) KaryoCreate experiment comparing the efficiency of different constructs in inducing chromosome gains and losses. hCECs were transduced with sgChr7-1 and the indicated constructs. FISH quantification for chr7 gains/losses is shown (mean and S.D. from triplicates), along with the aneuploidy level (% of chr7 gains/losses) normalized to the expression level of each construct (as in FIG. 7B). Note that after normalization, the induction of aneuploidy is greatest for NDC80CH2-dCas9 and is higher for KNL1^{S24A:S60A}-dCas9 than for KNL1^{RVSF/AAAA}-dCas9. (C) Left: Western blot analysis of the indicated constructs. Right: KaryoCreate experiment to compare the efficiency of different constructs in inducing chromosome gains and losses. hCECs were transduced with sgChr7-1 and the indicated constructs, and FISH quantification of chr7 gains/losses is shown. Gain and loss are the first and second bars in each set of two bars, from left to right, respectively. (D) Left: Western blot analysis of dCas9 expression in hCECs transduced with KNL1^{Mut}-dCas9 using different amount of virus (about 3 times more virus in the HIGH versus LOW sample, i.e. MOI of 6 for HIGH and 2 for LOW). The corresponding quantification (through ImageJ) is shown below. Right: FISH quantification of chr7 gains/losses in cells expressing KNL1^{Mut}-dCas9 transduced with sgChr7-1 using different amounts of virus at 9-10 days after transduction (mean and S.D. from duplicates). (E) FISH quantification of chr7 gains/losses in hCECs transduced with KNL1^{Mut}-dCas9 and with sgChr7-1 and/or sgChr7-3 (mean and S.D. from duplicates). (F) Single-cell sequencing quantification of chr9 gains/losses in hCECs were transduced with KNL1^{Mut}-dCas9 and with sgNC, sgChr9-3 and/or sgChr9-5 (mean and S.D. from technical duplicates). (G) Left: FACS sorting results for hCECs treated as in (D) using an MOI of 2 after sorting for low or high expression of the cell surface protein EPHB4, encoded by a gene on chr7. Right: FISH quantification of the % of chr7 gains or losses in each condition (N=100 nuclei; mean and S.D. from duplicates). *, p-value<0.05 (Welch two-sample t-test). Gain and loss are the first and second bars in each set of two bars, from left to right, respectively. (H) scRNA-seq analysis of chromosome or arm gains/losses (as in FIG. 4) in hCECs transduced with KNL1^{Mut}-dCas9 (via infection with pIND20-KNL1^{Mut}-dCas9 lentiviral vector) and sgChr7-1. Cells were treated with doxycycline for the indicated number of days to induce construct expression; experiment performed in duplicate.

FIGS. 9A-9I (related to FIG. 4). Analysis of KaryoCreate across chromosomes and conditions. (A) Analysis of hCEC clones with different aneuploidies by bulk WGS (top) and scRNA-seq (bottom). Arm-level copy number events were inferred from each method (see Methods) and the derived copy number profiles are shown for both methods. See also (B). (B) FISH and scRNA-seq analyses of hCEC clones with chr7 trisomy or more complex karyotypes and the percentage of aneuploid cells was quantified using both methods. Mean values from duplicates are shown. (C) A heatmap depicting gene copy numbers inferred from scRNA-seq analysis following KaryoCreate control experiments. hCECs were transduced either with empty vector or with KNL1^{Mut}-dCas9 together with a negative control sgRNA (sgNC), and scRNA-seq was performed as in (B) to estimate % of gains and losses across chromosomes. (D) A heatmap depicting gene copy numbers inferred from scRNA-seq analysis following KaryoCreate. KaryoCreate for different individual chromosomes (or combination of chromosomes) was performed on RPEs. scRNA-seq was used to estimate the presence of chromosome- or arm-level gains or losses using a modified version of CopyKat. The median expression of genes across each chromosome arm is used to estimate the DNA copy number. The % of gains/losses for each arm (reported below each heatmap) is estimated by comparing the DNA copy number distribution of each experimental sample (chromosome-specific sgRNA) to that of the negative control (sgRNA NC; see also Methods). Heatmap rows represent individual cells, columns represent different chromosomes, and the color represents the copy number change (gain in red and loss in blue). (E) Average proportions (%) of whole-chromosome and arm-level gains/losses. The percentage of the indicated events were calculated as the average among the aneuploid cells generated using KaryoCreate for chromosomes 6, 7, 8, 9, 12, 16, and X (mean values from duplicates). (F) A heatmap depicting chromosome copy numbers inferred from scRNA-seq analysis following KaryoCreate. KaryoCreate was performed on hCECs using two sgRNAs targeting chromosome 7 (sgChr7-1) and 18 (sgChr18-4). scRNA-seq was used to estimate the presence of chromosome- or arm-level gains/losses using a modified version of CopyKat as in (D). Heatmap rows represent individual cells, columns represent different chromosomes, and the color represents the copy number change (gain in red and loss in blue). (G) Immunofluorescence (IF) assay showing DNA damage in HCT116 cells expressing KNL1^{Mut}-dCas9 and sgNC, sgChr7-1, or sgChr18-4. IF was performed for γH2AX (green), CREST (red) to visualize centromeres, and DAPI (blue). Representative images are shown. (H) Quantification of experiment shown in (G). Left: number of DNA damage foci colocalizing with CREST in each cell, quantified and normalized to the total number of CREST foci in the cell. Right: total γH2AX signal per cell, quantified and normalized to the total DAPI signal. p-values are from Wilcoxon test. (I) Left: The total γH2AX signal per cell as determined by IF analysis of hCECs expressing KNL1^{Mut}-dCas9 (pIND20 vector) and sgNC or sgChr7-1 for γH2AX (green) and DAPI (blue), quantified and normalized to the total DAPI signal. Right: Western blot analysis of KNL1^{Mut}-dCas9 expression before or after treatment with doxycycline to induce construct expression. p-values are from Wilcoxon test.

FIGS. 10A-10H (related to FIGS. 5A-5G). Dissection of the consequences of 18q loss in colorectal cancer. (A) Schematic of experimental plan to apply KaryoCreate across different chromosomes to derive single-cell clones with specific gains or losses. (B) Shallow WGS analysis of single-cell-derived clones obtained by KaryoCreate using sgNC or sgChr7-1 performed on diploid hCECs (as indicated). (C) Representative FISH images and copy number plots from WGS analysis of hCEC sgChr7-1 clone 23 (B) before or after 25 population doublings in culture. (D) Survival analysis (Kaplan-Meier curve) for colorectal cancer patients (TCGA-COADREAD) displaying or not displaying 18q loss, after exclusion of patients with SMAD4 point mutation. (E) Proliferation rates of the indicated hCEC clones 13 and 14 (18q loss) (as in FIG. 5E) after the overexpression of the indicated genes. Mean and S.D. are shown for triplicates; p-values are from Wilcoxon test (*=p<0.05). Proliferation rates for hCEC clones 10 and 5 (18 loss) with and without TGFβ are also shown. (F) Western blot showing SMAD2 and SMAD4 levels in hCEC clone 13 after overexpression of GFP, SMAD2, SMAD4, or SMAD2+SMAD4. Related to FIG. 10E. (G) Proliferation rates of the indicated hCEC cell lines (clone 14 and hCEC transduced with dCas9 and a SMAD4 or NC sgRNA) when cultured in the presence of TGFβ (20 ng/ml) for 9 days; cells were counted every 3 days in triplicates. p-value is derived from the Wilcoxon test. Western blot showing SMAD4 levels in hCECs transduced with dCas9 and a SMAD4 or NC sgRNA. Related to FIG. 10G. (H) Western blot showing SMAD4 levels in hCECs transduced with dCas9 and a SMAD4 or NC sgRNA. Related to Fig. S5G.

### DETAILED DESCRIPTION

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example+/−10%.

This disclosure includes every amino acid sequence described herein and all nucleotide sequences encoding the amino acid sequences. Every sequence having from 80-99% similarity, inclusive, and including and all numbers and ranges of numbers there between, with the sequences provided here are included in the invention. All of the amino acid sequences described herein can include amino acid substitutions, such as conservative substitutions, that do not adversely affect the function of the protein that comprises the amino acid sequences. All amino acid sequences encoded by the described polynucleotides are expressly included within this disclosure. The disclosure includes all segments of described polynucleotides that contain open reading frames.

All sequences that are described by reference to a database are incorporated herein by reference as the sequences exist in the database as of the effective filing date of this application or patent. All sequences referred to in publications are incorporated herein by reference.

This disclosure provides compositions, methods, and systems referred to herein as noted above as KaryoCreate, a new method that includes CRISPR/Cas9 technology combined with chromosome specificity for human centromeric α-satellite repeats with interfering with normal functions of the KMN network (in particular KNL1) to generate chromosome-specific aneuploidy. The described approach involves use of a fusion protein comprising a mutated kinetochore protein and dCas9.

In an embodiment the kinetochore protein is KNL1 protein or a functional segment thereof. In embodiments, the KNL1 protein or the functional segment thereof comprises one or more mutations. In embodiments, the kinetochore protein comprises a segment of KNL1 protein, wherein the segment of the KNL1 protein comprises at least the first 86 N-terminal amino acids of the KNL1 protein, and wherein the first 86 N-terminal amino acids comprise a mutation of the sequence RVSF to AAAA, or S24A, or S60A, or a combination thereof.

The fusion protein may be modified to include a suitable nuclear localization signal. In an embodiment, a KNL1^{RVSF/AAAA}-dCas9 fusion protein is used. In another embodiment, a KNL1^{S24A:S60A}-dCas9 fusion protein is used.

Any suitable linker sequence may be present between the KNL1 protein segment and the dCas9 segment. In an embodiment a suitable linker comprises a GS sequence. In an embodiment, the linker has the sequence GGSGGGS (SEQ ID NO: 5).

In embodiments, the described fusion proteins have amino acid sequences that are encoded by the following DNA sequences:

KNL1
linker
dCas9
KNL1^{RVSF/AAAA}-dCas9
(SEQ ID NO: 3)
ATGGATGGGGTGTCTTCAGAGGCTAATGAAGAAAATGACAATATAGAGAG
ACCTGTTAGAAGACGGCATTCTTCAATATTGAAACCCCCAAGGAGTCCTC
TTCAGGACCTCAGAGGTGGGAATGAAACAGTTCAAGAGTCAAACGCGTTA
AGGAATAAGAAAAACTCTCGTGCAGCCGCCGCTGCAGATACTATAAAGGT
ATTCCAGACGGAGTCTCATATGAAAATAGTGAGAAAGTCAGAAATGGAAG
AAACAGAA ggcggttccggcggagggtcgGACAAGAAGTACAGCATCGG
CCTGGCCATCGGCACCAACTCTGTGGGCTGGGCCGTGATCACCGACGAGT
ACAAGGTGCCCAGCAAGAAATTCAAGGTGCTGGGCAACACCGACCGGCAC
AGCATCAAGAAGAACCTGATCGGCGCCCTGCTGTTCGACAGCGGAGAAAC
AGCCGAGGCCACCCGGCTGAAGAGAACCGCCAGAAGAAGATACACCAGAC
GGAAGAACCGGATCTGCTATCTGCAAGAGATCTTCAGCAACGAGATGGCC
AAGGTGGACGACAGCTTCTTCCACAGACTGGAAGAGTCCTTCCTGGTGGA
AGAGGATAAGAAGCACGAGCGGCACCCCATCTTCGGCAACATCGTGGACG
AGGTGGCCTACCACGAGAAGTACCCCACCATCTACCACCTGAGAAAGAAA
CTGGTGGACAGCACCGACAAGGCCGACCTGCGGCTGATCTATCTGGCCCT
GGCCCACATGATCAAGTTCCGGGGCCACTTCCTGATCGAGGGCGACCTGA
ACCCCGACAACAGCGACGTGGACAAGCTGTTCATCCAGCTGGTGCAGACC
TACAACCAGCTGTTCGAGGAAAACCCCATCAACGCCAGCGGCGTGGACGC
CAAGGCCATCCTGTCTGCCAGACTGAGCAAGAGCAGACGGCTGGAAAATC
TGATCGCCCAGCTGCCCGGCGAGAAGAAGAATGGCCTGTTCGGCAACCTG
ATTGCCCTGAGCCTGGGCCTGACCCCCAACTTCAAGAGCAACTTCGACCT
GGCCGAGGATGCCAAACTGCAGCTGAGCAAGGACACCTACGACGACGACC
TGGACAACCTGCTGGCCCAGATCGGCGACCAGTACGCCGACCTGTTTCTG
GCCGCCAAGAACCTGTCCGACGCCATCCTGCTGAGCGACATCCTGAGAGT
GAACACCGAGATCACCAAGGCCCCCCTGAGCGCCTCTATGATCAAGAGAT
ACGACGAGCACCACCAGGACCTGACCCTGCTGAAAGCTCTCGTGCGGCAG
CAGCTGCCTGAGAAGTACAAAGAGATTTTCTTCGACCAGAGCAAGAACGG
CTACGCCGGCTACATCGATGGCGGAGCCAGCCAGGAAGAGTTCTACAAGT
TCATCAAGCCCATCCTGGAAAAGATGGACGGCACCGAGGAACTGCTCGTG
AAGCTGAACAGAGAGGACCTGCTGCGGAAGCAGCGGACCTTCGACAACGG
CAGCATCCCCCACCAGATCCACCTGGGAGAGCTGCACGCCATTCTGCGGC
GGCAGGAAGATTTTTACCCATTCCTGAAGGACAACCGGGAAAAGATCGAG
AAGATCCTGACCTTCCGCATCCCCTACTACGTGGGCCCTCTGGCCAGGGG
AAACAGCAGATTCGCCTGGATGACCAGAAAGAGCGAGGAAACCATCACCC
CCTGGAACTTCGAGGAAGTGGTGGACAAGGGCGCCAGCGCCCAGAGCTTC
ATCGAGCGGATGACCAACTTCGATAAGAACCTGCCCAACGAGAAGGTGCT
GCCCAAGCACAGCCTGCTGTACGAGTACTTCACCGTGTACAACGAGCTGA
CCAAAGTGAAATACGTGACCGAGGGAATGAGAAAGCCCGCCTTCCTGAGC
GGCGAGCAGAAAAAAGCCATCGTGGACCTGCTGTTCAAGACCAACCGGAA
AGTGACCGTGAAGCAGCTGAAAGAGGACTACTTCAAGAAAATCGAGTGCT
TCGACTCCGTGGAAATCTCCGGCGTGGAAGATCGGTTCAACGCCTCCCTG
GGCACATACCACGATCTGCTGAAAATTATCAAGGACAAGGACTTCCTGGA
CAATGAGGAAAACGAGGACATTCTGGAAGATATCGTGCTGACCCTGACAC
TGTTTGAGGACAGAGAGATGATCGAGGAACGGCTGAAAACCTATGCCCAC
CTGTTCGACGACAAAGTGATGAAGCAGCTGAAGCGGCGGAGATACACCGG
CTGGGGCAGGCTGAGCCGGAAGCTGATCAACGGCATCCGGGACAAGCAGT
CCGGCAAGACAATCCTGGATTTCCTGAAGTCCGACGGCTTCGCCAACAGA
AACTTCATGCAGCTGATCCACGACGACAGCCTGACCTTTAAAGAGGACAT
CCAGAAAGCCCAGGTGTCCGGCCAGGGCGATAGCCTGCACGAGCACATTG
CCAATCTGGCCGGCAGCCCCGCCATTAAGAAGGGCATCCTGCAGACAGTG
AAGGTGGTGGACGAGCTCGTGAAAGTGATGGGCCGGCACAAGCCCGAGAA
CATCGTGATCGAAATGGCCAGAGAGAACCAGACCACCCAGAAGGGACAGA
AGAACAGCCGCGAGAGAATGAAGCGGATCGAAGAGGGCATCAAAGAGCTG
GGCAGCCAGATCCTGAAAGAACACCCCGTGGAAAACACCCAGCTGCAGAA
CGAGAAGCTGTACCTGTACTACCTGCAGAATGGGCGGGATATGTACGTGG
ACCAGGAACTGGACATCAACCGGCTGTCCGACTACGATGTGGACGCTATC
GTGCCTCAGAGCTTTCTGAAGGACGACTCCATCGATAACAAAGTGCTGAC
TCGGAGCGACAAGAACCGGGGCAAGAGCGACAACGTGCCCTCCGAAGAGG
TCGTGAAGAAGATGAAGAACTACTGGCGCCAGCTGCTGAATGCCAAGCTG
ATTACCCAGAGGAAGTTCGACAATCTGACCAAGGCCGAGAGAGGCGGCCT
GAGCGAACTGGATAAGGCCGGCTTCATCAAGAGACAGCTGGTGGAAACCC
GGCAGATCACAAAGCACGTGGCACAGATCCTGGACTCCCGGATGAACACT
AAGTACGACGAGAACGACAAACTGATCCGGGAAGTGAAAGTGATCACCCT
GAAGTCCAAGCTGGTGTCCGATTTCCGGAAGGATTTCCAGTTTTACAAAG
TGCGCGAGATCAACAACTACCACCACGCCCACGACGCCTACCTGAACGCC
GTCGTGGGAACCGCCCTGATCAAAAAGTACCCTAAGCTGGAAAGCGAGTT
CGTGTACGGCGACTACAAGGTGTACGACGTGCGGAAGATGATCGCCAAGA
GCGAGCAGGAAATCGGCAAGGCTACCGCCAAGTACTTCTTCTACAGCAAC
ATCATGAACTTTTTCAAGACCGAGATTACCCTGGCCAACGGCGAGATCCG
GAAGCGGCCTCTGATCGAGACAAACGGCGAAACAGGCGAGATCGTGTGGG
ATAAGGGCCGGGACTTTGCCACCGTGCGGAAAGTGCTGTCTATGCCCCAA
GTGAATATCGTGAAAAAGACCGAGGTGCAGACAGGCGGCTTCAGCAAAGA
GTCTATCCTGCCCAAGAGGAACAGCGACAAGCTGATCGCCAGAAAGAAGG
ACTGGGACCCTAAGAAGTACGGCGGCTTCGACAGCCCCACCGTGGCCTAT
TCTGTGCTGGTGGTGGCCAAAGTGGAAAAGGGCAAGTCCAAGAAACTGAA
GAGTGTGAAAGAGCTGCTGGGGATCACCATCATGGAAAGAAGCAGCTTCG
AGAAGAATCCCATCGACTTTCTGGAAGCCAAGGGCTACAAAGAAGTGAAA
AAGGACCTGATCATCAAGCTGCCTAAGTACTCCCTGTTCGAGCTGGAAAA
CGGCCGGAAGAGAATGCTGGCCTCTGCCGGCGAACTGCAGAAGGGAAACG
AACTGGCCCTGCCCTCCAAATATGTGAACTTCCTGTACCTGGCCAGCCAC
TATGAGAAGCTGAAGGGCTCCCCCGAGGATAATGAGCAGAAACAGCTGTT
TGTGGAACAGCACAAACACTACCTGGACGAGATCATCGAGCAGATCAGCG
AGTTCTCCAAGAGAGTGATCCTGGCCGACGCTAATCTGGACAAGGTGCTG
AGCGCCTACAACAAGCACAGAGACAAGCCTATCAGAGAGCAGGCCGAGAA
TATCATCCACCTGTTTACCCTGACCAATCTGGGAGCCCCTGCCGCCTTCA
AGTACTTTGACACCACCATCGACCGGAAGAGGTACACCAGCACCAAAGAG
GTGCTGGACGCCACCCTGATCCACCAGAGCATCACCGGCCTGTACGAGAC
ACGGATCGACCTGTCTCAGCTGGGAGGCGACGCCTATCCCTATGACGTGC
CCGATTATGCCAGCCTGGGCAGCGGCTCCCCCAAGAAAAAACGCAAGGTG
GAAGATCCTAAGAAAAAGCGGAAAGTGGACGGCATTGGTAGTGGGAGCAA
CGGCAGCAGCGGATCCtga

The KNL1^{RVSF/AAAA} segment of the fusion protein sequence encoded by the DNA sequence above is:

(SEQ ID NO: 1)
MDGVSSEANEENDNIERPVRRRHSSILKPPRSPLQDLRGGNETVQESNA
LRNKKNSRAAAAADTIKVFQTESHMKIVRKSEMEETE

In an embodiment, the KNL1^{S24A:S60A}-dCas9 (SEQ ID NO: 4) fusion protein is encoded by the following DNA sequence:

KNL1^{S24A; S60A}_linkerdCas9
(SEQ ID NO: 4)
ATGGATGGGGTGTCTTCAGAGGCTAATGAAGAAAATGACAATATAGAGAGACCTGTTAGAAGAC
GGCATGCCTCAATATTGAAACCCCCAAGGAGTCCTCTTCAGGACCTCAGAGGTGGGAATGAAA
CAGTTCAAGAGTCAAACGCGTTAAGGAATAAGAAAAACTCTCGTCGAGTCGCCTTTGCAGATAC
TATAAAGGTATTCCAGACGGAGTCTCATATGAAAATAGTGAGAAAGTCA
ggcggttccggcggagggtcg
GACAAGAAGTACAGCATCGGCCTGGCCATCGGCACCAACTCTGTGGGCTGGGCCGT
GATCACCGACGAGTACAAGGTGCCCAGCAAGAAATTCAAGGTGCTGGGCAACACCG
ACCGGCACAGCATCAAGAAGAACCTGATCGGCGCCCTGCTGTTCGACAGCGGAGAA
ACAGCCGAGGCCACCCGGCTGAAGAGAACCGCCAGAAGAAGATACACCAGACGGA
AGAACCGGATCTGCTATCTGCAAGAGATCTTCAGCAACGAGATGGCCAAGGTGGAC
GACAGCTTCTTCCACAGACTGGAAGAGTCCTTCCTGGTGGAAGAGGATAAGAAGCA
CGAGCGGCACCCCATCTTCGGCAACATCGTGGACGAGGTGGCCTACCACGAGAAGT
ACCCCACCATCTACCACCTGAGAAAGAAACTGGTGGACAGCACCGACAAGGCCGAC
CTGCGGCTGATCTATCTGGCCCTGGCCCACATGATCAAGTTCCGGGGCCACTTCCT
GATCGAGGGCGACCTGAACCCCGACAACAGCGACGTGGACAAGCTGTTCATCCAGC
TGGTGCAGACCTACAACCAGCTGTTCGAGGAAAACCCCATCAACGCCAGCGGCGTG
GACGCCAAGGCCATCCTGTCTGCCAGACTGAGCAAGAGCAGACGGCTGGAAAATCT
GATCGCCCAGCTGCCCGGCGAGAAGAAGAATGGCCTGTTCGGCAACCTGATTGCCC
TGAGCCTGGGCCTGACCCCCAACTTCAAGAGCAACTTCGACCTGGCCGAGGATGCC
AAACTGCAGCTGAGCAAGGACACCTACGACGACGACCTGGACAACCTGCTGGCCCA
GATCGGCGACCAGTACGCCGACCTGTTTCTGGCCGCCAAGAACCTGTCCGACGCCA
TCCTGCTGAGCGACATCCTGAGAGTGAACACCGAGATCACCAAGGCCCCCCTGAGC
GCCTCTATGATCAAGAGATACGACGAGCACCACCAGGACCTGACCCTGCTGAAAGC
TCTCGTGCGGCAGCAGCTGCCTGAGAAGTACAAAGAGATTTTCTTCGACCAGAGCA
AGAACGGCTACGCCGGCTACATCGATGGCGGAGCCAGCCAGGAAGAGTTCTACAA
GTTCATCAAGCCCATCCTGGAAAAGATGGACGGCACCGAGGAACTGCTCGTGAAGC
TGAACAGAGAGGACCTGCTGCGGAAGCAGCGGACCTTCGACAACGGCAGCATCCC
CCACCAGATCCACCTGGGAGAGCTGCACGCCATTCTGCGGCGGCAGGAAGATTTTT
ACCCATTCCTGAAGGACAACCGGGAAAAGATCGAGAAGATCCTGACCTTCCGCATC
CCCTACTACGTGGGCCCTCTGGCCAGGGGAAACAGCAGATTCGCCTGGATGACCAG
AAAGAGCGAGGAAACCATCACCCCCTGGAACTTCGAGGAAGTGGTGGACAAGGGC
GCCAGCGCCCAGAGCTTCATCGAGCGGATGACCAACTTCGATAAGAACCTGCCCAA
CGAGAAGGTGCTGCCCAAGCACAGCCTGCTGTACGAGTACTTCACCGTGTACAACG
AGCTGACCAAAGTGAAATACGTGACCGAGGGAATGAGAAAGCCCGCCTTCCTGAGC
GGCGAGCAGAAAAAAGCCATCGTGGACCTGCTGTTCAAGACCAACCGGAAAGTGAC
CGTGAAGCAGCTGAAAGAGGACTACTTCAAGAAAATCGAGTGCTTCGACTCCGTGG
AAATCTCCGGCGTGGAAGATCGGTTCAACGCCTCCCTGGGCACATACCACGATCTG
CTGAAAATTATCAAGGACAAGGACTTCCTGGACAATGAGGAAAACGAGGACATTCT
GGAAGATATCGTGCTGACCCTGACACTGTTTGAGGACAGAGAGATGATCGAGGAAC
GGCTGAAAACCTATGCCCACCTGTTCGACGACAAAGTGATGAAGCAGCTGAAGCGG
CGGAGATACACCGGCTGGGGCAGGCTGAGCCGGAAGCTGATCAACGGCATCCGGG
ACAAGCAGTCCGGCAAGACAATCCTGGATTTCCTGAAGTCCGACGGCTTCGCCAAC
AGAAACTTCATGCAGCTGATCCACGACGACAGCCTGACCTTTAAAGAGGACATCCA
GAAAGCCCAGGTGTCCGGCCAGGGCGATAGCCTGCACGAGCACATTGCCAATCTGG
CCGGCAGCCCCGCCATTAAGAAGGGCATCCTGCAGACAGTGAAGGTGGTGGACGA
GCTCGTGAAAGTGATGGGCCGGCACAAGCCCGAGAACATCGTGATCGAAATGGCCA
GAGAGAACCAGACCACCCAGAAGGGACAGAAGAACAGCCGCGAGAGAATGAAGCG
GATCGAAGAGGGCATCAAAGAGCTGGGCAGCCAGATCCTGAAAGAACACCCCGTG
GAAAACACCCAGCTGCAGAACGAGAAGCTGTACCTGTACTACCTGCAGAATGGGCG
GGATATGTACGTGGACCAGGAACTGGACATCAACCGGCTGTCCGACTACGATGTGG
ACGCTATCGTGCCTCAGAGCTTTCTGAAGGACGACTCCATCGATAACAAAGTGCTG
ACTCGGAGCGACAAGAACCGGGGCAAGAGCGACAACGTGCCCTCCGAAGAGGTCG
TGAAGAAGATGAAGAACTACTGGCGCCAGCTGCTGAATGCCAAGCTGATTACCCAG
AGGAAGTTCGACAATCTGACCAAGGCCGAGAGAGGCGGCCTGAGCGAACTGGATA
AGGCCGGCTTCATCAAGAGACAGCTGGTGGAAACCCGGCAGATCACAAAGCACGTG
GCACAGATCCTGGACTCCCGGATGAACACTAAGTACGACGAGAACGACAAACTGAT
CCGGGAAGTGAAAGTGATCACCCTGAAGTCCAAGCTGGTGTCCGATTTCCGGAAGG
ATTTCCAGTTTTACAAAGTGCGCGAGATCAACAACTACCACCACGCCCACGACGCC
TACCTGAACGCCGTCGTGGGAACCGCCCTGATCAAAAAGTACCCTAAGCTGGAAAG
CGAGTTCGTGTACGGCGACTACAAGGTGTACGACGTGCGGAAGATGATCGCCAAGA
GCGAGCAGGAAATCGGCAAGGCTACCGCCAAGTACTTCTTCTACAGCAACATCATG
AACTTTTTCAAGACCGAGATTACCCTGGCCAACGGCGAGATCCGGAAGCGGCCTCT
GATCGAGACAAACGGCGAAACAGGCGAGATCGTGTGGGATAAGGGCCGGGACTTT
GCCACCGTGCGGAAAGTGCTGTCTATGCCCCAAGTGAATATCGTGAAAAAGACCGA
GGTGCAGACAGGCGGCTTCAGCAAAGAGTCTATCCTGCCCAAGAGGAACAGCGACA
AGCTGATCGCCAGAAAGAAGGACTGGGACCCTAAGAAGTACGGCGGCTTCGACAG
CCCCACCGTGGCCTATTCTGTGCTGGTGGTGGCCAAAGTGGAAAAGGGCAAGTCCA
AGAAACTGAAGAGTGTGAAAGAGCTGCTGGGGATCACCATCATGGAAAGAAGCAGC
TTCGAGAAGAATCCCATCGACTTTCTGGAAGCCAAGGGCTACAAAGAAGTGAAAAA
GGACCTGATCATCAAGCTGCCTAAGTACTCCCTGTTCGAGCTGGAAAACGGCCGGA
AGAGAATGCTGGCCTCTGCCGGCGAACTGCAGAAGGGAAACGAACTGGCCCTGCC
CTCCAAATATGTGAACTTCCTGTACCTGGCCAGCCACTATGAGAAGCTGAAGGGCT
CCCCCGAGGATAATGAGCAGAAACAGCTGTTTGTGGAACAGCACAAACACTACCTG
GACGAGATCATCGAGCAGATCAGCGAGTTCTCCAAGAGAGTGATCCTGGCCGACGC
TAATCTGGACAAGGTGCTGAGCGCCTACAACAAGCACAGAGACAAGCCTATCAGAG
AGCAGGCCGAGAATATCATCCACCTGTTTACCCTGACCAATCTGGGAGCCCCTGCC
GCCTTCAAGTACTTTGACACCACCATCGACCGGAAGAGGTACACCAGCACCAAAGA
GGTGCTGGACGCCACCCTGATCCACCAGAGCATCACCGGCCTGTACGAGACACGGA
TCGACCTGTCTCAGCTGGGAGGCGACGCCTATCCCTATGACGTGCCCGATTATGCC
AGCCTGGGCAGCGGCTCCCCCAAGAAAAAACGCAAGGTGGAAGATCCTAAGAAAAA
GCGGAAAGTGGACGGCATTGGTAGTGGGAGCAACGGCAGCAGCGGATCCtga

The KNL1^{S24A:S60A} segment of the fusion protein encoded by the DNA sequence above is:

(SEQ ID NO: 2)
MDGVSSEANEENDNIERPVRRRHASILKPPRSPLQDLRGGNETVQESNA
LRNKKNSRRVAFADTIKVFQTESHMKIVRKS

The sequence of dCas9 is well known in the art. The sequence of the dCas9 used in this disclosure is evident from the DNA sequences described herein.

The described fusion protein can be provided in a composition that is suitable for introducing the fusion protein into cells. The composition may include one or more guide RNAs, or the fusion protein may be introduced concurrently or sequentially into cells with one or more guide RNAs. The guide RNA targets the fusion protein to a location of kinetochore assembly on a centromere such that the fusion protein interferes with chromosome segregation.

The described fusion protein and the RNAs are used in a method to produce aneuploidy in eukaryotic cells. The method comprises introducing into cells a described fusion protein and at least one guide RNA that targets the fusion protein to a location of kinetochore assembly on a centromere of a specific chromosome such that the fusion protein interferes with segregation of the chromosome. The cells are then allowed to divide in the presence of the fusion protein and the guide RNA such that cell division results in divided cells that comprise an aneuploidy karyotype. In an embodiment the aneuploidy karyotype comprises a gain of a chromosome. In an embodiment, the aneuploidy karyotype comprises a loss of a chromosome. In an embodiment, the aneuploidy karyotype is associated with a malignant cell phenotype. The disclosure also provides an isolated population of cells made by the described methods, as well as cell lines with the engineered aneuploidy karyotypes.

The disclosure also provides a kit comprising the described fusion protein. The kit may include one or a plurality of guide RNAs that target the fusion protein to one or more locations of kinetochore assembly on a centromere of one or more chromosomes, one or more expression vectors that encode one or a plurality of guide RNAs, and/or an expression vector that encodes the described fusion protein, or the fusion protein itself. The components of the kit may be provided in one or more containers. The container(s) may contain reagents used to practice a method of the disclosure. The reagents may be provided in a ready to use buffer, or may be adapted for reconstitution in a suitable buffer, such as by lyophilization. The kits may include printed material that instructs a user how to use the kit contents in order to perform a described method. As such, the disclosure includes articles of manufacture that comprise one or more containers containing the described proteins and/or polynucleotides encoding the proteins, and printed material that describes contents and/or how to use the components in a described method.

The disclosure also provides a method comprising selecting a guide RNA that targets a location of kinetochore assembly on a centromere of a specific chromosome, and introducing into cells a combination of the selected guide RNA and a fusion protein comprising a mutated kinetochore protein and dCas9, allowing cell divisional in the presence of the selected guide RNA and the fusion protein such that divided cells comprise an aneuploidy karyotype.

The described compositions, methods, and systems can be introduced into cells using a variety of approaches, such as by using mRNA, or a ribonucleoprotein (RNP) complex, or plasmids or other expression vectors, or combinations thereof. In embodiments, a viral vector can be used. In embodiments, a phagemid or modified bacteriophage can be used. The expression of the fusion protein may be driven by a promoter that is operably linked to the sequence coding the fusion protein. The promoter may be an inducible or constitutive promoter. Thus, in certain embodiments, such as by use of an inducible promoter, expression of the fusion protein and/or the guide RNA can be controlled such that the expression is transient.

Viral expression vectors may be used as naked polynucleotides, or may comprise viral particles. In embodiments, the expression vector comprises a modified viral polynucleotide, such as from an adenovirus, a herpesvirus, or a retrovirus, such as a lentiviral vector. In embodiments, a sequence encoding the described fusion protein and/or a guide RNA may be integrated into a chromosome of the same cell in which aneuploidy is induced.

In embodiments, one or more components of the described systems may be delivered to cells using, for example, a recombinant adeno-associated virus (rAAV) vector or a lentiviral vector. In embodiments, non-viral delivery systems may be used for introducing one or more of the components of the described system. Non-viral tools including hydrodynamic injection, electroporation and microinjection. In embodiments, and as described further below, more than one guide RNA can be used. In embodiments, the disclosure includes combining pairs of centromeric sgRNAs for use in a single cell. The guide RNAs used in the disclosure may be fully processed, or subjected to a processing step before they are used.

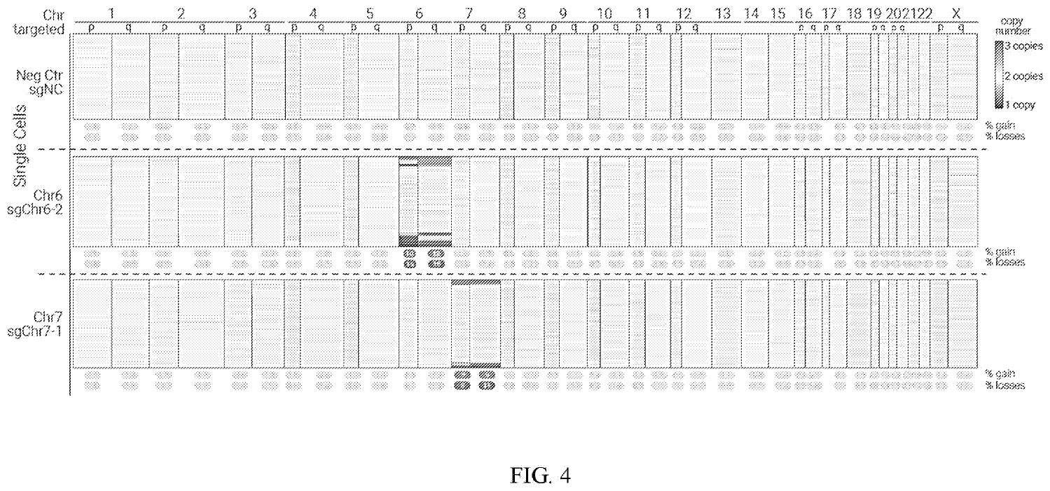

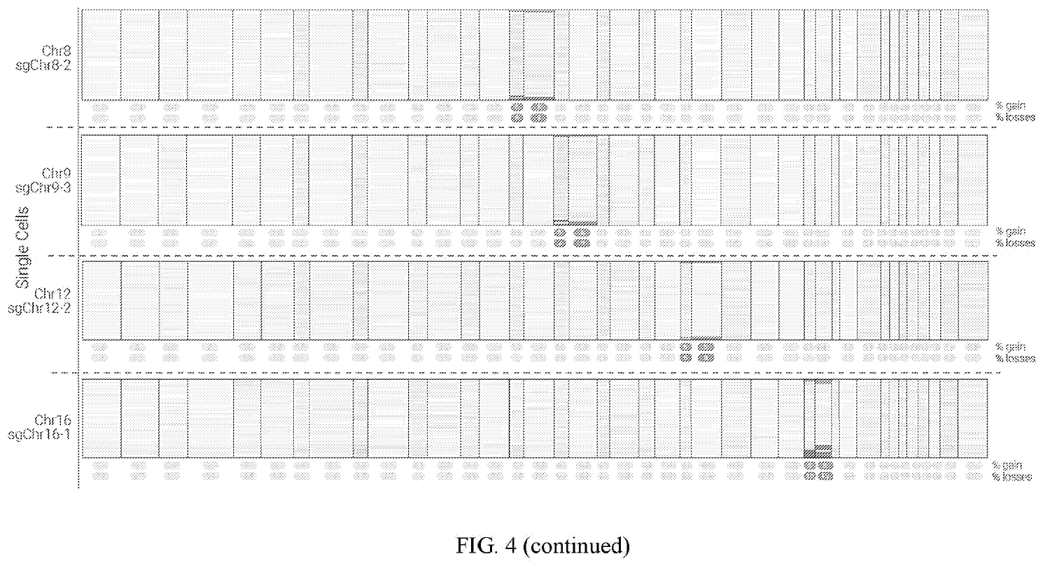

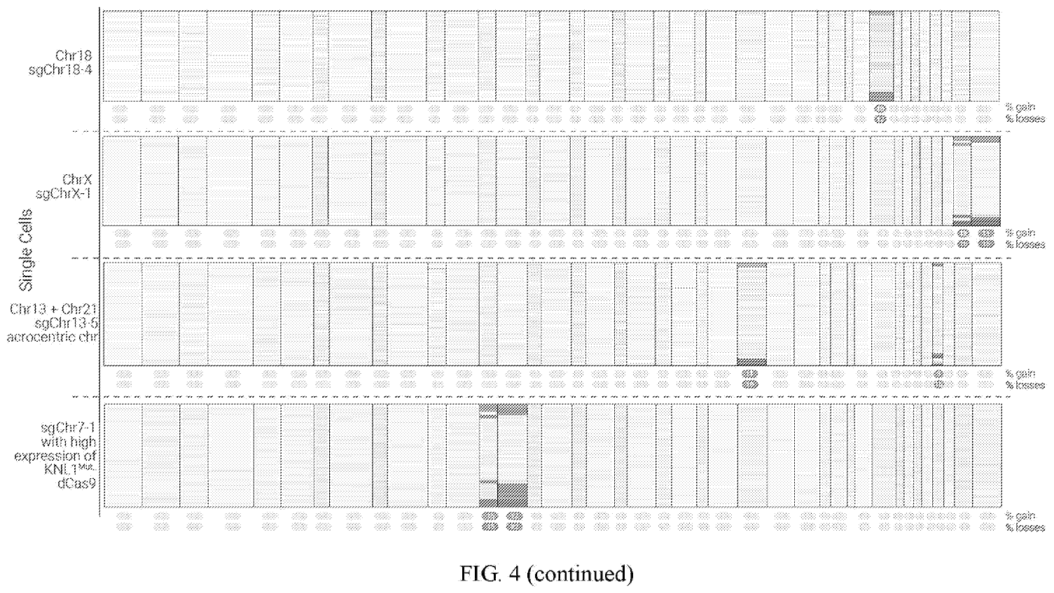

The gRNA binding sequences are provided in Table S1 (SELECTED_gRNAs) as DNA sequences. The disclosure expressly includes each DNA sequence in the form of RNA wherein each T is replaced by a U. This table contains all the gRNA binding that were tested and contains information on which gRNAs were validated by imaging through visualization of the centromeres. Furthermore, a subset of these gRNAs validated by imaging was also validated using scRNAseq and KaryoCreate as shown in FIG. 4. gRNAs normally are 20 bp long. In one embodiment, 19-bp 18-bp or 17-bp version of the gRNAs (omitting the first one, two or three base pairs) can be utilized to increase the proportion of whole chromosome (versus chromosome arms) events and gains events.

For 21 out of 24 chromosomes, we computationally predicted unique sgRNAs binding ≥400 times at the centromere with a specificity of 99%. Using KaryoCreate, we demonstrated the successful induction of chromosome-specific aneuploidy for 10 chromosomes tested. In principle, KaryoCreate can be used for 21 out of 24 chromosomes, with the exception of chromosomes sharing similar centromeric sequences such as acrocentric chromosomes.

However, the disclosure demonstrates that induction of gains and losses for the remaining chromosomes is still possible by using sgRNAs targeting both the chromosome of interest and other chromosomes sharing centromeric sgRNA binding sites (instead of single chromosomes). Furthermore, the disclosure demonstrates production of two highly recurrent aneuploidies in human gastro-intestinal cancers (chromosome 7 gain and 18q loss), and provides data supporting tumor-associated phenotypes associated with chromosome 18q loss in colorectal cells, as discussed in the Examples below.

The following Examples are intended to illustrate but not limit the disclosure.

### Example 1

Computational prediction of sgRNAs targeting chromosome-specific α-satellite centromeric repeats.

To design chromosome-specific centromeric sgRNAs, the genome assembly from the Telomere-to-Telomere (T2T) consortium²⁹ was referred to. For centromeres resolved in previous assemblies, we confirmed the sgRNA predictions from T2T using the hg38 reference genome³⁰, to reduce the risk of bias associated with a single assembly^{31,32}. To increase the likelihood of interfering with chromosome segregation, we focused the design on centromeric HORs found to bind to CENPA in chromatin immunoprecipitation (ChIP) experiments (defined as “Live”, or HOR_L, by the T2T)^{21,33}. For any given chromosome, a preferred sgRNA has 1) high on-target specificity (i.e. does not bind to centromeres on other chromosomes or to other genomic locations), 2) high number of binding sites on the repetitive HOR_L and 3) high efficiency in tethering dCas9 to the DNA. For each chromosome, we started by identifying all possible Cas9 sgRNAs targeting its HOR_L. We performed this analysis for all 24 human chromosomes (Tables S1, S2).

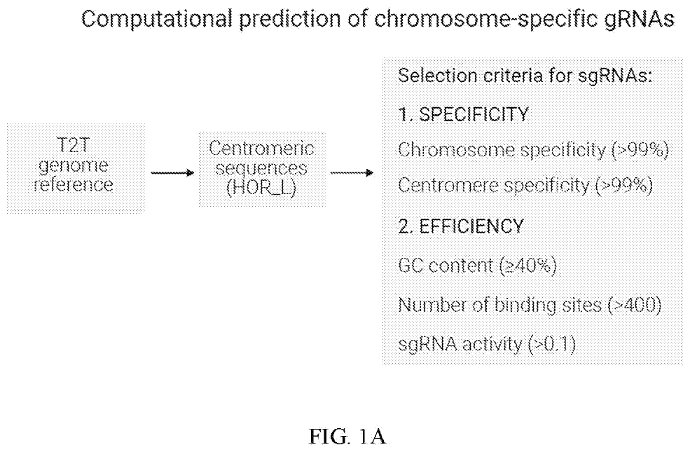

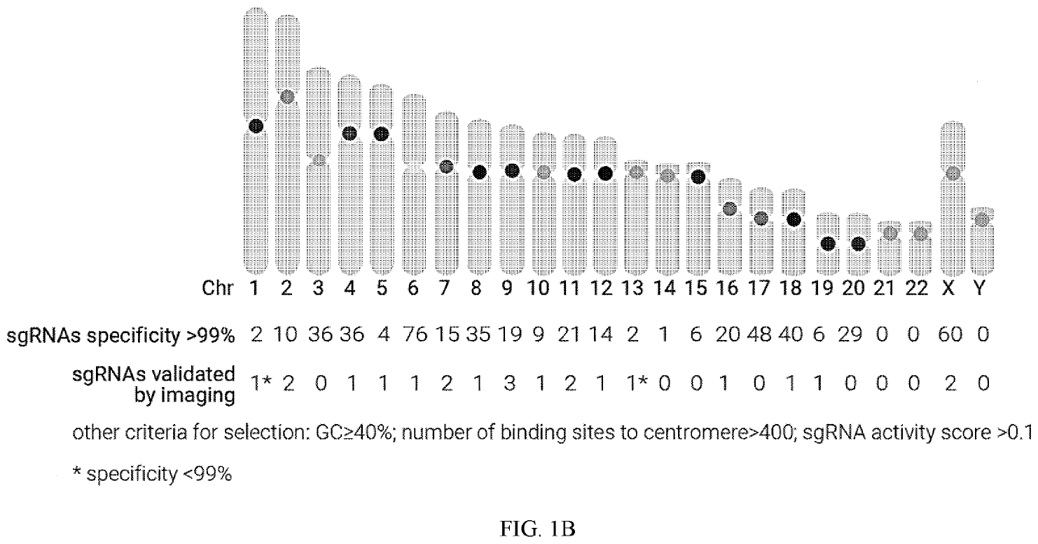

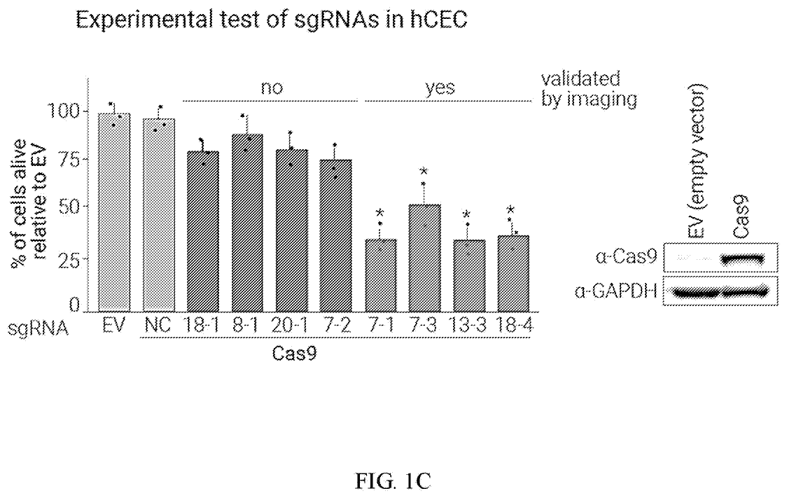

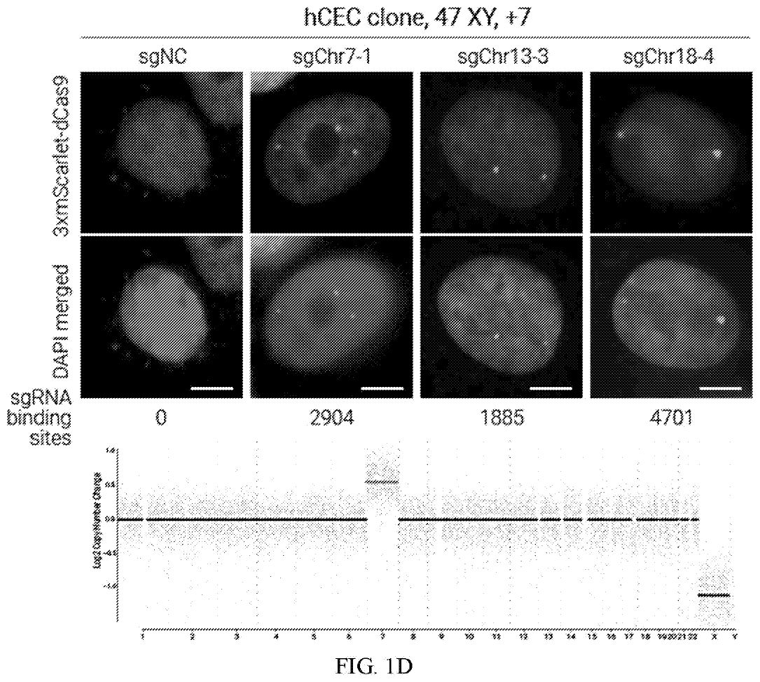

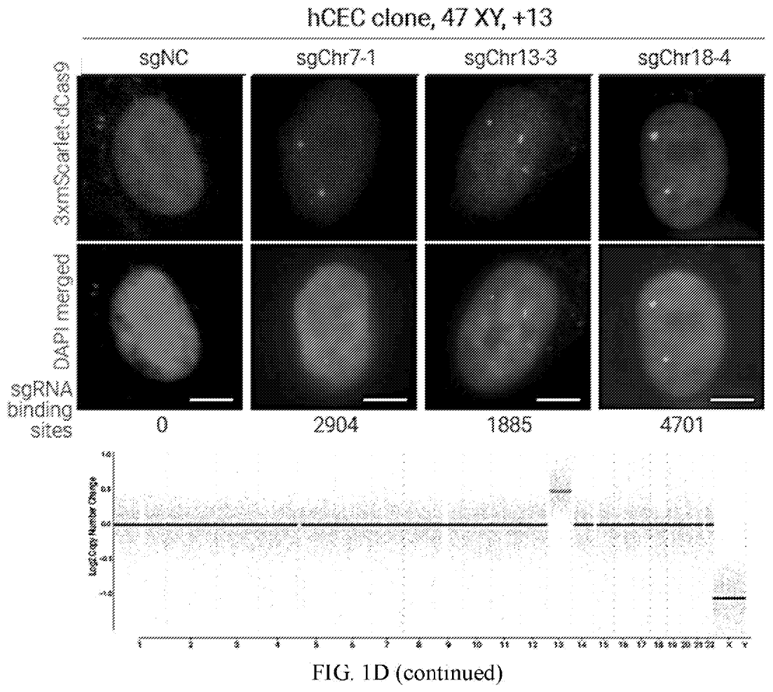

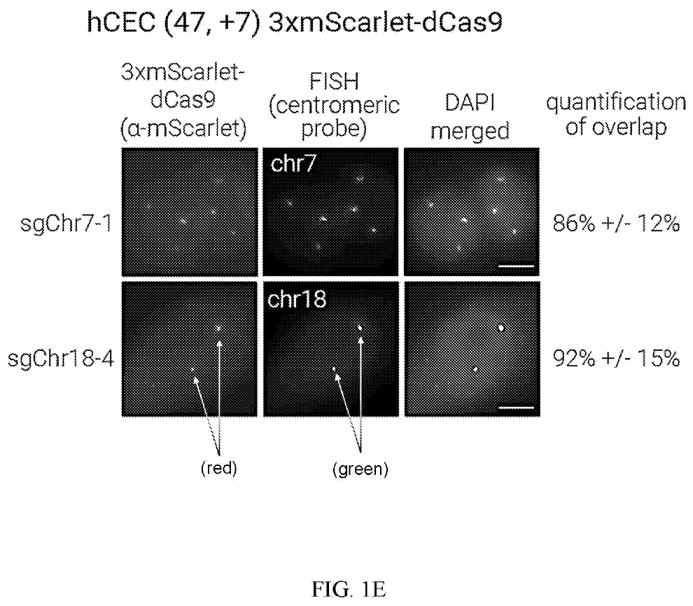

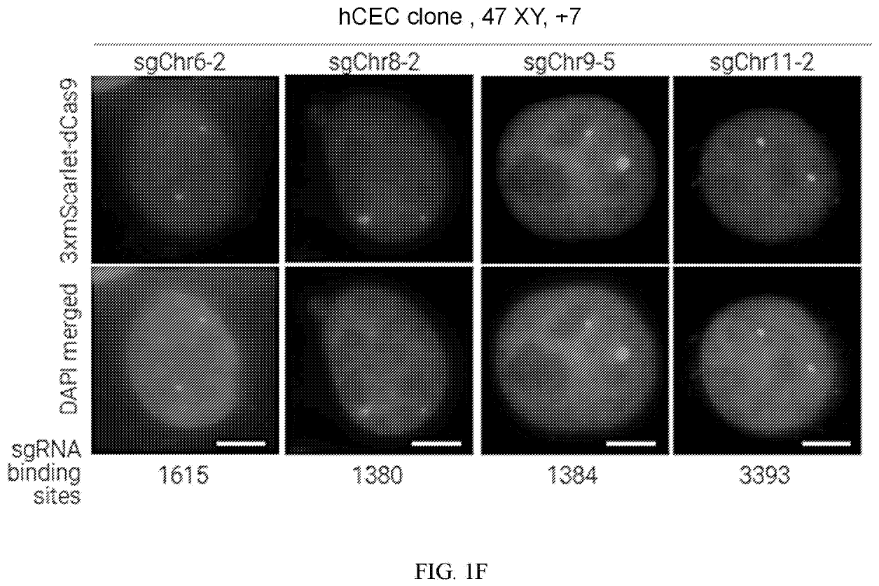

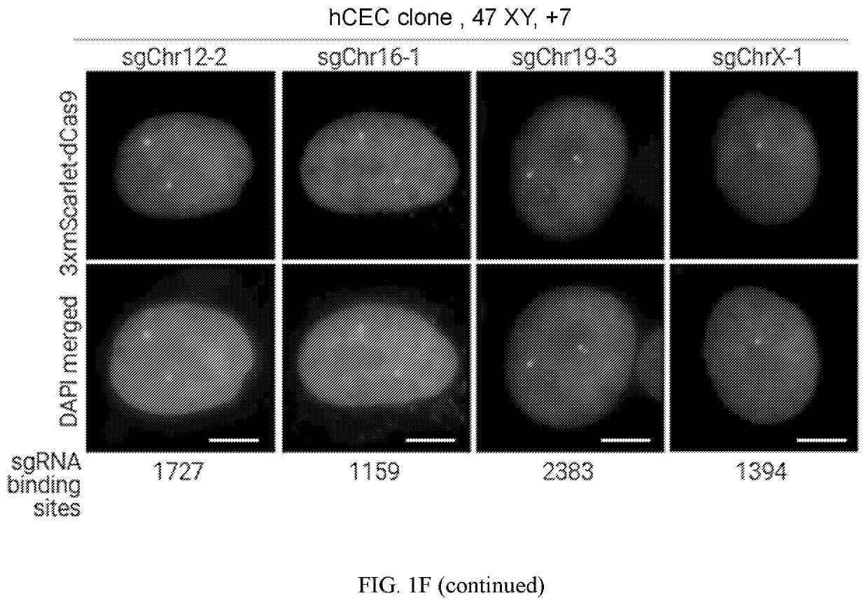

Next, we determined two parameters that define the specificity and efficiency of each sgRNA (both percentages, with 100% the best score): a chromosome specificity score, defined as the ratio of the number of binding sites on the target centromere to the total number of binding sites across all centromeres, and a centromere specificity score, defined as the ratio of the number of binding sites in centromeric regions to the number of sites across the whole genome. We also predicted the efficiency of each sgRNA based on GC content³⁴ sgRNA activity (see Methods), and total number of binding sites to the specific centromere (FIG. 1A).

Using thresholds of 99% for both chromosome and centromere specificity scores, a GC content ≥40%, a minimum of 400 sgRNA binding sites, sgRNA activity^{35,36}>0.1, and representation in hg38, we designed at least one sgRNA for 21 of the 24 human chromosomes (all except 21, 22, Y; FIG. 1B; Table S1), with 1590 binding sites per chromosome on average. Increasing the chromosome specificity score from 99% to 100% resulted in at least one sgRNA for 16 chromosomes.

### Example 2

Experimental validation of sgRNAs targeting α-satellite centromeric repeats on 15 human chromosomes.

To assess the activity of the predicted sgRNAs, we co-expressed selected sgRNAs with Cas9 and monitored cell proliferation, since the presence of several double-strand breaks at the centromere is likely to decrease cell viability³⁷. We used hTERT TP53^{-/-} human colonic epithelial cells (hCECs)³⁸ and hTERT TP53 WT retinal pigment epithelial cells (RPEs) expressing p21 ((DKN1A) and RB (RB1) shRNAs³⁹. We transduced Cas9-expressing RPEs and hCECs with a lentiviral vector expressing either a centromeric or a negative control sgRNA (sgNC) that does not target the human genome⁴⁰. Hereafter we refer to each centromeric sgRNA as sgChrα-β, where α is the specific targeted chromosome and β is the serial number of the designed sgRNA.

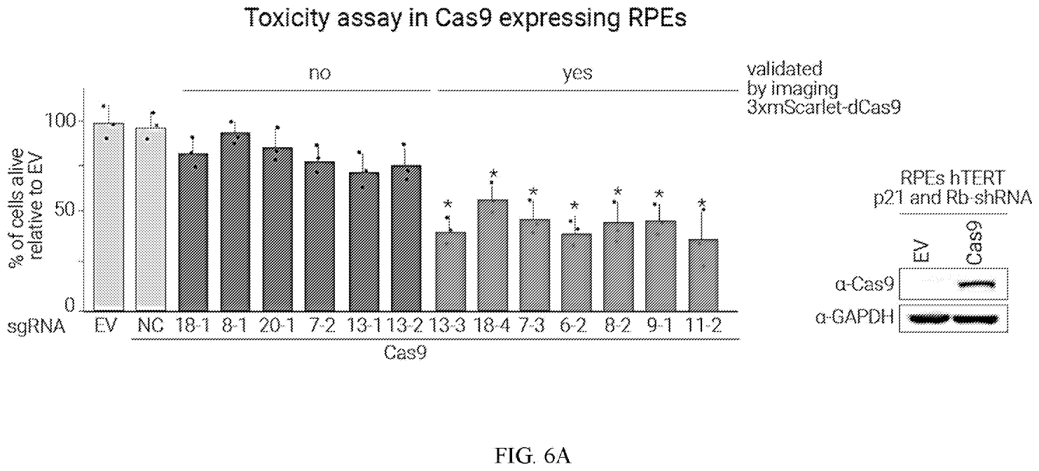

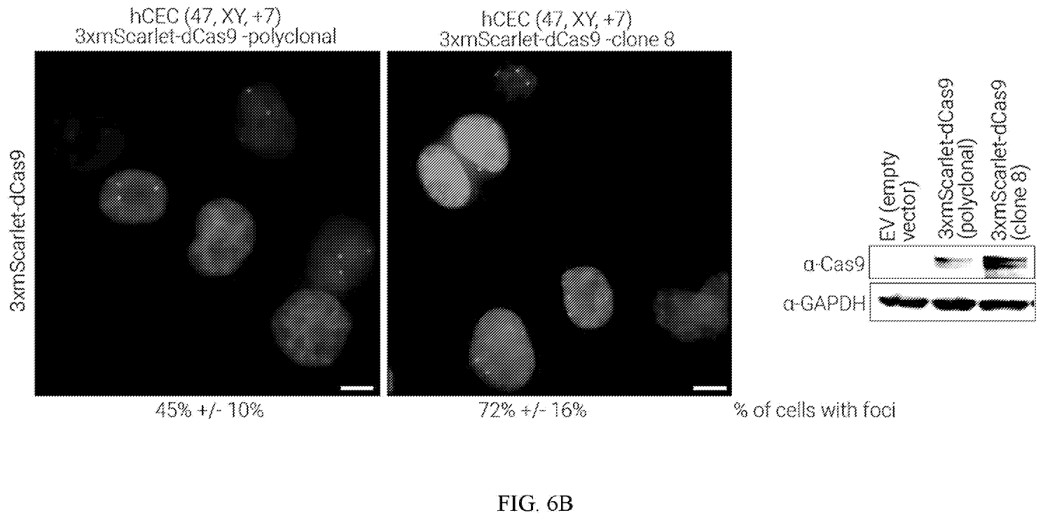

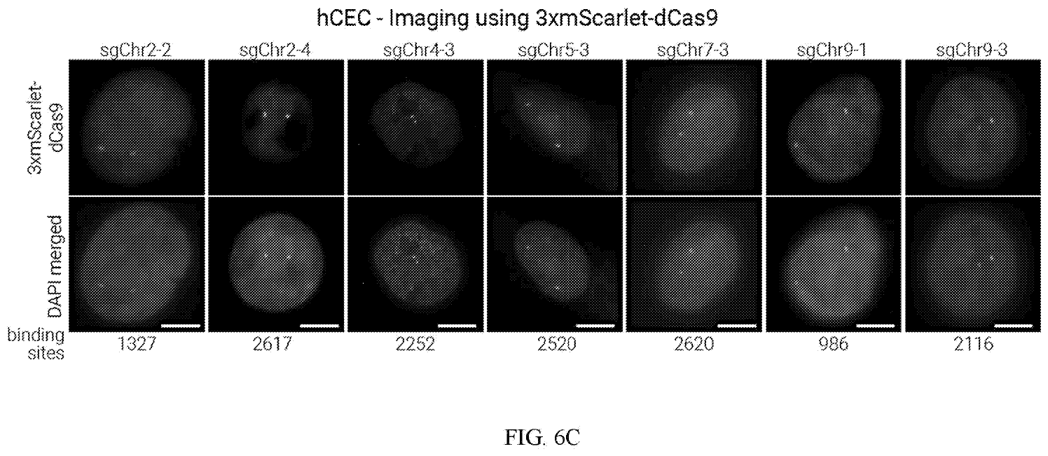

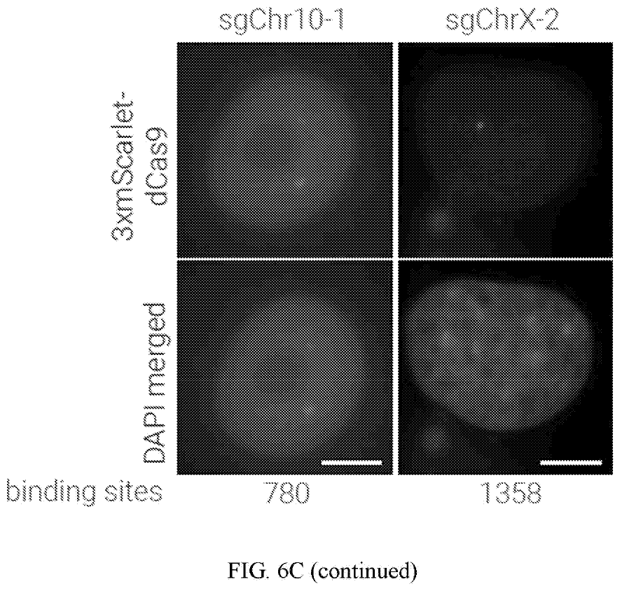

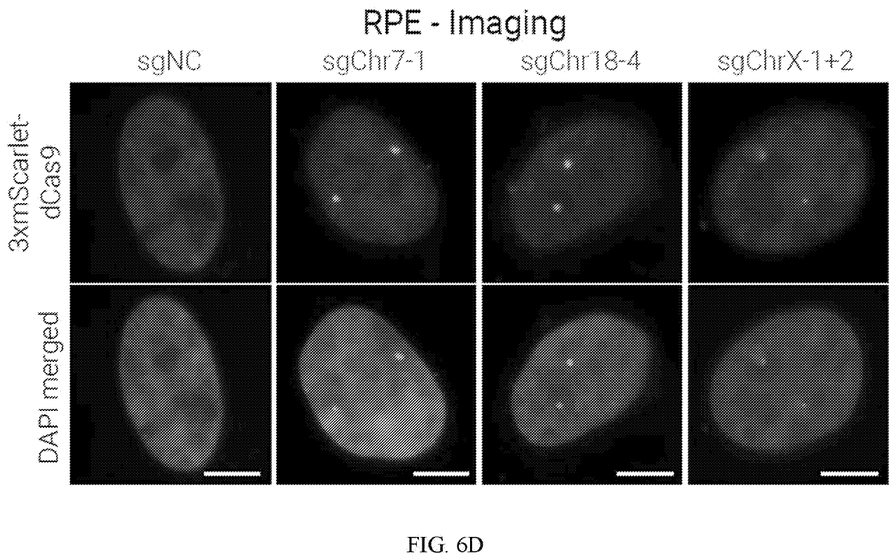

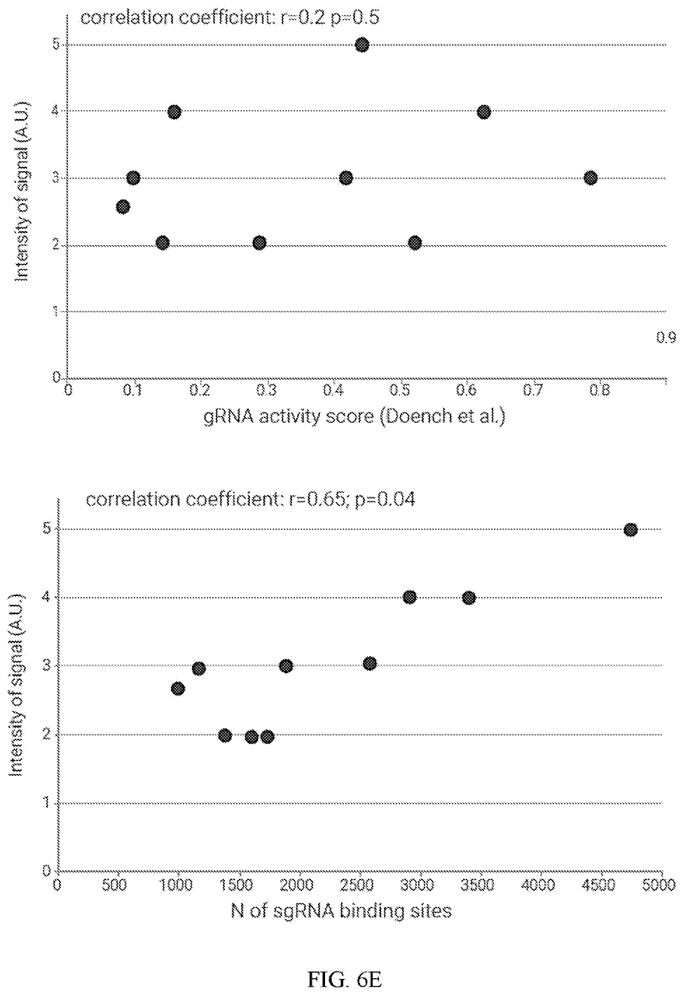

We first tested 3 sgRNAs predicted for chromosomes 7 and 13, and 4 for chromosome 18. Compared to sgNC, hCECs and RPEs expressing sgChr7-1, sgChr7-3, sgChr13-3, or sgChr18-4 exhibited at least 50% reduction in proliferation, while the other sgRNAs did not result in significant differences (FIG. 1C; FIG. 6A). We selected the sgRNAs exhibiting the greatest reduction in proliferation for additional testing.

To confirm that the sgRNAs targeted the intended centromeres, we designed a dCas9-based imaging system comprising three mScarlet fluorescent molecules fused to the N-terminus of endonuclease-dead Cas9 (3×mScarlet-dCas9). To achieve consistently high expression, we FACS-sorted 3×mScarlet-dCas9-transduced hCECs for strong fluorescent signal. hCECs co-expressing 3×mScarlet-dCas9 and sgChr7-1, sgChr13-3, or sgChr18-4 (but not sgNC) showed bright nuclear foci (FIG. 1D). Notably, the sgRNAs that did not cause a decrease in proliferation in the presence of Cas9 failed to form foci (FIG. 1C and data not shown).

To further confirm the chromosome specificity of the sgRNAs, we used two independent approaches. We first utilized hCEC clones with aneuploidies previously identified through whole-genome sequencing (WGS)-based copy number analysis to verify whether the observed number of foci was consistent with the expected DNA copy number. We found that hCEC clones carrying three copies of chromosome 7 or 13 each showed three foci when transduced with sgChr7-1 or sgChr13-3, respectively (FIG. 1D; FIG. 6B). Transduction with sgRNAs targeting chromosomes present in two copies led to the formation of two foci per nucleus (FIG. 1D). Next, we confirmed that the 3×mScarlet-dCas9 foci localized at specific centromeres by fluorescence in-situ hybridization (FISH) using centromeric probes. We confirmed colocalization of FISH signals for both chromosomes 7 (sgChr7-1) and 18 (sgChr18-4) with mScarlet foci (FIG. 1E). Altogether, these experiments indicate that the computationally predicted sgRNAs can recruit dCas9 to the expected specific centromere.

We tested 75 additional sgRNAs in hCECs and confirmed the formation of the expected number of foci for 24 sgRNAs targeting 15 different chromosomes (2, 4, 5, 6, 7, 8, 9, 10, 11, 12, 13, 16, 18, 19, X; FIG. 1F, FIG. 6C, Table S1). We also confirmed 4 sgRNAs in RPEs (FIG. 6D).

Altogether, we designed and validated 24 chromosome-specific sgRNAs targeting the centromeres of 15 different human chromosomes. Interestingly, the predicted sgRNA efficiency evaluated using a previously published algorithm³⁶ did not correlate with the ability of sgRNAs to form foci (r=0.2; p=0.5; FIG. 6E, top). Instead, for the sgRNAs that formed foci, there was a significant correlation between the intensity of the signal of the foci and the number of binding sites at the centromeres predicted based on the CHM13 genome reference (r=0.65, p=0.03; FIG. 6E, bottom).

### Example 3

Centromeric targeting of KNL1^{Mut}-dCas9 induces modest mitotic delay and chromosome missegregation.

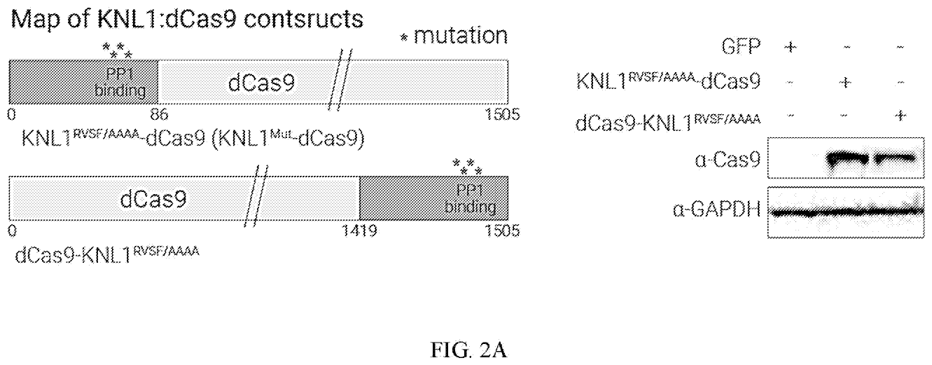

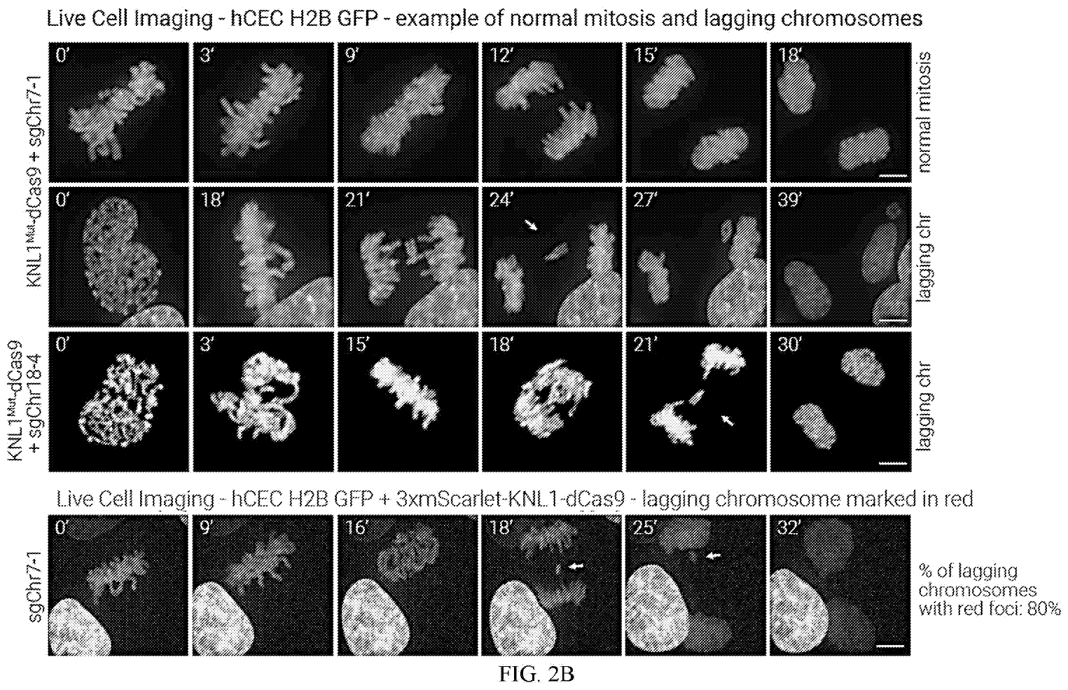

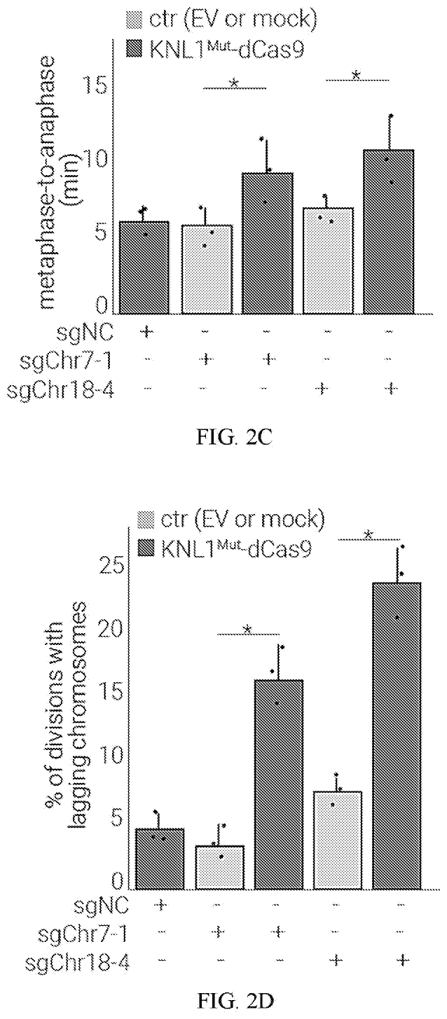

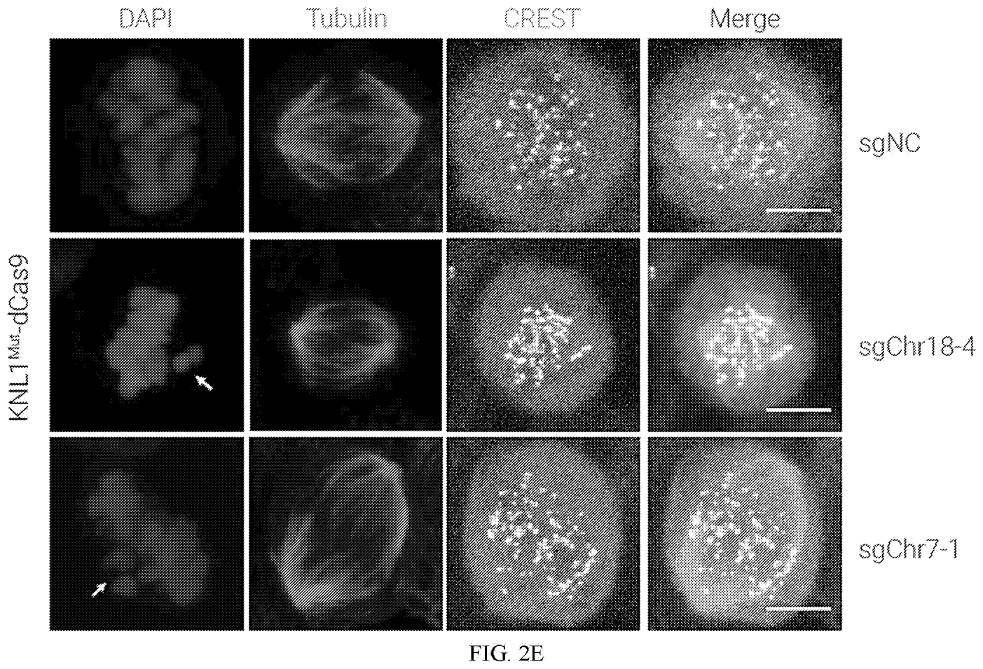

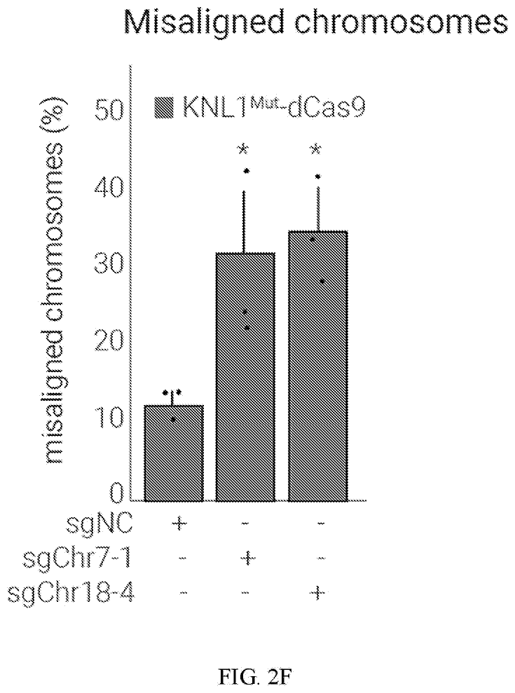

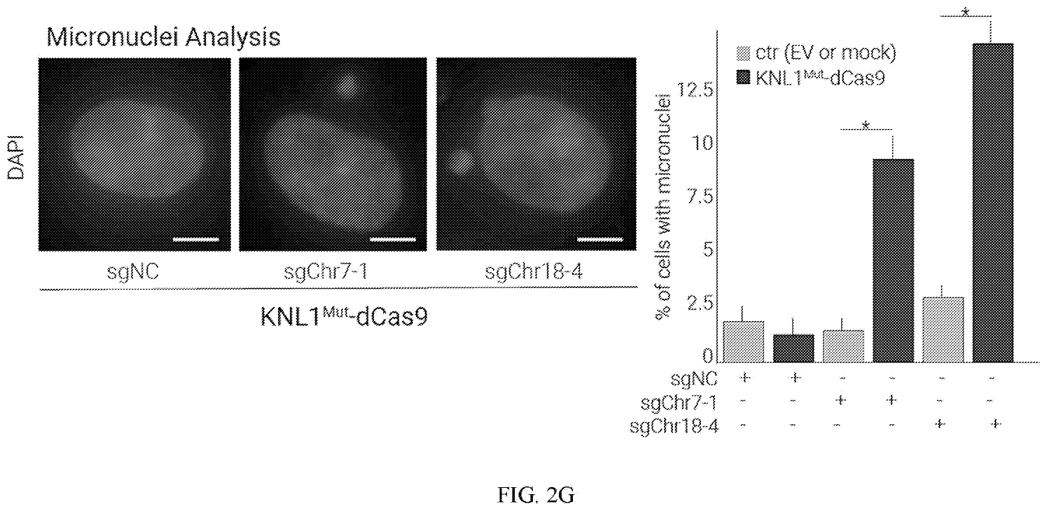

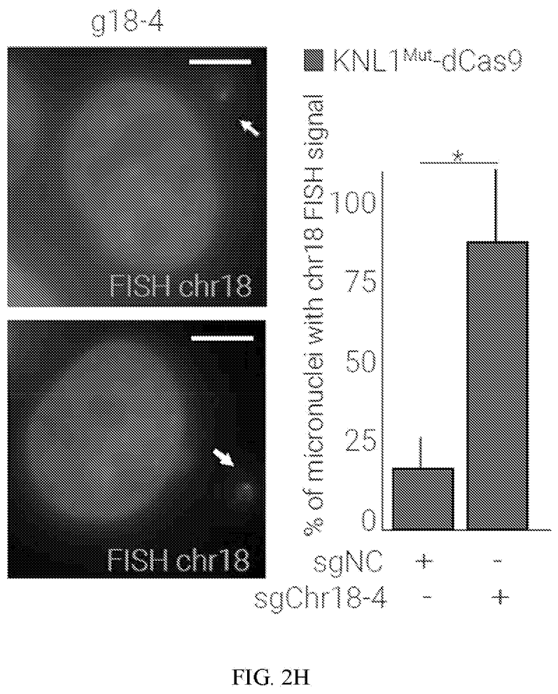

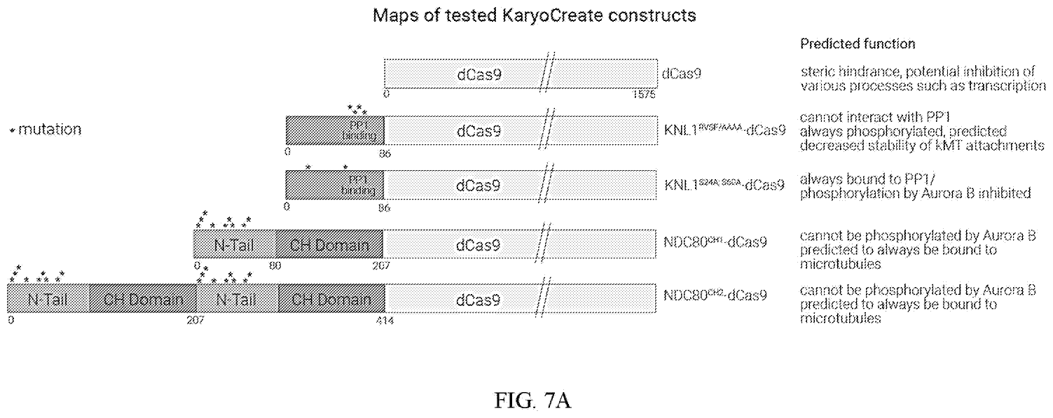

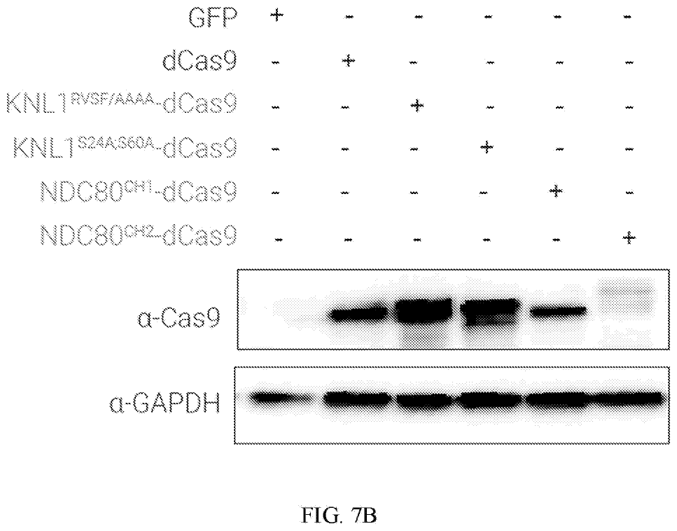

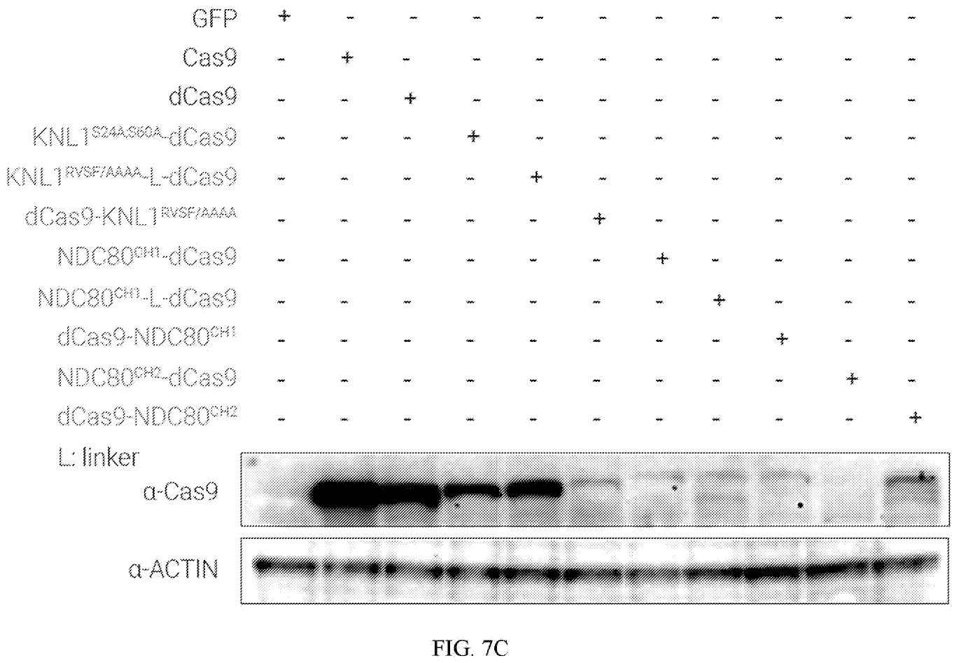

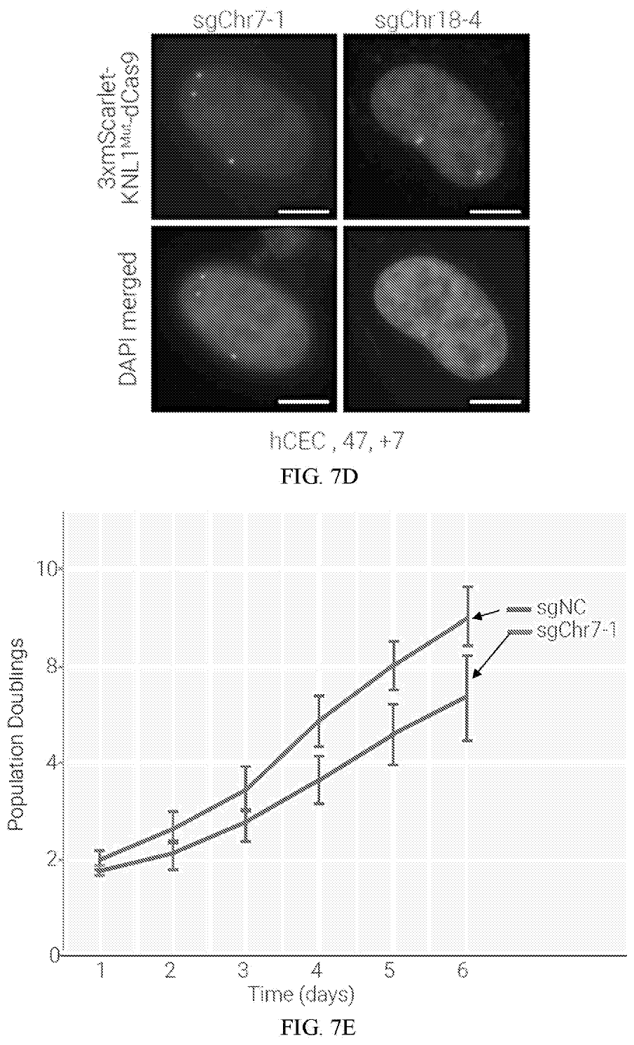

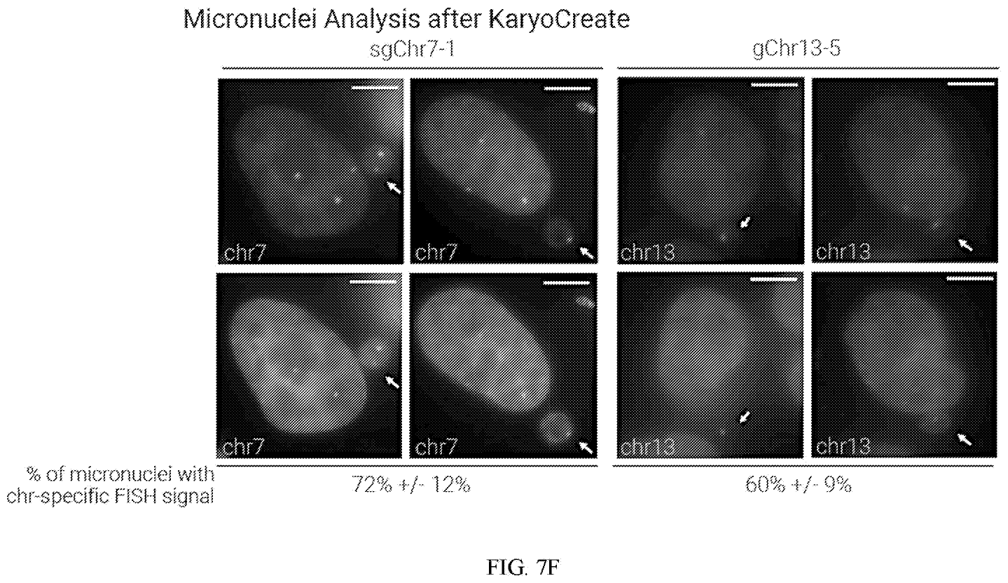

To induce chromosome missegregation, we built and tested four dCas9 fusion proteins to determine if they could disrupt kinetochore-microtubule attachments (FIG. 2A, FIG. 7A). KNL1^{S24A:S60A}-dCas9 and KNL1^{RVSF/AAAA}-dCas9 utilize the KNL1 N-terminal portion (amino acid (aa) 1-86)^{28,41} and contain mutations with opposing effects in disrupting the cross-regulation between Aurora B and PP1 (FIG. 7A). KNL1^{S24A:S60A} was predicted to be always bound to PP1 as its mutated residues cannot be phosphorylated by Aurora B⁴¹ (FIG. 7A); KNL1^{RVSF/AAAA} contains a mutation affecting the RVSF motif (aa 58-61) preventing it from interacting with PP1 and recruiting it to the centromere²⁸ (FIG. 7A). NDC80-CH1-dCas9 and NDC80-CH2-dCas9 were designed to render the interaction between kinetochores and microtubules hyperstable and refractory to Aurora B destabilization. These constructs contain one (NDC80-CH1) or two (NDC80-CH2) CH domains (aa 1-207), the region of NDC80 responsible for binding microtubules. CH domains normally contain 6 residues whose phosphorylation by Aurora B inhibits the interaction with microtubules; our constructs have all 6 residues mutated, preventing Aurora-B-mediated regulation⁴² (FIG. 7A).

Western blot analysis showed that KNL1^{RVSF/AAAA}-dCas9 and KNL1^{S24A:S60A}-dCas9 expression levels were higher than those of NDC80-CH1-dCas9 and NDC80-CH2-dCas9 (FIG. 7B). For the KNL1 constructs, the N-terminal fusions were generally more stable than the C-terminal fusions (FIG. 7C, FIG. 2A). Given their higher protein expression and greater efficiency in inducing chromosome gains and losses compared to the other constructs, we focused on the KNL1 constructs, particularly KNL1^{RVSF/AAAA}-dCas9, also referred to herein as KNL1^{Mut}-dCas9.

To confirm centromeric localization of the fusion protein, we transduced hCECs expressing a fluorescently tagged version of KNL1^{Mut}-dCas9 (3×mScarlet-KNL1^{Mut}-dCas9) with centromeric sgRNAs, as described above. We observed the expected number of foci in the presence of sgChr7-1 and sgChr18-4 (FIG. 7D), indicating that fusing KNL1^{Mut} with dCas9 does not alter the ability of dCas9 to be recruited to centromeres. Next, using live-cell imaging, we examined the effect of KNL1^{Mut}-dCas9 on mitosis duration and chromosome segregation. hCECs constitutively expressing GFP-tagged histone H2B were transduced with KNL1^{Mut}-dCas9 or empty vector (EV) and with sgChr7-1, sgChr18-4, or sgNC. Cells expressing KNL1^{Mut}-dCas9 and either sgChr7-1 or sgChr18-4 progressed more slowly through mitosis than cells transduced with EV and either sgChr7-1 or sgChr18-4 (FIG. 2C): the average time spent in the metaphase-to-anaphase transition increased from 6 minutes to 9 or 10 minutes in the sgChr7-1 or sgChr18-4 condition, respectively (FIG. 2B, 2C). Nonetheless, cells transduced with sgChr7-1 or sgChr18-4 did not arrest in metaphase and completed mitosis, and their proliferation rate was only slightly and non significantly lower than that of cells transduced with sgNC (FIG. 7E). The number of cell divisions with lagging chromosomes increased from <5% to 15% between EV+sgChr7-1 and KNL1^{Mut}-dCas9+sgChr7-1 and from 7% to 23% between EV+sgChr18-4 and KNL1^{Mut}-dCas9+sgChr18-4 (FIG. 2B, upper panel, 2D). Furthermore, live-cell imaging of cells expressing 3×mScarlet-KNL1^{Mut}-dCas9 and sgChr7-1, where mScarlet marks chromosome 7 as in FIG. 7D (polyclonal population), showed that about 80% of the lagging chromosomes observed during mitosis had red foci, consistent with chromosome-specific missegregation (FIG. 2B). In this experiment sgNC could not be used as a control as it did not cause foci formation.

To corroborate these data in a different cell line, we performed a similar experiment in the HCT116 (TP53 WT) colon cancer cell line, transducing them with KNL1^{Mut}-dCas9 and either sgNC, sgChr7-1, or sgChr18-4. Immunofluorescence for α-tubulin to visualize the mitotic spindle, CREST serum to visualize the centromeres, and DAPI to assess chromosome alignment showed that the percentage of mitoses with misaligned chromosomes increased from 12% in the sgNC samples to 32% and 35% in the sgChr7-1 and sgChr18-4 conditions, respectively (FIG. 2E, 2F).

Finally, we scored the fraction of KNL1^{Mut}-dCas9-expressing hCECs containing micronuclei (a well-known consequence of missegregation⁴³) 7-9 days after transduction with sgRNAs. The percentage of cells showing micronuclei increased from <2.5% for sgNC to 9% for sgChr7-1 and 14% for sgChr18-4 (FIG. 2G). Furthermore, FISH using a chr18 centromeric probe on cells co-expressing KNL1^{Mut}-dCas9 and sgChr18-4 showed that 85% of micronuclei had a FISH signal (FIG. 2H). We also confirmed this result for chromosomes 7 and 13 (FIG. 7F).

Altogether, these data indicate that tethering KNL1^{Mut}-dCas9 to the centromeres through chromosome-specific sgRNAs can induce chromosome misalignment, lagging chromosomes, modest mitotic delay, and formation of micronuclei containing the targeted chromosome without substantially affecting the rate of cell division.

### Example 4

KaryoCreate allows induction of chromosome-specific gains and losses in human cells.

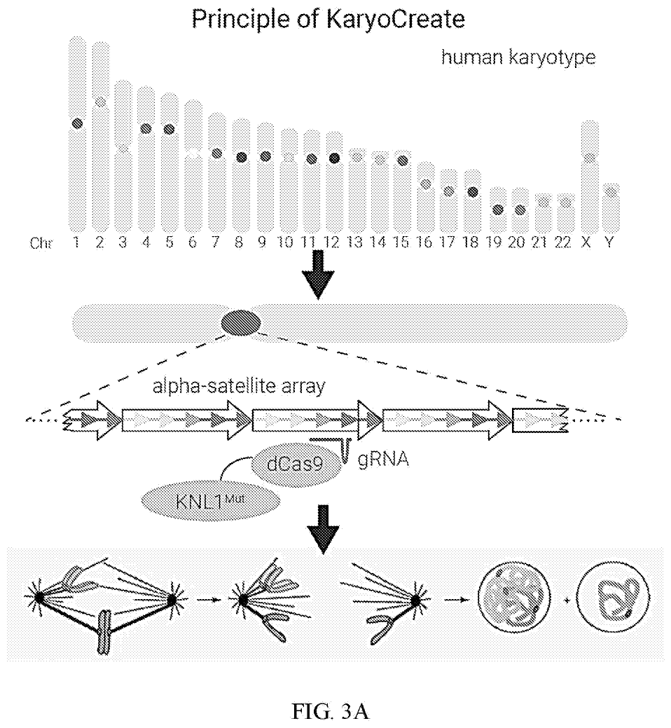

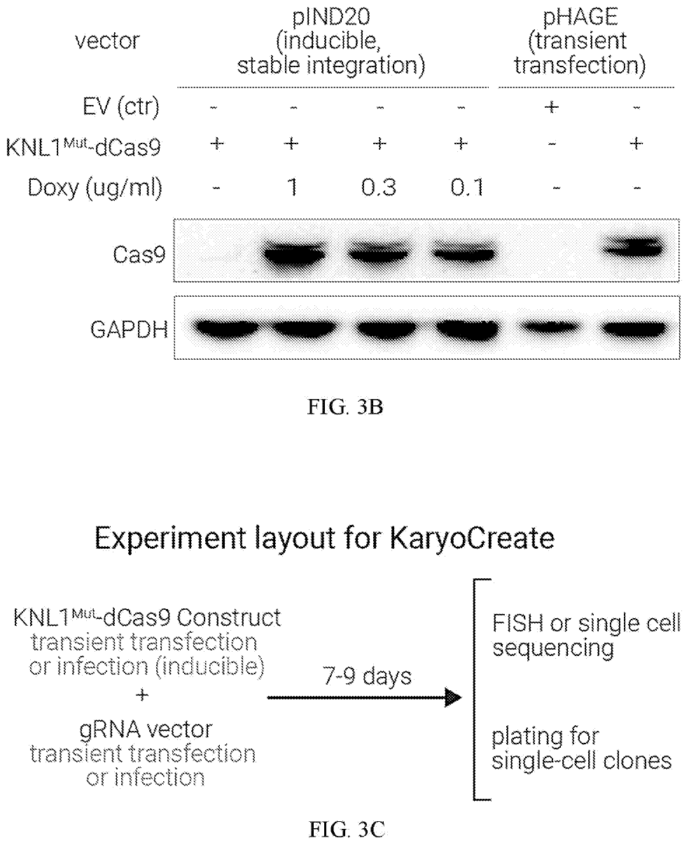

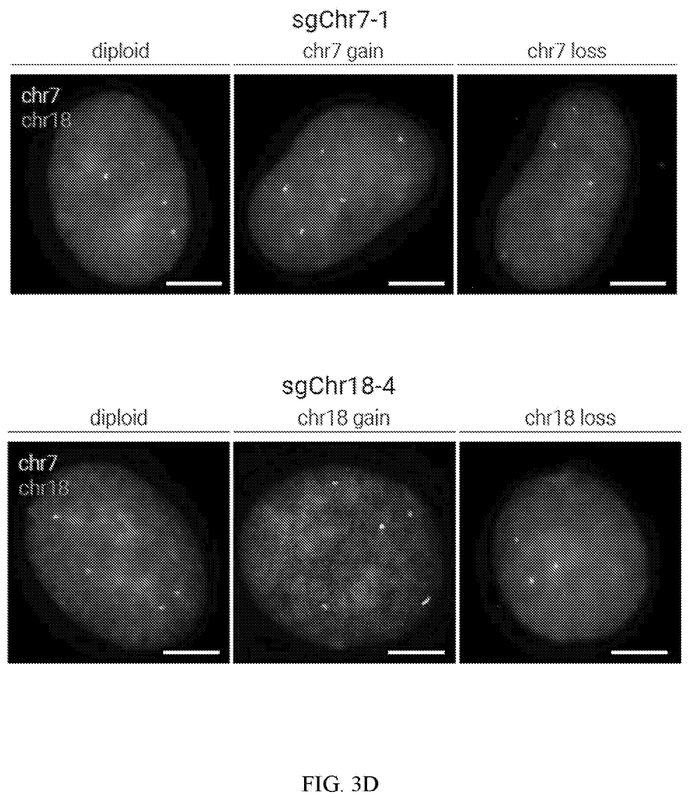

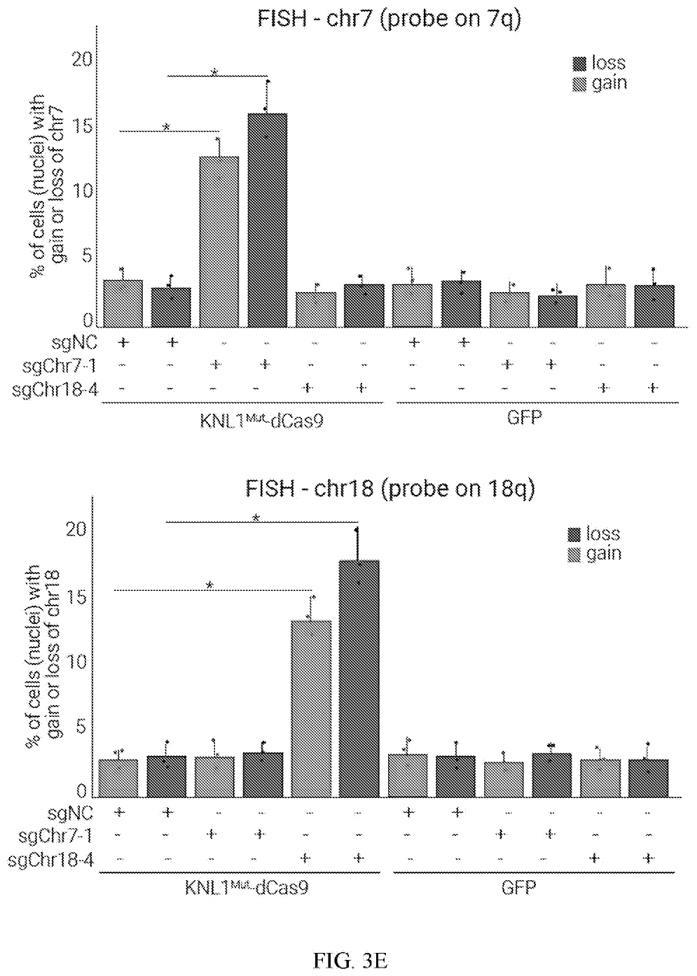

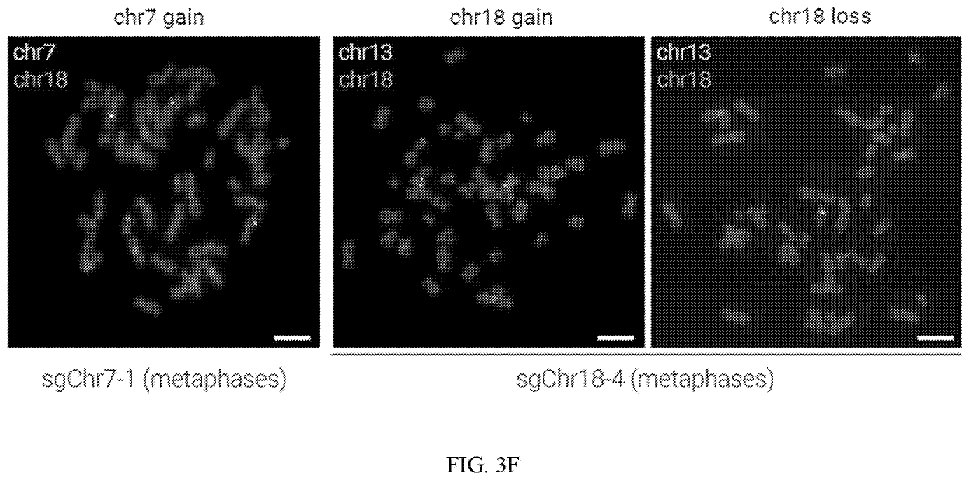

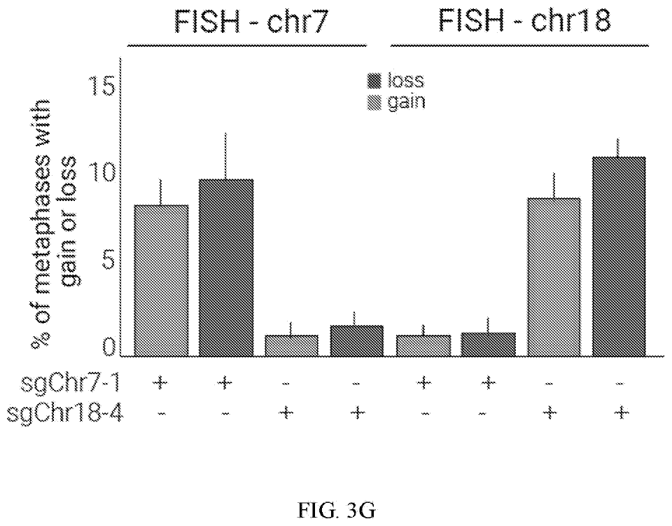

Having designed and validated chromosome-specific sgRNAs and dCas9-based constructs to induce chromosome missegregation, we next tested the capability of this system, designated “KaryoCreate” for Karyotype CRISPR Engineered Aneuploidy Technology, to generate specific aneuploidies in human cell lines (FIG. 3A). We reasoned that transient targeting of the dCas9-based construct to the centromere would generate chromosome gains and losses and allow isolation of stable aneuploid lines.

We first designed a system based on doxycycline-inducible expression of KNL1^{Mut}-dCas9 (constructed in the pIND20 vector⁴⁴) and constitutive sgRNA expression (pLentiGuide-Puro-FE, FIG. 3B, 3C; see Methods). We tested KaryoCreate in hCECs co-transduced with pIND20-KNL1^{Mut}-dCas9 or pIND20-GFP (control) and with sgNC, sgChr7-1, or sgChr18-4. Cells were treated with doxycycline for 7-9 days, and analyzed by FISH. 95% of control cells (GFP with sgNC) showed two copies of chromosomes 7 and 18 (FIG. 3D, 3E). This percentage did not significantly change in cells expressing KNL1^{Mut}-dCas9 and sgNC, indicating that in the absence of a centromere-specific sgRNA, KNL1^{Mut}-dCas9 does not induce chromosome missegregation (FIG. 3D, 3E; see Table S2 for automated quantification). Compared to sgNC, sgChr7-1 expression in hCECs transduced with KNL1^{Mut}-dCas9 significantly increased the percentages of cells showing chromosome loss, i.e. <2 copies (from 3% to 16%; p=0.01), or gain, i.e. >2 copies (from 2.8% to 12.5%; p=0.03), of chromosome 7, but not loss or gain of chromosome 18 (3% versus 3.2%). We next tested sgChr18-4, finding significant increases in loss (from 2% to 17.5%; p=0.01) and gain (from 2.5% to 14%; p=0.02) of chromosome 18 but not chromosome 7 (FIG. 3D, 3E; see Table S2 for automated quantification). Furthermore, we obtained comparable results when we restricted the FISH analysis to metaphase spreads as opposed to nuclei (FIG. 3F, 3G).

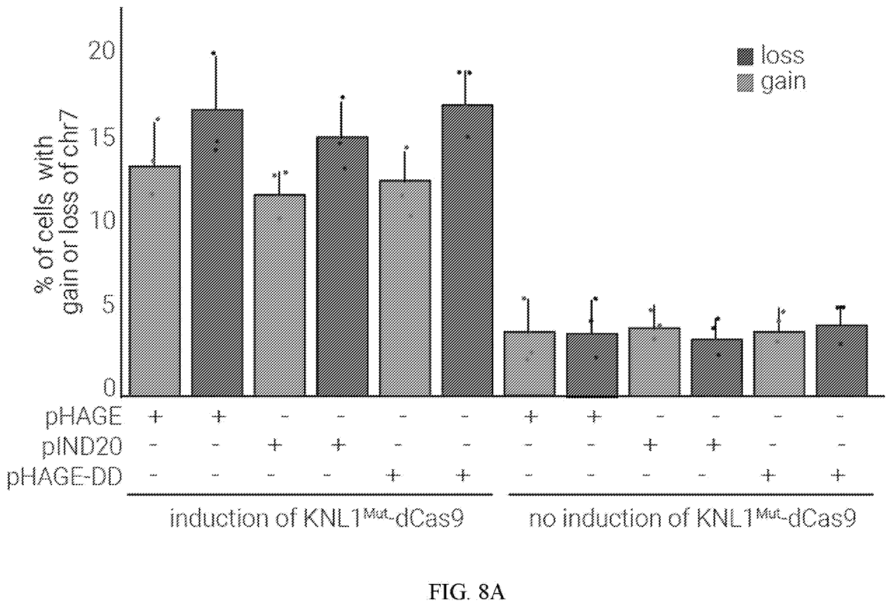

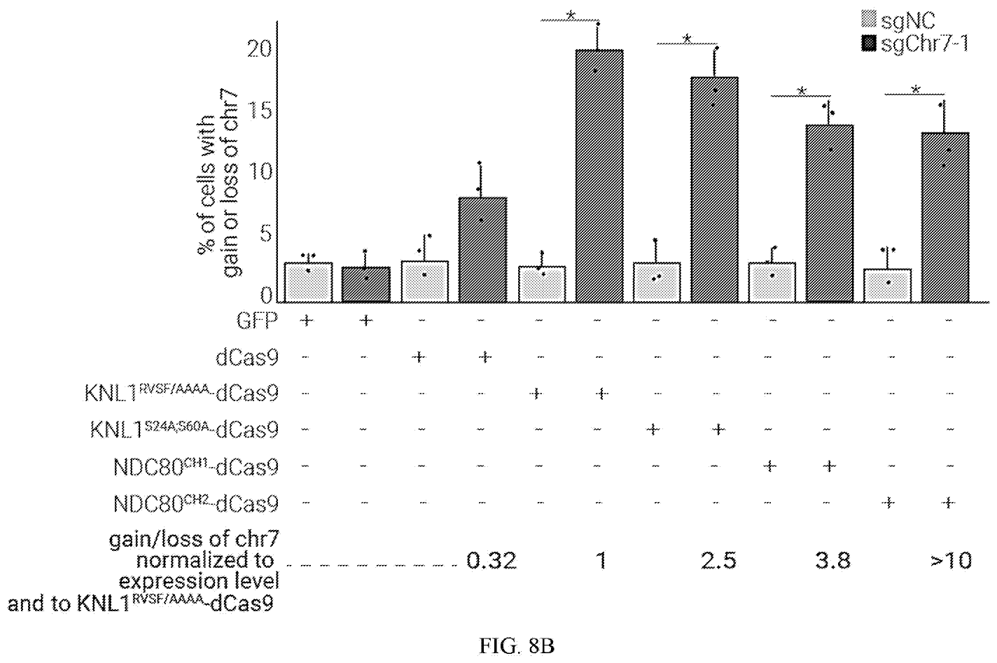

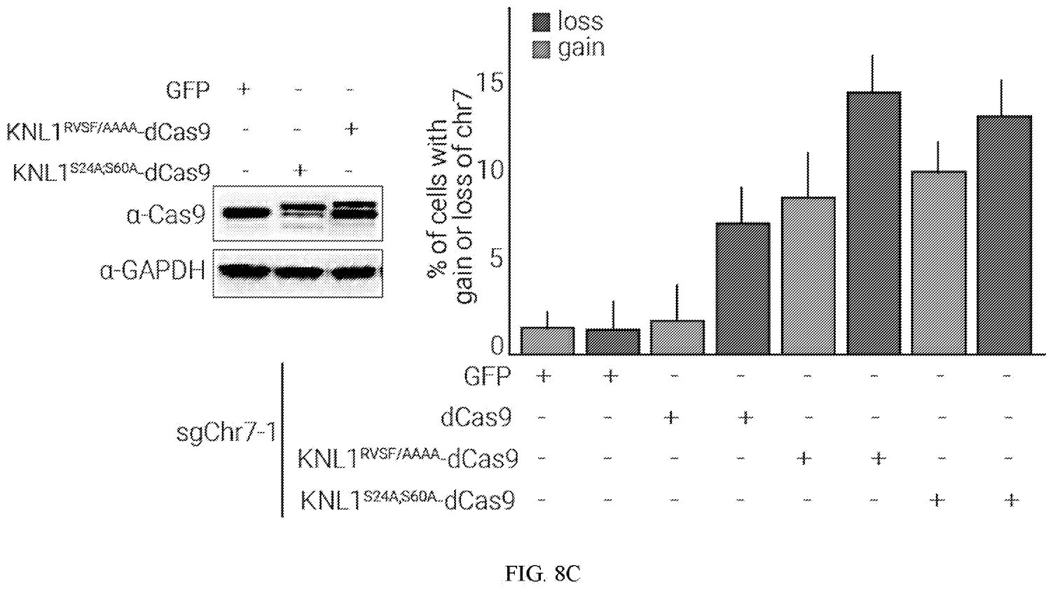

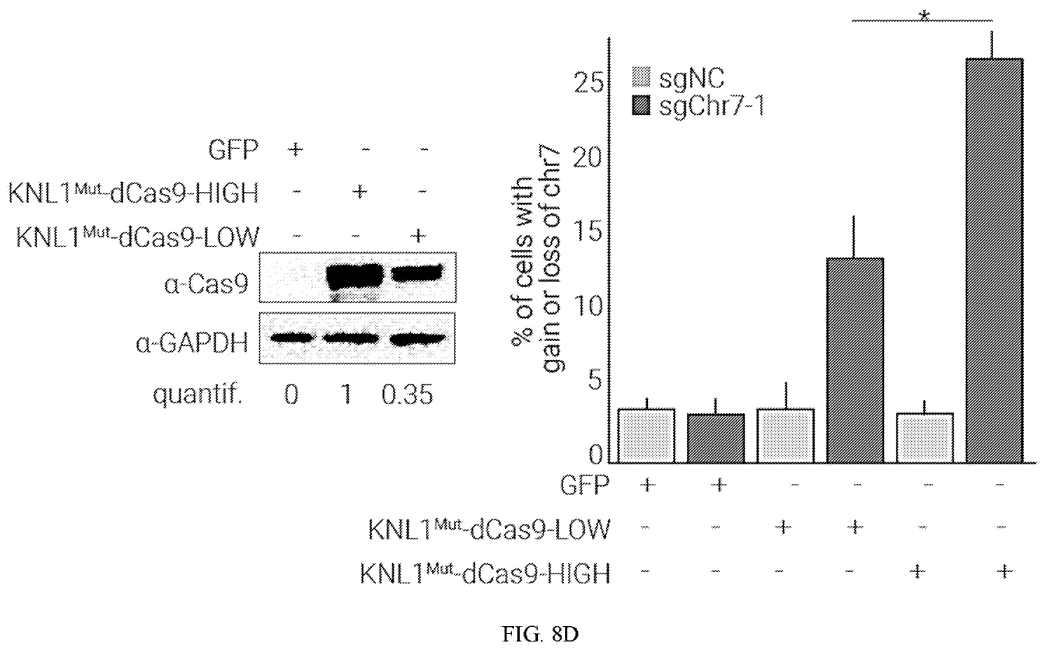

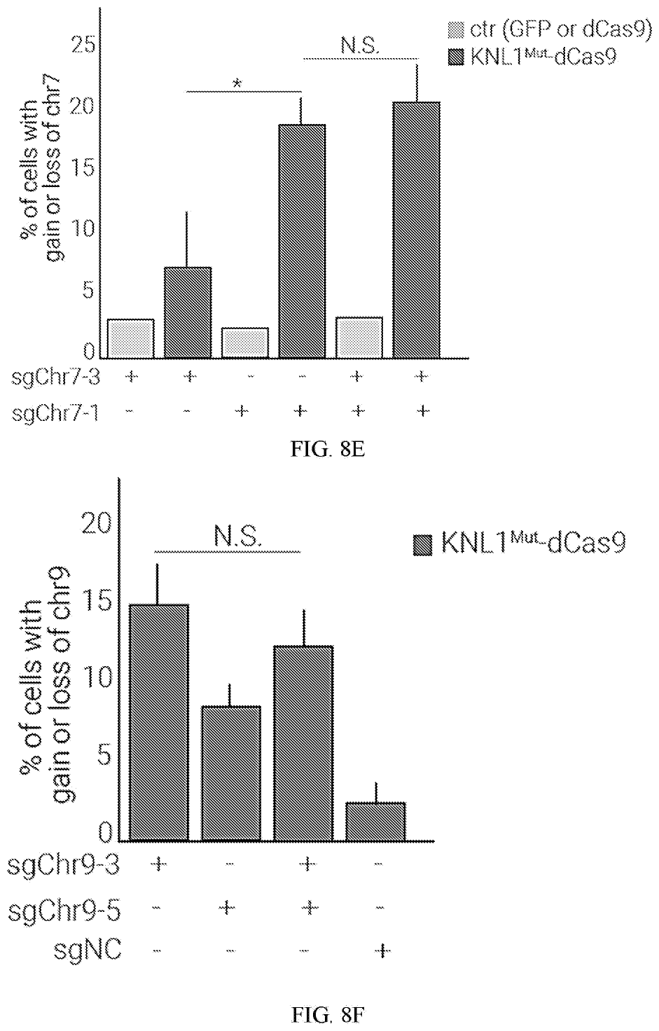

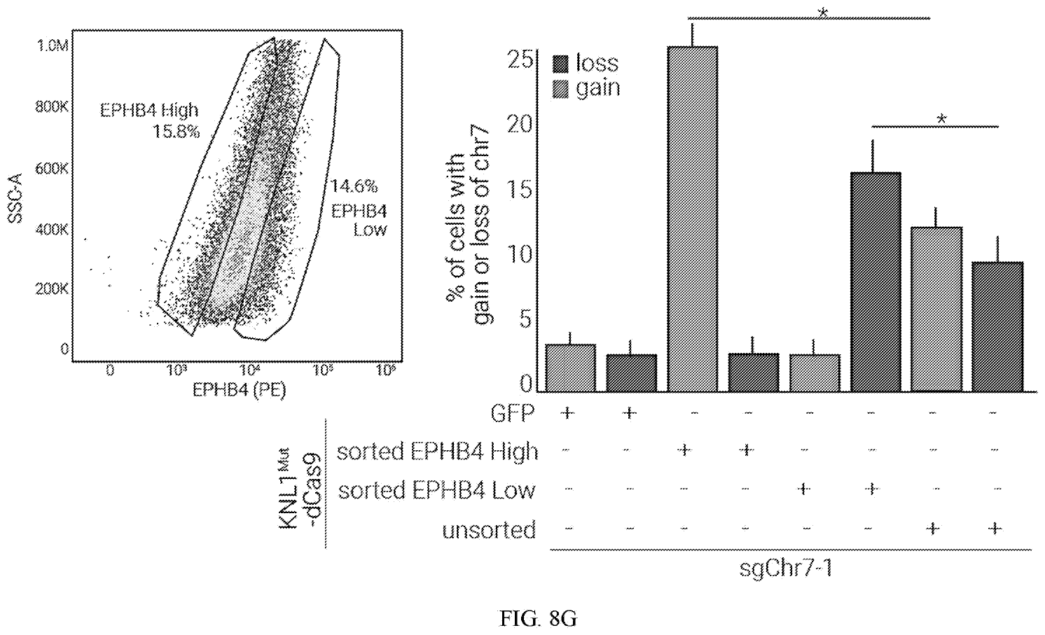

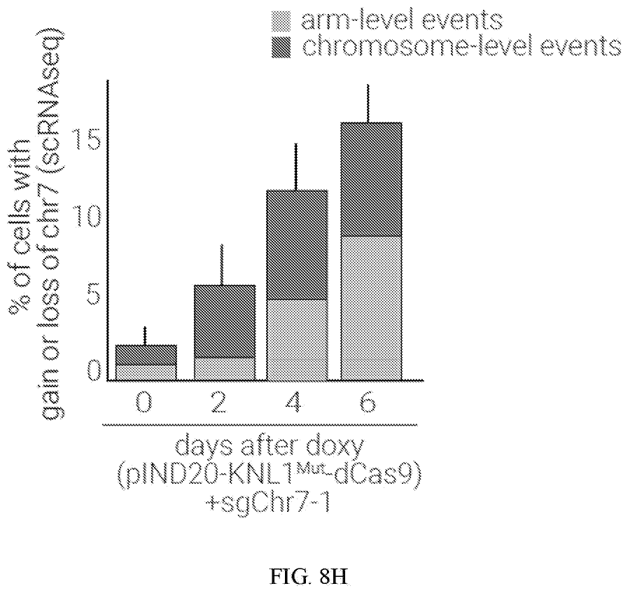

We also developed two additional KaryoCreate systems: one based on transient co-transfection of KNL1^{Mut}-dCas9 driven by a constitutive promoter (pHAGE vector) and an sgRNA-expressing vector (pLentiGuide-Puro-FE) and another based on a degrader approach whereby KNL1^{Mut}-dCas9 is fused to an FKBP-based degradation domain⁴⁵ and is stabilized only after treatment with the small molecule Shield-1 (see Methods). Overall, the three methods gave similar results (FIG. 8A).

We next analyzed the frequency of aneuploidy induced by other constructs generated for KaryoCreate (NDC80-CH1-dCas9 and NDC80-CH2-dCas9, described above; see FIG. 7A-7C, finding that the other fusion proteins induced aneuploidy with similar or lower efficiency than KNL1^{Mut}-dCas9 (KNL1^{RVSF/AAAA}-dCas9; FIG. 8B). KNL1^{S24A:S60A}-dCas9 produced similar levels of induced aneuploidy to KNL1^{Mut}-dCas9 (KNL1^{RVSF/AAAA}-dCas9), while NDC80-CH1-dCas9 and NDC80-CH2-dCas9 showed lower but appreciable efficiency (see FIG. 7B). Notably, after normalization for the corresponding expression level (shown in FIG. 7B), KNL1^{S24A:S60A}-dCas9 induced a higher absolute level of aneuploidy than KNL1^{RVSF/AAAA}-dCas9, while NDC80-CH1-dCas9 and NDC80-CH2-dCas9 showed the highest induction of aneuploidy (FIG. 8B). We measured aneuploidy induced by expression of dCas9 (with sgRNAs), finding this to be approximately 30% of the level induced by KNL1^{RVSF/AAAA}-dCas9 (FIG. 8B). About 90% of the aneuploidy events induced by dCas9 were losses and 10% were gains, whereas for KNL1^{RVSF/AAAA}-dCas9 and especially KNL1^{S24A:S60A}-dCas9, 55-65% were losses (FIG. 8C). This indicates that just the recruitment of dCas9 to centromeres at least partially inhibits its normal function, leading mainly to chromosome losses, and that the simultaneous expression of mutant forms of KNL1 (especially KNL1^{S24A:S60A}-dCas9) has a significant additive effect on aneuploidy induction that is biased toward chromosome gains.

We evaluated which parameters and conditions affect KaryoCreate's efficiency, focusing on KNL1^{Mut}-dCas9 due to its higher absolute level of aneuploidy induction compared to other constructs. Higher levels of KNL1^{Mut}-dCas9 expression induced greater aneuploidy: a 3-fold increase in KNL1^{Mut}-dCas9 expression led to a 2-fold increase in gains or losses (FIG. 8D). Next, combining multiple sgRNAs targeting the same chromosome (sgChr7-1+sgChr7-3 or sgChr9-3+sgChr9-5) did not increase the percentage of aneuploid cells over that due to individual sgRNAs, despite the increase in predicted binding sites achieved by combining the sgRNAs (FIG. 8E, 3F). We also tested whether FACS sorting, based on a cell surface marker encoded on the target chromosome, could increase the percentage of cells with gains or losses. We sorted cells transduced with KNL1^{Mut}-dCas9 and sgChr7-1 based on high (top 15%) or low (bottom 15%) expression of EPHB4, a gene on chromosome 7 encoding a cell surface ephrin receptor. The percentage of cells with chromosome 7 gain increased from 12% to 26% from unsorted to high-EPHB4 cells (FIG. 8G), and the percentage of cells with chromosome 7 loss increased from 8% to 16% from unsorted to low-EPHB4 cells. Finally, a time-course experiment showed that sustained KaryoCreate activity increased aneuploidy progressively after 1, 2, or 3 cell cycles (2, 4, and 6 days after doxycycline; FIG. 8H). Altogether, the results indicate that KaryoCreate can induce chromosome-specific aneuploidy.

### Example 5

KaryoCreate allows induction of arm-level and chromosome-level gains and losses across human chromosomes.

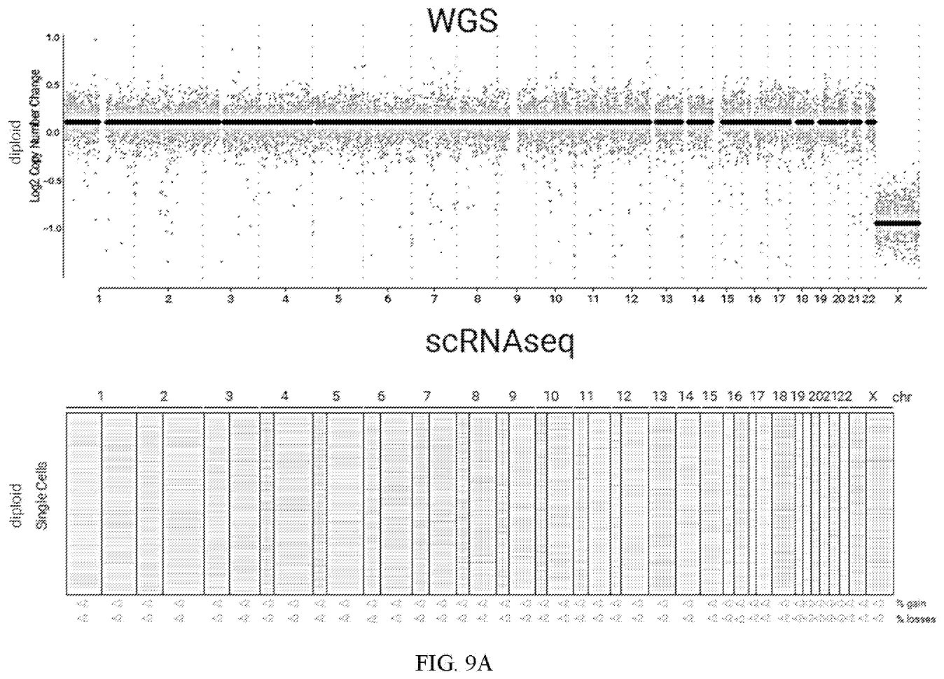

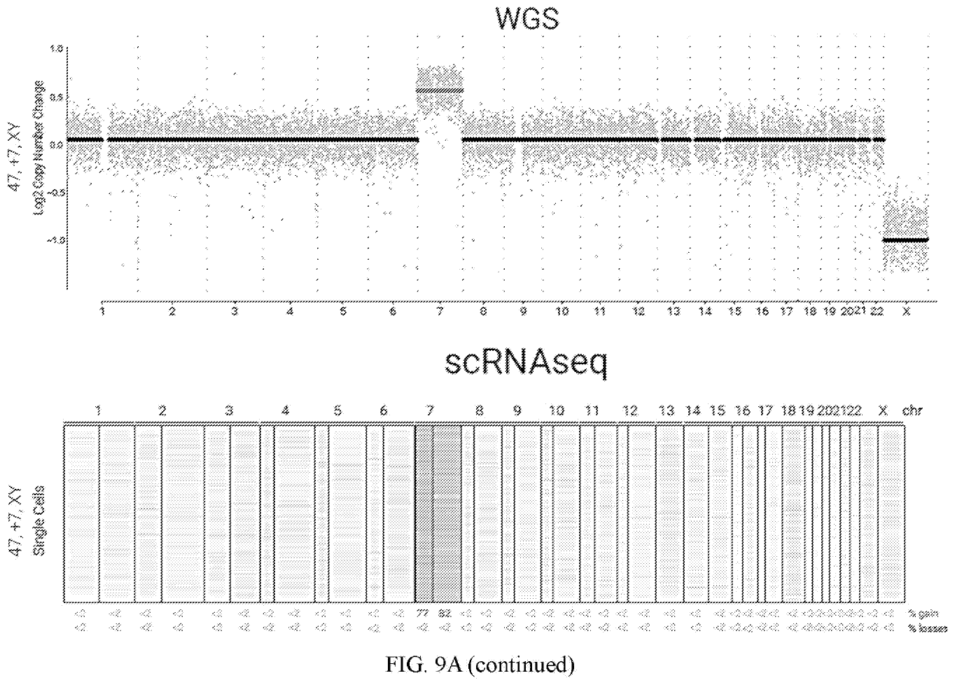

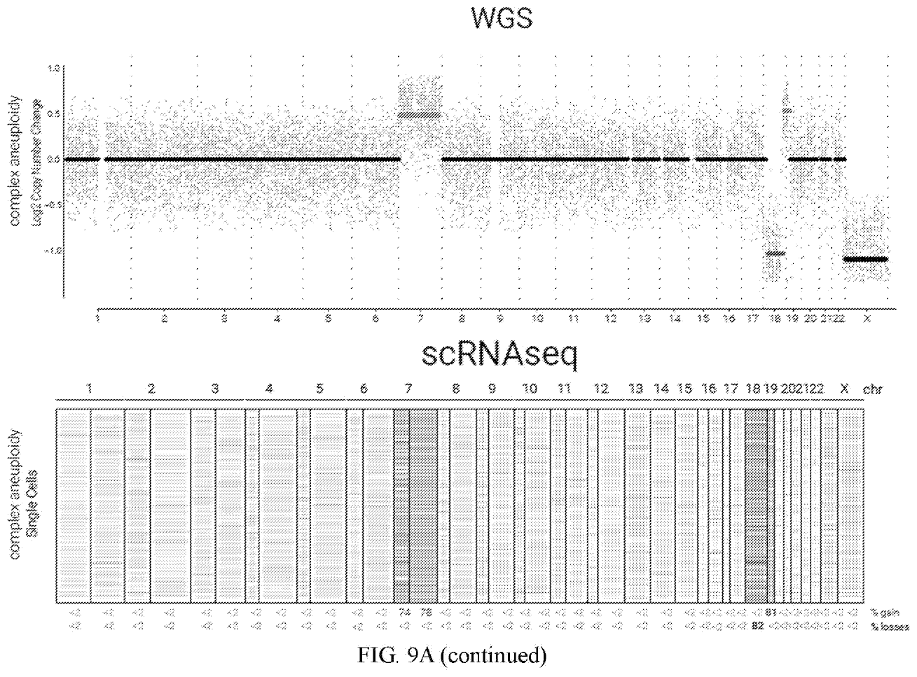

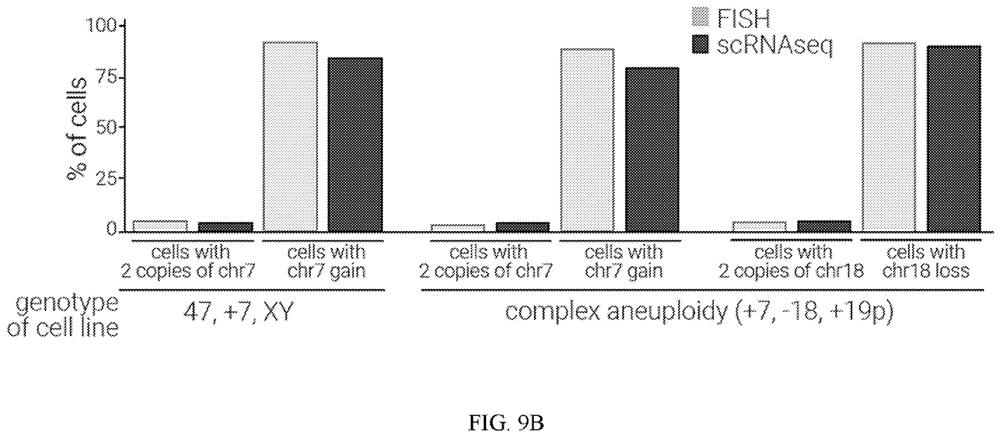

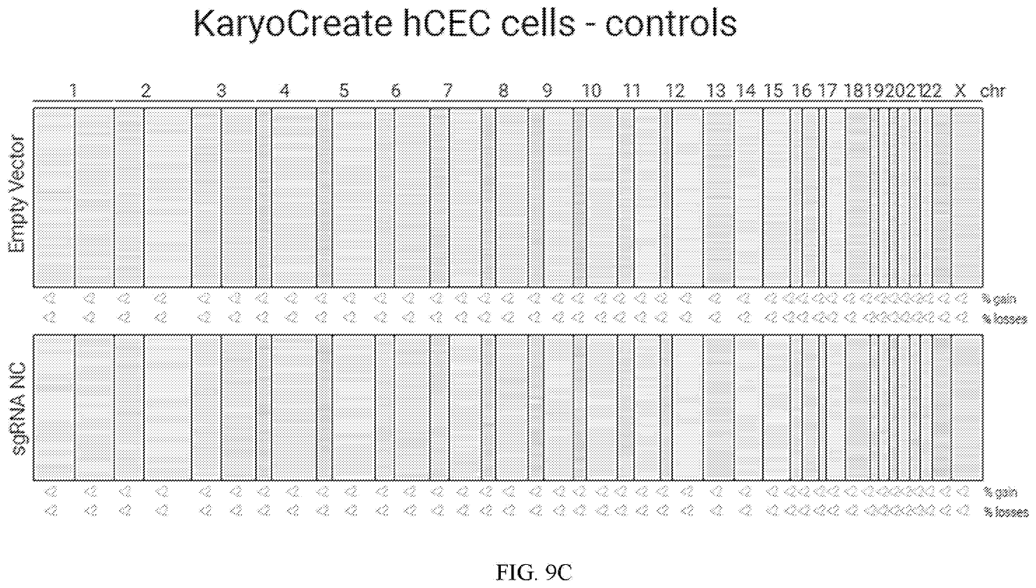

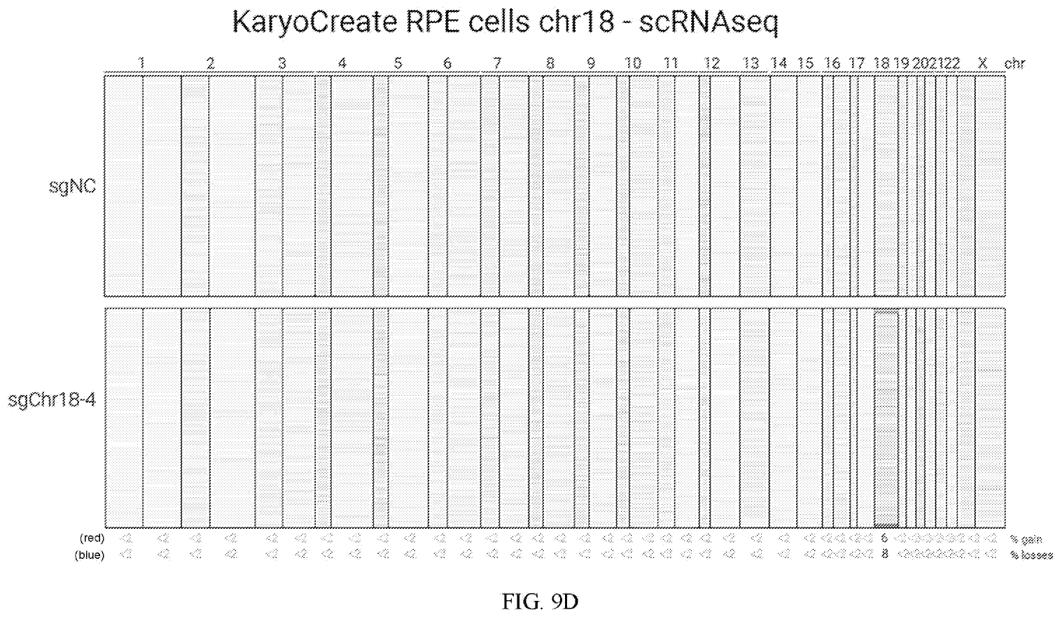

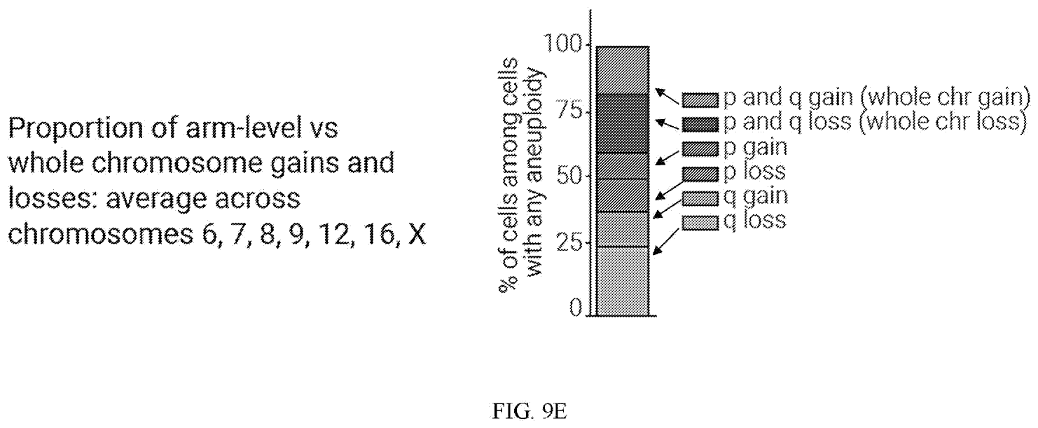

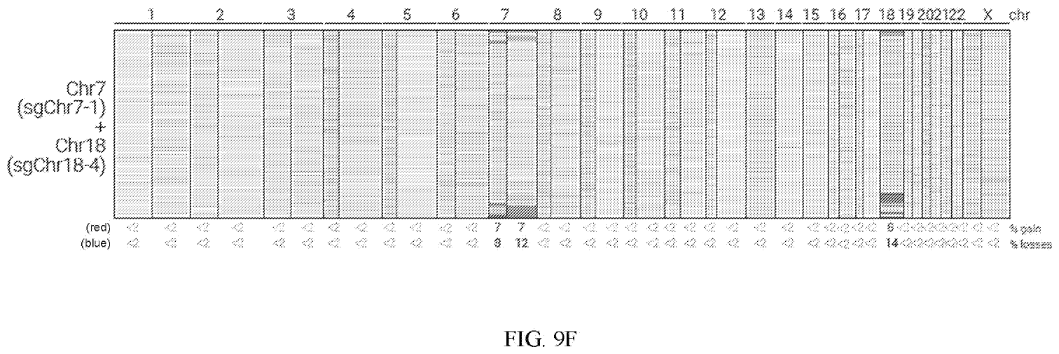

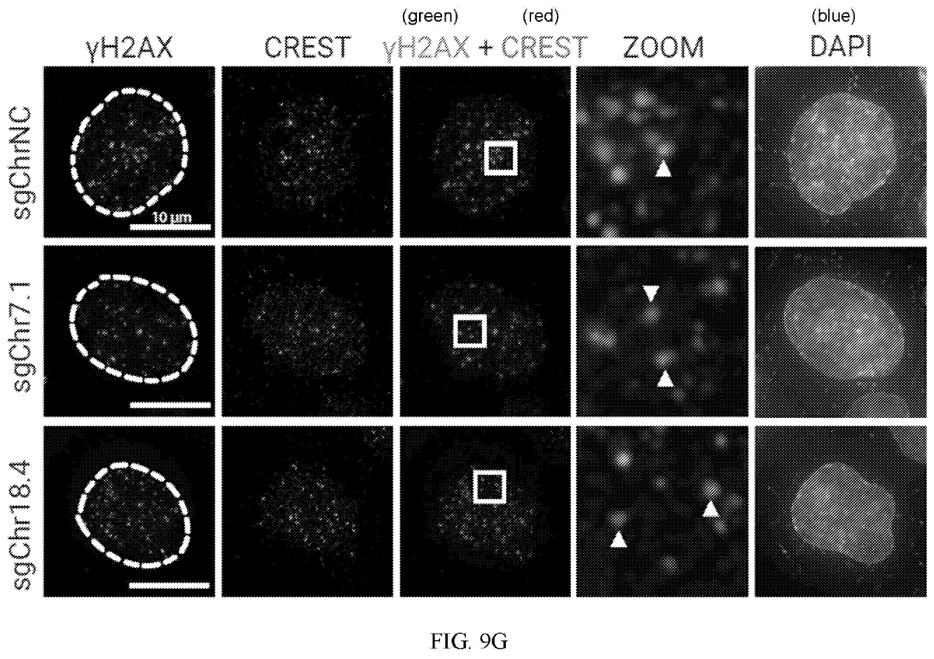

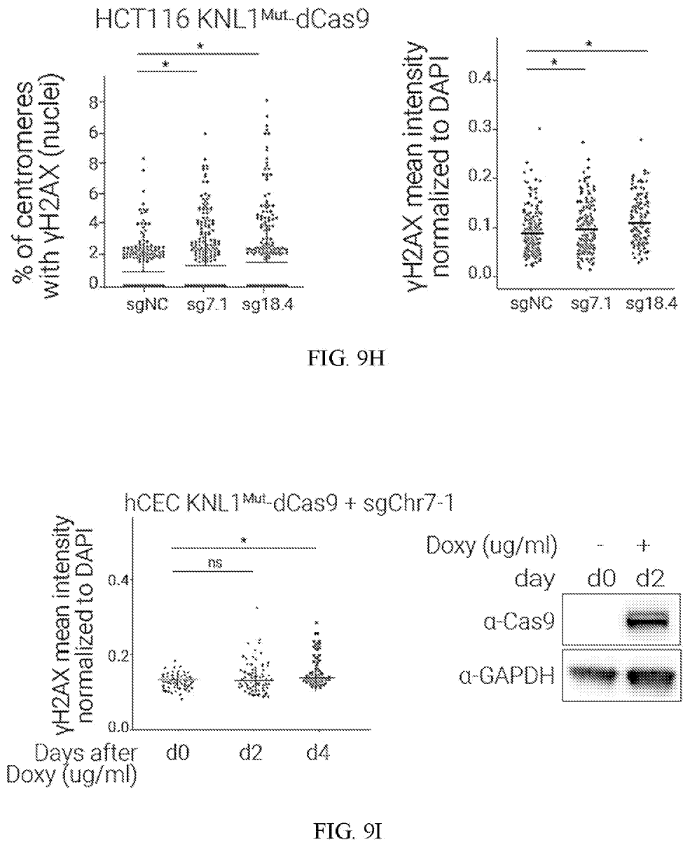

FISH analyses showed that targeting chromosome 7 does not affect chromosome 18 and vice versa, but did not rule out erroneous targeting of other chromosomes. To extend analysis of KaryoCreate's specificity across all chromosomes, we performed high-throughput single-cell RNA sequencing (scRNA-seq) to estimate genome-wide DNA copy number profiles across thousands of cells⁴⁶⁻⁴⁸. To infer copy number, we use the mean expression of genes across each chromosome or arm as a proxy for DNA copy number and then estimated the percentage of gains and losses for each arm by comparing the DNA copy number distribution of each experimental sample to that of the control population (e.g. sgNC or untreated cells). To prove the ability to infer arm-level copy number through scRNA-seq, we compared scRNA-seq and bulk shallow WGS results for hCEC cell lines with specific gains and losses. Analysis of a trisomic chromosome 7 clone showed that the percentage of cells with chromosome 7 gain was 91% by FISH and 80% by scRNA-seq. Similarly, analysis of the more complex karyotype (+chr7, −chr18, +19p) showed that the percentage of cells with chromosome 7 gain was 88% by FISH and 76% by scRNA-seq, and that for chromosome 18 loss was 87% by FISH and 81% by scRNA-seq (FIG. 9A, 9B). scRNA-seq slightly underestimated aneuploidy, especially gains, likely because a change from 2 to 3 copies represents an increase in DNA and RNA of 33%, while loss of 1 copy from 2 copies corresponds to a decrease of 50%. Overall, the patterns of aneuploidy inferred by scRNA-seq recapitulated those revealed by bulk WGS, confirming the validity of scRNA-seq for analyzing genome-wide gains and losses in single cells.

We performed scRNA-seq on diploid hCECs 7 days after KaryoCreate for chromosome 7 (sgChr7-1), chromosome 18 (sgChr18-4), and sgNC to estimate the frequency of induced aneuploidy (FIG. 4; pIND20 vector, expression level intermediate compared to those in FIG. 8D). For each sample, we estimated arm-level gains or losses for most chromosomes, except those with few (<20) genes detected on the p arm. First, we confirmed that the expression of KNL1^{Mut}-dCas9 with the sgNC construct did not significantly induce aneuploidy compared to that in cells treated with the EV control (FIG. 4, FIG. 9C), as it led to very low percentages of gains and losses across chromosomes, averaging 0.9% for gains and 1.2% for losses. We confirmed the induction of chromosome-specific gains or losses after KaryoCreate, consistent with our FISH experiments (FIG. 3D, 3E). For example, scRNA-seq showed 10% gains and 17% losses for chromosome 18 (sgChr18-4) (FIG. 4, Table S3) and 9% and 11% gains and losses for chromosome 7 (sgChr7-1), respectively (FIG. 4, Table S3). scRNA-seq confirmed that KaryoCreate-induced aneuploidy was highly specific, with an average background level of nonspecific aneuploidy of 1% (FIG. 4, Table S3). Notably, the gains (0.9%) and losses (1.2%) observed in the sgNC sample across chromosomes are about 3 times lower than those observed by DNA FISH (3% for both gains and losses) (FIG. 3E), again suggesting that scRNA-seq underestimates aneuploidy, and especially gains, compared to FISH (Table S3).

We further tested KaryoCreate using sgRNAs targeting additional chromosomes, including 6, 8, 9, 12, 16, and X, that were previously confirmed to induce foci with mScarlet-dCas9 (FIG. 4; see also FIG. 1 and FIG. 6). We performed KaryoCreate with the diploid hCECs expressing KNL1^{Mut}-dCas9 (pIND20) and analyzed the cells through scRNA-seq 7 days after doxycycline induction. In all cases, cells expressing the chromosome-specific sgRNAs showed more gains and losses of the targeted chromosome than those expressing sgNC. The chromosome-specific gains and losses differed among the chromosomes and ranged between 5% and 12% for gains (average across 10 chromosomes: 8%) and between 7% and 17% for losses (average across 10 chromosomes: 12%) (FIG. 4, Table S3). Notably, gains or losses of the non-targeted chromosomes never exceeded those in the sgNC control.

In agreement with our previous findings (FIG. 8D), the expression levels of the KNL1^{Mut}-dCas9 construct correlated with the efficiency of KaryoCreate: a 3-fold increase in KNL1^{Mut}-dCas9 expression (FIG. 8D) resulted in a 40-50% increase in both gains (from 9% to 16%) and losses (from 11% to 22%) (FIG. 4, compare sgChr7-1 and sgChr7-1 with high KNL1^{Mut}-dCas9 expression). Furthermore, we successfully utilized KaryoCreate for inducing multiple chromosomal gains or losses in the same cells, by transducing cells simultaneously with multiple sgRNAs targeting different chromosomes (sgChr7-1+sgChr18-4; 8% of cells had changes in both chromosomes 7 and 18 (FIG. 9F) or by utilizing a single sgRNA targeting multiple chromosomes (e.g. sgRNA 13-5 which targets both chromosomes 13 and 21 in hCEC; FIG. 4, Table S3). Finally, we obtained similar results using KaryoCreate in TP53 WT RPEs (FIG. 9D), suggesting that the method can be applied to different cell lines and in cells with an intact TP53 pathway.

Throughout the scRNA-seq analysis, we noted that in addition to whole-chromosome gains and losses, KaryoCreate also induced arm-level events, in which only one chromosomal arm (p or q) is gained or lost. Across the chromosomes tested, approximately 60% of aneuploidy events involved chromosome arms and 40% affected whole chromosomes (FIG. 9E). On average, there were 28% whole-chromosome losses, 17% whole-chromosome gains, 32% arm-level gains, and 23% arm-level losses (FIG. 9E, Table S3). Consistent with arm-level aneuploidy, we observed a modest increase in centromeric foci detected with the DNA damage marker γH2AX after expression of KNL1^{Mut}-dCas9 and sgChr7-1 or sgChr18-4 (but not sgNC) for 10 days in HCT116 cells, in both interphase nuclei and mitotic cells; the average γH2AX signal intensity per cell, normalized to DAPI, also increased (FIGS. 9G-9H and data not shown). In a time-course experiment, γH2AX signal had increased after 4 days of doxycycline treatment (approximately two cell cycles) but not after 2 days (approximately one cell cycle) (FIG. 9I). Notably, the ratio between arm-level and chromosome-level events also increased significantly after 4 (and 6) compared to 2 days of doxycycline treatment (FIG. 8H), indicating that DNA damage signal increases over prolonged binding of KNL1^{Mut}-dCas9 to the centromere and proportionally to arm-level events (see Discussion).

Altogether these data show that KaryoCreate can generate chromosomal gains and losses across individual chromosomes as well as combinations of the human autosomes and sex chromosomes.

### Example 6

18q loss in colon cells promotes resistance to TGFβ signaling likely due to haploinsufficiency of multiple genes.

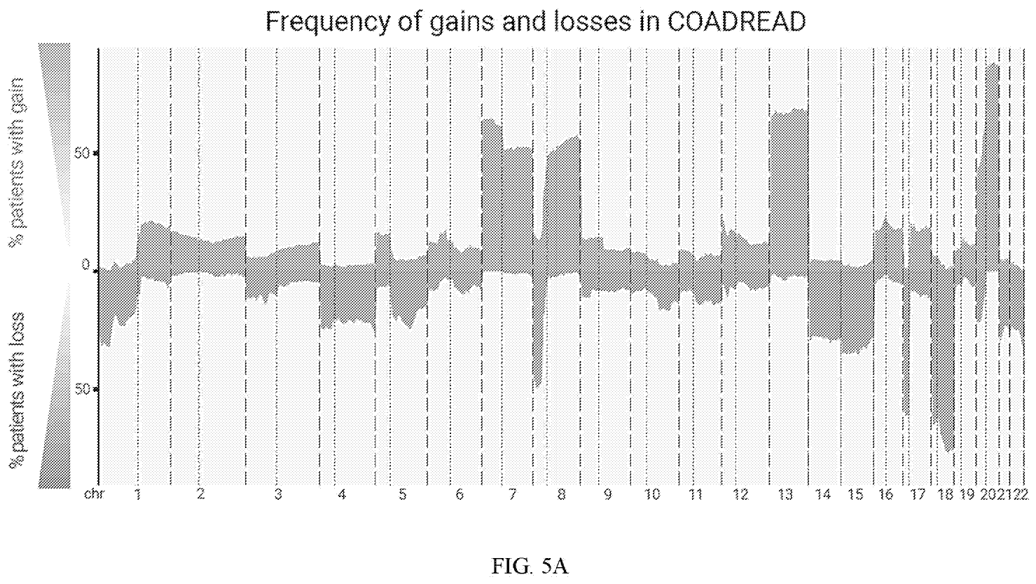

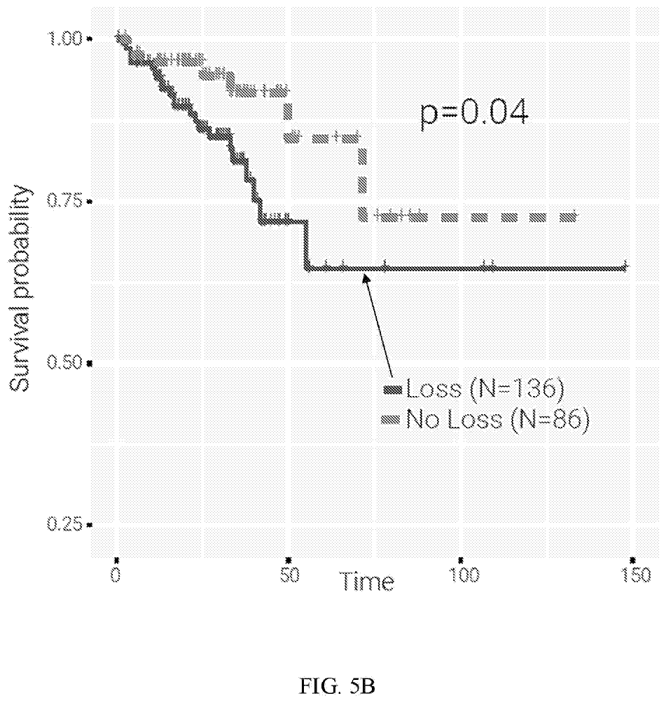

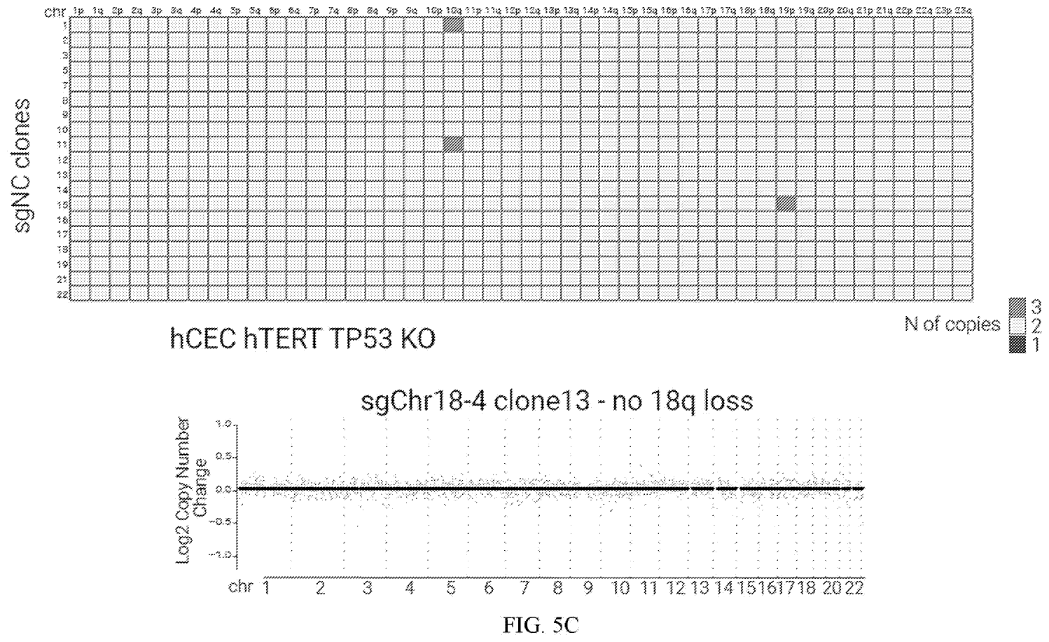

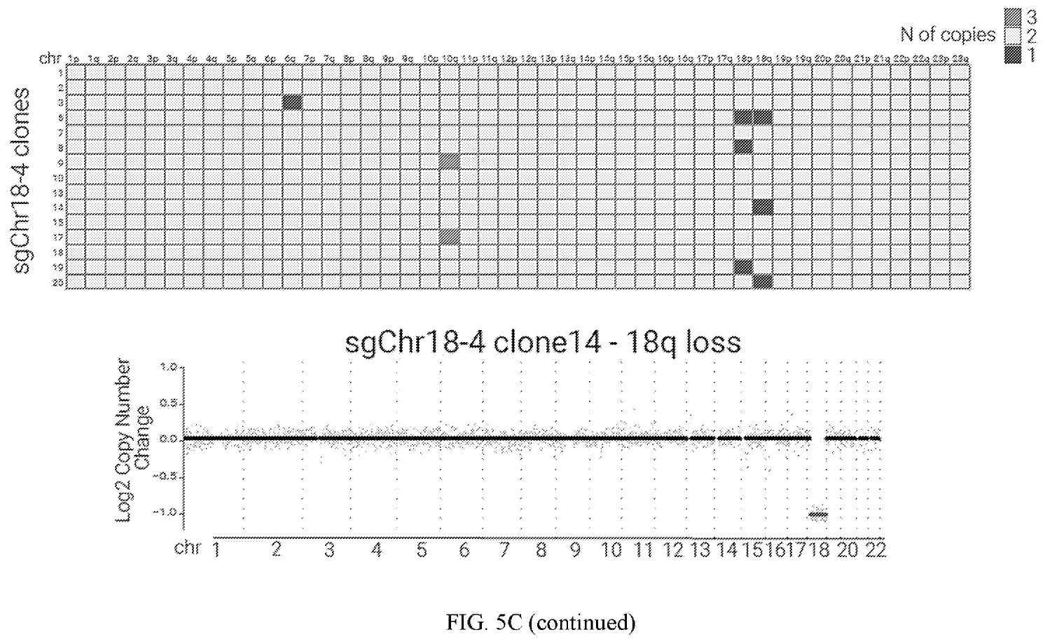

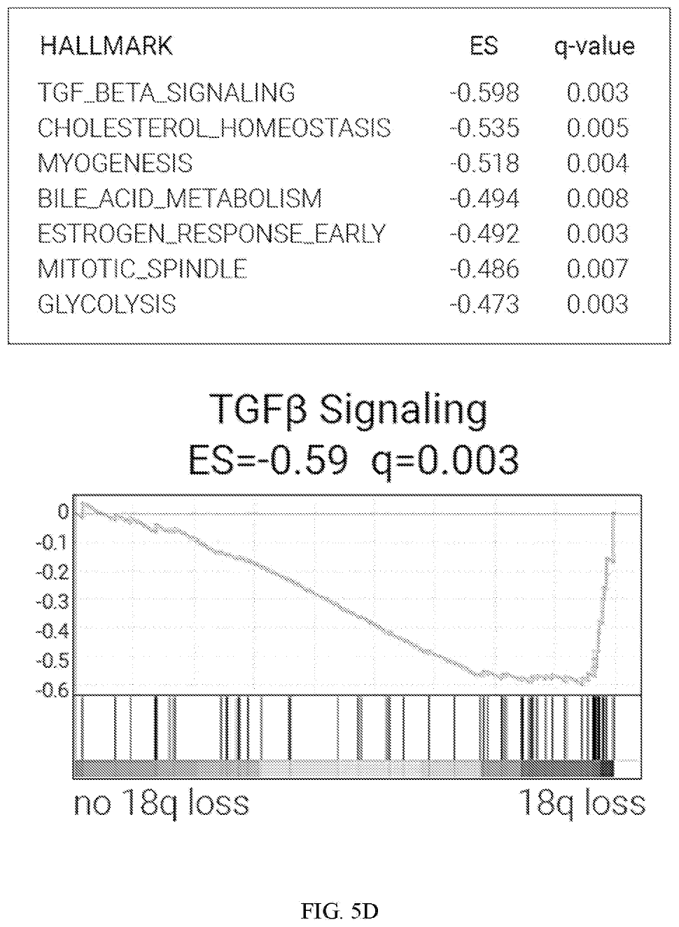

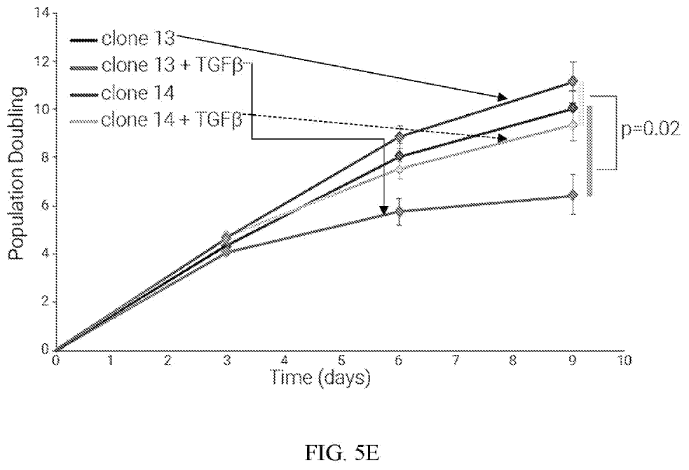

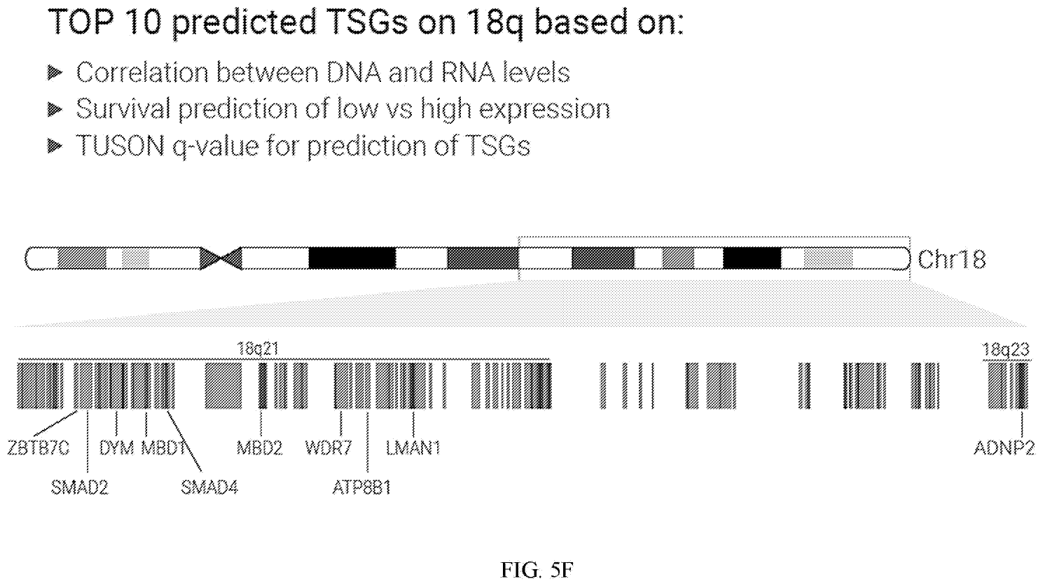

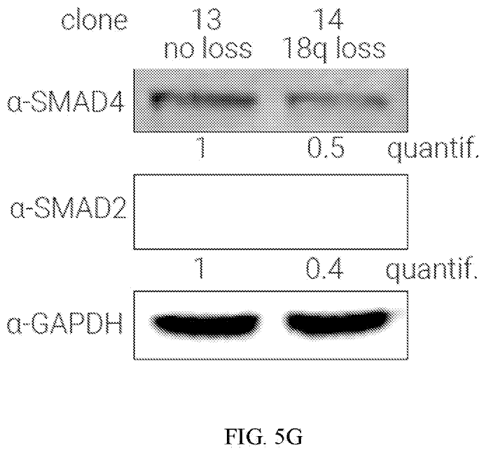

We used KaryoCreate to model 18q loss and chromosome 7 gain, aneuploidy events frequently found in colorectal cancer. Chromosome 18q is lost in about 62% of colorectal cancer (TCGA Dataset;⁴⁹, FIG. 5A), and patients with 18q loss (N=136) show poorer survival than those without (N=86) (p=0.04, log-rank test, FIG. 5B). Chromosome 7 gain is present in 50% of patients (FIG. 5A).

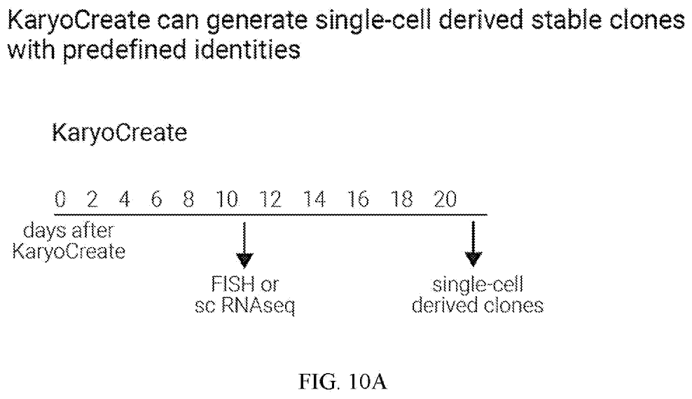

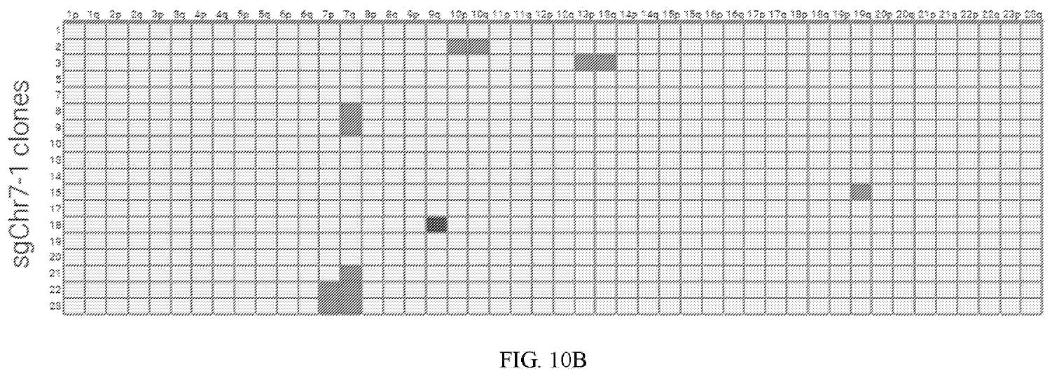

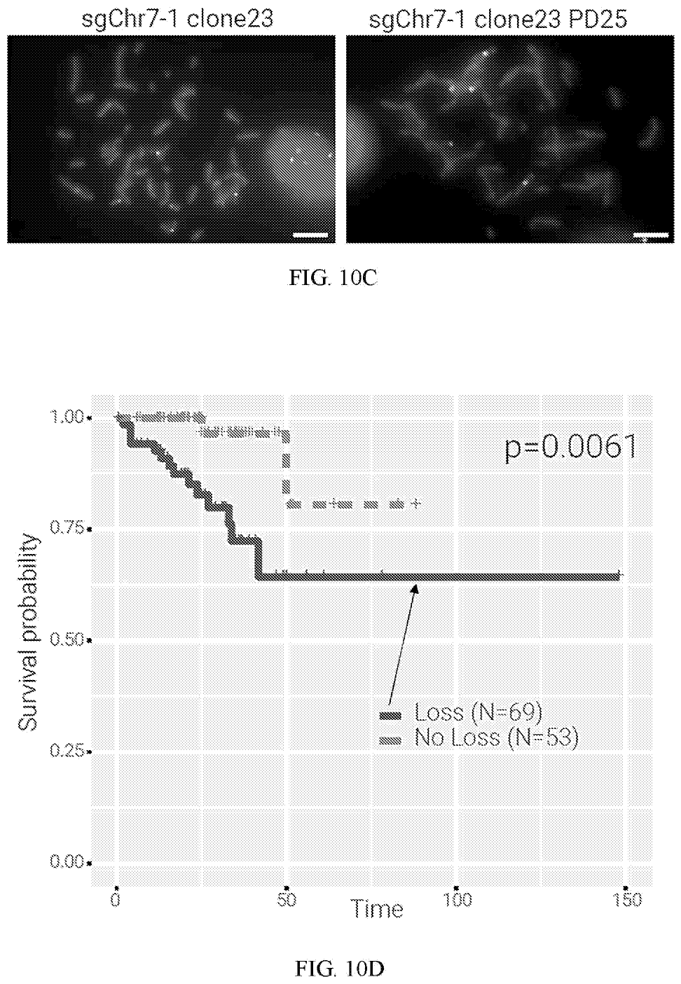

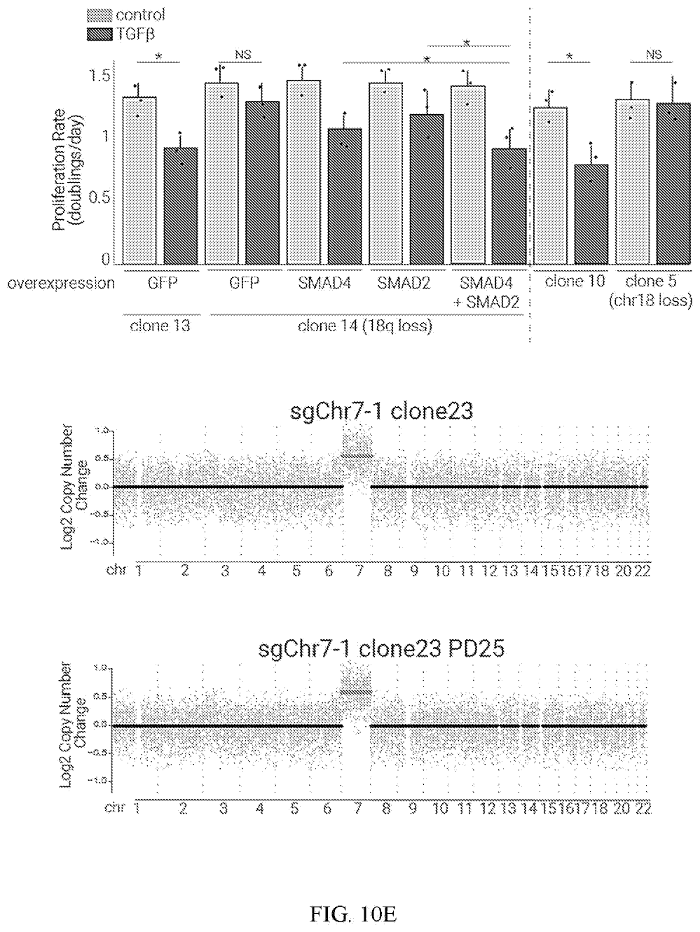

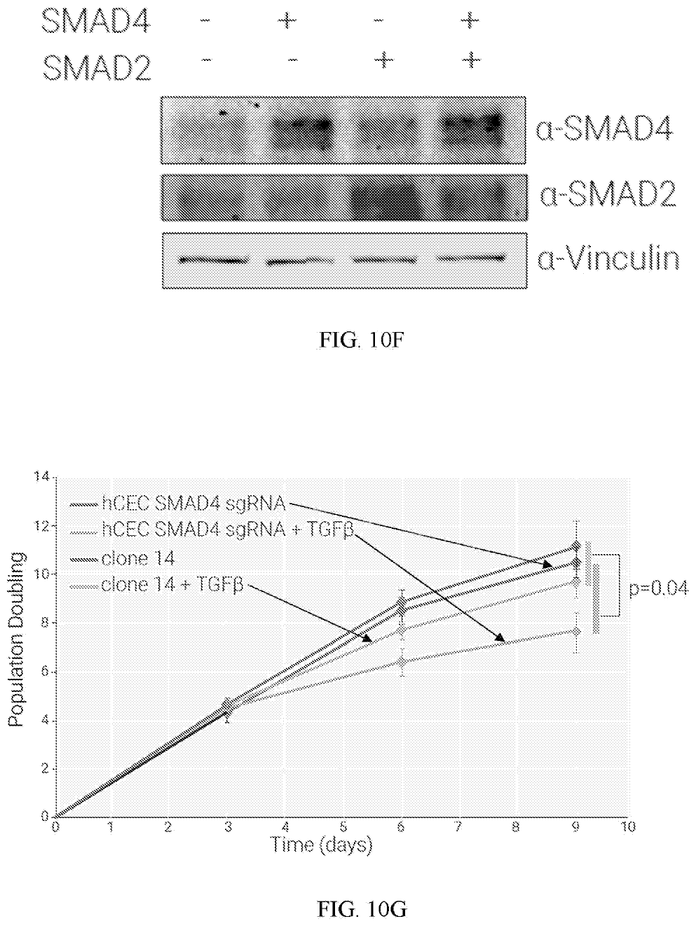

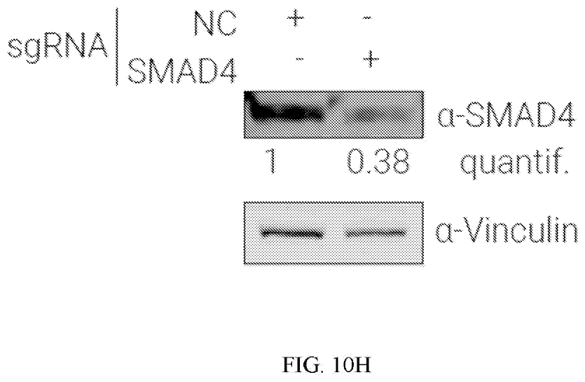

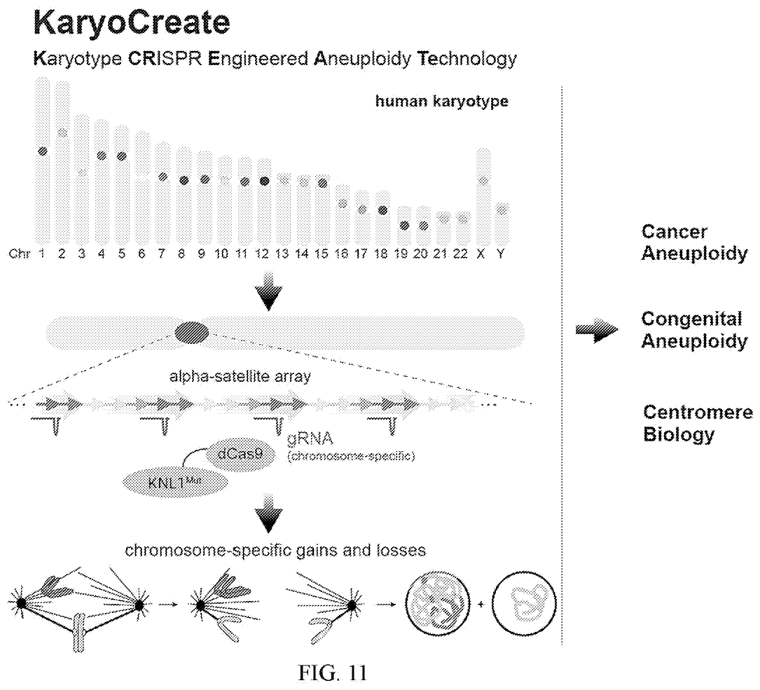

To model these events, we performed KaryoCreate on hCECs using sgChr7-1, sgChr18-4, or sgNC as above (see also Methods). About 20 single-cell-derived clones were derived for each condition and their copy number profiles evaluated by WGS. After KaryoCreate, cells were seeded at low density and allowed to grow into colonies for 3-4 weeks, a longer time than in the experiments above (FIG. 4), during which cells likely experienced selective pressure for the ability to grow as single colonies (FIG. 10A).

Compared to clones derived from the sgNC control population, clones derived from sgChr7-1 showed an increase from 0% in sgNC to 22% in chr7 gains but no losses (0 for both conditions) (FIG. 10B). Clones derived from sgChr18-4 showed an increase from 0% in sgNC to 30% in chr18 loss losses but not gains (0 for both conditions) (FIG. 5C). This recapitulates the recurrent patterns observed in human tumors, where chromosome 18 is frequently lost but virtually never gained (2%), whereas chromosome 7 is frequently gained and almost never lost (0.3%). We did not observe aneuploidy of chromosomes not targeted by KaryoCreate except for 10q gain, which was present in ˜20% of clones for all conditions, including sgNC, and was likely present in the initial population. Next, to test whether KaryoCreate clones can be stably propagated, we cultured a chromosome 7 trisomic clone (sgChr7-1 clone 23) for several weeks; we confirmed chromosome 7 gain by FISH and WGS analysis before and after 25 population doublings (FIG. 10C). We obtained similar results for sgChr18-4 clone 14.

Given the association of chromosome 18q loss with poor survival (FIG. 5B), we characterized the phenotypes of clones with or without this loss, starting from two clones derived from the KaryoCreate hCECs with sgChr18-4: one disomic control (clone 13) and one with 18q loss (clone 14). We performed bulk RNA sequencing analyses of each clone and conducted differential expression analysis using DESeq2⁵⁰. Gene-set enrichment analysis (GSEA) for cancer hallmarks showed that the top pathway downregulated in clone 14 compared to clone 13 was TGFβ signaling (enrichment score=−0.59; q-value=0.006), followed by cholesterol homeostasis, myogenesis, and bile acid metabolism (FIG. 5D). TGFβ (transforming growth factor beta) normally inhibits the proliferation of colon epithelial cells by promoting their differentiation; its inhibition through intestine niche factors such as Noggin is essential for the proliferation and expansion of colon epithelial cells⁵¹. We tested the effect of TGFβ activation in our clones through an in vitro cell proliferation assay in which we cultured clones 13 and 14 in the presence of TGFβ (20 ng/ml) for 10 days. At day 9, TGFβ treatment had reduced cell growth by about 45% for the control clone 13 but <10% for clone 14 (FIG. 5E; p=0.02). Altogether, these data suggest that 18q deletion leads to decreased response to the growth-inhibitory signals derived from TGFβ treatment. We obtained similar results with an independent pair of different clones, clone 10 (diploid) and clone 5 (lacking chromosome 18) (FIG. 10E).

Chromosome 18q harbors the tumor-suppressor gene SMAD4 (located on 18q21.2), encoding a transcription factor critical for mediating response to TGFβ signaling^{52,53}. In colorectal cancer, SMAD4 can be inactivated through point mutation (29% of patients)⁵⁴ or genomic loss (62% of patients); in 96% of cases of genomic loss, the deletion encompasses the entire chromosome arm. A previous study suggested that mutations may occur before chromosomal instability⁵⁴. Independently of the timing of SMAD4 mutations versus 18q loss, it is unknown whether the decreased survival in 18q loss patients (FIG. 5B) is a consequence of the complete loss of SMAD4 (due to co-occurring point mutation in the other allele) or is independent of SMAD4 mutation and possibly due to simultaneous loss of several tumor-suppressor genes on 18q, as previously suggested⁵⁵. To distinguish between these possibilities, we assessed the contribution of 18q loss to patient survival after excluding patients with point mutations in SMAD4: if 18q loss serves to abolish SMAD4 function through deletion of the wild-type allele when one copy of SMAD4 carries a point mutation, we would predict that 18q loss would lose its association with patient survival after patients with SMAD4 mutations are excluded. 18q loss remained a significant predictor of survival after SMAD4-mutated patients were removed, indicating that decreased survival could be a consequence of the deletion of several tumor-suppressor genes on 18q (FIG. 10D, p-value of 0.006, lower than in the analysis including all patients, see FIG. 5B).

To systematically predict tumor-suppressor genes located on 18q, we developed a score using three computational parameters based on the TCGA dataset: 1. correlation between DNA and RNA level of each gene across patients⁵⁶; 2. association of expression level of each gene with patients' survival; 3. TUSON-based prediction of the likelihood for a gene to behave as a tumor-suppressor gene based on its pattern of point mutations⁴. The top ten predicted genes were SMAD2, ADNP2, MBD1, ATP8B1, WDR7, MBD2, DYM, SMAD4, ZBTB7C, and LMAN1 (FIG. 5F). SMAD2, a paralogue of SMAD4 located on 18q21.1, is also a transcription factor acting downstream of TGFβ signaling^{51,57}. Thus, concomitant decreases in gene dosage of both SMAD4 and SMAD2 could synergistically mediate the unresponsiveness of cells to TGFβ signaling.

We tested the role of decreased dosage of SMAD2 and SMAD4 proteins in our clone containing 18q loss. We confirmed by both RNA-seq and Western blotting a decrease in both SMAD2 and SMAD4 in clone 14 compared to control clone 13 (FIG. 5G; SMAD) 4 log 2FC:−0.78, p<0.0001; SMAD2 log₂FC: −0.75, p<0.0001). Furthermore, overexpression of SMAD2 and SMAD4 in clone 14 decreased proliferation rate after TGFβ treatment to a level similar to clone 13 (FIG. 10E, 10F). To further test whether the increased resistance to TGFβ treatment after 18q loss was due to the synergistic effects of decreases in both SMAD2 and SMAD4 (as opposed to SMAD4 only), we derived hCECs with a ˜50% decrease in SMAD4 protein level by CRISPR interference (FIG. 10G, 10H). In proliferation assays, cells with 18q loss (clone 14) were more resistant to TGFβ treatment than hCECs with decreased SMAD4 levels (FIG. 10G, 10H), indicating that 18q loss has a greater effect than a ˜50% decrease in SMAD4 expression.

These computational and experimental data suggest that chromosome 18q loss, one of the most frequent events in gastro-intestinal cancers, is associated with poor survival and promotes resistance to TGFβ signaling, likely because of the synergistic effect of simultaneous deletion of haploinsufficient genes.

### Discussion of Examples

Chromosome-Specific Centromeric sgRNAs

KaryoCreate includes the design of sgRNAs targeting chromosome-specific α-satellite DNA. Among 75 tested, we validated 24 sgRNAs specific for 16 different chromosomes (FIG. 1, FIG. 6, Table S1). Since centromere sequences vary across the human population, we designed sgRNAs using two genome assemblies (CHM13 and GRCh38) and tested them in different cell lines (hCECs, RPEs, and HCT116), increasing their likelihood of targeting conserved regions.

The disclosure demonstrates the design and use of sgRNAs to target human centromeres for most human chromosomes. Some chromosomes are not included due to centromeric sequences sharing high similarity across specific chromosome groups (i.e. acrocentric), to the low GC content of centromeric sequences likely decreasing the gRNA activity, or to a lack of sufficient predicted binding sites (e.g. D21Z1, D15Z3, and D3Z1 in the CHM13 assembly have relatively small active centromere regions)^{21,58}. The efficiency of centromeric sgRNAs is not accurately predicted using algorithms for non-centromeric regions³⁵ (FIG. 6E). Using more than one sgRNA simultaneously did not improve aneuploidy induction (FIG. 8E, 8F). Because of the repetitive nature of centromeres, any pair of sgRNAs is predicted to bind multiple times and relatively close together, potentially inducing competition or interference among KNL1^{Mut}-dCas9 molecules.

Comparison of KaryoCreate with Similar Technologies

Other strategies have been recently described to induce chromosome-specific aneuploidy targeting non-centromeric repeats and have been successful for chromosome 1 using a sub-telomeric repeat and chromosome 9 using a pericentromeric repeat^{16,17}. Tovini et al. used dCas9 fused to the kinetochore-nucleating domain of CENPT to form an ectopic kinetochore. Truong et al. tethered a plant kinesin to pull the chromatids towards one pole of the mitotic spindle, potentially generating a pseudo-dicentric chromosome, as suggested by the fact that most aneuploidies observed were of part of the targeted chromosome (chromosome 9). KaryoCreate is distinct in that it uses endogenous centromeric sequences to allow the generation of nearly any karyotype of interest. We found that cells progressed normally through the cell cycle with an expected brief delay in metaphase, likely due to attempts at correcting merotelic attachments^{59,60}. Also, in contrast to existing technologies, KaryoCreate can induce specific aneuploidies across several chromosomes or combinations thereof (Table S3). KaryoCreate also enables induction of aneuploidy not only in TP53^{-/-} cells but also in TP53 WT cells such as HCT116 cells (FIG. 2E) and RPEs (FIG. 9D).

### Targeting Mutant Kinetochore Proteins to Centromeric α-Satellites to Engineer Chromosome-Specific Aneuploidy

Tethering of chimeric dCas9 with mutant forms of KNL1 or NDC80 to human centromeres induces chromosome- and arm-level gains and losses (FIG. 8B). Data in this disclosure suggest that dCas9 itself may induce low-frequency aneuploidy, possibly due to tethering of a bulky protein to the centromeric repeats^{16,17,42}. Remarkably, the expression of chimeric mutants of kinetochore proteins at centromeric regions induces about 3 times as many aneuploidy events compared to dCas9 alone, which may be due to the disruption of their proper kinetochore functions (FIG. 8B). We noted that different mutants show different efficiency of aneuploidy induction relative to their expression level (FIG. 7B, 7B). NDC80 mutants induced aneuploidy efficiently relative to their low expression level, suggesting a higher degree of kinetochore disruption compared to KNL1 fusion (FIG. 7B, 8B). Of the two chimeras containing KNL1 mutants, we predicted that KNL1^{S24A:S60A}-dCas9 would result in a more efficient induction of chromosome gains and losses than KNL1^{RVSF/AAAA}-dCas9, owing to a more efficient inhibition of Aurora-B-mediated error correction through recruitment of PP1^{28,41}. Although this was not the case in terms of absolute level of aneuploidy, KNL1^{S24A:S60A}-dCas9 efficiency was higher when normalized for protein expression level (FIG. 8B).

### Induction of Arm-Level Gains and Losses

About 55% of the aneuploidy generated by KaryoCreate are arm-level events. In addition, we observed more losses (60%) than gains (40%) for both chromosome and arm events. Our data reveal a small fraction of centromeres positive for γH2AX upon aneuploidy induction with KaryoCreate (FIG. 9G-9I), especially upon prolonged centromere recruitment of KNL1^{Mut}-dCas9 and proportionally to the ratio between arm-level and chromosome-level events (FIG. 8H). The mere recruitment of a bulky protein to the centromere may influence centromere function, as our data on the effect of dCas9 alone suggest (FIG. 8B)^{18,31,61-63}. When recruited to the highly repetitive centromeric regions, dCas9 may influence chromosome segregation through impaired replication or transcription affecting chromatin, transcripts, and R-loops and, in turn, centromere function⁶²⁻⁶⁶.

### Chromosome-Specific Aneuploidy as a Driver of Cancer Hallmarks

We used KaryoCreate to induce missegregation of chromosomes 7 and 18, two of the chromosomes most frequently aneuploid in colorectal tumors. Among the single-cell-derived clones, chromosome 7 tended to be gained and chromosome 18 tended to be lost (FIG. 5C, FIG. 9B), indicating that the selective pressure acting during tumor evolution to shape recurrent patterns of aneuploidy may also act in vitro^{4,7}. In our analyses, 18q loss was a strong predictor of poor survival, consistent with previous studies^{67,68}, in addition the association of 18q loss with survival was independent of SMAD4 point mutations. We showed that chr18q loss can promote resistance to TGFβ signaling in colon cells. While SMAD4 is a frequently mutated tumor-suppressor gene⁵⁴ on chr18q, the TGFβ resistance phenotype determined by 18q loss may be due not solely to its loss but to the cumulative effect of losing multiple tumor suppressors on the arm. In fact, ˜50% reduction in SMAD4 alone was not sufficient to recapitulate resistance to TGFβ signaling seen after 18q loss, and dosage increases in both SMAD4 and SMAD2 could rescue TGFβ resistance in 18q loss cells (FIG. 5E, FIG. 10E-10H). Thus, chromosome 18 loss may drive TGFβ resistance through hemizygous deletion of (at least) two haploinsufficient genes acting in the same pathway.

Previous studies have proposed that a single cancer-driver gene may confer the strong phenotypic effect of whole-chromosome gain or loss^{69,70}. Other studies, including previous work on chromosome 18, have proposed that the selective advantage of aneuploidy is instead conferred by the cumulative effect of gene dosages of multiple genes^{4,6,55,71}. The present data support this latter hypothesis. Altogether, these data suggest that 18q loss may drive tumor phenotypes in colorectal cancer through the cumulative loss of several tumor-suppressor genes located on the chromosome arm.

### Cell Lines

All cells were grown at 37° C. with 5% CO₂ levels. hTERT TP53^{-/-} human colonic epithelial cells (hCECs)³⁸ were cultured in a 4:1 mix of DMEM: Medium 199, supplemented with 2% FBS, 5 ng/mL EGF, 1 μg/mL hydrocortisone, 10 μg/mL insulin, 2 μg/mL transferrin, 5 nM sodium selenite, pen-strep, and L-glutamine. hTERT retinal pigment epithelial cells (RPEs) 39 either WT (FIG. 9D) or expressing p21 ((DKN1A) and RB (RB1) shRNAs (FIG. 6D), and human colorectal carcinoma-116 cells (HCT116s) were incubated in DMEM, supplemented with 10% FBS, pen-strep, and L-glutamine. For long-term storage, cells were cryopreserved at −80° C. in 70% medium (according to cell line), 20% FBS, 10% DMSO. TP53 was knocked-out in hCECs by transfection with a Cas9-containing plasmid (Addgene #42230) and plLentiGuide-Puro expressing the following sgRNA: GCATGGGCGGCATGAACCGG (SEQ ID NO: 6). Clones were derived and tested for the expression of TP53.

### Methods Details

### Cloning of KaryoCreate Constructs

Cas9 and dCas9 without ATG and without stop codon (for N-terminal and C-terminal tagging respectively) were cloned into D-TOPO vector (Thermo #K240020). Cloning of KNL1^{RVSF/AAAA}-dCas9 was achieved by inserting KNL1 PCR product (aa1-86, amplified from Addgene plasmid #4522528) into XhoI-digested pENTR-dCas9 (no ATG) using Gibson assembly. The GGSGGGS (SEQ ID NO: 5) linker was added between KNL1 and dCas9. Cloning of KNL1^{S24A:S60A}-dCas9 was achieved starting from KNL1^{RVSF/AAAA}-dCas9 and inserting the appropriate mutations using Gibson assembly. Cloning of NDC80-CH1-dCas9 was achieved by Gibson assembly of NDC80 aa1-207 (generously provided by Dr. Jennifer DeLuca) with BamHI-digested pENTR dCas9 (ATG). Cloning of NDC80-CH2-dCas9 was achieved in a similar way except that 2 CH domains were cloned in tandem separated by a linker (see also FIG. 7A).

To generate an inducible KNL1^{Mut}-dCas9 construct, the FKBP12 degradation domain (DD, Banaszynski 2006⁴⁵) was first amplified from Degron-KI-donor backbone (Addgene #65483) and inserted at the N-terminus of the fusion protein sequence in pENTR-KNL1^{RVSF/AAAA}-dCas9 using Gibson cloning. Gateway LR cloning was then used to yield the expression vector, pHAGE-DD-KNL1^{RVSF/AAAA}-dCas9.

pHAGE-3×mScarlet-dCas9 was generated by first assembling three mScarlets in series and inserting them into the BsaI-digested pAV10 vector by Golden Gate cloning. The assembled 3×mScarlet was then inserted into XhoI-digested pENTR-dCas9 using Gibson cloning to form pENTR-3×mScarlet-dCas9.

All pENTR vectors were cloned into specific pDEST vectors by LR reaction (Thermo #11791020) following the manufacturer's instructions. pDEST vectors used in this study were pHAGE (blast resistance, CMV promoter) or pINDUCER20 (or pIND20, neomycin resistance, doxycycline inducible promoter)⁴⁴.

Cloning of sgRNAs

We modified the scaffold sequence of pLentiGuide-Puro (Addgene #52963) by Gibson assembly to contain the A-U flip (F) and hairpin extension (E) described by Chen et al⁷². for improved sgRNA-dCas9 assembly, obtaining pLentiGuide-Puro-FE. sgRNAs were designed and cloned into this pLentiGuide-Puro-FE vector according to the Zhang Lab General Cloning Protocol⁷³ (also addgene.org/crispr/zhang/) (see also Table S1 for sgRNA sequences). To be suitable for cloning into BbsI-digested vectors, sense oligos were designed with a CACC 5′ overhang and antisense oligos were designed with an AAAC 5′ overhang. The sense and antisense oligos were annealed, phosphorylated, and ligated into either BbsI-digested pLentiGuide-Puro-FE for KaryoCreate and imaging purposes or pX330-U6-Chimeric_BB-CBh-hSpCas9⁷⁴ (Addgene #42230) for CRISPR/Cas9 editing applications. Sequences were confirmed by Sanger sequencing.

### Lentivirus Production and Nucleofection

For transduction of cells, lentivirus was generated as follows: 1 million 293T cells were seeded in a 6-well plate 24 hours before transfection. The cells were transfected with a mixture of gene transfer plasmid (2 μg) and packaging plasmids including 0.6 μg ENV (VSV-G; addgene #8454), 1 μg Packaging (pMDLg/pRRE; addgene #12251), and 0.5 μg pRSV-REV (addgene #12253) along with CaCl₂) and 2×HBS or using Lipofectamine 3000 (Thermo #L3000075). The medium was changed 6 hours later and virus was collected 48 hours after transfection by filtering the medium through a 0.45-μm filter. Polybrene (1:1000) was added to filtered medium before infection.

Nucleofection of hCECs was carried out using the Amaxa Nucleofector II (Lonza), using the program optimized for the HCT116 cell line. Approximately 1 million cells suspended in 100 μL of electroporation buffer (80% 125 mM Na₂HPO₄·7H₂O), 12.5 nM KCl, 20% 55 mM MgCl₂) were subjected to electroporation in the presence of a vector and then immediately returned to normal medium.

### KaryoCreate Experiments

The disclosure includes three representative approaches to perform the described KaryoCreate process. One difference between these methods is the way KNL1^{Mut}-dCas9 and the sgRNA are expressed in the cell.

### Representative Methods to Express KNL1^{Mut}-dCas9:

A) KNL1^{Mut}-dCas9 is expressed from a doxycycline-inducible promoter (pIND20-KNL1^{Mut}-dCas9) through a viral vector constitutively integrated in the genome of the target cell. Cells are treated with doxycycline (1 μg/ul) for 7-9 days.
B) KNL1^{Mut}-dCas9 is expressed from a constitutive promoter (pHAGE-KNL1^{Mut}-dCas9; CMV promoter) through transient transfection.
C) KNL1^{Mut}-dCas9 is expressed through a viral vector constitutively integrated in the genome of the target cell; the expression level of KNL1^{Mut}-dCas9 is regulated through a degron (pHAGE-DD-KNL1^{Mut}-dCas9; see above)

For the sgRNA, expression is mediated by pLentiGuide-Puro-FE vector through infection or transient transfection. In this disclosure, unless otherwise specified, the sgRNA was introduced through infection. For a comparison of the three different methods, see FIG. 8A.

### Western Blot Analysis

Cells were harvested by trypsinization, lysed in 2×NuPAGE LDS buffer (Thermo #NP0007) at 10⁶ cells in 100 μl of buffer. DNA was sheared using a 28½-gauge insulin syringe and lysate was denatured by heating at 80° C. for 10 min. Lysate equivalent to 10⁵ cells was resolved by SDS/PAGE using a NuPAGE 4-12% Bis-Tris mini gel and transferred to a PVDF membrane (Bio-Rad #1704274). The membrane was then blocked in 5% milk in TBS with 0.1% Tween-20 (TBS-T) for 1 hour at room temperature. Afterward, the membrane was probed with Cas9 (Abcam #ab191468, 1:1000 dilution) and GAPDH (Santa Cruz #sc-47724, 1:10,000 or 1:100,000 dilution) or β-actin (Cell Signaling Technology #8844) primary antibodies and incubated in 1% milk in TBS at 4° C. overnight. For SMAD2 and SMAD4 western blots, Abcam Ab40855 and Santa Cruz Biotechnology #Sc-7966 were used.

Subsequently, the membrane was washed three times with TBS-T and incubated with HRP-anti-Mouse secondary Ab (Abcam #ab205719, 1:1000 dilution) in 1% milk/TBS for 1 hour at room temperature. Signals were detected using an ECL system using 1:1 detection solution (Thermo Scientific #32209) after three 10-min washes in TBS-T. Images were acquired using a BIORAD transilluminator.

### Fluorescence In Situ Hybridization (FISH)

For the analyses confirming centromeric localization of 3×mScarlet-dCas9 and localization of specific chromosomes within micronuclei, FISH was performed using an Empire Genomics chromosome 7 control probe (CHR07-10-GR) or chromosome 18 control probe (CHR18-10-GR) on PFA-fixed cells according to the manufacturer's manual hybridization protocol.

FISH analysis was carried out on interphase nuclei and metaphase spreads prepared as follows: Cells at 70% confluence were harvested by trypsinization (after 3- to 4-hour treatment with 100 ng/ml colcemid (Roche #10295892001) for metaphase spreads), washed with PBS, suspended in 0.075 M KCl at 37° C., and fixed in methanol-acetic acid (3:1) at 4° C. Fixed cells were dropped onto glass slides and then allowed to air dry overnight.

The slides were next incubated with RNase solution (20 μg RNase A in 2×SSC) for one hour at 37° C. in a dark moist chamber. Denaturing was performed using a 70% formamide solution (in 2×SSC) for 3 min at 80° C. prior to hybridization. Biotinylated/digoxigeninated probes were obtained by nick translation from BAC DNA (RP11-22N19 for chromosome 7, RP11-76N11 for chromosome 13, and RP11-787K12 for chromosome 18 from the BACPAC Resource Center). 200 ng of each labeled probe, together with 8 μg Human Cot-I DNA (Thermo #15279011) and 3 μg Herring Sperm DNA (Thermo #15634017) were precipitated for 1 hour at −20° C. in 1/10 volume of 3 M sodium acetate and 3 volumes of ethanol. The pelleted probe was washed with 70% ethanol, air dried, and resuspended in hybridization solution (50% deionized formamide, 10× dextran sulfate, 2× SSC). The hybridization solution containing the probes was then denatured at 80° C. for 10 min and then incubated at 37° C. for 20 min to allow annealing of the Cot-I competitor DNA. The sealed hybridized slides were then incubated at 37° C. in a dark moist chamber overnight. The following day, slides were washed in 1×SSC at 60° C. (3 times, 5 min each) and incubated with a blocking solution (BSA, 2×SSC, 0.1% Tween-20) for 1 hour at 37° C. in a moist chamber. Following blocking, the slides were incubated with detection solution containing BSA, 2×SSC, 0.1% Tween-20, and FITC-Avidin conjugated (Thermo #21221), and 10 μl Rhodamine-Anti-Digoxigenin (Sigma #11207750910) to detect the biotin and digoxigenin signals. Finally, slides were washed 3 times (5 min each) with 4×SSC and 0.1% Tween-20 solution at 42° C. and then mounted with DAPI to stain DNA (Vector Laboratories #H-1200-10).

Images were acquired using an Invitrogen™ Evos™ M700 imaging system or Nikon TI Eclipse. The number of fluorescent signals was counted in 100 intact nuclei per slide. Adobe Photoshop was used to count the signals and correct the images.

### Live-Cell Imaging

Cells were plated on 35-mm glass-bottom microwell dishes (MatTek P35G-1.5-14-C) 1 day prior to imaging. Imaging was performed at 37° C. and 5% CO₂ using an Andor Yokogawa CSU-X confocal spinning disc on a Nikon TI Eclipse microscope. Samples were exposed to 488-nm (30-ms) and 561-nm (100-ms) lasers and fluorescence was recorded with a sCMOS Prime95B camera (Photometrics). A 100× objective was used to acquire images at 0.9-μm steps (total range size=9 μm) every 1 or 3 min as indicated in the figure legends. Image analysis was performed using ImageJ and formatting (cropping, contrast adjustment, labeling) was performed in Adobe Photoshop.

### Chromosome Misalignment Staining

HCT116 cells were plated onto coverslips coated with 5 μg/ml fibronectin (Sigma-Aldrich) at 60-70% confluence and synchronized with 7.5 μM RO-3306 (Sigma-Aldrich) for 16 hours at 37° C. Cells were released from RO-3306 for 40 min and then treated with 10 μM MG-132 (Tocris) for 90 min at 37° C. Cells were then fixed with 4% paraformaldehyde for 12 min at room temperature and blocked in 5% BSA for 30 min. Samples were stained with the following antibodies for 90 min at room temperature: anti-α-Tubulin (Sigma-Aldrich #T9026, 1:1500 dilution) and anti-centromeric antibody (Antibodies Incorporated SKU 15-234, 1:100 dilution). CyTM3 AffiniPure (Jackson ImmunResearch #715-165-150) and Alexa 647-labeled (Jackson ImmunoResearch #709-606-149) secondary antibodies were used 1:400 for 45 min at room temperature. Coverslips were mounted using Mowiol. Cells were imaged using a Leica SP5 confocal microscope with a magnification objective of 63×. FIJI software was used for image analysis.

### Low-Pass Whole-Genome Sequencing

Genomic DNA was extracted from trypsinized cells using 0.3 μg/μL Proteinase K (Qiagen #19131) in 10 mM Tris, pH 8.0, for 1 hour at 55° C. and then heat inactivated at 70° C. for 10 min. DNA was digested using NEBNext® dsDNA Fragmentase® (NEB #M0348S) for 25 min at 37° C. and then subjected to magnetic DNA bead cleanup with Sera-Mag Select Beads (Cytiva #293430452), 2:1 bead/lysate ratio by volume. DNA libraries with an average library size of 320 bp were created using the NEBNext® Ultra™ II DNA Library Prep Kit for Illumina® (NEB #E7645L) according to the manufacturer's instructions. Quantification was performed using a Qubit 2.0 fluorometer (Invitrogen #Q32866) and the Qubit dsDNA HS kit (Invitrogen #Q32854). Libraries were sequenced on an Illumina NextSeq 500 at a target depth of 4 million reads in either paired-end mode (2×36 cycles) or single-end mode (1×75 cycles).

### RNA Bulk Sequencing

Clones were plated in 6-well plates 1 day before collection. On the day of collection, cells were checked for confluency within 70-90% and normal morphology. Cells were washed twice with PBS and stored at −80° C. immediately. RNA was purified for bulk sequencing using the Qiagen RNeasy Mini Kit (Qiagen #74106). RNA concentration and integrity were assessed using a 2100 BioAnalyzer (Agilent #G2939BA). Sequencing libraries were constructed using the TruSeq Stranded Total RNA Library Prep Gold (Illumina #20020598) with an input of 250 ng and 13 cycles final amplification. Final libraries were quantified using High Sensitivity D1000 ScreenTape (Agilent #5067-5584) on a 2200 TapeStation (Agilent #G2964AA) and Qubit 1× dsDNA HS Assay Kit (Invitrogen #Q32854). Samples were pooled equimolar with sequencing performed on an Illumina NovaSeq6000 SP 100 Cycle Flow Cell v1.5 as Paired-end 50 reads.

### Clone Derivation

hCECs were transduced with pHAGE-DD-KNL1^{Mut}-dCas9 and a sgRNA vector and DD-KNL1^{Mut}-dCas9 was stabilized with 100 nM Shield-1 (CheminPharma #CIP-S1, 0.5 nM) for 9 days. Three days after Shield-1 treatment, 20-500 cells were plated per 15-cm plate and were incubated in normal culture conditions until colonies were visible (˜2-3 weeks). Colonies were then picked by applying wax cylinders to the area surrounding each clone, trypsinizing the cells, and moving them to separate wells in 48-well plates for further expansion.

### Single-Cell RNA Sequencing

scRNA-seq libraries were prepared using the 10× Chromium Single-Cell 3′ v3 Gene Expression kit according to the manufacturer's instructions, including the manufacturer's protocol for cell surface protein (hashtag antibody) feature barcoding. Up to 10 TotalSeq-B hashtag antibodies (BioLegend) were used for multiplexing samples in each sequencing run.

### Immunofluorescence for Centromeric Damage

Cells were grown on poly-L-lysine coverslips, fixed in PFA (Sigma-Aldrich 8187081000) 2% in 1×PBS, and washed three times in 1×PBS. Fixed cells were permeabilized with 1×PBS and 0.2% Triton (Sigma-Aldrich X100, 500 ml) for 5 min at room temperature and washed again before being blocked with PBS-0.1% Tween 20 (Sigma-Aldrich P1379, 500 ml) plus 5% BSA for 10 min. Cells were then incubated with primary antibodies, γH2AX (Sigma-Aldrich 05-636) diluted 1:200 and CREST (Antibodies Incorporated 15-234-0001). After 45 min, cells were washed three times with 1×PBS and 0.1% Tween 20 and then incubated with the secondary antibodies anti-Mouse Alexa-488 (Jackson ImmunoResearch 711-545-152) and anti-Human Alexa 647 (Jackson ImmunoResearch 109-605-044). After 30 min, cells were washed twice with 1×PBS and 0.1% Tween 20 and once with 1×PBS with DAPI (Sigma-Aldrich 28718-90-3) diluted 1:750 from a 0.5 mg/ml stock. After 5 min, cells were washed one last time with 1×PBS and mounted using ProLong Glass Antifade Mountant (Thermo Scientific P36980). Images were acquired using a Thunder Leica fluorescent microscope at a 100× magnification and with a 0.2 μm z-stack and then processed using FIJI-ImageJ⁷⁵ to obtain a maximum projection.

### Quantification of Centromeric Damage

For each cell, the number of γH2AX and CREST colocalizing foci was scored using maximum projection images.

### Quantification of the Fluorescent Mean Intensity Signal

FIJI software was used to select the area of each cell and measure the signal mean intensity of the maximum projection images.

### Overexpression or Downregulation of SMAD2 and SMAD4

To overexpress human SMAD2 and SMAD4, cDNA for each gene was cloned into pHAGE vectors. CRISPRi (CRISPR-inhibition) was used to downregulate SMAD4 expression by transducing dCas9 into the cells using a pHAGE-dCas9 vector together with a CRISPR-interference sgRNA (GGCAGCGGCGACGACGACCA (SEQ ID NO: 7)) from Gilbert et al⁷⁶ cloned into pLentiGuide-Puro-FE.

### Quantification and Statistical Analysis

### Replicates, Statistical Analyses and Scale Bars

For each experiment we report in the figure legends the sample size and whether triplicates or duplicates were performed. Unless otherwise specified, triplicates or duplicates were biological, not technical. Unless otherwise specified; p-values are from the Wilcoxon test. If not otherwise specified; at least 50 nuclei or cells were analyzed in the FISH or IF experiments. Also, if not otherwise specified the scale bars in the FISH and IF images represent 5 μM.

Computational sgRNA Prediction

The CHM13 centromeric sequences and whole-genome reference were downloaded from the T2T Consortium (github.com/marbl/CHM13) 29 and the hg38 reference genome from the UCSC genome browser. For the CHM13 centromeric sequences, the HOR region with the classification “Live” or “HOR_L” was selected. For each HOR_L region, all possible SpCas9 sgRNA sites with a pattern comprising 20 nucleotides followed by NGG as PAM were searched. For each possible sgRNA, the numbers of binding sites in the centromeric HOR_L regions of each chromosome and in the whole genome were counted. The number of sgRNA binding sites was also determined using the hg38 reference. The GC content for each sgRNA was also determined.

For each sgRNA, two scores were determined: the chromosome specificity score, defined as the ratio between the number of binding sites on the centromere (HOR_L) of the target chromosome (chromosome that we intend to target) and the total number of sites across all centromeres (HOR_L) (given as a fraction or as a percentage after multiplication by 100), and the centromere specificity score, defined as the ratio between the number of binding sites on the centromere (HOR_L) of the target chromosome and the number of binding sites across the whole genome (given as a fraction or as a percentage after multiplication by 100).

The sgRNA efficiency was evaluated based on 3 parameters: 1) GC content, 2) total number of binding sites in the centromere of the target chromosome, and 3) sgRNA activity predicted from previous studies by Doench et al^{35,36}. With that method, the sgRNA activity is calculated based on 72 genetic features³⁶, which include the presence of certain nucleotides at specific positions along the sgRNA and the GC content. For a particular guide sⱼ, the model weights for the features i will be wᵢⱼ and the intercept will be int. The activity f(sⱼ) is then given via logistic regression as:

$$( {s}_{j} ) = int + {\sum }_{i} {w}_{i j}$$

$$f ( {s}_{j} ) = \frac{1}{1 + {e}^{- g ( {s}_{j} )}}$$

Predicted sgRNA activity f(sⱼ) falls into the range [0,1], with 0 as the worst score and 1 as the best score. Since CHM13 is a female-derived (XX) cell line, all binding sites for chromosome Y were evaluated based on hg38. Predicted sgRNAs are listed in Table S1.

### Automated Image Quantification of FISH Foci

In addition to manual counting of FISH foci (shown in FIG. 3 and FIG. 8), an automated image quantification was also performed (Table S2). FISH counts were calculated automatically using an in-house-developed python script, available publicly at github.com/davolilab/FISH-counting. Individual nuclei were segmented by applying an automatic threshold to the DAPI channel after smoothing and contrast enhancement. Thresholded objects were filtered for area and solidity to remove erroneously segmented regions. For probe detection within segmented nuclei, a white tophat filter was applied to remove small spurious regions, and then the “blob_log” function from scikit-image package⁷⁷ was utilized to identify and count fluorescent spots. Since it was observed that some FISH probes were incorrectly doubly counted, a distance cutoff was applied so that spots within a set (minimal) distance count as one spot. Then, the probe numbers were aggregated and the percentages for different spot counts were calculated. The script was run under a python 3.7 environment; for more details, see the github repository.

### Quantification of Foci Intensity

The regions corresponding to the FISH foci were determined by the threshold function of Fiji. Then, the average intensity of each determined region was calculated as the representative of the brightness of the focus by Fiji (used in FIG. 6E).

### Low-Pass Whole-Genome Sequencing Analysis

Low-pass (˜0.1-0.5×) whole-genome sequencing reads of cells were aligned to reference human genome hg38 by using BWA-mem (v0.7.17; github.com/lh3/bwa/releases/tag/v0.7.17)=⁷⁸, and duplicates were removed using GATK (Genome Analysis Toolkit, v4.1.7.0) (https://gatk.broadinstitute.org/hc/en-us)⁷⁹ with default parameters to generate analysis-ready BAM files. BAM files were processed by the R Package CopywriteR (v1.18.0; https://github.com/PeeperLab/CopywriteR)₈₀ to call the arm-level copy numbers.

### Bulk RNA-Seq Analysis Pipeline

RNA sequencing reads were processed, quality controlled, aligned, and quantified using the Seq-N-Slide software (github.com/igordot/sns)⁸¹. In brief, total RNA sequencing reads were trimmed using Trimmomatic (https://github.com/timflutre/trimmomatic)⁸² and mapped to the GENCODE human genome hg38 by STAR (github.com/alexdobin/STAR)⁸³. featureCounts (github.com/byee4/featureCounts)⁸⁴ was used to quantify reads and generate a genes-sample counts matrix. Differential gene expression (DGE) analysis was completed with DESeq2 in R (bioconductor.org/packages/release/bioc/html/DESeq2.html)⁵⁰. Gene ranks from DGE were used for pathway analysis using the GSEA preranked utility (www.gsea-msigdb.org/gsea/doc/GSEAUserGuideFrame.html)⁸⁵. Further plotting and statistical analyses were completed in R.

### Single-Cell RNA Sequencing Data Pre-Processing

The CellRanger v6.1 pipeline (10× Genomics) was used to process single-cell RNA sequencing data. CellRanger count was used to align sequences and generate gene expression matrices. Sequences were aligned to the pre-built GRCh38-2020-A human reference for CellRanger. Gene expression matrices were generated with each column representing a cell barcode and each row representing a gene or hashtag oligo sequences (HTO).

To identify the sample of origin for each cell barcode, the HTO count data from each 10× Chromium experiment were demultiplexed using the Seurat v4.0.3 package for R v4.1 (https://github.com/satijalab/seurat)⁸⁶. Cell barcodes that could be confidently assigned to a single sample were kept. Several quality control thresholds were applied uniquely to each dataset on total gene number, total UMI counts, and total HTO counts to remove low-quality cells and potential cell doublets. Cells were also discarded if their proportion of total gene counts that could be attributed to mitochondrial genes exceeded 10%.

### Modified CopyKat Analysis

A modified version of the CopyKat v1.0.5 (github.com/navinlabcode/copykat)⁴⁶ pipeline for R was used to generate a copy number alteration (SCNA) score for each chromosome arm in each cell. Hashtagged samples from the same cell line in each 10×Chromium dataset were grouped together for analysis. Each such group of samples contained a diploid control sample used to set the SCNA value baseline centered around 0. For each analysis, genes expressed in less than 5% of the cells, HLA genes, and cell-cycle genes were excluded. The log-Freeman-Tukey transformation was used to stabilize variance and dlmSmooth( ) was used to smooth outliers. The diploid control sample for each set was used to calculate a baseline expression level for each gene. This value was subtracted from the samples in the set, centering the control sample expression around 0. Genes expressed in less than 10% of cells were then excluded from further analysis. The original CopyKat pipeline splits the transcriptome into artificial segments based on similar expression, and calculates a SCNA value for each segment. Instead, we generated a SCNA value for each chromosome arm by calculating the mean gene expression for the genes on that arm.

A single SCNA value for the entire chromosome 18 was calculated using genes on both the p and q arms of the chromosome instead of each arm individually, due to its relatively small size. SCNA values for chromosomes 13, 14, 15, 21, and 22 were calculated only using genes on their respective q arms. Gains or losses of a chromosome arm relative to the control sample (diploid) were called based on a threshold calculated from the control sample for each chromosome arm. The threshold is calculated as

$$median \pm ( 2.5 \times M A D )$$

where the median is calculated from the SCNA values for each arm in the control sample, and the median absolute deviation (MAD) is calculated by the mad( ) function from the stats R package. Gains (or losses) are then called for a chromosome arm if its SCNA value is above (or below) the threshold for its sample set.

### CopyKat Data Visualization

Heatmaps were generated using the ComplexHeatmap v2.8 R package⁸⁷. Each row represents one cell, each column represents a chromosome arm, and each value is the corresponding SCNA score. Column widths were scaled to the number of genes on the arm. For the heatmaps, cells were clustered by row of the chromosome of interest. Bar graphs were generated using the ggplot2 v3.3.5 R package.

### Survival Analysis

For survival analysis, the disease-free interval (DFI) and related clinical data were downloaded from cBioPortal⁸⁸. Arm-level copy number was downloaded from TCGA Firehose Legacy (https://gdac.broadinstitute.org). For each patient, purity α, ploidy τ, and integer copy number q(x) data were downloaded from GDC (https://gdc.cancer.gov/about-data/publications/pancanatlas). Before the analysis, the arm-level copy number values R(x) were adjusted using the formula below:

$${R}^{′} ( x ) = \frac{q ( x )}{τ} = \frac{α \times τ \times R ( x ) + 2 ( 1 - α ) \times R ( x ) - 2 ( 1 - α )}{α \times τ}$$

Patients with arm-level log 2 ratio less than −0.3 would be regarded as an arm-level loss event to evaluate patients based on the presence or absence of 18q arm loss. A log-rank test between the stratified patients and the Kaplan-Meier method was used to calculate the p-value and plot survival curves. Patients for whom clinical survival information was unavailable were excluded from the analysis. In addition, a Cox proportional hazards (PH) regression model was used to calculate each gene's hazard ratio (HR) between the top 50% and bottom 50% expression.

### Gene Rank Score Analysis

For each gene on chromosome 18, we calculated the DNA-RNA Spearman's correlation (rho value) from the TCGA-COADREAD dataset. Genes with no or very low frequency of SCNA (−0.02<DNA log₂ FC<0.02 in >70% of the patients) were removed because for those genes very little or no variance at the DNA level is likely to influence the correlation value. The Cox proportional-hazards model was then applied to estimate the association between the expression level of each gene and patients' survival. The TUSON algorithm for predicting the likelihood for a gene to behave as a tumor-suppressor gene (TSG) based on its pattern of point mutation was from Davoli et al.⁴ and was applied to the latest available TCGA dataset of point mutations. A gene rank score was generated based on the rank sum of the following three parameters: DNA-RNA correlation, hazard ratio from Cox proportional hazards regression, and q-value from TUSON-based TSG prediction. In other words, for each gene, the (three) rank position values determined based on the three parameters listed above were summed.

### Supplementary Material

### Legends to Supplementary Tables

Table S1. Prediction of sgRNA for each chromosome with CHM13 genome, Related to FIG. 1 (see also Methods).
Table S1 contains in the first tab the sgRNA prediction for 76 selected sgRNAs across all chromosomes except chromosome Y. This table contains the sgRNA sequence, chromosome location, binding sites for specific CHM13 chromosome centromere, total binding sites across all centromeres, chromosome specificity (ratio between the number of binding sites on the centromere of that chromosome and the total number of sites across all centromeres), centromere specificity (ratio between the number of binding sites on the centromere of that chromosome and the number of binding sites across the whole genome), binding sites across whole CHM13 genome and hg38 genome, activity score (Doench score) and validation results by imaging. The table contains predictions of sgRNAs for every single chromosome, as indicated.
Table S2. Acrocentric chromosome sgRNA prediction in CHM13 and hg38 genome, Related to FIG. 4. This table contains the specific sgRNA across different acrocentric chromosomes and includes predicted binding sites across different chromosomes, total binding sites across all centromeres with hg38 genome, and total binding sites across the whole hg38 genome.
Table S3. Automated quantification of FISH foci after KaryoCreate, Related to FIG. 3. This table contains the number of FISH foci quantified using an Automated FISH counting (see Methods) designed to score FISH signals in interphase cells.

TABLE S1
SELECTED gRNAs

| | | | bind- | | | | | | | | | | | | | | |
|---|---|---|---|---|---|---|---|---|---|---|---|---|---|---|---|---|---|
| | | | ing | | | | | | | | | bind- | | Per- | | | |
| | | | sites | | | | | | | | | ing | | cent- | | | |
| | | | (CHM | | | | | | | | | sites | | age | | | |
| | | | 13- | | | | | | | | | (CHM13- | | of | | | |
| | | | spe- | bind- | | | | | | | | spe- | | cells | | | |
| | | | cif- | ing | | | | | | | | cif- | | show- | | | |
| | | | ic | sites | | | | | | | | ic | | ing | | | |
| | | | chro- | (CHM | | bind- | | | | | | chro- | | foci | | | |
| | | | mo- | 13- | chro- | ing | bind- | | | | | mo- | Val- | (hCEC) | | HOR_ | |
| | | | some | all | mo- | sites | ing | centro- | | | | some | i- | re- | | L | HOR |
| | | | cen- | cen- | some | (CHM | sites | mere | | | | centro- | dated_ | la- | | in | in |
| | | sg | tro- | tro- | spe- | 13- | (hg38 | spe- | | Ac- | CHM13 | mere | by | ted | bind- | this | this |
| chro- | | RNA | mere | mere | cif- | whole | whole | cif- | GC_ | tiv- | HOR_ | Not | Im- | to | ing | chro- | chro- |
| mo- | | _ | HOR_ | HOR_ | ic- | ge- | ge- | ic- | cont- | ity | L_ | HOR_ | ag- | FIG.1/ | HOR_ | mo- | mo- |
| some | Name | seq | L) | L) | ity | nome) | nome) | ity | ent | score | length | L) | ing | S1 | L | some | some |
| Chr1 | gRNA | AGTTG | 9028 | 22999 | 0.393 | 23007 | 10774 | 0.392 | 35 | 0.655 | 4504439 | 0 | NO | | hor_ | hor_ | hor_ |
| | 1-3 | AATAC | | | | | | | | | | | | | 1_ | 1_ | 1_ |
| | | ACACA | | | | | | | | | | | | | 5 | 5 | 1 |
| | | ACACA | | | | | | | | | | | | | (S1C1 | (S1C1 | (S3C1 |
| | | (SEQ | | | | | | | | | | | | | / | / | H2-A, |
| | | ID | | | | | | | | | | | | | 5/ | 5/ | B, |
| | | NO: | | | | | | | | | | | | | 19 | 19 | C); |
| | | 8) | | | | | | | | | | | | | H1L) | H1L) | hor_ |
| | | | | | | | | | | | | | | | | | 1_2_ |
| | | | | | | | | | | | | | | | | | (S3C1 |
| | | | | | | | | | | | | | | | | | pH2-A, |
| | | | | | | | | | | | | | | | | | B); |
| | | | | | | | | | | | | | | | | | hor_ |
| | | | | | | | | | | | | | | | | | 1_ |
| | | | | | | | | | | | | | | | | | 3 |
| | | | | | | | | | | | | | | | | | (S3C1 |
| | | | | | | | | | | | | | | | | | pH2-B); |
| | | | | | | | | | | | | | | | | | hor_ |
| | | | | | | | | | | | | | | | | | 1_ |
| | | | | | | | | | | | | | | | | | 4 |
| | | | | | | | | | | | | | | | | | (S3C1 |
| | | | | | | | | | | | | | | | | | pH2-A); |
| | | | | | | | | | | | | | | | | | hor_ |
| | | | | | | | | | | | | | | | | | 1_ |
| | | | | | | | | | | | | | | | | | 5 |
| | | | | | | | | | | | | | | | | | (S1C1 |
| | | | | | | | | | | | | | | | | | / |
| | | | | | | | | | | | | | | | | | 5/ |
| | | | | | | | | | | | | | | | | | 19 |
| | | | | | | | | | | | | | | | | | H1L); |
| | | | | | | | | | | | | | | | | | hor_ |
| | | | | | | | | | | | | | | | | | 1_ |
| | | | | | | | | | | | | | | | | | 6 |
| | | | | | | | | | | | | | | | | | (S3C1 |
| | | | | | | | | | | | | | | | | | pH2-A); |
| | | | | | | | | | | | | | | | | | hor_ |
| | | | | | | | | | | | | | | | | | 1_ |
| | | | | | | | | | | | | | | | | | 7 |
| | | | | | | | | | | | | | | | | | (S3C1 |
| | | | | | | | | | | | | | | | | | qH2-C, |
| | | | | | | | | | | | | | | | | | D); |
| | | | | | | | | | | | | | | | | | hor_ |
| | | | | | | | | | | | | | | | | | 1_ |
| | | | | | | | | | | | | | | | | | 8 |
| | | | | | | | | | | | | | | | | | (S3C1 |
| | | | | | | | | | | | | | | | | | qH2-D); |
| | | | | | | | | | | | | | | | | | hor_ |
| | | | | | | | | | | | | | | | | | 1_ |
| | | | | | | | | | | | | | | | | | 9 |
| | | | | | | | | | | | | | | | | | (S3C1 |
| | | | | | | | | | | | | | | | | | qH2-C) |
| Chr1 | gRNA | TTCTA | 6008 | 15541 | 0.387 | 15543 | 6026 | 0.387 | 40 | 0.388 | 4504439 | 0 | YES | 62 | hor_ | hor_ | hor_ |
| | 1-4 | CCATT | | | | | | | | | | | but | | 1_ | 1_ | 1_ |
| | | GACCT | | | | | | | | | | | more | | 5 | 5 | 1 |
| | | CAAAG | | | | | | | | | | | than | | (S1C1 | (S1C1 | (S3C1 |
| | | (SEQ | | | | | | | | | | | 2 | | / | / | H2-A, |
| | | ID | | | | | | | | | | | foci | | 5/ | 5/ | B, |
| | | NO: | | | | | | | | | | | per | | 19 | 19 | C); |
| | | 9) | | | | | | | | | | | cell | | H1L) | H1L) | hor_ |
| | | | | | | | | | | | | | | | | | 1_ |
| | | | | | | | | | | | | | | | | | 2 |
| | | | | | | | | | | | | | | | | | (S3C1 |
| | | | | | | | | | | | | | | | | | pH2-A, |
| | | | | | | | | | | | | | | | | | B); |
| | | | | | | | | | | | | | | | | | hor_ |
| | | | | | | | | | | | | | | | | | 1_ |
| | | | | | | | | | | | | | | | | | 3 |
| | | | | | | | | | | | | | | | | | (S3C1 |
| | | | | | | | | | | | | | | | | | pH2-B); |
| | | | | | | | | | | | | | | | | | hor_ |
| | | | | | | | | | | | | | | | | | 1_ |
| | | | | | | | | | | | | | | | | | 4 |
| | | | | | | | | | | | | | | | | | (S3C1 |
| | | | | | | | | | | | | | | | | | pH2-A); |
| | | | | | | | | | | | | | | | | | hor_ |
| | | | | | | | | | | | | | | | | | 1_ |
| | | | | | | | | | | | | | | | | | 5 |
| | | | | | | | | | | | | | | | | | (S1C1 |
| | | | | | | | | | | | | | | | | | / |
| | | | | | | | | | | | | | | | | | 5/ |
| | | | | | | | | | | | | | | | | | 19 |
| | | | | | | | | | | | | | | | | | H1L); |
| | | | | | | | | | | | | | | | | | hor_ |
| | | | | | | | | | | | | | | | | | 1_ |
| | | | | | | | | | | | | | | | | | 6 |
| | | | | | | | | | | | | | | | | | (S3C1 |
| | | | | | | | | | | | | | | | | | pH2-A); |
| | | | | | | | | | | | | | | | | | hor_ |
| | | | | | | | | | | | | | | | | | 1_ |
| | | | | | | | | | | | | | | | | | 7 |
| | | | | | | | | | | | | | | | | | (S3C1 |
| | | | | | | | | | | | | | | | | | qH2-C, |
| | | | | | | | | | | | | | | | | | D); |
| | | | | | | | | | | | | | | | | | hor_ |
| | | | | | | | | | | | | | | | | | 18 |
| | | | | | | | | | | | | | | | | | (S3C1 |
| | | | | | | | | | | | | | | | | | qH2-D); |
| | | | | | | | | | | | | | | | | | hor_ |
| | | | | | | | | | | | | | | | | | 1_ |
| | | | | | | | | | | | | | | | | | 9 |
| | | | | | | | | | | | | | | | | | (S3C1 |
| | | | | | | | | | | | | | | | | | qH2-C) |
| Chr2 | gRNA | TGGAC | 1327 | 1335 | 0.994 | 1340 | 953 | 0.99 | 55 | 0.07 | 2339480 | 0 | YES | | hor_ | hor_ | hor_ |
| | 2-2 | ATTTG | | | | | | | | | | | | | 2_ | 22 | 2_ |
| | | GAGCG | | | | | | | | | | | | | 2 | (S2C2 | 1 |
| | | CTCTC | | | | | | | | | | | | | (S2C2 | H1L) | (S2C2 |
| | | (SEQ | | | | | | | | | | | | | H1L) | | pH2-B); |
| | | ID | | | | | | | | | | | | | | | hor_ |
| | | NO: | | | | | | | | | | | | | | | 2_ |
| | | 10) | | | | | | | | | | | | | | | 2 |
| | | | | | | | | | | | | | | | | | (S2C2 |
| | | | | | | | | | | | | | | | | | H1L); |
| | | | | | | | | | | | | | | | | | hor_ |
| | | | | | | | | | | | | | | | | | 2_ |
| | | | | | | | | | | | | | | | | | 3 |
| | | | | | | | | | | | | | | | | | (S2C2 |
| | | | | | | | | | | | | | | | | | qH2-A) |
| Chr2 | gRNA | AACAG | 2230 | 2231 | 1 | 2233 | 2240 | 0.999 | 45 | 0.127 | 2339480 | 0 | NO | | hor_ | hor_ | hor_ |
| | 2-3 | TCCCT | | | | | | | | | | | | | 2_ | 2_ | 2_ |
| | | TTCAT | | | | | | | | | | | | | 2 | 2 | 1 |
| | | AGAGC | | | | | | | | | | | | | (S2C2 | (S2C2 | (S2C2 |
| | | (SEQ | | | | | | | | | | | | | H1L) | H1L) | pH2-B); |
| | | ID | | | | | | | | | | | | | | | hor_ |
| | | NO: | | | | | | | | | | | | | | | 2_ |
| | | 11) | | | | | | | | | | | | | | | 2 |
| | | | | | | | | | | | | | | | | | (S2C2 |
| | | | | | | | | | | | | | | | | | H1L); |
| | | | | | | | | | | | | | | | | | hor_ |
| | | | | | | | | | | | | | | | | | 2_ |
| | | | | | | | | | | | | | | | | | 3 |
| | | | | | | | | | | | | | | | | | (S2C2 |
| | | | | | | | | | | | | | | | | | qH2-A) |
| Chr2 | gRNA | GCTTC | 2617 | 2617 | 1 | 2617 | 2790 | 1 | 40 | 0.506 | 2339480 | 0 | YES | 65 | hor_ | hor_ | hor_ |
| | 2-4 | AACAC | | | | | | | | | | | | | 2_ | 2_ | 2_ |
| | | TGTTA | | | | | | | | | | | | | 2 | 2 | 1 |
| | | GTTGA | | | | | | | | | | | | | (S2C2 | (S2C2 | (S2C2 |
| | | (SEQ | | | | | | | | | | | | | H1L) | H1L) | pH2-B); |
| | | ID | | | | | | | | | | | | | | | hor_ |
| | | NO: | | | | | | | | | | | | | | | 2_ |
| | | 12) | | | | | | | | | | | | | | | 2 |
| | | | | | | | | | | | | | | | | | (S2C2 |
| | | | | | | | | | | | | | | | | | H1L); |
| | | | | | | | | | | | | | | | | | hor_ |
| | | | | | | | | | | | | | | | | | 2_ |
| | | | | | | | | | | | | | | | | | 3 |
| | | | | | | | | | | | | | | | | | (S2C2 |
| | | | | | | | | | | | | | | | | | qH2-A) |
| Chr3 | gRNA | TTCCA | 453 | 453 | 1 | 453 | 692 | 1 | 50 | 0.248 | 1443021 | 0 | NO | | hor_ | hor_ | hor_ |
| | 3-1 | ATCTG | | | | | | | | | | | | | 3_ | 3_ | 3_ |
| | | CTCCG | | | | | | | | | | | | | 2 | 2 | 1 |
| | | CCTAA | | | | | | | | | | | | | (S01/ | (S01/ | (S1C3 |
| | | (SEQ | | | | | | | | | | | | | 1C3H1L); | 1C3H1L); | H2); |
| | | ID | | | | | | | | | | | | | hor_ | hor_ | hor_ |
| | | NO: | | | | | | | | | | | | | 3_ | 3_ | 3_ |
| | | 13) | | | | | | | | | | | | | 3 | 3 | 2 |
| | | | | | | | | | | | | | | | (S01/ | (S01/ | (S01/ |
| | | | | | | | | | | | | | | | 1C3H1L); | 1C3H1L); | 1C3H1L); |
| | | | | | | | | | | | | | | | hor_ | hor_ | hor_ |
| | | | | | | | | | | | | | | | 3_ | 3_ | 3_ |
| | | | | | | | | | | | | | | | 4 | 4 | 3 |
| | | | | | | | | | | | | | | | (S01/ | (S01/ | (S01/ |
| | | | | | | | | | | | | | | | 1C3H1L) | 1C3H1L) | 1C3H1L); |
| | | | | | | | | | | | | | | | | | hor_ |
| | | | | | | | | | | | | | | | | | 3_ |
| | | | | | | | | | | | | | | | | | 4 |
| | | | | | | | | | | | | | | | | | (S01/ |
| | | | | | | | | | | | | | | | | | 1C3H1L) |
| Chr3 | gRNA | TTCCT | 450 | 450 | 1 | 450 | 691 | 1 | 50 | 0.112 | 1443021 | 0 | NO | | hor_ | hor_ | hor_ |
| | 3-2 | TTAGG | | | | | | | | | | | | | 3_ | 3_ | 3_ |
| | | CGGAG | | | | | | | | | | | | | 2 | 2 | 1 |
| | | CAGAT | | | | | | | | | | | | | (S01/ | (S01/ | (S1C3 |
| | | (SEQ | | | | | | | | | | | | | 1C3H1L); | 1C3H1L); | H2); |
| | | ID | | | | | | | | | | | | | hor_ | hor_ | hor_ |
| | | NO: | | | | | | | | | | | | | 33 | 3_ | 3_ |
| | | 14) | | | | | | | | | | | | | (S01/ | 3 | 2 |
| | | | | | | | | | | | | | | | 1C3H1L); | (S01/ | (S01/ |
| | | | | | | | | | | | | | | | hor_ | 1C3H1L); | 1C3H1L); |
| | | | | | | | | | | | | | | | 3_ | hor_ | hor_ |
| | | | | | | | | | | | | | | | 4 | 3_ | 3_ |
| | | | | | | | | | | | | | | | (S01/ | 4 | 3 |
| | | | | | | | | | | | | | | | 1C3H1L) | (S01/ | (S01/ |
| | | | | | | | | | | | | | | | | 1C3H1L) | 1C3H1L); |
| | | | | | | | | | | | | | | | | | hor_ |
| | | | | | | | | | | | | | | | | | 3_ |
| | | | | | | | | | | | | | | | | | 4 |
| | | | | | | | | | | | | | | | | | (S01/ |
| | | | | | | | | | | | | | | | | | 1C3H1L) |
| Chr3 | gRNA | CTTTT | 1373 | 1396 | 0.984 | 2163 | 2814 | 0.635 | 40 | 0.652 | 1443021 | 0 | NO | | hor_ | hor_ | hor_ |
| | 3-3 | TGCAG | | | | | | | | | | | | | 3_ | 3_ | 3_ |
| | | AATCT | | | | | | | | | | | | | 2 | 2 | 1 |
| | | GCAAG | | | | | | | | | | | | | (S01/ | (S01/ | (S1C3 |
| | | (SEQ | | | | | | | | | | | | | 1C3H1L); | 1C3H1L); | H2); |
| | | ID | | | | | | | | | | | | | hor_ | hor_ | hor_ |
| | | NO: | | | | | | | | | | | | | 3_ | 3_ | 3_ |
| | | 15) | | | | | | | | | | | | | 3 | 3 | 2 |
| | | | | | | | | | | | | | | | (S01/ | (S01/ | (S01/ |
| | | | | | | | | | | | | | | | 1C3H1L); | 1C3H1L); | 1C3H1L); |
| | | | | | | | | | | | | | | | hor_ | hor_ | hor_ |
| | | | | | | | | | | | | | | | 3_ | 3_ | 3_ |
| | | | | | | | | | | | | | | | 4 | 4 | 3 |
| | | | | | | | | | | | | | | | (S01/ | (S01/ | (S01/ |
| | | | | | | | | | | | | | | | 1C3H1L) | 1C3H1L) | 1C3H1L); |
| | | | | | | | | | | | | | | | | | hor_ |
| | | | | | | | | | | | | | | | | | 3_ |
| | | | | | | | | | | | | | | | | | 4 |
| | | | | | | | | | | | | | | | | | (S01/ |
| | | | | | | | | | | | | | | | | | 1C3H1L) |
| Chr3 | gRNA | TTCAA | 535 | 3914 | 0.137 | 4178 | 4455 | 0.128 | 50 | 0.164 | 1443021 | 0 | NO | | hor_ | hor_ | hor_ |
| | 3-4 | GCGCT | | | | | | | | | | | | | 3_ | 3_ | 3_ |
| | | TTGAG | | | | | | | | | | | | | 2 | 2 | 1 |
| | | GCCAA | | | | | | | | | | | | | (S01/ | (S01/ | (S1C3 |
| | | (SEQ | | | | | | | | | | | | | 1C3H1L); | 1C3H1L); | H2); |
| | | ID | | | | | | | | | | | | | hor_ | hor_ | hor_ |
| | | NO: | | | | | | | | | | | | | 3_ | 3_ | 3_ |
| | | 16) | | | | | | | | | | | | | 3 | 3 | 2 |
| | | | | | | | | | | | | | | | (S01/ | (S01/ | (S01/ |
| | | | | | | | | | | | | | | | 1C3H1L); | 1C3H1L); | 1C3H1L); |
| | | | | | | | | | | | | | | | hor_ | hor_ | hor_ |
| | | | | | | | | | | | | | | | 34 | 34 | 3_ |
| | | | | | | | | | | | | | | | (S01/ | (S01/ | 3 |
| | | | | | | | | | | | | | | | 1C3H1L) | 1C3H1L) | (S01/ |
| | | | | | | | | | | | | | | | | | 1C3H1L); |
| | | | | | | | | | | | | | | | | | hor_ |
| | | | | | | | | | | | | | | | | | 3_ |
| | | | | | | | | | | | | | | | | | 4 |
| | | | | | | | | | | | | | | | | | (S01/ |
| | | | | | | | | | | | | | | | | | 1C3H1L) |
| Chr4 | gRNA | TTCGA | 2066 | 2071 | 0.998 | 2082 | 1311 | 0.992 | 55 | 0.064 | 3702932 | 0 | NO | | hor_ | hor_ | hor_ |
| | 4-1 | GCGCT | | | | | | | | | | | | | 4_ | 4_ | 4_ |
| | | TTGAG | | | | | | | | | | | | | 1 | 1 | 1 |
| | | GCCTA | | | | | | | | | | | | | (S2C4 | (S2C4 | (S2C4 |
| | | (SEQ | | | | | | | | | | | | | H1L); | H1L); | H1L); |
| | | ID | | | | | | | | | | | | | hor_ | hor_ | hor_ |
| | | NO: | | | | | | | | | | | | | 4_ | 4_ | 4_ |
| | | 17) | | | | | | | | | | | | | 2 | 2 | 2 |
| | | | | | | | | | | | | | | | (S2C4 | (S2C4 | (S2C4 |
| | | | | | | | | | | | | | | | H1L); | H1L); | H1L); |
| | | | | | | | | | | | | | | | hor_ | hor_ | hor_ |
| | | | | | | | | | | | | | | | 4_ | 4_ | 4_ |
| | | | | | | | | | | | | | | | 3 | 3 | 3 |
| | | | | | | | | | | | | | | | (S2C4 | (S2C4 | (S2C4 |
| | | | | | | | | | | | | | | | H1L) | H1L) | H1L); |
| | | | | | | | | | | | | | | | | | hor_ |
| | | | | | | | | | | | | | | | | | 4_ |
| | | | | | | | | | | | | | | | | | 4 |
| | | | | | | | | | | | | | | | | | (S5C4 |
| | | | | | | | | | | | | | | | | | H2) |
| Chr4 | gRNA | CCACC | 2301 | 2302 | 1 | 2305 | 1218 | 0.998 | 45 | 0.625 | 3702932 | 0 | NO | | hor_ | hor_ | hor_ |
| | 4-2 | TGCAG | | | | | | | | | | | | | 4_ | 4_ | 41 |
| | | ATTCT | | | | | | | | | | | | | 1 | 1 | (S2C4 |
| | | ACAAA | | | | | | | | | | | | | (S2C4 | (S2C4 | H1L); |
| | | (SEQ | | | | | | | | | | | | | H1L); | H1L); | hor_ |
| | | ID | | | | | | | | | | | | | hor_ | hor_ | 42_ |
| | | NO: | | | | | | | | | | | | | 4_ | 4_ | (S2C4 |
| | | 18) | | | | | | | | | | | | | 2 | 2 | H1L); |
| | | | | | | | | | | | | | | | (S2C4 | (S2C4 | hor_ |
| | | | | | | | | | | | | | | | H1L); | H1L); | 43 |
| | | | | | | | | | | | | | | | hor_ | hor_ | (S2C4 |
| | | | | | | | | | | | | | | | 43_ | 4_ | H1L); |
| | | | | | | | | | | | | | | | (S2C4 | 3 | hor_ |
| | | | | | | | | | | | | | | | H1L) | (S2C4 | 4_ |
| | | | | | | | | | | | | | | | | H1L) | 4 |
| | | | | | | | | | | | | | | | | | (S5C4 |
| | | | | | | | | | | | | | | | | | H2) |
| Chr4 | gRNA | CCTTT | 2252 | 2253 | 1 | 2315 | 1233 | 0.973 | 45 | 0.837 | 3702932 | 0 | YES | 62 | hor_ | hor_ | hor_ |
| | 4-3 | TGTAG | | | | | | | | | | | | | 4_ | 4_ | 4_ |
| | | AATCT | | | | | | | | | | | | | 1 | 1 | 1 |
| | | GCAGG | | | | | | | | | | | | | (S2C4 | (S2C4 | (S2C4 |
| | | (SEQ | | | | | | | | | | | | | H1L); | H1L); | H1L); |
| | | ID | | | | | | | | | | | | | hor_ | hor_ | hor_ |
| | | NO: | | | | | | | | | | | | | 4_ | 4_ | 42_ |
| | | 19) | | | | | | | | | | | | | 2 | 2 | (S2C4 |
| | | | | | | | | | | | | | | | (S2C4 | (S2C4 | H1L); |
| | | | | | | | | | | | | | | | H1L); | H1L); | hor_ |
| | | | | | | | | | | | | | | | hor_ | hor_ | 4_ |
| | | | | | | | | | | | | | | | 4_ | 4_ | 3 |
| | | | | | | | | | | | | | | | 3 | 3 | (S2C4 |
| | | | | | | | | | | | | | | | (S2C4 | (S2C4 | H1L); |
| | | | | | | | | | | | | | | | H1L) | H1L) | hor_ |
| | | | | | | | | | | | | | | | | | 4_ |
| | | | | | | | | | | | | | | | | | 4 |
| | | | | | | | | | | | | | | | | | (S5C4 |
| | | | | | | | | | | | | | | | | | H2) |
| Chr4 | gRNA | CTTTC | 2179 | 4057 | 0.537 | 4057 | 2451 | 0.537 | 50 | 0.432 | 3702932 | 0 | NO | | hor_ | hor_ | hor_ |
| | 4-4 | TGCAC | | | | | | | | | | | | | 1 | 4_ | 4_ |
| | | TACCT | | | | | | | | | | | | | (S2C4 | 1 | 1 |
| | | GGAAG | | | | | | | | | | | | | H1L); | (S2C4 | (S2C4 |
| | | (SEQ | | | | | | | | | | | | | hor_ | H1L); | H1L); |
| | | ID | | | | | | | | | | | | | 4_ | hor_ | hor_ |
| | | NO: | | | | | | | | | | | | | 2 | 4_ | 42_ |
| | | 20) | | | | | | | | | | | | | (S2C4 | 2 | (S2C4 |
| | | | | | | | | | | | | | | | H1L); | (S2C4 | H1L); |
| | | | | | | | | | | | | | | | hor_ | H1L); | hor_ |
| | | | | | | | | | | | | | | | 4_ | hor_ | 4_ |
| | | | | | | | | | | | | | | | 3 | 4_ | 3 |
| | | | | | | | | | | | | | | | (S2C4 | 3 | (S2C4 |
| | | | | | | | | | | | | | | | H1L) | (S2C4 | H1L); |
| | | | | | | | | | | | | | | | | H1L) | hor_ |
| | | | | | | | | | | | | | | | | | 44 |
| | | | | | | | | | | | | | | | | | (S5C4 |
| | | | | | | | | | | | | | | | | | H2) |
| Chr5 | gRNA | AGTTG | 2520 | 2530 | 0.996 | 2530 | 2595 | 0.996 | 40 | 0.534 | 2529952 | 0 | YES | 59 | hor_ | hor_ | hor_ |
| | 5-3 | AACAC | | | | | | | | | | | | | 5_ | 5_ | 5_ |
| | | ACACA | | | | | | | | | | | | | 3 | 3 | 1 |
| | | ACACA | | | | | | | | | | | | | (S1C1 | (S1C1 | (S5C5 |
| | | (SEQ | | | | | | | | | | | | | / | / | pH5); |
| | | ID | | | | | | | | | | | | | 5/ | 5/ | hor_ |
| | | NO: | | | | | | | | | | | | | 19 | 19 | 52_ |
| | | 21) | | | | | | | | | | | | | H1L); | H1L); | (S1C5 |
| | | | | | | | | | | | | | | | hor_ | hor_ | pH2); |
| | | | | | | | | | | | | | | | 5_ | 55 | hor_ |
| | | | | | | | | | | | | | | | 5 | (S1C1 | 5_ |
| | | | | | | | | | | | | | | | (S1C1 | / | 3 |
| | | | | | | | | | | | | | | | / | 5/ | (S1C1 |
| | | | | | | | | | | | | | | | 5/ | 19 | / |
| | | | | | | | | | | | | | | | 19 | H1L) | 5/ |
| | | | | | | | | | | | | | | | H1L) | | 19 |
| | | | | | | | | | | | | | | | | | H1L); |
| | | | | | | | | | | | | | | | | | hor_ |
| | | | | | | | | | | | | | | | | | 5_ |
| | | | | | | | | | | | | | | | | | 4 |
| | | | | | | | | | | | | | | | | | (S1C5 |
| | | | | | | | | | | | | | | | | | pH2); |
| | | | | | | | | | | | | | | | | | hor_ |
| | | | | | | | | | | | | | | | | | 5_ |
| | | | | | | | | | | | | | | | | | 5 |
| | | | | | | | | | | | | | | | | | (S1C1 |
| | | | | | | | | | | | | | | | | | / |
| | | | | | | | | | | | | | | | | | 5/ |
| | | | | | | | | | | | | | | | | | 19 |
| | | | | | | | | | | | | | | | | | H1L); |
| | | | | | | | | | | | | | | | | | hor_ |
| | | | | | | | | | | | | | | | | | 56_ |
| | | | | | | | | | | | | | | | | | (S5C5 |
| | | | | | | | | | | | | | | | | | pH6); |
| | | | | | | | | | | | | | | | | | hor_ |
| | | | | | | | | | | | | | | | | | 5_ |
| | | | | | | | | | | | | | | | | | 7 |
| | | | | | | | | | | | | | | | | | (S5C5 |
| | | | | | | | | | | | | | | | | | pH7-B); |
| | | | | | | | | | | | | | | | | | hor_ |
| | | | | | | | | | | | | | | | | | 5_ |
| | | | | | | | | | | | | | | | | | 8 |
| | | | | | | | | | | | | | | | | | (S5C5 |
| | | | | | | | | | | | | | | | | | / |
| | | | | | | | | | | | | | | | | | 19qH4-B) |
| Chr5 | gRNA | TACAA | 1613 | 1616 | 0.998 | 1616 | 781 | 0.998 | 40 | 0.128 | 2529952 | 0 | NO | | hor_ | hor_ | hor_ |
| | 5-4 | GTCTG | | | | | | | | | | | | | 5_ | 5_ | 5_ |
| | | CTCTG | | | | | | | | | | | | | 3 | 3 | 1 |
| | | TGTAA | | | | | | | | | | | | | (S1C1 | (S1C1 | (S5C5 |
| | | (SEQ | | | | | | | | | | | | | / | / | pH5); |
| | | ID | | | | | | | | | | | | | 5/ | 5/ | hor_ |
| | | NO: | | | | | | | | | | | | | 19 | 19H1L); | 52_ |
| | | 22) | | | | | | | | | | | | | H1L); | hor_ | (S1C5 |
| | | | | | | | | | | | | | | | hor_ | 5_ | pH2); |
| | | | | | | | | | | | | | | | 5_ | 5 | hor_ |
| | | | | | | | | | | | | | | | 5 | (S1C1 | 5_ |
| | | | | | | | | | | | | | | | (S1C1 | / | 3 |
| | | | | | | | | | | | | | | | / | 5/ | (S1C1 |
| | | | | | | | | | | | | | | | 5/ | 19 | / |
| | | | | | | | | | | | | | | | 19 | H1L) | 5/ |
| | | | | | | | | | | | | | | | H1L) | | 19 |
| | | | | | | | | | | | | | | | | | H1L); |
| | | | | | | | | | | | | | | | | | hor_ |
| | | | | | | | | | | | | | | | | | 5_ |
| | | | | | | | | | | | | | | | | | 4 |
| | | | | | | | | | | | | | | | | | (S1C5 |
| | | | | | | | | | | | | | | | | | pH2); |
| | | | | | | | | | | | | | | | | | hor_ |
| | | | | | | | | | | | | | | | | | 5_ |
| | | | | | | | | | | | | | | | | | 5 |
| | | | | | | | | | | | | | | | | | (S1C1 |
| | | | | | | | | | | | | | | | | | / |
| | | | | | | | | | | | | | | | | | 5/ |
| | | | | | | | | | | | | | | | | | 19 |
| | | | | | | | | | | | | | | | | | H1L); |
| | | | | | | | | | | | | | | | | | hor_ |
| | | | | | | | | | | | | | | | | | 56_ |
| | | | | | | | | | | | | | | | | | (S5C5 |
| | | | | | | | | | | | | | | | | | pH6); |
| | | | | | | | | | | | | | | | | | hor_ |
| | | | | | | | | | | | | | | | | | 5_ |
| | | | | | | | | | | | | | | | | | 7 |
| | | | | | | | | | | | | | | | | | (S5C5 |
| | | | | | | | | | | | | | | | | | pH7-B); |
| | | | | | | | | | | | | | | | | | hor_ |
| | | | | | | | | | | | | | | | | | 5_ |
| | | | | | | | | | | | | | | | | | 8 |
| | | | | | | | | | | | | | | | | | (S5C5 |
| | | | | | | | | | | | | | | | | | / |
| | | | | | | | | | | | | | | | | | 19qH4-B) |
| Chr6 | gRNA | TTCCT | 272 | 274 | 0.993 | 279 | 1029 | 0.975 | 40 | 0.15 | 2771684 | 0 | NO | | hor_ | hor_ | hor_ |
| | 6-1 | CTTGA | | | | | | | | | | | | | 6_ | 6_ | 51 |
| | | TAGAG | | | | | | | | | | | | | 1 | 1 | (S5C5 |
| | | CAGTT | | | | | | | | | | | | | (S1C6 | (S1C6 | pH5); |
| | | (SEQ | | | | | | | | | | | | | H1L) | H1L) | hor_ |
| | | ID | | | | | | | | | | | | | | | 52_ |
| | | NO: | | | | | | | | | | | | | | | (S1C5 |
| | | 23) | | | | | | | | | | | | | | | pH2); |
| | | | | | | | | | | | | | | | | | hor_ |
| | | | | | | | | | | | | | | | | | 5_ |
| | | | | | | | | | | | | | | | | | 3 |
| | | | | | | | | | | | | | | | | | (S1C1 |
| | | | | | | | | | | | | | | | | | / |
| | | | | | | | | | | | | | | | | | 5/ |
| | | | | | | | | | | | | | | | | | 19 |
| | | | | | | | | | | | | | | | | | H1L); |
| | | | | | | | | | | | | | | | | | hor_ |
| | | | | | | | | | | | | | | | | | 5_ |
| | | | | | | | | | | | | | | | | | 4 |
| | | | | | | | | | | | | | | | | | (S1C5 |
| | | | | | | | | | | | | | | | | | pH2); |
| | | | | | | | | | | | | | | | | | hor_ |
| | | | | | | | | | | | | | | | | | 55 |
| | | | | | | | | | | | | | | | | | (S1C1 |
| | | | | | | | | | | | | | | | | | / |
| | | | | | | | | | | | | | | | | | 5/ |
| | | | | | | | | | | | | | | | | | 19 |
| | | | | | | | | | | | | | | | | | H1L); |
| | | | | | | | | | | | | | | | | | hor_ |
| | | | | | | | | | | | | | | | | | 56_ |
| | | | | | | | | | | | | | | | | | (S5C5 |
| | | | | | | | | | | | | | | | | | pH6); |
| | | | | | | | | | | | | | | | | | hor_ |
| | | | | | | | | | | | | | | | | | 5_ |
| | | | | | | | | | | | | | | | | | 7 |
| | | | | | | | | | | | | | | | | | (S5C5 |
| | | | | | | | | | | | | | | | | | pH7-B); |
| | | | | | | | | | | | | | | | | | hor_ |
| | | | | | | | | | | | | | | | | | 5_ |
| | | | | | | | | | | | | | | | | | 8 |
| | | | | | | | | | | | | | | | | | (S5C5 |
| | | | | | | | | | | | | | | | | | / |
| | | | | | | | | | | | | | | | | | 19qH4-B) |
| Chr6 | gRNA | ATGGC | 1615 | 1615 | 1 | 1615 | 804 | 1 | 50 | 0.286 | 2771684 | 0 | YES | 69 | hor_ | hor_ | hor_ |
| | 6-2 | TGCAT | | | | | | | | | | | | | 6_ | 6_ | 6_ |
| | | TCCAC | | | | | | | | | | | | | 1 | 1 | 1 |
| | | ACACA | | | | | | | | | | | | | (S1C6 | (S1C6 | (S1C6 |
| | | (SEQ | | | | | | | | | | | | | H1L) | H1L) | H1L) |
| | | ID | | | | | | | | | | | | | | | |
| | | NO: | | | | | | | | | | | | | | | |
| | | 24) | | | | | | | | | | | | | | | |
| Chr7 | gRNA | TGGAT | 2904 | 2905 | 1 | 2905 | 2162 | 1 | 45 | 0.159 | 3300127 | 0 | YES | 72 | hor_ | hor_ | hor_ |
| | 7-1 | ATATG | | | | | | | | | | | | | 7_ | 72 | 7_ |
| | | GACCG | | | | | | | | | | | | | 2 | (S1C7 | 1 |
| | | CATTG | | | | | | | | | | | | | (S1C7 | H1L) | (S5C7 |
| | | (SEQ | | | | | | | | | | | | | H1L) | | H2); |
| | | ID | | | | | | | | | | | | | | | hor_ |
| | | NO: | | | | | | | | | | | | | | | 72_ |
| | | 25) | | | | | | | | | | | | | | | (S1C7 |
| | | | | | | | | | | | | | | | | | H1L) |
| Chr7 | gRNA | CTGCT | 2556 | 2556 | 1 | 2556 | 2377 | 1 | 45 | 0.219 | 3300127 | 0 | NO | | hor_ | hor_ | hor_ |
| | 7-2 | TGTTA | | | | | | | | | | | | | 7_ | 7_ | 7_ |
| | | TGTCT | | | | | | | | | | | | | 2 | 2 | 1 |
| | | GCAAG | | | | | | | | | | | | | (S1C7 | (S1C7 | (S5C7 |
| | | (SEQ | | | | | | | | | | | | | H1L) | H1L) | H2); |
| | | ID | | | | | | | | | | | | | | | hor_ |
| | | NO: | | | | | | | | | | | | | | | 72_ |
| | | 26) | | | | | | | | | | | | | | | (S1C7 |
| | | | | | | | | | | | | | | | | | H1L) |
| Chr7 | gRNA | ACTCT | 2620 | 2620 | 1 | 2620 | 2204 | 1 | 45 | 0.097 | 3300127 | 0 | YES | 68 | hor_ | hor_ | hor_ |
| | 7-3 | TGCTG | | | | | | | | | | | | | 7_ | 72 | 7_ |
| | | TGGCA | | | | | | | | | | | | | 2 | (S1C7 | 1 |
| | | TTTTC | | | | | | | | | | | | | (S1C7 | H1L) | (S5C7 |
| | | (SEQ | | | | | | | | | | | | | H1L) | | H2); |
| | | ID | | | | | | | | | | | | | | | hor_ |
| | | NO: | | | | | | | | | | | | | | | 72_ |
| | | 27) | | | | | | | | | | | | | | | (S1C7 |
| | | | | | | | | | | | | | | | | | H1L) |
| Chr8 | gRNA | AACCT | 1454 | 1454 | 1 | 1454 | 1194 | 1 | 45 | 0.59 | 2083397 | 0 | NO | | hor_ | hor_ | hor_ |
| | 8-1 | GCTCT | | | | | | | | | | | | | 8_ | 8_ | 8_ |
| | | ATGAA | | | | | | | | | | | | | 1 | 1 | 1 |
| | | ACGGA | | | | | | | | | | | | | (S2C8 | (S2C8 | (S2C8 |
| | | (SEQ | | | | | | | | | | | | | H1L); | H1L); | H1L); |
| | | ID | | | | | | | | | | | | | hor_ | hor_ | hor_ |
| | | NO: | | | | | | | | | | | | | 8_ | 8_ | 82_ |
| | | 28) | | | | | | | | | | | | | 2 | 2 | (S2C8 |
| | | | | | | | | | | | | | | | (S2C8 | (S2C8 | H1L) |
| | | | | | | | | | | | | | | | H1L) | H1L) | |
| Chr8 | gRNA | GAATG | 1380 | 1380 | 1 | 1380 | 1163 | 1 | 45 | 0.143 | 2083397 | 0 | YES | 76 | hor_ | hor_ | hor_ |
| | 8-2 | TTCAA | | | | | | | | | | | | | 8_ | 8_ | 81 |
| | | CTCTG | | | | | | | | | | | | | 1 | 1 | (S2C8 |
| | | AGAGC | | | | | | | | | | | | | (S2C8 | (S2C8 | H1L); |
| | | (SEQ | | | | | | | | | | | | | H1L); | H1L); | hor_ |
| | | ID | | | | | | | | | | | | | hor_ | hor_ | 82_ |
| | | NO: | | | | | | | | | | | | | 8_ | 8_ | (S2C8 |
| | | 29) | | | | | | | | | | | | | 2 | 2 | H1L) |
| | | | | | | | | | | | | | | | (S2C8 | (S2C8 | |
| | | | | | | | | | | | | | | | H1L) | H1L) | |
| Chr9 | gRNA | CAGAA | 986 | 986 | 1 | 986 | 1016 | 1 | 45 | 0.091 | 2630820 | 0 | YES | 70 | hor_ | hor_ | hor_ |
| | 9-1 | AGAGT | | | | | | | | | | | | | 9_ | 9_ | 9_ |
| | | GTCTC | | | | | | | | | | | | | 1 | 1 | 1 |
| | | AAACC | | | | | | | | | | | | | (S2C9 | (S2C9 | (S2C9 |
| | | (SEQ | | | | | | | | | | | | | H1L) | H1L) | H1L) |
| | | ID | | | | | | | | | | | | | | | |
| | | NO: | | | | | | | | | | | | | | | |
| | | 30) | | | | | | | | | | | | | | | |
| Chr9 | gRNA | AACAC | 1390 | 1390 | 1 | 1390 | 1014 | 1 | 45 | 0.187 | 2630820 | 0 | NO | | hor_ | hor_ | hor_ |
| | 9-2 | TTCCC | | | | | | | | | | | | | 9_ | 9_ | 9_ |
| | | TTCAT | | | | | | | | | | | | | 1 | 1 | 1 |
| | | ACAGC | | | | | | | | | | | | | (S2C9 | (S2C9 | (S2C9 |
| | | (SEQ | | | | | | | | | | | | | H1L) | H1L) | H1L) |
| | | ID | | | | | | | | | | | | | | | |
| | | NO: | | | | | | | | | | | | | | | |
| | | 31) | | | | | | | | | | | | | | | |
| Chr9 | gRNA | GATAG | 2116 | 2116 | 1 | 2116 | 2070 | 1 | 40 | 0.417 | 2630820 | 0 | YES | 75 | hor_ | hor_ | hor_ |
| | 9-3 | CTTTG | | | | | | | | | | | | | 9_ | 9_ | 9_ |
| | | AAGGT | | | | | | | | | | | | | 1 | 1 | 1 |
| | | TTCGT | | | | | | | | | | | | | (S2C9 | (S2C9 | (S2C9 |
| | | (SEQ | | | | | | | | | | | | | H1L) | H1L) | H1L) |
| | | ID | | | | | | | | | | | | | | | |
| | | NO: | | | | | | | | | | | | | | | |
| | | 32) | | | | | | | | | | | | | | | |
| Chr9 | gRNA | GTTTC | 1384 | 1384 | 1 | 1384 | 1033 | 1 | 40 | 0.351 | 2630820 | 0 | YES | 70 | hor_ | hor_ | hor_ |
| | 9-5 | AAACC | | | | | | | | | | | | | 9_ | 9_ | 9_ |
| | | TGCTG | | | | | | | | | | | | | 1 | 1 | 1 |
| | | TATGA | | | | | | | | | | | | | (S2C9 | (S2C9 | (S2C9 |
| | | (SEQ | | | | | | | | | | | | | H1L) | H1L) | H1L) |
| | | ID | | | | | | | | | | | | | | | |
| | | NO: | | | | | | | | | | | | | | | |
| | | 33) | | | | | | | | | | | | | | | |
| Chr9 | gRNA | ACTTG | 987 | 987 | 1 | 987 | 487 | 1 | 40 | 0.709 | 2630820 | 0 | NO | | hor_ | hor_ | hor_ |
| | 9-6 | AGTAC | | | | | | | | | | | | | 9_ | 9_ | 9_ |
| | | ACACA | | | | | | | | | | | | | 1 | 1 | 1 |
| | | TCACA | | | | | | | | | | | | | (S2C9 | (S2C9 | (S2C9 |
| | | (SEQ | | | | | | | | | | | | | H1L) | H1L) | H1L) |
| | | ID | | | | | | | | | | | | | | | |
| | | NO: | | | | | | | | | | | | | | | |
| | | 34) | | | | | | | | | | | | | | | |
| Chr10 | gRNA | TTTGA | 17 | 52 | 0.327 | 52 | 780 | 0.327 | 45 | 0.277 | 2030796 | 0 | YES | 63 | hor_ | hor_ | hor_ |
| | 10-1 | GGACT | | | | | | | | | | | | | 10_ | 10_ | 10_ |
| | | TCGTT | | | | | | | | | | | | | 1 | 1 | 1 |
| | | GGAAG | | | | | | | | | | | | | (S1C1 | (S1C1 | (S1C1 |
| | | (SEQ | | | | | | | | | | | | | 0H1L) | 0H1L) | 0H1L); |
| | | ID | | | | | | | | | | | | | | | hor_ |
| | | NO: | | | | | | | | | | | | | | | 10_ |
| | | 35) | | | | | | | | | | | | | | | 2 |
| | | | | | | | | | | | | | | | | | (S1C1 |
| | | | | | | | | | | | | | | | | | 0H1-B); |
| | | | | | | | | | | | | | | | | | hor_ |
| | | | | | | | | | | | | | | | | | 10_ |
| | | | | | | | | | | | | | | | | | 3 |
| | | | | | | | | | | | | | | | | | (S1C1 |
| | | | | | | | | | | | | | | | | | 0H1- |
| | | | | | | | | | | | | | | | | | C); |
| | | | | | | | | | | | | | | | | | hor_ |
| | | | | | | | | | | | | | | | | | 10_ |
| | | | | | | | | | | | | | | | | | 4 |
| | | | | | | | | | | | | | | | | | (S1C1 |
| | | | | | | | | | | | | | | | | | 0H2) |
| Chr10 | gRNA | GCTTC | 17 | 17 | 1 | 40 | 731 | 0.425 | 50 | 0.765 | 2030796 | 0 | NO | | hor_ | hor_ | hor_ |
| | 10-2 | CAACG | | | | | | | | | | | | | 10_ | 10_ | 10_ |
| | | AAGTC | | | | | | | | | | | | | 1 | 1 | 1 |
| | | CTCAA | | | | | | | | | | | | | (S1C1 | (S1C1 | (S1C1 |
| | | (SEQ | | | | | | | | | | | | | 0H1L) | 0H1L) | 0H1L); |
| | | ID | | | | | | | | | | | | | | | hor_ |
| | | NO: | | | | | | | | | | | | | | | 10_ |
| | | 36) | | | | | | | | | | | | | | | 2 |
| | | | | | | | | | | | | | | | | | (S1C1 |
| | | | | | | | | | | | | | | | | | 0H1- |
| | | | | | | | | | | | | | | | | | B); |
| | | | | | | | | | | | | | | | | | hor_ |
| | | | | | | | | | | | | | | | | | 10_ |
| | | | | | | | | | | | | | | | | | 3 |
| | | | | | | | | | | | | | | | | | (S1C1 |
| | | | | | | | | | | | | | | | | | 0H1- |
| | | | | | | | | | | | | | | | | | C); |
| | | | | | | | | | | | | | | | | | hor_ |
| | | | | | | | | | | | | | | | | | 10_ |
| | | | | | | | | | | | | | | | | | 4 |
| | | | | | | | | | | | | | | | | | (S1C1 |
| | | | | | | | | | | | | | | | | | 0H2) |
| Chr10 | gRNA | GACTT | 1787 | 1792 | 0.997 | 1792 | 545 | 0.997 | 50 | 0.246 | 2030796 | 0 | YES | | hor_ | hor_ | hor_ |
| | 10-3 | CATTG | | | | | | | | | | | | | 10_ | 10_ | 10_ |
| | | AGGCC | | | | | | | | | | | | | 1 | 1 | 1 |
| | | TTCGT | | | | | | | | | | | | | (S1C1 | (S1C1 | (S1C1 |
| | | (SEQ | | | | | | | | | | | | | 0H1L) | 0H1L) | 0H1L); |
| | | ID | | | | | | | | | | | | | | | hor_ |
| | | NO: | | | | | | | | | | | | | | | 10_ |
| | | 37) | | | | | | | | | | | | | | | 2 |
| | | | | | | | | | | | | | | | | | (S1C1 |
| | | | | | | | | | | | | | | | | | 0H1- |
| | | | | | | | | | | | | | | | | | B); |
| | | | | | | | | | | | | | | | | | hor_ |
| | | | | | | | | | | | | | | | | | 10_ |
| | | | | | | | | | | | | | | | | | 3 |
| | | | | | | | | | | | | | | | | | (S1C1 |
| | | | | | | | | | | | | | | | | | 0H1- |
| | | | | | | | | | | | | | | | | | C); |
| | | | | | | | | | | | | | | | | | hor_ |
| | | | | | | | | | | | | | | | | | 10_ |
| | | | | | | | | | | | | | | | | | 4 |
| | | | | | | | | | | | | | | | | | (S1C1 |
| | | | | | | | | | | | | | | | | | 0H2) |
| Chr11 | gRNA | TTCAG | 3308 | 3308 | 1 | 3308 | 3128 | 1 | 50 | 0.32 | 3385188 | 0 | YES | 78 | hor_ | hor_ | hor_ |
| | 11-1 | AGCTG | | | | | | | | | | | | | 11_ | 11_ | 11_ |
| | | CTCTG | | | | | | | | | | | | | 2 | 2 | 1 |
| | | TCAAG | | | | | | | | | | | | | (S3C1 | (S3C1 | (S3C1 |
| | | (SEQ | | | | | | | | | | | | | 1H1L); | 1H1L); | 1H2); |
| | | ID | | | | | | | | | | | | | hor_ | hor_ | hor_ |
| | | NO: | | | | | | | | | | | | | 11_ | 11_ | 11_ |
| | | 38) | | | | | | | | | | | | | 4 | 4 | 2 |
| | | | | | | | | | | | | | | | (S3C1 | (S3C1 | (S3C1 |
| | | | | | | | | | | | | | | | 1H1L) | 1H1L) | 1H1L); |
| | | | | | | | | | | | | | | | | | hor_ |
| | | | | | | | | | | | | | | | | | 11_ |
| | | | | | | | | | | | | | | | | | 3 |
| | | | | | | | | | | | | | | | | | (S3C1 |
| | | | | | | | | | | | | | | | | | 1H2); |
| | | | | | | | | | | | | | | | | | hor_ |
| | | | | | | | | | | | | | | | | | 11_ |
| | | | | | | | | | | | | | | | | | 4 |
| | | | | | | | | | | | | | | | | | (S3C1 |
| | | | | | | | | | | | | | | | | | 1H1L); |
| | | | | | | | | | | | | | | | | | hor_ |
| | | | | | | | | | | | | | | | | | 11_ |
| | | | | | | | | | | | | | | | | | 5 |
| | | | | | | | | | | | | | | | | | (S3C1 |
| | | | | | | | | | | | | | | | | | 1H2) |
| Chr11 | gRNA | TTCCA | 3393 | 3408 | 0.996 | 3408 | 3203 | 0.996 | 40 | 0.625 | 3385188 | 0 | YES | 82 | hor_ | hor_ | hor_ |
| | 11-2 | ACGAA | | | | | | | | | | | | | 11_ | 11_ | 11_ |
| | | ATCTT | | | | | | | | | | | | | 2 | 2 | 1 |
| | | CACAG | | | | | | | | | | | | | (S3C1 | (S3C1 | (S3C1 |
| | | (SEQ | | | | | | | | | | | | | 1H1L) | 1H1L); | 1H2); |
| | | ID | | | | | | | | | | | | | | hor_ | hor_ |
| | | NO: | | | | | | | | | | | | | | 11_ | 11_ |
| | | 39) | | | | | | | | | | | | | | 4 | 2 |
| | | | | | | | | | | | | | | | | (S3C1 | (S3C1 |
| | | | | | | | | | | | | | | | | 1H1L) | 1H1L); |
| | | | | | | | | | | | | | | | | | hor_ |
| | | | | | | | | | | | | | | | | | 11_ |
| | | | | | | | | | | | | | | | | | 3 |
| | | | | | | | | | | | | | | | | | (S3C1 |
| | | | | | | | | | | | | | | | | | 1H2); |
| | | | | | | | | | | | | | | | | | hor_ |
| | | | | | | | | | | | | | | | | | 11_ |
| | | | | | | | | | | | | | | | | | 4 |
| | | | | | | | | | | | | | | | | | (S3C1 |
| | | | | | | | | | | | | | | | | | 1H1L); |
| | | | | | | | | | | | | | | | | | hor_ |
| | | | | | | | | | | | | | | | | | 11_ |
| | | | | | | | | | | | | | | | | | 5 |
| | | | | | | | | | | | | | | | | | (S3C1 |
| | | | | | | | | | | | | | | | | | 1H2) |
| Chr12 | gRNA | TGCCT | 1741 | 1741 | 1 | 1742 | 1541 | 0.999 | 45 | 0.612 | 2581652 | 0 | NO | | hor_ | hor_ | hor_ |
| | 12-1 | CTATT | | | | | | | | | | | | | 12_ | 12_ | 12_ |
| | | CAACT | | | | | | | | | | | | | 2 | 2 | 2 |
| | | CACAG | | | | | | | | | | | | | (S1C1 | (S1C1 | (S1C1 |
| | | (SEQ | | | | | | | | | | | | | 2H1L) | 2H1L) | 2H1L) |
| | | ID | | | | | | | | | | | | | | | |
| | | NO: | | | | | | | | | | | | | | | |
| | | 40) | | | | | | | | | | | | | | | |
| Chr12 | gRNA | CACCT | 1727 | 1727 | 1 | 1728 | 1527 | 0.999 | 45 | 0.521 | 2581652 | 0 | YES | 79 | hor_ | hor_ | hor_ |
| | 12-2 | CTGTG | | | | | | | | | | | | | 12_ | 12_ | 12_ |
| | | AGTTG | | | | | | | | | | | | | 2 | 2 | 2 |
| | | AATAG | | | | | | | | | | | | | (S1C1 | (S1C1 | (S1C1 |
| | | (SEQ | | | | | | | | | | | | | 2H1L) | 2H1L) | 2H1L) |
| | | ID | | | | | | | | | | | | | | | |
| | | NO: | | | | | | | | | | | | | | | |
| | | 41) | | | | | | | | | | | | | | | |
| Chr13 | gRNA | CTTTC | 1863 | 2088 | 0.892 | 2090 | 1300 | 0.891 | 45 | 0.206 | 1950698 | 0 | NO | | hor_ | hor_ | hor_ |
| | 13-1 | TGGAG | | | | | | | | | | | | | 13_ | 13_ | 13_ |
| | | TATCT | | | | | | | | | | | | | 3 | 3 | 1 |
| | | GGATG | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 42) | | | | | | | | | | | | | | | 22H2); |
| | | | | | | | | | | | | | | | | | hor_ |
| | | | | | | | | | | | | | | | | | 13_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 13_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L); |
| | | | | | | | | | | | | | | | | | hor_ |
| | | | | | | | | | | | | | | | | | 13_ |
| | | | | | | | | | | | | | | | | | 4 |
| | | | | | | | | | | | | | | | | | (S2C1 |
| | | | | | | | | | | | | | | | | | 3/ |
| | | | | | | | | | | | | | | | | | 21H1-B) |
| Chr13 | gRNA | AACAC | 1876 | 2081 | 0.901 | 2083 | 1232 | 0.901 | 40 | 0.014 | 1950698 | 0 | NO | | hor_ | hor_ | hor_ |
| | 13-2 | TCTTT | | | | | | | | | | | | | 13_ | 13_ | 13_ |
| | | CTGGA | | | | | | | | | | | | | 3 | 3 | 1 |
| | | GTATC | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 43) | | | | | | | | | | | | | | | 22H2); |
| | | | | | | | | | | | | | | | | | hor_ |
| | | | | | | | | | | | | | | | | | 13_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 13_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L); |
| | | | | | | | | | | | | | | | | | hor_ |
| | | | | | | | | | | | | | | | | | 13_ |
| | | | | | | | | | | | | | | | | | 4 |
| | | | | | | | | | | | | | | | | | (S2C1 |
| | | | | | | | | | | | | | | | | | 3/ |
| | | | | | | | | | | | | | | | | | 21H1-B) |
| Chr13 | gRNA | TGTGT | 1885 | 2201 | 0.856 | 2209 | 2397 | 0.853 | 45 | 0.782 | 1950698 | 0 | YES | 68 | hor_ | hor_ | hor_ |
| | 13-3 | ACTCA | | | | | | | | | | | | | 13_ | 13_ | 13_ |
| | | GCTAA | | | | | | | | | | | | | 3 | 3 | 1 |
| | | CAGAG | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 44) | | | | | | | | | | | | | | | 22H2); |
| | | | | | | | | | | | | | | | | | hor_ |
| | | | | | | | | | | | | | | | | | 13_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 13_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L); |
| | | | | | | | | | | | | | | | | | hor_ |
| | | | | | | | | | | | | | | | | | 13_ |
| | | | | | | | | | | | | | | | | | 4 |
| | | | | | | | | | | | | | | | | | (S2C1 |
| | | | | | | | | | | | | | | | | | 3/ |
| | | | | | | | | | | | | | | | | | 21H1-B) |
| Chr13 | gRNA | GTTCA | 1257 | 1257 | 1 | 1258 | 324 | 1 | 40 | 0.697 | 1950698 | 1 | NO | | hor_ | hor_ | hor_ |
| | 13-4 | TCTCT | | | | | | | | | | | | | 13_ | 13_ | 13_ |
| | | ATGAG | | | | | | | | | | | | | 3 | 3 | 1 |
| | | TCGAA | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 45) | | | | | | | | | | | | | | | 22H2); |
| | | | | | | | | | | | | | | | | | hor_ |
| | | | | | | | | | | | | | | | | | 13_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 13_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L); |
| | | | | | | | | | | | | | | | | | hor_ |
| | | | | | | | | | | | | | | | | | 13_ |
| | | | | | | | | | | | | | | | | | 4 |
| | | | | | | | | | | | | | | | | | (S2C1 |
| | | | | | | | | | | | | | | | | | 3/ |
| | | | | | | | | | | | | | | | | | 21H1-B) |
| Chr13 | gRNA | GCACG | 1276 | 1276 | 1 | 1277 | 332 | 0.999 | 40 | 0.336 | 1950698 | 0 | YES | 65 | hor_ | hor_ | hor_ |
| | 13-5 | TTTCA | | | | | | | | | | | but | | 13_ | 13_ | 13_ |
| | | AACAC | | | | | | | | | | | more | | 3 | 3 | 1 |
| | | TCTTT | | | | | | | | | | | than | | (S2C1 | (S2C1 | (S4/ |
| | | (SEQ | | | | | | | | | | | 2 | | 3/ | 3/ | 6C13/ |
| | | ID | | | | | | | | | | | foci | | 21H1L) | 21H1L) | 14/ |
| | | NO: | | | | | | | | | | | per | | | | 21/ |
| | | 46) | | | | | | | | | | | cell | | | | 22H2); |
| | | | | | | | | | | | | | | | | | hor_ |
| | | | | | | | | | | | | | | | | | 13_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 13_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L); |
| | | | | | | | | | | | | | | | | | hor_ |
| | | | | | | | | | | | | | | | | | 13_ |
| | | | | | | | | | | | | | | | | | 4 |
| | | | | | | | | | | | | | | | | | (S2C1 |
| | | | | | | | | | | | | | | | | | 3/ |
| | | | | | | | | | | | | | | | | | 21H1-B) |
| Chr13 | gRNA | TTGAA | 1258 | 1258 | 1 | 1259 | 328 | 1 | 35 | 0.238 | 1950698 | 1 | NO | | hor_ | hor_ | hor_ |
| | 13-6 | ACGTG | | | | | | | | | | | | | 13_ | 13_ | 13_ |
| | | CTCAA | | | | | | | | | | | | | 3 | 3 | 1 |
| | | AGTAA | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 47) | | | | | | | | | | | | | | | 22H2); |
| | | | | | | | | | | | | | | | | | hor_ |
| | | | | | | | | | | | | | | | | | 13_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 13_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L); |
| | | | | | | | | | | | | | | | | | hor_ |
| | | | | | | | | | | | | | | | | | 13_ |
| | | | | | | | | | | | | | | | | | 4 |
| | | | | | | | | | | | | | | | | | (S2C1 |
| | | | | | | | | | | | | | | | | | 3/ |
| | | | | | | | | | | | | | | | | | 21H1-B) |
| Chr13 | gRNA | TTTGA | 1258 | 1258 | 1 | 1259 | 328 | 1 | 35 | 0.245 | 1950698 | 1 | NO | | hor_ | hor_ | hor_ |
| | 13-7 | AACGT | | | | | | | | | | | | | 13_ | 13_ | 13_ |
| | | GCTCA | | | | | | | | | | | | | 3 | 3 | 1 |
| | | AAGTA | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 48) | | | | | | | | | | | | | | | 22H2); |
| | | | | | | | | | | | | | | | | | hor_ |
| | | | | | | | | | | | | | | | | | 13_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 13_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L); |
| | | | | | | | | | | | | | | | | | hor_ |
| | | | | | | | | | | | | | | | | | 13_ |
| | | | | | | | | | | | | | | | | | 4 |
| | | | | | | | | | | | | | | | | | (S2C1 |
| | | | | | | | | | | | | | | | | | 3/ |
| | | | | | | | | | | | | | | | | | 21H1-B) |
| Chr13 | gRNA | TCGAC | 1257 | 1257 | 1 | 1258 | 324 | 0.999 | 40 | 0.291 | 1950698 | 0 | NO | | hor_ | hor_ | hor_ |
| | 13-8 | TCATA | | | | | | | | | | | | | 13_ | 13_ | 13_ |
| | | GAGAT | | | | | | | | | | | | | 3 | 3 | 1 |
| | | GAACA | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 49) | | | | | | | | | | | | | | | 22H2); |
| | | | | | | | | | | | | | | | | | hor_ |
| | | | | | | | | | | | | | | | | | 13_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 13_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L); |
| | | | | | | | | | | | | | | | | | hor_ |
| | | | | | | | | | | | | | | | | | 13_ |
| | | | | | | | | | | | | | | | | | 4 |
| | | | | | | | | | | | | | | | | | (S2C1 |
| | | | | | | | | | | | | | | | | | 3/ |
| | | | | | | | | | | | | | | | | | 21H1-B) |
| Chr14 | gRNA | ACTTG | 937 | 937 | 1 | 937 | 312 | 1 | 35 | 0.58 | 2616299 | 0 | NO | | hor_ | hor_ | hor_ |
| | 14-3 | AATGC | | | | | | | | | | | | | 14_ | 14_ | 14_ |
| | | ACATA | | | | | | | | | | | | | 3 | 3 | 1 |
| | | TCACA | | | | | | | | | | | | | (S2C1 | (S2C1 | (S5C1 |
| | | (SEQ | | | | | | | | | | | | | 4/ | 4/ | 3/ |
| | | ID | | | | | | | | | | | | | 22H1L) | 22H1L) | 14/ |
| | | NO: | | | | | | | | | | | | | | | 21/ |
| | | 50) | | | | | | | | | | | | | | | 22H6); |
| | | | | | | | | | | | | | | | | | hor_ |
| | | | | | | | | | | | | | | | | | 14_ |
| | | | | | | | | | | | | | | | | | 2 |
| | | | | | | | | | | | | | | | | | (S4/ |
| | | | | | | | | | | | | | | | | | 6C13/ |
| | | | | | | | | | | | | | | | | | 14/ |
| | | | | | | | | | | | | | | | | | 21/ |
| | | | | | | | | | | | | | | | | | 22H2); |
| | | | | | | | | | | | | | | | | | hor_ |
| | | | | | | | | | | | | | | | | | 14_ |
| | | | | | | | | | | | | | | | | | 3 |
| | | | | | | | | | | | | | | | | | (S2C1 |
| | | | | | | | | | | | | | | | | | 4/ |
| | | | | | | | | | | | | | | | | | 22H1L) |
| Chr15 | gRNA | TCTTA | 407 | 407 | 1 | 407 | 559 | 1 | 40 | 0.349 | 1015672 | 0 | NO | | hor_ | hor_ | hor_ |
| | 15-1 | GGCCT | | | | | | | | | | | | | 15_ | 15_ | 15_ |
| | | AAGGT | | | | | | | | | | | | | 3 | 3 | 1 |
| | | GAAAA | | | | | | | | | | | | | (S2C1 | (S2C1 | (S4C1 |
| | | (SEQ | | | | | | | | | | | | | 5H1L) | 5H1L) | 5H3); |
| | | ID | | | | | | | | | | | | | | | hor_ |
| | | NO: | | | | | | | | | | | | | | | 15_ |
| | | 51) | | | | | | | | | | | | | | | 2 |
| | | | | | | | | | | | | | | | | | (S4C1 |
| | | | | | | | | | | | | | | | | | 5H2); |
| | | | | | | | | | | | | | | | | | hor_ |
| | | | | | | | | | | | | | | | | | 15_ |
| | | | | | | | | | | | | | | | | | 3 |
| | | | | | | | | | | | | | | | | | (S2C1 |
| | | | | | | | | | | | | | | | | | 5H1L) |
| Chr15 | gRNA | TGAGT | 403 | 403 | 1 | 412 | 558 | 0.978 | 40 | 0.536 | 1015672 | 0 | NO | | hor_ | hor_ | hor_ |
| | 15-2 | ACACA | | | | | | | | | | | | | 15_ | 15_ | 15_ |
| | | CATCA | | | | | | | | | | | | | 3 | 3 | 1 |
| | | CAAAG | | | | | | | | | | | | | (S2C1 | (S2C1 | (S4C1 |
| | | (SEQ | | | | | | | | | | | | | 5H1L) | 5H1L) | 5H3); |
| | | ID | | | | | | | | | | | | | | | hor_ |
| | | NO: | | | | | | | | | | | | | | | 15_ |
| | | 52) | | | | | | | | | | | | | | | 2 |
| | | | | | | | | | | | | | | | | | (S4C1 |
| | | | | | | | | | | | | | | | | | 5H2); |
| | | | | | | | | | | | | | | | | | hor_ |
| | | | | | | | | | | | | | | | | | 15_ |
| | | | | | | | | | | | | | | | | | 3 |
| | | | | | | | | | | | | | | | | | (S2C1 |
| | | | | | | | | | | | | | | | | | 5H1L |
| Chr15 | gRNA | GATAG | 721 | 721 | 1 | 734 | 900 | 0.982 | 40 | 0.659 | 1015672 | 0 | NO | | hor_ | hor_ | hor_ |
| | 15-3 | TTCTG | | | | | | | | | | | | | 15_ | 15_ | 15_ |
| | | AGGAT | | | | | | | | | | | | | 3 | 3 | 1 |
| | | TTCGT | | | | | | | | | | | | | (S2C1 | (S2C1 | (S4C1 |
| | | (SEQ | | | | | | | | | | | | | 5H1L) | 5H1L) | 5H3); |
| | | ID | | | | | | | | | | | | | | | hor_ |
| | | NO: | | | | | | | | | | | | | | | 15_ |
| | | 53) | | | | | | | | | | | | | | | 2 |
| | | | | | | | | | | | | | | | | | (S4C1 |
| | | | | | | | | | | | | | | | | | 5H2); |
| | | | | | | | | | | | | | | | | | hor_ |
| | | | | | | | | | | | | | | | | | 15_ |
| | | | | | | | | | | | | | | | | | 3 |
| | | | | | | | | | | | | | | | | | (S2C1 |
| | | | | | | | | | | | | | | | | | 5H1L) |
| Chr16 | gRNA | TGGAT | 1159 | 1159 | 1 | 1159 | 1098 | 1 | 45 | 0.417 | 1981235 | 0 | YES | 75 | hor_ | hor_ | hor_ |
| | 16-1 | ATCTT | | | | | | | | | | | | | 16_ | 16_ | 16_ |
| | | GGCCT | | | | | | | | | | | | | 2 | 2 | 1 |
| | | CTTAG | | | | | | | | | | | | | (S1C1 | (S1C1 | (S2C1 |
| | | (SEQ | | | | | | | | | | | | | 6H1L) | 6H1L) | 6pH2-A); |
| | | ID | | | | | | | | | | | | | | | hor_ |
| | | NO: | | | | | | | | | | | | | | | 16_ |
| | | 54) | | | | | | | | | | | | | | | 2 |
| | | | | | | | | | | | | | | | | | (S1C1 |
| | | | | | | | | | | | | | | | | | 6H1L); |
| | | | | | | | | | | | | | | | | | hor_ |
| | | | | | | | | | | | | | | | | | 16_ |
| | | | | | | | | | | | | | | | | | 3 |
| | | | | | | | | | | | | | | | | | (S2C1 |
| | | | | | | | | | | | | | | | | | 6pH2-B/ |
| | | | | | | | | | | | | | | | | | A) |
| Chr16 | gRNA | CTGTT | 1093 | 1093 | 1 | 1093 | 1051 | 1 | 55 | 0.376 | 1981235 | 0 | NO | | hor_ | hor_ | hor_ |
| | 16-2 | TGTGA | | | | | | | | | | | | | 16_ | 16_ | 16_ |
| | | AGCCT | | | | | | | | | | | | | 2 | 2 | 1 |
| | | GCCAG | | | | | | | | | | | | | (S1C1 | (S1C1 | (S2C1 |
| | | (SEQ | | | | | | | | | | | | | 6H1L) | 6H1L) | 6pH2-A); |
| | | ID | | | | | | | | | | | | | | | hor_ |
| | | NO: | | | | | | | | | | | | | | | 16_ |
| | | 55) | | | | | | | | | | | | | | | 2 |
| | | | | | | | | | | | | | | | | | (S1C1 |
| | | | | | | | | | | | | | | | | | 6H1L); |
| | | | | | | | | | | | | | | | | | hor_ |
| | | | | | | | | | | | | | | | | | 16_ |
| | | | | | | | | | | | | | | | | | 3 |
| | | | | | | | | | | | | | | | | | (S2C1 |
| | | | | | | | | | | | | | | | | | 6pH2-B/ |
| | | | | | | | | | | | | | | | | | A) |
| Chr17 | gRNA | GATAT | 1863 | 1864 | 0.999 | 2040 | 2230 | 0.926 | 45 | 0.662 | 3594520 | 28 | NO | | hor_ | hor_ | hor_ |
| | 17-1 | ACCCG | | | | | | | | | | | | | 17_ | 17_ | 17_ |
| | | TTTCG | | | | | | | | | | | | | 2 | 2 | 1 |
| | | AACGA | | | | | | | | | | | | | (S3C1 | (S3C1 | (S3C1 |
| | | (SEQ | | | | | | | | | | | | | 7H1L) | 7H1L) | 7H1-B); |
| | | ID | | | | | | | | | | | | | | | hor_ |
| | | NO: | | | | | | | | | | | | | | | 17_ |
| | | 56) | | | | | | | | | | | | | | | 2 |
| | | | | | | | | | | | | | | | | | (S3C1 |
| | | | | | | | | | | | | | | | | | 7H1L); |
| | | | | | | | | | | | | | | | | | hor_ |
| | | | | | | | | | | | | | | | | | 17_ |
| | | | | | | | | | | | | | | | | | 3 |
| | | | | | | | | | | | | | | | | | (S3C1 |
| | | | | | | | | | | | | | | | | | 7H1-C) |
| Chr17 | gRNA | TGCTT | 1635 | 1635 | 1 | 1850 | 1145 | 0.994 | 40 | 0.313 | 3594520 | 205 | NO | | hor_ | hor_ | hor_ |
| | 17-3 | CTGTT | | | | | | | | | | | | | 17_ | 17_ | 17_ |
| | | TAGTT | | | | | | | | | | | | | 2 | 2 | 1 |
| | | CTGTG | | | | | | | | | | | | | (S3C1 | (S3C1 | (S3C1 |
| | | (SEQ | | | | | | | | | | | | | 7H1L) | 7H1L) | 7H1-B); |
| | | ID | | | | | | | | | | | | | | | hor_ |
| | | NO: | | | | | | | | | | | | | | | 17_ |
| | | 57) | | | | | | | | | | | | | | | 2 |
| | | | | | | | | | | | | | | | | | (S3C1 |
| | | | | | | | | | | | | | | | | | 7H1L); |
| | | | | | | | | | | | | | | | | | hor_ |
| | | | | | | | | | | | | | | | | | 17_ |
| | | | | | | | | | | | | | | | | | 3 |
| | | | | | | | | | | | | | | | | | (S3C1 |
| | | | | | | | | | | | | | | | | | 7H1-C) |
| Chr17 | gRNA | CACAG | 2510 | 2510 | 1 | 3027 | 2214 | 0.883 | 45 | 0.339 | 3594520 | 185 | NO | | hor_ | hor_ | hor_ |
| | 17-4 | AGCTG | | | | | | | | | | | | | 17_ | 17_ | 17_ |
| | | AACAT | | | | | | | | | | | | | 2 | 2 | 1 |
| | | TCCTT | | | | | | | | | | | | | (S3C1 | (S3C1 | (S3C1 |
| | | (SEQ | | | | | | | | | | | | | 7H1L) | 7H1L) | 7H1-B); |
| | | ID | | | | | | | | | | | | | | | hor_ |
| | | NO: | | | | | | | | | | | | | | | 17_ |
| | | 58) | | | | | | | | | | | | | | | 2 |
| | | | | | | | | | | | | | | | | | (S3C1 |
| | | | | | | | | | | | | | | | | | 7H1L); |
| | | | | | | | | | | | | | | | | | hor_ |
| | | | | | | | | | | | | | | | | | 17_ |
| | | | | | | | | | | | | | | | | | 3 |
| | | | | | | | | | | | | | | | | | (S3C1 |
| | | | | | | | | | | | | | | | | | 7H1-C) |
| Chr18 | gRNA | GAATT | 4250 | 4254 | 0.999 | 4254 | 4207 | 0.999 | 40 | 0.099 | 4967851 | 0 | NO | | hor_ | hor_ | hor_ |
| | 18-1 | GAACC | | | | | | | | | | | | | 18_ | 18_ | 18_ |
| | | ACCGT | | | | | | | | | | | | | 3 | 3 | 1 |
| | | TTTGA | | | | | | | | | | | | | (S2C1 | (S2C1 | (S2C1 |
| | | (SEQ | | | | | | | | | | | | | 8H1L) | 8H1L) | 8pH2-A); |
| | | ID | | | | | | | | | | | | | | | hor_ |
| | | NO: | | | | | | | | | | | | | | | 18_ |
| | | 59) | | | | | | | | | | | | | | | 2 |
| | | | | | | | | | | | | | | | | | (S2C1 |
| | | | | | | | | | | | | | | | | | 8qH2-B; |
| | | | | | | | | | | | | | | | | | S2C1 |
| | | | | | | | | | | | | | | | | | 8pH2-A); |
| | | | | | | | | | | | | | | | | | hor_ |
| | | | | | | | | | | | | | | | | | 18_ |
| | | | | | | | | | | | | | | | | | 3 |
| | | | | | | | | | | | | | | | | | (S2C1 |
| | | | | | | | | | | | | | | | | | 8H1L); |
| | | | | | | | | | | | | | | | | | hor_ |
| | | | | | | | | | | | | | | | | | 18_ |
| | | | | | | | | | | | | | | | | | 4 |
| | | | | | | | | | | | | | | | | | (S2C1 |
| | | | | | | | | | | | | | | | | | 8qH2-D); |
| | | | | | | | | | | | | | | | | | hor_ |
| | | | | | | | | | | | | | | | | | 18_ |
| | | | | | | | | | | | | | | | | | 5 |
| | | | | | | | | | | | | | | | | | (S2C1 |
| | | | | | | | | | | | | | | | | | 8qH2-B, |
| | | | | | | | | | | | | | | | | | S2C1 |
| | | | | | | | | | | | | | | | | | 8qH2-E) |
| Chr18 | gRNA | AGGAT | 607 | 607 | 1 | 607 | 584 | 1 | 45 | 0.163 | 4967851 | 0 | NO | | hor_ | hor_ | hor_ |
| | 18-2 | ATTTG | | | | | | | | | | | | | 18_ | 18_ | 18_ |
| | | CCTAG | | | | | | | | | | | | | 3 | 3 | 1 |
| | | CCTTG | | | | | | | | | | | | | (S2C1 | (S2C1 | (S2C1 |
| | | (SEQ | | | | | | | | | | | | | 8H1L) | 8H1L) | 8pH2-A); |
| | | ID | | | | | | | | | | | | | | | hor_ |
| | | NO: | | | | | | | | | | | | | | | 18_ |
| | | 60) | | | | | | | | | | | | | | | 2 |
| | | | | | | | | | | | | | | | | | (S2C1 |
| | | | | | | | | | | | | | | | | | 8qH2-B; |
| | | | | | | | | | | | | | | | | | S2C1 |
| | | | | | | | | | | | | | | | | | 8pH2-A); |
| | | | | | | | | | | | | | | | | | hor_ |
| | | | | | | | | | | | | | | | | | 18_ |
| | | | | | | | | | | | | | | | | | 3 |
| | | | | | | | | | | | | | | | | | (S2C1 |
| | | | | | | | | | | | | | | | | | 8H1L); |
| | | | | | | | | | | | | | | | | | hor_ |
| | | | | | | | | | | | | | | | | | 18_ |
| | | | | | | | | | | | | | | | | | 4 |
| | | | | | | | | | | | | | | | | | (S2C1 |
| | | | | | | | | | | | | | | | | | 8qH2-D); |
| | | | | | | | | | | | | | | | | | hor_ |
| | | | | | | | | | | | | | | | | | 18_ |
| | | | | | | | | | | | | | | | | | 5 |
| | | | | | | | | | | | | | | | | | (S2C1 |
| | | | | | | | | | | | | | | | | | 8qH2-B, |
| | | | | | | | | | | | | | | | | | S2C1 |
| | | | | | | | | | | | | | | | | | 8qH2-E) |
| Chr18 | gRNA | GATCG | 234 | 234 | 1 | 234 | 161 | 1 | 55 | 0.343 | 4967851 | 0 | NO | | hor_ | hor_ | hor_ |
| | 18-3 | CTTTC | | | | | | | | | | | | | 18_ | 18_ | 18_ |
| | | AGGCC | | | | | | | | | | | | | 3 | 3 | 1 |
| | | TACGT | | | | | | | | | | | | | (S2C1 | (S2C1 | (S2C1 |
| | | (SEQ | | | | | | | | | | | | | 8H1L) | 8H1L) | 8pH2-A); |
| | | ID | | | | | | | | | | | | | | | hor_ |
| | | NO: | | | | | | | | | | | | | | | 18_ |
| | | 61) | | | | | | | | | | | | | | | 2 |
| | | | | | | | | | | | | | | | | | (S2C1 |
| | | | | | | | | | | | | | | | | | 8qH2-B; |
| | | | | | | | | | | | | | | | | | S2C1 |
| | | | | | | | | | | | | | | | | | 8pH2-A); |
| | | | | | | | | | | | | | | | | | hor_ |
| | | | | | | | | | | | | | | | | | 18_ |
| | | | | | | | | | | | | | | | | | 3 |
| | | | | | | | | | | | | | | | | | (S2C1 |
| | | | | | | | | | | | | | | | | | 8H1L); |
| | | | | | | | | | | | | | | | | | hor_ |
| | | | | | | | | | | | | | | | | | 18_ |
| | | | | | | | | | | | | | | | | | 4 |
| | | | | | | | | | | | | | | | | | (S2C1 |
| | | | | | | | | | | | | | | | | | 8qH2-D); |
| | | | | | | | | | | | | | | | | | hor_ |
| | | | | | | | | | | | | | | | | | 18_ |
| | | | | | | | | | | | | | | | | | 5 |
| | | | | | | | | | | | | | | | | | (S2C1 |
| | | | | | | | | | | | | | | | | | 8qH2-B, |
| | | | | | | | | | | | | | | | | | S2C1 |
| | | | | | | | | | | | | | | | | | 8qH2-E) |
| Chr18 | gRNA | ACAGA | 4701 | 4701 | 1 | 4701 | 4211 | 1 | 40 | 0.442 | 4967851 | 0 | YES | 82 | hor_ | hor_ | hor_ |
| | 18-4 | GTAGA | | | | | | | | | | | | | 18_ | 18_ | 18_ |
| | | ACATT | | | | | | | | | | | | | 3 | 3 | 1 |
| | | CCCTT | | | | | | | | | | | | | (S2C1 | (S2C1 | (S2C1 |
| | | (SEQ | | | | | | | | | | | | | 8H1L) | 8H1L) | 8pH2-A); |
| | | ID | | | | | | | | | | | | | | | hor_ |
| | | NO: | | | | | | | | | | | | | | | 18_ |
| | | 62) | | | | | | | | | | | | | | | 2 |
| | | | | | | | | | | | | | | | | | (S2C1 |
| | | | | | | | | | | | | | | | | | 8qH2-B; |
| | | | | | | | | | | | | | | | | | S2C1 |
| | | | | | | | | | | | | | | | | | 8pH2-A); |
| | | | | | | | | | | | | | | | | | hor_ |
| | | | | | | | | | | | | | | | | | 18_ |
| | | | | | | | | | | | | | | | | | 3 |
| | | | | | | | | | | | | | | | | | (S2C1 |
| | | | | | | | | | | | | | | | | | 8H1L); |
| | | | | | | | | | | | | | | | | | hor_ |
| | | | | | | | | | | | | | | | | | 18_ |
| | | | | | | | | | | | | | | | | | 4 |
| | | | | | | | | | | | | | | | | | (S2C1 |
| | | | | | | | | | | | | | | | | | 8qH2-D); |
| | | | | | | | | | | | | | | | | | hor_ |
| | | | | | | | | | | | | | | | | | 18_ |
| | | | | | | | | | | | | | | | | | 5 |
| | | | | | | | | | | | | | | | | | (S2C1 |
| | | | | | | | | | | | | | | | | | 8qH2-B, |
| | | | | | | | | | | | | | | | | | S2C1 |
| | | | | | | | | | | | | | | | | | 8qH2-E) |
| Chr19 | gRNA | GACAT | 2383 | 2383 | 1 | 2383 | 405 | 1 | 50 | 0.352 | 3950495 | 0 | YES | 62 | hor_ | hor_ | hor_ |
| | 19-3 | CCTTG | | | | | | | | | | | | | 19_ | 19_ | 19_ |
| | | AGGCT | | | | | | | | | | | | | 7 | 7 | 1 |
| | | TTCGT | | | | | | | | | | | | | (S1C5 | (S1C5 | (S5C5 |
| | | (SEQ | | | | | | | | | | | | | pH2, | pH2, | / |
| | | ID | | | | | | | | | | | | | S1C1 | S1C1 | 19qH4-A); |
| | | NO: | | | | | | | | | | | | | / | / | hor_ |
| | | 63) | | | | | | | | | | | | | 5/ | 5/ | 19_ |
| | | | | | | | | | | | | | | | 19 | 19H1L, | 2 |
| | | | | | | | | | | | | | | | H1L, | S1C1 | (S5C5 |
| | | | | | | | | | | | | | | | S1C1 | 6H1L); | / |
| | | | | | | | | | | | | | | | 6H1L); | hor_ | 19qH4-B); |
| | | | | | | | | | | | | | | | hor_ | 19_ | hor_ |
| | | | | | | | | | | | | | | | 19_ | 8 | 19_ |
| | | | | | | | | | | | | | | | 8 | (S1C1 | 3 |
| | | | | | | | | | | | | | | | (S1C1 | / | (S5C5 |
| | | | | | | | | | | | | | | | / | 5/ | / |
| | | | | | | | | | | | | | | | 5/ | 19 | 19qH4-A); |
| | | | | | | | | | | | | | | | 19 | H1L) | hor_ |
| | | | | | | | | | | | | | | | H1L) | | 19_ |
| | | | | | | | | | | | | | | | | | 4 |
| | | | | | | | | | | | | | | | | | (S5C5 |
| | | | | | | | | | | | | | | | | | pH7-A); |
| | | | | | | | | | | | | | | | | | hor_ |
| | | | | | | | | | | | | | | | | | 19_ |
| | | | | | | | | | | | | | | | | | 5 |
| | | | | | | | | | | | | | | | | | (S5C5 |
| | | | | | | | | | | | | | | | | | pH7-A/ |
| | | | | | | | | | | | | | | | | | S5C5 |
| | | | | | | | | | | | | | | | | | pH7-B); |
| | | | | | | | | | | | | | | | | | hor_ |
| | | | | | | | | | | | | | | | | | 19_ |
| | | | | | | | | | | | | | | | | | 6 |
| | | | | | | | | | | | | | | | | | (S5C5 |
| | | | | | | | | | | | | | | | | | pH5); |
| | | | | | | | | | | | | | | | | | hor_ |
| | | | | | | | | | | | | | | | | | 19_ |
| | | | | | | | | | | | | | | | | | 7 |
| | | | | | | | | | | | | | | | | | (S1C5 |
| | | | | | | | | | | | | | | | | | pH2, |
| | | | | | | | | | | | | | | | | | S1C1 |
| | | | | | | | | | | | | | | | | | / |
| | | | | | | | | | | | | | | | | | 5/ |
| | | | | | | | | | | | | | | | | | 19 |
| | | | | | | | | | | | | | | | | | H1L, |
| | | | | | | | | | | | | | | | | | S1C1 |
| | | | | | | | | | | | | | | | | | 6H1L); |
| | | | | | | | | | | | | | | | | | hor_ |
| | | | | | | | | | | | | | | | | | 19_ |
| | | | | | | | | | | | | | | | | | 8 |
| | | | | | | | | | | | | | | | | | (S1C1 |
| | | | | | | | | | | | | | | | | | / |
| | | | | | | | | | | | | | | | | | 5/ |
| | | | | | | | | | | | | | | | | | 19 |
| | | | | | | | | | | | | | | | | | H1L) |
| Chr20 | gRNA | AAACT | 1525 | 1525 | 1 | 1525 | 1251 | 1 | 40 | 0.612 | 2173803 | 0 | NO | | hor_ | hor_ | hor_ |
| | 20-11 | GCTCC | | | | | | | | | | | | | 20_ | 20_ | 20_ |
| | | TTCAA | | | | | | | | | | | | | 2 | 2 | 1 |
| | | AACGA | | | | | | | | | | | | | (S2C2 | (S2C2 | (S2C2 |
| | | (SEQ | | | | | | | | | | | | | 0H1L) | 0H1L) | 0H2); |
| | | ID | | | | | | | | | | | | | | | hor_ |
| | | NO: | | | | | | | | | | | | | | | 20_ |
| | | 64) | | | | | | | | | | | | | | | 2 |
| | | | | | | | | | | | | | | | | | (S2C2 |
| | | | | | | | | | | | | | | | | | 0H1L); |
| | | | | | | | | | | | | | | | | | hor_ |
| | | | | | | | | | | | | | | | | | 20_ |
| | | | | | | | | | | | | | | | | | 3 |
| | | | | | | | | | | | | | | | | | (S02C |
| | | | | | | | | | | | | | | | | | 20H3); |
| | | | | | | | | | | | | | | | | | hor_ |
| | | | | | | | | | | | | | | | | | 20_ |
| | | | | | | | | | | | | | | | | | 4 |
| | | | | | | | | | | | | | | | | | (S5C2 |
| | | | | | | | | | | | | | | | | | 0H6); |
| | | | | | | | | | | | | | | | | | hor_ |
| | | | | | | | | | | | | | | | | | 20_ |
| | | | | | | | | | | | | | | | | | 5 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 6 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 7 |
| | | | | | | | | | | | | | | | | | (S4C2 |
| | | | | | | | | | | | | | | | | | 0H7/ |
| | | | | | | | | | | | | | | | | | 8); |
| | | | | | | | | | | | | | | | | | hor_ |
| | | | | | | | | | | | | | | | | | 20_ |
| | | | | | | | | | | | | | | | | | 8 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 9 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 10 |
| | | | | | | | | | | | | | | | | | (S4C2 |
| | | | | | | | | | | | | | | | | | 0H8) |
| Chr20 | gRNA | AGCAT | 749 | 759 | 0.987 | 892 | 782 | 0.84 | 40 | 0.189 | 2173803 | 0 | NO | | hor_ | hor_ | hor_ |
| | 20-2 | TCTCA | | | | | | | | | | | | | 20_ | 20_ | 20_ |
| | | GAAAC | | | | | | | | | | | | | 2 | 2 | 1 |
| | | TGCTT | | | | | | | | | | | | | (S2C2 | (S2C2 | (S2C2 |
| | | (SEQ | | | | | | | | | | | | | 0H1L) | 0H1L) | 0H2); |
| | | ID | | | | | | | | | | | | | | | hor_ |
| | | NO: | | | | | | | | | | | | | | | 20_ |
| | | 65) | | | | | | | | | | | | | | | 2 |
| | | | | | | | | | | | | | | | | | (S2C2 |
| | | | | | | | | | | | | | | | | | 0H1L); |
| | | | | | | | | | | | | | | | | | hor_ |
| | | | | | | | | | | | | | | | | | 20_ |
| | | | | | | | | | | | | | | | | | 3 |
| | | | | | | | | | | | | | | | | | (S02 |
| | | | | | | | | | | | | | | | | | C20H3); |
| | | | | | | | | | | | | | | | | | hor_ |
| | | | | | | | | | | | | | | | | | 20_ |
| | | | | | | | | | | | | | | | | | 4 |
| | | | | | | | | | | | | | | | | | (S5C2 |
| | | | | | | | | | | | | | | | | | 0H6); |
| | | | | | | | | | | | | | | | | | hor_ |
| | | | | | | | | | | | | | | | | | 20_ |
| | | | | | | | | | | | | | | | | | 5 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 6 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 7 |
| | | | | | | | | | | | | | | | | | (S4C2 |
| | | | | | | | | | | | | | | | | | 0H7/ |
| | | | | | | | | | | | | | | | | | 8); |
| | | | | | | | | | | | | | | | | | hor_ |
| | | | | | | | | | | | | | | | | | 20_ |
| | | | | | | | | | | | | | | | | | 8 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 9 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 10 |
| | | | | | | | | | | | | | | | | | (S4C2 |
| | | | | | | | | | | | | | | | | | 0H8) |
| Chr20 | gRNA | GGCAG | 790 | 790 | 1 | 791 | 635 | 0.999 | 50 | 0.425 | 2173803 | 0 | NO | | hor_ | hor_ | hor_ |
| | 20-3 | CTTTG | | | | | | | | | | | | | 20_ | 20_ | 20_ |
| | | AGGAT | | | | | | | | | | | | | 2 | 2 | 1 |
| | | TTCGT | | | | | | | | | | | | | (S2C2 | (S2C2 | (S2C2 |
| | | (SEQ | | | | | | | | | | | | | 0H1L) | 0H1L) | 0H2); |
| | | ID | | | | | | | | | | | | | | | hor_ |
| | | NO: | | | | | | | | | | | | | | | 20_ |
| | | 66) | | | | | | | | | | | | | | | 2 |
| | | | | | | | | | | | | | | | | | (S2C2 |
| | | | | | | | | | | | | | | | | | 0H1L); |
| | | | | | | | | | | | | | | | | | hor_ |
| | | | | | | | | | | | | | | | | | 20_ |
| | | | | | | | | | | | | | | | | | 3 |
| | | | | | | | | | | | | | | | | | (S02C |
| | | | | | | | | | | | | | | | | | 20H3); |
| | | | | | | | | | | | | | | | | | hor_ |
| | | | | | | | | | | | | | | | | | 20_ |
| | | | | | | | | | | | | | | | | | 4 |
| | | | | | | | | | | | | | | | | | (S5C2 |
| | | | | | | | | | | | | | | | | | 0H6); |
| | | | | | | | | | | | | | | | | | hor_ |
| | | | | | | | | | | | | | | | | | 20_ |
| | | | | | | | | | | | | | | | | | 5 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 6 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 7 |
| | | | | | | | | | | | | | | | | | (S4C2 |
| | | | | | | | | | | | | | | | | | 0H7/ |
| | | | | | | | | | | | | | | | | | 8); |
| | | | | | | | | | | | | | | | | | hor_ |
| | | | | | | | | | | | | | | | | | 20_ |
| | | | | | | | | | | | | | | | | | 8 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 9 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 10 |
| | | | | | | | | | | | | | | | | | (S4C2 |
| | | | | | | | | | | | | | | | | | 0H8) |
| Chr20 | gRNA | GGTTC | 772 | 772 | 11 | 772 | 667 | 1 | 45 | 0.373 | 2173803 | 0 | NO | | hor_ | hor_ | hor_ |
| | 20-4 | AACAC | | | | | | | | | | | | | 20_ | 20_ | 20_ |
| | | TGTCA | | | | | | | | | | | | | 2 | 2 | 1 |
| | | GTTGA | | | | | | | | | | | | | (S2C2 | (S2C2 | (S2C2 |
| | | (SEQ | | | | | | | | | | | | | 0H1L) | 0H1L) | 0H2); |
| | | ID | | | | | | | | | | | | | | | hor_ |
| | | NO: | | | | | | | | | | | | | | | 20_ |
| | | 67) | | | | | | | | | | | | | | | 2 |
| | | | | | | | | | | | | | | | | | (S2C2 |
| | | | | | | | | | | | | | | | | | 0H1L); |
| | | | | | | | | | | | | | | | | | hor_ |
| | | | | | | | | | | | | | | | | | 20_ |
| | | | | | | | | | | | | | | | | | 3 |
| | | | | | | | | | | | | | | | | | (S02C |
| | | | | | | | | | | | | | | | | | 20H3); |
| | | | | | | | | | | | | | | | | | hor_ |
| | | | | | | | | | | | | | | | | | 20_ |
| | | | | | | | | | | | | | | | | | 4 |
| | | | | | | | | | | | | | | | | | (S5C2 |
| | | | | | | | | | | | | | | | | | 0H6); |
| | | | | | | | | | | | | | | | | | hor_ |
| | | | | | | | | | | | | | | | | | 20_ |
| | | | | | | | | | | | | | | | | | 5 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 6 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 7 |
| | | | | | | | | | | | | | | | | | (S4C2 |
| | | | | | | | | | | | | | | | | | 0H7/ |
| | | | | | | | | | | | | | | | | | 8); |
| | | | | | | | | | | | | | | | | | hor_ |
| | | | | | | | | | | | | | | | | | 20_ |
| | | | | | | | | | | | | | | | | | 8 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 9 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 10 |
| | | | | | | | | | | | | | | | | | (S4C2 |
| | | | | | | | | | | | | | | | | | 0H8) |
| Chr20 | gRNA | TTGGA | 763 | 763 | 1 | 763 | 616 | 1 | 55 | 0.232 | 2173803 | 0 | NO | | hor_ | hor_ | hor_ |
| | 20-5 | GCGCT | | | | | | | | | | | | | 20_ | 20_ | 20_ |
| | | TTCAG | | | | | | | | | | | | | 2 | 2 | 1 |
| | | GACGA | | | | | | | | | | | | | (S2C2 | (S2C2 | (S2C2 |
| | | (SEQ | | | | | | | | | | | | | 0H1L) | 0H1L) | 0H2); |
| | | ID | | | | | | | | | | | | | | | hor_ |
| | | NO: | | | | | | | | | | | | | | | 20_ |
| | | 68) | | | | | | | | | | | | | | | 2 |
| | | | | | | | | | | | | | | | | | (S2C2 |
| | | | | | | | | | | | | | | | | | 0H1L); |
| | | | | | | | | | | | | | | | | | hor_ |
| | | | | | | | | | | | | | | | | | 20_ |
| | | | | | | | | | | | | | | | | | 3 |
| | | | | | | | | | | | | | | | | | (S02C |
| | | | | | | | | | | | | | | | | | 20H3); |
| | | | | | | | | | | | | | | | | | hor_ |
| | | | | | | | | | | | | | | | | | 20_ |
| | | | | | | | | | | | | | | | | | 4 |
| | | | | | | | | | | | | | | | | | (S5C2 |
| | | | | | | | | | | | | | | | | | 0H6); |
| | | | | | | | | | | | | | | | | | hor_ |
| | | | | | | | | | | | | | | | | | 20_ |
| | | | | | | | | | | | | | | | | | 5 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 6 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 7 |
| | | | | | | | | | | | | | | | | | (S4C2 |
| | | | | | | | | | | | | | | | | | 0H7/ |
| | | | | | | | | | | | | | | | | | 8); |
| | | | | | | | | | | | | | | | | | hor_ |
| | | | | | | | | | | | | | | | | | 20_ |
| | | | | | | | | | | | | | | | | | 8 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 9 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 10 |
| | | | | | | | | | | | | | | | | | (S4C2 |
| | | | | | | | | | | | | | | | | | 0H8) |
| Chr20 | gRNA | AACAT | 758 | 758 | 1 | 758 | 634 | 1 | 45 | 0.109 | 2173803 | 0 | NO | | hor_ | hor_ | hor_ |
| | 20-6 | TCCCT | | | | | | | | | | | | | 20_ | 20_ | 20_ |
| | | TTGAG | | | | | | | | | | | | | 2 | 2 | 1 |
| | | AGAGC | | | | | | | | | | | | | (S2C2 | (S2C2 | (S2C2 |
| | | (SEQ | | | | | | | | | | | | | 0H1L) | 0H1L) | 0H2); |
| | | ID | | | | | | | | | | | | | | | hor_ |
| | | NO: | | | | | | | | | | | | | | | 20_ |
| | | 577) | | | | | | | | | | | | | | | 2 |
| | | | | | | | | | | | | | | | | | (S2C2 |
| | | | | | | | | | | | | | | | | | 0H1L); |
| | | | | | | | | | | | | | | | | | hor_ |
| | | | | | | | | | | | | | | | | | 20_ |
| | | | | | | | | | | | | | | | | | 3 |
| | | | | | | | | | | | | | | | | | (S02C |
| | | | | | | | | | | | | | | | | | 20H3); |
| | | | | | | | | | | | | | | | | | hor_ |
| | | | | | | | | | | | | | | | | | 20_ |
| | | | | | | | | | | | | | | | | | 4 |
| | | | | | | | | | | | | | | | | | (S5C2 |
| | | | | | | | | | | | | | | | | | 0H6); |
| | | | | | | | | | | | | | | | | | hor_ |
| | | | | | | | | | | | | | | | | | 20_ |
| | | | | | | | | | | | | | | | | | 5 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 6 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 7 |
| | | | | | | | | | | | | | | | | | (S4C2 |
| | | | | | | | | | | | | | | | | | 0H7/ |
| | | | | | | | | | | | | | | | | | 8); |
| | | | | | | | | | | | | | | | | | hor_ |
| | | | | | | | | | | | | | | | | | 20_ |
| | | | | | | | | | | | | | | | | | 8 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 9 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 10 |
| | | | | | | | | | | | | | | | | | (S4C2 |
| | | | | | | | | | | | | | | | | | 0H8) |
| Chr20 | gRNA | GCATT | 734 | 737 | 0.996 | 737 | 607 | 0.996 | 40 | 0.669 | 2173803 | 0 | NO | | hor_ | hor_ | hor_ |
| | 20-7 | CTCAG | | | | | | | | | | | | | 20_ | 20_ | 20_ |
| | | AAACT | | | | | | | | | | | | | 2 | 2 | 1 |
| | | TCGTT | | | | | | | | | | | | | (S2C2 | (S2C2 | (S2C2 |
| | | (SEQ | | | | | | | | | | | | | 0H1L) | 0H1L) | 0H2); |
| | | ID | | | | | | | | | | | | | | | hor_ |
| | | NO: | | | | | | | | | | | | | | | 20_ |
| | | 69) | | | | | | | | | | | | | | | 2 |
| | | | | | | | | | | | | | | | | | (S2C2 |
| | | | | | | | | | | | | | | | | | 0H1L); |
| | | | | | | | | | | | | | | | | | hor_ |
| | | | | | | | | | | | | | | | | | 20_ |
| | | | | | | | | | | | | | | | | | 3 |
| | | | | | | | | | | | | | | | | | (S02C |
| | | | | | | | | | | | | | | | | | 20H3); |
| | | | | | | | | | | | | | | | | | hor_ |
| | | | | | | | | | | | | | | | | | 20_ |
| | | | | | | | | | | | | | | | | | 4 |
| | | | | | | | | | | | | | | | | | (S5C2 |
| | | | | | | | | | | | | | | | | | 0H6); |
| | | | | | | | | | | | | | | | | | hor_ |
| | | | | | | | | | | | | | | | | | 20_ |
| | | | | | | | | | | | | | | | | | 5 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 6 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 7 |
| | | | | | | | | | | | | | | | | | (S4C2 |
| | | | | | | | | | | | | | | | | | 0H7/ |
| | | | | | | | | | | | | | | | | | 8); |
| | | | | | | | | | | | | | | | | | hor_ |
| | | | | | | | | | | | | | | | | | 20_ |
| | | | | | | | | | | | | | | | | | 8 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 9 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 10 |
| | | | | | | | | | | | | | | | | | (S4C2 |
| | | | | | | | | | | | | | | | | | 0H8) |
| Chr20 | gRNA | AACAC | 709 | 709 | 1 | 709 | 602 | 1 | 45 | 0.079 | 2173803 | 0 | NO | | hor_ | hor_ | hor_ |
| | 20-8 | TCTTT | | | | | | | | | | | | | 20_ | 20_ | 20_ |
| | | CTGCA | | | | | | | | | | | | | 2 | 2 | 1 |
| | | TTCCC | | | | | | | | | | | | | (S2C2 | (S2C2 | (S2C2 |
| | | (SEQ | | | | | | | | | | | | | 0H1L) | 0H1L) | 0H2); |
| | | ID | | | | | | | | | | | | | | | hor_ |
| | | NO: | | | | | | | | | | | | | | | 20_ |
| | | 70) | | | | | | | | | | | | | | | 2 |
| | | | | | | | | | | | | | | | | | (S2C2 |
| | | | | | | | | | | | | | | | | | 0H1L); |
| | | | | | | | | | | | | | | | | | hor_ |
| | | | | | | | | | | | | | | | | | 20_ |
| | | | | | | | | | | | | | | | | | 3 |
| | | | | | | | | | | | | | | | | | (S02C |
| | | | | | | | | | | | | | | | | | 20H3); |
| | | | | | | | | | | | | | | | | | hor_ |
| | | | | | | | | | | | | | | | | | 20_ |
| | | | | | | | | | | | | | | | | | 4 |
| | | | | | | | | | | | | | | | | | (S5C2 |
| | | | | | | | | | | | | | | | | | 0H6); |
| | | | | | | | | | | | | | | | | | hor_ |
| | | | | | | | | | | | | | | | | | 20_ |
| | | | | | | | | | | | | | | | | | 5 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 6 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 7 |
| | | | | | | | | | | | | | | | | | (S4C2 |
| | | | | | | | | | | | | | | | | | 0H7/ |
| | | | | | | | | | | | | | | | | | 8); |
| | | | | | | | | | | | | | | | | | hor_ |
| | | | | | | | | | | | | | | | | | 20_ |
| | | | | | | | | | | | | | | | | | 8 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 9 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 10 |
| | | | | | | | | | | | | | | | | | (S4C2 |
| | | | | | | | | | | | | | | | | | 0H8) |
| Chr | gRNA | TGGAT | 665 | 665 | 1 | 665 | 606 | 1 | 50 | 0.422 | 2173803 | 0 | NA | | hor_ | hor_ | hor_ |
| 20 | 20-9 | ATTTG | | | | | | | | | | | | | 20_ | 20_ | 20_ |
| | | GCTAG | | | | | | | | | | | | | 2 | 2 | 1 |
| | | CTGGG | | | | | | | | | | | | | (S2C2 | (S2C2 | (S2C2 |
| | | (SEQ | | | | | | | | | | | | | 0H1L) | 0H1L) | 0H2); |
| | | ID | | | | | | | | | | | | | | | hor_ |
| | | NO: | | | | | | | | | | | | | | | 20_ |
| | | 71) | | | | | | | | | | | | | | | 2 |
| | | | | | | | | | | | | | | | | | (S2C2 |
| | | | | | | | | | | | | | | | | | 0H1L); |
| | | | | | | | | | | | | | | | | | hor_ |
| | | | | | | | | | | | | | | | | | 20_ |
| | | | | | | | | | | | | | | | | | 3 |
| | | | | | | | | | | | | | | | | | (S02C |
| | | | | | | | | | | | | | | | | | 20H3); |
| | | | | | | | | | | | | | | | | | hor_ |
| | | | | | | | | | | | | | | | | | 20_ |
| | | | | | | | | | | | | | | | | | 4 |
| | | | | | | | | | | | | | | | | | (S5C2 |
| | | | | | | | | | | | | | | | | | 0H6); |
| | | | | | | | | | | | | | | | | | hor_ |
| | | | | | | | | | | | | | | | | | 20_ |
| | | | | | | | | | | | | | | | | | 5 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 6 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 7 |
| | | | | | | | | | | | | | | | | | (S4C2 |
| | | | | | | | | | | | | | | | | | 0H7/ |
| | | | | | | | | | | | | | | | | | 8); |
| | | | | | | | | | | | | | | | | | hor_ |
| | | | | | | | | | | | | | | | | | 20_ |
| | | | | | | | | | | | | | | | | | 8 |
| | | | | | | | | | | | | | | | | | (S4C2 |
| | | | | | | | | | | | | | | | | | 0H8); |
| | | | | | | | | | | | | | | | | | hor_ |
| | | | | | | | | | | | | | | | | | 20_ |
| | | | | | | | | | | | | | | | | | 9 |
| | | | | | | | | | | | | | | | | | (S4C2 |
| | | | | | | | | | | | | | | | | | 0H7); |
| | | | | | | | | | | | | | | | | | hor_ |
| | | | | | | | | | | | | | | | | | 20_ |
| | | | | | | | | | | | | | | | | | 10 |
| | | | | | | | | | | | | | | | | | (S4C2 |
| | | | | | | | | | | | | | | | | | 0H8) |
| Chr | gRNA | AAATT | 152 | 152 | 1 | 152 | 540 | 1 | 30 | 0.272 | 343352 | 0 | NO | | hor_ | hor_ | hor_ |
| 21 | 21-1 | GCTGC | | | | | | | | | | | | | 21_ | 21_ | 21_ |
| | | ATCAA | | | | | | | | | | | | | 3 | 3 | 1 |
| | | AAGAA | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 72) | | | | | | | | | | | | | | | 22H2); |
| | | | | | | | | | | | | | | | | | hor_ |
| | | | | | | | | | | | | | | | | | 21_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 21_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L) |
| Chr | gRNA | GACGT | 123 | 123 | 1 | 123 | 276 | 1 | 45 | 0.31 | 343352 | 0 | NO | | hor_ | hor_ | hor_ |
| 21 | 21-2 | TCCCT | | | | | | | | | | | | | 21_ | 21_ | 21_ |
| | | TTTTC | | | | | | | | | | | | | 3 | 3 | 1 |
| | | ACCAA | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 73) | | | | | | | | | | | | | | | 22H2); |
| | | | | | | | | | | | | | | | | | hor_ |
| | | | | | | | | | | | | | | | | | 21_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 21_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L) |
| Chr | gRNA | TCAAC | 170 | 177 | 0.96 | 177 | 752 | 0.96 | 35 | 0.332 | 343352 | 0 | NO | | hor_ | hor_ | hor_ |
| 21 | 21-4 | TCATA | | | | | | | | | | | | | 21_ | 21_ | 21_ |
| | | GAGAT | | | | | | | | | | | | | 3 | 3 | 1 |
| | | GAACA | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 74) | | | | | | | | | | | | | | | 22H2); |
| | | | | | | | | | | | | | | | | | hor_ |
| | | | | | | | | | | | | | | | | | 21_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 21_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L) |
| Chr | gRNA | GTTCA | 181 | 194 | 0.933 | 194 | 896 | 0.933 | 35 | 0.553 | 343352 | 0 | NO | | hor_ | hor_ | hor_ |
| 21 | 21-5 | TCTCT | | | | | | | | | | | | | 21_ | 21_ | 21_ |
| | | ATGAG | | | | | | | | | | | | | 3 | 3 | 1 |
| | | TTGAA | | | | | | | | | | | | | (S2C1 | (S2C1 | (S4/ |
| | | (SEQ | | | | | | | | | | | | | 3/ | 3/ | 6C13/ |
| | | ID | | | | | | | | | | | | | 21H1L) | 21H1L) | 14/ |
| | | NO: | | | | | | | | | | | | | | | 21/ |
| | | 75) | | | | | | | | | | | | | | | 22H2); |
| | | | | | | | | | | | | | | | | | hor_ |
| | | | | | | | | | | | | | | | | | 21_ |
| | | | | | | | | | | | | | | | | | 2 |
| | | | | | | | | | | | | | | | | | (S5C1 |
| | | | | | | | | | | | | | | | | | 3/ |
| | | | | | | | | | | | | | | | | | 14/ |
| | | | | | | | | | | | | | | | | | 21/ |
| | | | | | | | | | | | | | | | | | 22H6); |
| | | | | | | | | | | | | | | | | | hor_ |
| | | | | | | | | | | | | | | | | | 21_ |
| | | | | | | | | | | | | | | | | | 3 |
| | | | | | | | | | | | | | | | | | (S2C1 |
| | | | | | | | | | | | | | | | | | 3/ |
| | | | | | | | | | | | | | | | | | 21H1L) |
| Chr | gRNA | CTTGA | 1527 | 4126 | 0.37 | 4127 | 2392 | 0.37 | 50 | 0.287 | 2922885 | 0 | NO | | hor_ | hor_ | |
| 22 | 22-2 | CGCCT | | | | | | | | | | | | | 22_ | 22_ | |
| | | ACGGT | | | | | | | | | | | | | 9 | 1 | |
| | | GAAAA | | | | | | | | | | | | | (S2C1 | (S6C1 | |
| | | (SEQ | | | | | | | | | | | | | 4/ | 3/ | |
| | | ID | | | | | | | | | | | | | 22H1L) | 14/ | |
| | | NO: | | | | | | | | | | | | | | 21/ | |
| | | 76) | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 2 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3-B); | |
| | | | | | | | | | | | | | | | | hor_ | |
| | | | | | | | | | | | | | | | | 22_ | |
| | | | | | | | | | | | | | | | | 3 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 4 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 5 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 6 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 7 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 8 | |
| | | | | | | | | | | | | | | | | (S4C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H5); | |
| | | | | | | | | | | | | | | | | hor_ | |
| | | | | | | | | | | | | | | | | 22_ | |
| | | | | | | | | | | | | | | | | 9 | |
| | | | | | | | | | | | | | | | | (S2C1 | |
| | | | | | | | | | | | | | | | | 4/ | |
| | | | | | | | | | | | | | | | | 22H1L) | |
| Chr | gRNA | GTATA | 1674 | 1822 | 0.919 | 1822 | 1312 | 0.919 | 40 | 0.241 | 2922885 | 0 | NO | | hor_ | hor_ | |
| 22 | 22-3 | TGGAA | | | | | | | | | | | | | 22_ | 22_ | |
| | | GTGGA | | | | | | | | | | | | | 9 | 1 | |
| | | CGTTT | | | | | | | | | | | | | (S2C1 | (S6C1 | |
| | | (SEQ | | | | | | | | | | | | | 4/ | 3/ | |
| | | ID | | | | | | | | | | | | | 22H1L) | 14/ | |
| | | NO: | | | | | | | | | | | | | | 21/ | |
| | | 77) | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 2 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 3 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 4 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 5 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 6 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 7 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 8 | |
| | | | | | | | | | | | | | | | | (S4C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H5); | |
| | | | | | | | | | | | | | | | | hor_ | |
| | | | | | | | | | | | | | | | | 22_ | |
| | | | | | | | | | | | | | | | | 9 | |
| | | | | | | | | | | | | | | | | (S2C1 | |
| | | | | | | | | | | | | | | | | 4/ | |
| | | | | | | | | | | | | | | | | 22H1L) | |
| Chr | gRNA | TGGAC | 1026 | 1143 | 0.898 | 1143 | 1260 | 0.898 | 55 | 0.043 | 2922885 | 0 | NO | | hor_ | hor_ | |
| 22 | 22-4 | GTTTC | | | | | | | | | | | | | 22_ | 22_ | |
| | | GGACG | | | | | | | | | | | | | 9 | 1 | |
| | | GTTTG | | | | | | | | | | | | | (S2C1 | (S6C1 | |
| | | (SEQ | | | | | | | | | | | | | 4/ | 3/ | |
| | | ID | | | | | | | | | | | | | 22H1L) | 14/ | |
| | | NO: | | | | | | | | | | | | | | 21/ | |
| | | 78) | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 2 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 3 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 4 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 5 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 6 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 7 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 8 | |
| | | | | | | | | | | | | | | | | (S4C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H5); | |
| | | | | | | | | | | | | | | | | hor_ | |
| | | | | | | | | | | | | | | | | 22_ | |
| | | | | | | | | | | | | | | | | 9 | |
| | | | | | | | | | | | | | | | | (S2C1 | |
| | | | | | | | | | | | | | | | | 4/ | |
| | | | | | | | | | | | | | | | | 22H1L) | |
| Chr | gRNA | AACAT | 962 | 1018 | 0.945 | 1018 | 408 | 0.945 | 45 | 0.093 | 2922885 | 0 | NO | | hor_ | hor_ | |
| 22 | 22-5 | TGCCT | | | | | | | | | | | | | 22_ | 22_ | |
| | | TTCCT | | | | | | | | | | | | | 9 | 1 | |
| | | AGAGC | | | | | | | | | | | | | (S2C1 | (S6C1 | |
| | | (SEQ | | | | | | | | | | | | | 4/ | 3/ | |
| | | ID | | | | | | | | | | | | | 22H1L) | 14/ | |
| | | NO: | | | | | | | | | | | | | | 21/ | |
| | | 79) | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 2 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 3 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 4 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 5 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 6 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | B); | |
| | | | | | | | | | | | | | | | | hor_ | |
| | | | | | | | | | | | | | | | | 22_ | |
| | | | | | | | | | | | | | | | | 7 | |
| | | | | | | | | | | | | | | | | (S6C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H3- | |
| | | | | | | | | | | | | | | | | A); | |
| | | | | | | | | | | | | | | | | hor_ | |
| | | | | | | | | | | | | | | | | 22_ | |
| | | | | | | | | | | | | | | | | 8 | |
| | | | | | | | | | | | | | | | | (S4C1 | |
| | | | | | | | | | | | | | | | | 3/ | |
| | | | | | | | | | | | | | | | | 14/ | |
| | | | | | | | | | | | | | | | | 21/ | |
| | | | | | | | | | | | | | | | | 22H5); | |
| | | | | | | | | | | | | | | | | hor_ | |
| | | | | | | | | | | | | | | | | 22_ | |
| | | | | | | | | | | | | | | | | 9 | |
| | | | | | | | | | | | | | | | | (S2C1 | |
| | | | | | | | | | | | | | | | | 4/ | |
| | | | | | | | | | | | | | | | | 22H1L) | |
| ChrX | gRNA | CTCTT | 1394 | 1394 | 1 | 1394 | 1777 | 1 | 55 | 0.678 | 3106919 | 0 | YES | 70 | hor_ | hor_ | hor_ |
| | X-1 | TCTGT | | | | | | | | | | | | | X_ | X_ | X_ |
| | | GGGAT | | | | | | | | | | | | | 1 | 1 | 1 |
| | | CCGCA | | | | | | | | | | | | | (S3CX | (S3CX | (S3CX |
| | | (SEQ | | | | | | | | | | | | | H1L) | H1L) | H1L) |
| | | ID | | | | | | | | | | | | | | | |
| | | NO: | | | | | | | | | | | | | | | |
| | | 80) | | | | | | | | | | | | | | | |
| ChrX | gRNA | GAGGT | 1358 | 1358 | 1 | 1358 | 1689 | 1 | 50 | 0.193 | 3106919 | 0 | YES | 66 | hor_ | hor_ | hor_ |
| | X-2 | CCAAA | | | | | | | | | | | | | X_ | X_ | X1 |
| | | TATCC | | | | | | | | | | | | | 1 | 1 | (S3CX |
| | | CCTTG | | | | | | | | | | | | | (S3CX | (S3CX | H1L) |
| | | (SEQ | | | | | | | | | | | | | H1L) | H1L) | |
| | | ID | | | | | | | | | | | | | | | |
| | | NO: | | | | | | | | | | | | | | | |
| | | 81) | | | | | | | | | | | | | | | |
| ChrX | gRNA | TCTGC | 1398 | 2688 | 0.52 | 2691 | 2983 | 0.52 | 50 | 0.536 | 3106919 | 0 | NO | | hor_ | hor_ | hor_ |
| | X-3 | AAGTG | | | | | | | | | | | | | X_ | X_ | X_ |
| | | GACGT | | | | | | | | | | | | | 1 | 1 | 1 |
| | | TTGGA | | | | | | | | | | | | | (S3CX | (S3CX | (S3CX |
| | | (SEQ | | | | | | | | | | | | | H1L) | H1L) | H1L) |
| | | ID | | | | | | | | | | | | | | | |
| | | NO: | | | | | | | | | | | | | | | |
| | | 82) | | | | | | | | | | | | | | | |

CHR1

| | | | | | | | | | bind- |
|---|---|---|---|---|---|---|---|---|---|
| | bind- | | | | | | | | ing |
| | ing | | | | | | | | sites |
| | sites | | | | | | | | (CHM13- |
| | (CHM13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| TGGAT | 1804 | 1807 | 0.998 | 45 | 1804 | 1807 | 0.99833979 | 0.213778913 | 0 |
| ATTCA | | | | | | | | | |
| GACCC | | | | | | | | | |
| CTTTG | | | | | | | | | |
| (SEQ | | | | | | | | | |
| ID | | | | | | | | | |
| NO: | | | | | | | | | |
| 83) | | | | | | | | | |
| AAGGA | 1074 | 1074 | 1 | 45 | 1074 | 1074 | 1 | 0.180523682 | 0 |
| TCCTT | | | | | | | | | |
| TACAG | | | | | | | | | |
| AGAGC | | | | | | | | | |
| (SEQ | | | | | | | | | |
| ID | | | | | | | | | |
| NO: | | | | | | | | | |
| 84) | | | | | | | | | |

CHR2

| | | | | | | | | | bind- |
|---|---|---|---|---|---|---|---|---|---|
| | bind- | | | | | | | | ing |
| | ing | | | | | | | | sites |
| | sites | | | | | | | | (CHM13- |
| | (CHM13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| TCTAG | 2651 | 2651 | 1 | 40 | 2655 | 2655 | 0.99849341 | 0.47184718 | 0 |
| CTTTG | | | | | | | | | |
| AGGAT | | | | | | | | | |
| TTCGT | | | | | | | | | |
| (SEQ | | | | | | | | | |
| ID | | | | | | | | | |
| NO: | | | | | | | | | |
| 85) | | | | | | | | | |
| GCTTC | 2617 | 2617 | 1 | 40 | 2617 | 2617 | 1 | 0.50579891 | 0 |
| AACAC | | | | | | | | | |
| TGTTA | | | | | | | | | |
| GTTGA | | | | | | | | | |
| (SEQ | | | | | | | | | |
| ID | | | | | | | | | |
| NO: | | | | | | | | | |
| 12) | | | | | | | | | |
| GCATT | 2598 | 2598 | 1 | 40 | 2598 | 2598 | 1 | 0.52568992 | 0 |
| CTCAG | | | | | | | | | |
| AAGCT | | | | | | | | | |
| TCATT | | | | | | | | | |
| (SEQ | | | | | | | | | |
| ID | | | | | | | | | |
| NO: | | | | | | | | | |
| 86) | | | | | | | | | |
| AGCAT | 2557 | 2557 | 1 | 40 | 2557 | 2557 | 1 | 0.20446595 | 0 |
| TCTCA | | | | | | | | | |
| GAAGC | | | | | | | | | |
| TTCAT | | | | | | | | | |
| (SEQ | | | | | | | | | |
| ID | | | | | | | | | |
| NO: | | | | | | | | | |
| 87) | | | | | | | | | |
| AACAG | 2230 | 2231 | 1 | 45 | 2232 | 2233 | 0.99865652 | 0.1269202 | 0 |
| TCCCT | | | | | | | | | |
| TTCAT | | | | | | | | | |
| AGAGC | | | | | | | | | |
| (SEQ | | | | | | | | | |
| ID | | | | | | | | | |
| NO: | | | | | | | | | |
| 11) | | | | | | | | | |
| TTGGA | 809 | 809 | 1 | 55 | 809 | 809 | 1 | 0.11027372 | 0 |
| GCGCT | | | | | | | | | |
| CTCAG | | | | | | | | | |
| GACTA | | | | | | | | | |
| (SEQ | | | | | | | | | |
| ID | | | | | | | | | |
| NO: | | | | | | | | | |
| 88) | | | | | | | | | |
| TCTCA | 748 | 748 | 1 | 45 | 748 | 748 | 1 | 0.2179976 | 0 |
| GGACT | | | | | | | | | |
| ACGGT | | | | | | | | | |
| GAAAA | | | | | | | | | |
| (SEQ | | | | | | | | | |
| ID | | | | | | | | | |
| NO: | | | | | | | | | |
| 89) | | | | | | | | | |
| GGTTC | 645 | 646 | 0.998 | 40 | 649 | 650 | 0.99230769 | 0.40727945 | 0 |
| AACAC | | | | | | | | | |
| TGTTA | | | | | | | | | |
| GTTGA | | | | | | | | | |
| (SEQ | | | | | | | | | |
| ID | | | | | | | | | |
| NO: | | | | | | | | | |
| 90) | | | | | | | | | |
| TCTCA | 511 | 511 | 1 | 40 | 511 | 511 | 1 | 0.11809792 | 0 |
| GGAAT | | | | | | | | | |
| ACGGT | | | | | | | | | |
| GATAA | | | | | | | | | |
| (SEQ | | | | | | | | | |
| ID | | | | | | | | | |
| NO: | | | | | | | | | |
| 91) | | | | | | | | | |
| TCTCA | 510 | 510 | 1 | 50 | 510 | 510 | 1 | 0.23864461 | 0 |
| GGACT | | | | | | | | | |
| GCGGT | | | | | | | | | |
| GAAAA | | | | | | | | | |
| (SEQ | | | | | | | | | |
| ID | | | | | | | | | |
| NO: | | | | | | | | | |
| 92) | | | | | | | | | |

CHR3

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| TGTGT | 500 | 500 | 1 | 40 | 500 | 500 | 1 | 0.31483826 | 0 |
| GTGTA | | | | | | | | | |
| TTCAA | | | | | | | | | |
| CTCAC | | | | | | | | | |
| (SEQ | | | | | | | | | |
| ID | | | | | | | | | |
| NO: | | | | | | | | | |
| 93) | | | | | | | | | |
| GGGAG | 482 | 482 | 1 | 45 | 482 | 482 | 1 | 0.18290363 | 0 |
| ATTTC | | | | | | | | | |
| AAGCA | | | | | | | | | |
| CTTTG | | | | | | | | | |
| (SEQ | | | | | | | | | |
| ID | | | | | | | | | |
| NO: | | | | | | | | | |
| 94) | | | | | | | | | |
| TATGA | 482 | 482 | 1 | 40 | 482 | 482 | 1 | 0.30462167 | 0 |
| GGCCA | | | | | | | | | |
| ATGGT | | | | | | | | | |
| ACAAA | | | | | | | | | |
| (SEQ | | | | | | | | | |
| ID | | | | | | | | | |
| NO: | | | | | | | | | |
| 95) | | | | | | | | | |
| TGAAT | 478 | 478 | 1 | 45 | 478 | 478 | 1 | 0.77134816 | 0 |
| GCAGA | | | | | | | | | |
| GATCA | | | | | | | | | |
| CAACG | | | | | | | | | |
| (SEQ | | | | | | | | | |
| ID | | | | | | | | | |
| NO: | | | | | | | | | |
| 96) | | | | | | | | | |
| CACAG | 470 | 470 | 1 | 40 | 471 | 471 | 0.99787686 | 0.22559285 | 0 |
| AGTTG | | | | | | | | | |
| AACCT | | | | | | | | | |
| TACTT | | | | | | | | | |
| (SEQ | | | | | | | | | |
| ID | | | | | | | | | |
| NO: | | | | | | | | | |
| 97) | | | | | | | | | |
| GATGT | 470 | 470 | 1 | 45 | 470 | 470 | 1 | 0.35900752 | 0 |
| ATTTG | | | | | | | | | |
| AGGCC | | | | | | | | | |
| TTCGT | | | | | | | | | |
| (SEQ | | | | | | | | | |
| ID | | | | | | | | | |
| NO: | | | | | | | | | |
| 98) | | | | | | | | | |
| TTTGA | 470 | 470 | 1 | 40 | 470 | 472 | 0.99576271 | 0.21745241 | 0 |
| GTGCT | | | | | | | | | |
| TTGAA | | | | | | | | | |
| GCCTA | | | | | | | | | |
| (SEQ | | | | | | | | | |
| ID | | | | | | | | | |
| NO: | | | | | | | | | |
| 99) | | | | | | | | | |
| AGAAA | 468 | 468 | 1 | 40 | 468 | 468 | 1 | 0.49259342 | 0 |
| TCCCG | | | | | | | | | |
| TTTAC | | | | | | | | | |
| TACGA | | | | | | | | | |
| (SEQ | | | | | | | | | |
| ID | | | | | | | | | |
| NO: | | | | | | | | | |
| 100) | | | | | | | | | |
| TGGAG | 468 | 468 | 1 | 50 | 468 | 468 | 1 | 0.16186147 | 0 |
| GTATC | | | | | | | | | |
| AAGCG | | | | | | | | | |
| CTTTG | | | | | | | | | |
| (SEQ | | | | | | | | | |
| ID | | | | | | | | | |
| NO: | | | | | | | | | |
| 101) | | | | | | | | | |
| GAACC | 466 | 467 | 0.998 | 45 | 466 | 469 | 0.99360341 | 0.3728499 | 0 |
| TTCCT | | | | | | | | | |
| TTAGA | | | | | | | | | |
| CAGAG | | | | | | | | | |
| (SEQ | | | | | | | | | |
| ID | | | | | | | | | |
| NO: | | | | | | | | | |
| 102) | | | | | | | | | |
| AAGCA | 465 | 465 | 1 | 50 | 465 | 465 | 1 | 0.29560094 | 0 |
| CTTTG | | | | | | | | | |
| AGGCC | | | | | | | | | |
| ATTGG | | | | | | | | | |
| (SEQ | | | | | | | | | |
| ID | | | | | | | | | |
| NO: | | | | | | | | | |
| 103) | | | | | | | | | |
| TTCCT | 465 | 465 | 1 | 45 | 465 | 465 | 1 | 0.17734788 | 0 |
| TTAGA | | | | | | | | | |
| CAGAG | | | | | | | | | |
| CGGAT | | | | | | | | | |
| (SEQ | | | | | | | | | |
| ID | | | | | | | | | |
| NO: | | | | | | | | | |
| 104) | | | | | | | | | |
| TTGAG | 463 | 463 | 1 | 45 | 463 | 463 | 1 | 0.28037324 | 0 |
| GCCTT | | | | | | | | | |
| CGTAG | | | | | | | | | |
| TAAAC | | | | | | | | | |
| (SEQ | | | | | | | | | |
| ID | | | | | | | | | |
| NO: | | | | | | | | | |
| 105) | | | | | | | | | |
| TTTGA | 463 | 463 | 1 | 45 | 463 | 466 | 0.99356223 | 0.1654108 | 0 |
| GGCCA | | | | | | | | | |
| TTGGT | | | | | | | | | |
| GGAAA | | | | | | | | | |
| (SEQ | | | | | | | | | |
| ID | | | | | | | | | |
| NO: | | | | | | | | | |
| 106) | | | | | | | | | |
| TTCCA | 462 | 462 | 1 | 45 | 462 | 462 | 1 | 0.14581255 | 0 |
| ATCCG | | | | | | | | | |
| CTCTG | | | | | | | | | |
| TCTAA | | | | | | | | | |
| (SEQ | | | | | | | | | |
| ID | | | | | | | | | |
| NO: | | | | | | | | | |
| 107) | | | | | | | | | |
| AATCC | 460 | 461 | 0.998 | 45 | 460 | 464 | 0.99137931 | 0.70409894 | 0 |
| GCTCT | | | | | | | | | |
| GTCTA | | | | | | | | | |
| AAGGA | | | | | | | | | |
| (SEQ | | | | | | | | | |
| ID | | | | | | | | | |
| NO: | | | | | | | | | |
| 108) | | | | | | | | | |
| CAGTT | 460 | 460 | 1 | 40 | 460 | 460 | 1 | 0.27171858 | 0 |
| TGTAA | | | | | | | | | |
| AGTCA | | | | | | | | | |
| GCAAC | | | | | | | | | |
| (SEQ | | | | | | | | | |
| ID | | | | | | | | | |
| NO: | | | | | | | | | |
| 109) | | | | | | | | | |
| TTTGT | 458 | 458 | 1 | 40 | 458 | 458 | 1 | 0.27498205 | 0 |
| GGAAT | | | | | | | | | |
| TTTCA | | | | | | | | | |
| GGTGG | | | | | | | | | |
| (SEQ | | | | | | | | | |
| ID | | | | | | | | | |
| NO: | | | | | | | | | |
| 110) | | | | | | | | | |
| AACGT | 457 | 457 | 1 | 50 | 457 | 457 | 1 | 0.20356074 | 0 |
| CTTTG | | | | | | | | | |
| AGGCC | | | | | | | | | |
| TTCGT | | | | | | | | | |
| (SEQ | | | | | | | | | |
| ID | | | | | | | | | |
| NO: | | | | | | | | | |
| 111) | | | | | | | | | |
| TCACT | 454 | 454 | 1 | 40 | 454 | 454 | 1 | 0.18452955 | 0 |
| GAGAA | | | | | | | | | |
| TTCTT | | | | | | | | | |
| CTGTC | | | | | | | | | |
| (SEQ | | | | | | | | | |
| ID | | | | | | | | | |
| NO: | | | | | | | | | |
| 112) | | | | | | | | | |
| TTTGA | 454 | 454 | 1 | 40 | 454 | 454 | 1 | 0.40487776 | 0 |
| GGCCT | | | | | | | | | |
| TCGTA | | | | | | | | | |
| GTAAA | | | | | | | | | |
| (SEQ | | | | | | | | | |
| ID | | | | | | | | | |
| NO: | | | | | | | | | |
| 113) | | | | | | | | | |
| ACAGA | 453 | 453 | 1 | 40 | 453 | 453 | 1 | 0.41744193 | 0 |
| GTTGA | | | | | | | | | |
| AGCTT | | | | | | | | | |
| CCTTT | | | | | | | | | |
| (SEQ | | | | | | | | | |
| ID | | | | | | | | | |
| NO: | | | | | | | | | |
| 114) | | | | | | | | | |
| ATTGA | 453 | 453 | 1 | 40 | 454 | 454 | 0.99779736 | 0.24022227 | 0 |
| AGCCT | | | | | | | | | |
| ACGGT | | | | | | | | | |
| AGAAA | | | | | | | | | |
| (SEQ | | | | | | | | | |
| ID | | | | | | | | | |
| NO: | | | | | | | | | |
| 115) | | | | | | | | | |
| TTACT | 453 | 453 | 1 | 45 | 453 | 453 | 1 | 0.80875521 | 0 |
| ACGAA | | | | | | | | | |
| GGCCT | | | | | | | | | |
| CAAAG | | | | | | | | | |
| (SEQ | | | | | | | | | |
| ID | | | | | | | | | |
| NO: | | | | | | | | | |
| 116) | | | | | | | | | |
| TTCCA | 453 | 453 | 1 | 50 | 453 | 453 | 1 | 0.24812839 | 0 |
| ATCTG | | | | | | | | | |
| CTCCG | | | | | | | | | |
| CCTAA | | | | | | | | | |
| (SEQ | | | | | | | | | |
| ID | | | | | | | | | |
| NO: | | | | | | | | | |
| 13) | | | | | | | | | |
| TTCCT | 450 | 450 | 1 | 50 | 450 | 450 | 1 | 0.1116799 | 0 |
| TTAGG | | | | | | | | | |
| CGGAG | | | | | | | | | |
| CAGAT | | | | | | | | | |
| (SEQ | | | | | | | | | |
| ID | | | | | | | | | |
| NO: | | | | | | | | | |
| 14) | | | | | | | | | |
| TTCCA | 439 | 439 | 1 | 40 | 439 | 439 | 1 | 0.58805317 | 0 |
| ACGAA | | | | | | | | | |
| GACTT | | | | | | | | | |
| CAAAG | | | | | | | | | |
| (SEQ | | | | | | | | | |
| ID | | | | | | | | | |
| NO: | | | | | | | | | |
| 117) | | | | | | | | | |
| CAGTT | 437 | 437 | 1 | 45 | 437 | 437 | 1 | 0.25091077 | 0 |
| TGTAA | | | | | | | | | |
| TGTCT | | | | | | | | | |
| GCAGC | | | | | | | | | |
| (SEQ | | | | | | | | | |
| ID | | | | | | | | | |
| NO: | | | | | | | | | |
| 118) | | | | | | | | | |
| GACCT | 437 | 437 | 1 | 45 | 437 | 437 | 1 | 0.33127154 | 0 |
| CTTTG | | | | | | | | | |
| AAGTC | | | | | | | | | |
| TTCGT | | | | | | | | | |
| (SEQ | | | | | | | | | |
| ID | | | | | | | | | |
| NO: | | | | | | | | | |
| 119) | | | | | | | | | |
| GAGTT | 434 | 434 | 1 | 45 | 434 | 434 | 1 | 0.33630605 | 0 |
| GAAGC | | | | | | | | | |
| TTCCT | | | | | | | | | |
| TTAGG | | | | | | | | | |
| (SEQ | | | | | | | | | |
| ID | | | | | | | | | |
| NO: | | | | | | | | | |
| 120) | | | | | | | | | |
| AATCT | 432 | 432 | 1 | 40 | 432 | 432 | 1 | 0.42601474 | 0 |
| GCACT | | | | | | | | | |
| GTCTA | | | | | | | | | |
| AAGGA | | | | | | | | | |
| (SEQ | | | | | | | | | |
| ID | | | | | | | | | |
| NO: | | | | | | | | | |
| 121) | | | | | | | | | |
| TCAGT | 429 | 429 | 1 | 40 | 429 | 429 | 1 | 0.13682221 | 0 |
| AACTT | | | | | | | | | |
| CTTTG | | | | | | | | | |
| GGTTG | | | | | | | | | |
| (SEQ | | | | | | | | | |
| ID | | | | | | | | | |
| NO: | | | | | | | | | |
| 122) | | | | | | | | | |
| CTTGT | 426 | 426 | 1 | 40 | 426 | 426 | 1 | 0.59310238 | 0 |
| CTGTG | | | | | | | | | |
| GAATT | | | | | | | | | |
| TGCAA | | | | | | | | | |
| (SEQ | | | | | | | | | |
| ID | | | | | | | | | |
| NO: | | | | | | | | | |
| 123) | | | | | | | | | |
| ACTTG | 425 | 425 | 1 | 40 | 425 | 425 | 1 | 0.56050115 | 0 |
| TCTGT | | | | | | | | | |
| GGAAT | | | | | | | | | |
| TTGCA | | | | | | | | | |
| (SEQ | | | | | | | | | |
| ID | | | | | | | | | |
| NO: | | | | | | | | | |
| 124) | | | | | | | | | |
| TTGTC | 422 | 422 | 1 | 40 | 422 | 422 | 1 | 0.61015084 | 0 |
| TGTGG | | | | | | | | | |
| AATTT | | | | | | | | | |
| GCAAG | | | | | | | | | |
| (SEQ | | | | | | | | | |
| ID | | | | | | | | | |
| NO: | | | | | | | | | |
| 125) | | | | | | | | | |
| TTCAA | 402 | 402 | 1 | 40 | 402 | 402 | 1 | 0.21529739 | 0 |
| GCGCT | | | | | | | | | |
| TTGAA | | | | | | | | | |
| GTGAA | | | | | | | | | |
| (SEQ | | | | | | | | | |
| ID | | | | | | | | | |
| NO: | | | | | | | | | |
| 126) | | | | | | | | | |

CHR4

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| CCACC | 2301 | 2302 | 1 | 45 | 2301 | 2305 | 0.99826464 | 0.62458876 | 0 |
| TGCAG | | | | | | | | | |
| ATTCT | | | | | | | | | |
| ACAAA | | | | | | | | | |
| (SEQ | | | | | | | | | |
| ID | | | | | | | | | |
| NO: | | | | | | | | | |
| 18) | | | | | | | | | |
| AAACT | 1973 | 1974 | 0.999 | 40 | 1973 | 1975 | 0.99898734 | 0.33919873 | 0 |
| GCTGT | | | | | | | | | |
| GTCAA | | | | | | | | | |
| AAGGA | | | | | | | | | |
| (SEQ | | | | | | | | | |
| ID | | | | | | | | | |
| NO: | | | | | | | | | |
| 127) | | | | | | | | | |
| GCAGA | 1582 | 1586 | 0.997 | 40 | 1582 | 1586 | 0.99747793 | 0.57779023 | 0 |
| AAGAG | | | | | | | | | |
| TGTTT | | | | | | | | | |
| CAAAC | | | | | | | | | |
| (SEQ | | | | | | | | | |
| ID | | | | | | | | | |
| NO: | | | | | | | | | |
| 128) | | | | | | | | | |
| TGATG | 1233 | 1233 | 1 | 40 | 1233 | 1233 | 1 | 0.10846441 | 0 |
| TTTGC | | | | | | | | | |
| ATTCA | | | | | | | | | |
| GCTCA | | | | | | | | | |
| (SEQ | | | | | | | | | |
| ID | | | | | | | | | |
| NO: | | | | | | | | | |
| 129) | | | | | | | | | |
| AGAAT | 1226 | 1226 | 1 | 45 | 1226 | 1226 | 1 | 0.14553785 | 0 |
| CTGCA | | | | | | | | | |
| GGTGG | | | | | | | | | |
| ATATG | | | | | | | | | |
| (SEQ | | | | | | | | | |
| ID | | | | | | | | | |
| NO: | | | | | | | | | |
| 130) | | | | | | | | | |
| CTTTC | 1224 | 1224 | 1 | 40 | 1224 | 1227 | 0.99755501 | 0.50791256 | 0 |
| TCTAG | | | | | | | | | |
| TATCT | | | | | | | | | |
| GGAAG | | | | | | | | | |
| (SEQ | | | | | | | | | |
| ID | | | | | | | | | |
| NO: | | | | | | | | | |
| 131) | | | | | | | | | |
| TCAGA | 1186 | 1186 | 1 | 40 | 1186 | 1186 | 1 | 0.69807476 | 0 |
| AAGTG | | | | | | | | | |
| GATAT | | | | | | | | | |
| TCGGA | | | | | | | | | |
| (SEQ | | | | | | | | | |
| ID | | | | | | | | | |
| NO: | | | | | | | | | |
| 132) | | | | | | | | | |
| CATTC | 1167 | 1167 | 1 | 40 | 1167 | 1172 | 0.99573379 | 0.51701666 | 0 |
| TGTAG | | | | | | | | | |
| TATCT | | | | | | | | | |
| GGAAG | | | | | | | | | |
| (SEQ | | | | | | | | | |
| ID | | | | | | | | | |
| NO: | | | | | | | | | |
| 133) | | | | | | | | | |
| TTCGA | 1158 | 1158 | 1 | 50 | 1158 | 1160 | 0.99827586 | 0.18565007 | 0 |
| GCGCT | | | | | | | | | |
| TTGAG | | | | | | | | | |
| TCCTA | | | | | | | | | |
| (SEQ | | | | | | | | | |
| ID | | | | | | | | | |
| NO: | | | | | | | | | |
| 134) | | | | | | | | | |
| GATGG | 1145 | 1145 | 1 | 50 | 1145 | 1145 | 1 | 0.74334262 | 0 |
| CTCTG | | | | | | | | | |
| AGGAT | | | | | | | | | |
| TTCGT | | | | | | | | | |
| (SEQ | | | | | | | | | |
| ID | | | | | | | | | |
| NO: | | | | | | | | | |
| 135) | | | | | | | | | |
| TCTGA | 1144 | 1145 | 0.999 | 45 | 1144 | 1153 | 0.99219428 | 0.29685998 | 0 |
| GGATT | | | | | | | | | |
| TCGTT | | | | | | | | | |
| GGAAG | | | | | | | | | |
| (SEQ | | | | | | | | | |
| ID | | | | | | | | | |
| NO: | | | | | | | | | |
| 136) | | | | | | | | | |
| TGGAT | 1134 | 1134 | 1 | 50 | 1134 | 1134 | 1 | 0.24343619 | 0 |
| ATTCG | | | | | | | | | |
| GATGG | | | | | | | | | |
| CTCTG | | | | | | | | | |
| (SEQ | | | | | | | | | |
| ID | | | | | | | | | |
| NO: | | | | | | | | | |
| 137) | | | | | | | | | |
| GATAG | 1128 | 1129 | 0.999 | 40 | 1128 | 1129 | 0.99911426 | 0.71723426 | 0 |
| CTCTG | | | | | | | | | |
| AAGAT | | | | | | | | | |
| TTCGT | | | | | | | | | |
| (SEQ | | | | | | | | | |
| ID | | | | | | | | | |
| NO: | | | | | | | | | |
| 138) | | | | | | | | | |
| CAACT | 1059 | 1060 | 0.999 | 40 | 1059 | 1060 | 0.9990566 | 0.45044688 | 0 |
| AACAG | | | | | | | | | |
| AGTTG | | | | | | | | | |
| AACCT | | | | | | | | | |
| (SEQ | | | | | | | | | |
| ID | | | | | | | | | |
| NO: | | | | | | | | | |
| 139) | | | | | | | | | |
| GATAG | 1050 | 1050 | 1 | 45 | 1050 | 1050 | 1 | 0.7534398 | 0 |
| CTTAG | | | | | | | | | |
| AGGGA | | | | | | | | | |
| TTCGT | | | | | | | | | |
| (SEQ | | | | | | | | | |
| ID | | | | | | | | | |
| NO: | | | | | | | | | |
| 140) | | | | | | | | | |
| TTTCA | 1044 | 1048 | 0.996 | 45 | 1044 | 1048 | 0.99618321 | 0.29912161 | 0 |
| GGCCT | | | | | | | | | |
| ATGGA | | | | | | | | | |
| GAGAA | | | | | | | | | |
| (SEQ | | | | | | | | | |
| ID | | | | | | | | | |
| NO: | | | | | | | | | |
| 141) | | | | | | | | | |
| AGGTT | 1040 | 1040 | 1 | 45 | 1040 | 1040 | 1 | 0.16767199 | 0 |
| CAGCT | | | | | | | | | |
| CTGTG | | | | | | | | | |
| AATTG | | | | | | | | | |
| (SEQ | | | | | | | | | |
| ID | | | | | | | | | |
| NO: | | | | | | | | | |
| 142) | | | | | | | | | |
| TTAGA | 1040 | 1040 | 1 | 40 | 1040 | 1040 | 1 | 0.44633557 | 0 |
| GGGAT | | | | | | | | | |
| TCGTT | | | | | | | | | |
| GGAAA | | | | | | | | | |
| (SEQ | | | | | | | | | |
| ID | | | | | | | | | |
| NO: | | | | | | | | | |
| 143) | | | | | | | | | |
| TAGAG | 1039 | 1039 | 1 | 45 | 1039 | 1039 | 1 | 0.22322107 | 0 |
| GGATT | | | | | | | | | |
| CGTTG | | | | | | | | | |
| GAAAG | | | | | | | | | |
| (SEQ | | | | | | | | | |
| ID | | | | | | | | | |
| NO: | | | | | | | | | |
| 144) | | | | | | | | | |
| CACAG | 1031 | 1031 | 1 | 45 | 1031 | 1031 | 1 | 0.23855624 | 0 |
| AGCTG | | | | | | | | | |
| AACCT | | | | | | | | | |
| TTGTT | | | | | | | | | |
| (SEQ | | | | | | | | | |
| ID | | | | | | | | | |
| NO: | | | | | | | | | |
| 145) | | | | | | | | | |
| TTTCT | 1030 | 1030 | 1 | 45 | 1030 | 1033 | 0.99709584 | 0.12382033 | 0 |
| GAGAA | | | | | | | | | |
| TGCTC | | | | | | | | | |
| CTGTC | | | | | | | | | |
| (SEQ | | | | | | | | | |
| ID | | | | | | | | | |
| NO: | | | | | | | | | |
| 146) | | | | | | | | | |
| GGTAT | 1022 | 1022 | 1 | 40 | 1022 | 1022 | 1 | 0.31973646 | 0 |
| TTCCT | | | | | | | | | |
| TTCTC | | | | | | | | | |
| TCCAT | | | | | | | | | |
| (SEQ | | | | | | | | | |
| ID | | | | | | | | | |
| NO: | | | | | | | | | |
| 147) | | | | | | | | | |
| CTTTG | 1014 | 1014 | 1 | 40 | 1014 | 1016 | 0.9980315 | 0.20455576 | 0 |
| AGGCC | | | | | | | | | |
| TATGG | | | | | | | | | |
| TTAAA | | | | | | | | | |
| (SEQ | | | | | | | | | |
| ID | | | | | | | | | |
| NO: | | | | | | | | | |
| 148) | | | | | | | | | |
| AACAG | 1009 | 1009 | 1 | 40 | 1009 | 1009 | 1 | 0.24797915 | 0 |
| AGTTG | | | | | | | | | |
| AACCA | | | | | | | | | |
| TTGCT | | | | | | | | | |
| (SEQ | | | | | | | | | |
| ID | | | | | | | | | |
| NO: | | | | | | | | | |
| 149) | | | | | | | | | |
| TGGAT | 1009 | 1009 | 1 | 40 | 1009 | 1011 | 0.99802176 | 0.12980738 | 0 |
| ATTTC | | | | | | | | | |
| GAGCT | | | | | | | | | |
| CTTTG | | | | | | | | | |
| (SEQ | | | | | | | | | |
| ID | | | | | | | | | |
| NO: | | | | | | | | | |
| 150) | | | | | | | | | |
| TTCGA | 1006 | 1006 | 1 | 50 | 1006 | 1006 | 1 | 0.117938 | 0 |
| GCTCT | | | | | | | | | |
| TTGAG | | | | | | | | | |
| GCCTA | | | | | | | | | |
| (SEQ | | | | | | | | | |
| ID | | | | | | | | | |
| NO: | | | | | | | | | |
| 151) | | | | | | | | | |
| ACTCC | 986 | 986 | 1 | 45 | 986 | 986 | 1 | 0.12892132 | 0 |
| TTTTG | | | | | | | | | |
| TAGGA | | | | | | | | | |
| TCTGC | | | | | | | | | |
| (SEQ | | | | | | | | | |
| ID | | | | | | | | | |
| NO: | | | | | | | | | |
| 152) | | | | | | | | | |
| CCTTT | 980 | 980 | 1 | 50 | 980 | 980 | 1 | 0.729451 | 0 |
| TGTAG | | | | | | | | | |
| GATCT | | | | | | | | | |
| GCAGG | | | | | | | | | |
| (SEQ | | | | | | | | | |
| ID | | | | | | | | | |
| NO: | | | | | | | | | |
| 153) | | | | | | | | | |
| CCACC | 979 | 979 | 1 | 50 | 979 | 979 | 1 | 0.67077609 | 0 |
| TGCAG | | | | | | | | | |
| ATCCT | | | | | | | | | |
| ACAAA | | | | | | | | | |
| (SEQ | | | | | | | | | |
| ID | | | | | | | | | |
| NO: | | | | | | | | | |
| 154) | | | | | | | | | |
| GATAT | 892 | 892 | 1 | 45 | 892 | 892 | 1 | 0.57112971 | 0 |
| TTCCT | | | | | | | | | |
| TTCTC | | | | | | | | | |
| CCCGT | | | | | | | | | |
| (SEQ | | | | | | | | | |
| ID | | | | | | | | | |
| NO: | | | | | | | | | |
| 155) | | | | | | | | | |
| TTTCA | 885 | 885 | 1 | 55 | 885 | 885 | 1 | 0.17647391 | 0 |
| GGCCT | | | | | | | | | |
| ACGGG | | | | | | | | | |
| GAGAA | | | | | | | | | |
| (SEQ | | | | | | | | | |
| ID | | | | | | | | | |
| NO: | | | | | | | | | |
| 156) | | | | | | | | | |
| TCAAG | 884 | 884 | 1 | 55 | 884 | 884 | 1 | 0.30386285 | 0 |
| CGCTT | | | | | | | | | |
| TCAGG | | | | | | | | | |
| CCTAC | | | | | | | | | |
| (SEQ | | | | | | | | | |
| ID | | | | | | | | | |
| NO: | | | | | | | | | |
| 157) | | | | | | | | | |
| CAAGC | 879 | 879 | 1 | 60 | 879 | 879 | 1 | 0.20087395 | 0 |
| GCTTT | | | | | | | | | |
| CAGGC | | | | | | | | | |
| CTACG | | | | | | | | | |
| (SEQ | | | | | | | | | |
| ID | | | | | | | | | |
| NO: | | | | | | | | | |
| 158) | | | | | | | | | |
| CTTTC | 842 | 844 | 0.998 | 55 | 842 | 844 | 0.99763033 | 0.39199255 | 0 |
| GGCAC | | | | | | | | | |
| TACCT | | | | | | | | | |
| GGAAG | | | | | | | | | |
| (SEQ | | | | | | | | | |
| ID | | | | | | | | | |
| NO: | | | | | | | | | |
| 159) | | | | | | | | | |
| AACAC | 840 | 840 | 1 | 50 | 840 | 840 | 1 | 0.12228215 | 0 |
| TCTTT | | | | | | | | | |
| CGGCA | | | | | | | | | |
| CTACC | | | | | | | | | |
| (SEQ | | | | | | | | | |
| ID | | | | | | | | | |
| NO: | | | | | | | | | |
| 160) | | | | | | | | | |
| CAACT | 746 | 748 | 0.997 | 40 | 746 | 748 | 0.9973262 | 0.10427631 | 0 |
| TGCAG | | | | | | | | | |
| ATTCT | | | | | | | | | |
| ACTCA | | | | | | | | | |
| (SEQ | | | | | | | | | |
| ID | | | | | | | | | |
| NO: | | | | | | | | | |
| 161) | | | | | | | | | |

CHR5

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| AGTTG | 2520 | 2530 | 0.996 | 40 | 2520 | 2530 | 0.99604743 | 0.53381999 | 0 |
| AACAC | | | | | | | | | |
| ACACA | | | | | | | | | |
| ACACA | | | | | | | | | |
| (SEQ | | | | | | | | | |
| ID | | | | | | | | | |
| NO: | | | | | | | | | |
| 21) | | | | | | | | | |
| TACAA | 1613 | 1616 | 0.998 | 40 | 1613 | 1616 | 0.99814356 | 0.12770753 | 0 |
| GTCTG | | | | | | | | | |
| CTCTG | | | | | | | | | |
| TGTAA | | | | | | | | | |
| (SEQ | | | | | | | | | |
| ID | | | | | | | | | |
| NO: | | | | | | | | | |
| 22) | | | | | | | | | |
| TTCTA | 769 | 773 | 0.995 | 45 | 769 | 773 | 0.99482536 | 0.64630859 | 0 |
| CCATT | | | | | | | | | |
| GACCT | | | | | | | | | |
| CAACG | | | | | | | | | |
| (SEQ | | | | | | | | | |
| ID | | | | | | | | | |
| NO: | | | | | | | | | |
| 162) | | | | | | | | | |
| TTCAG | 752 | 757 | 0.993 | 55 | 752 | 758 | 0.99208443 | 0.13139334 | 0 |
| CCGCG | | | | | | | | | |
| TTGAG | | | | | | | | | |
| GTCAA | | | | | | | | | |
| (SEQ | | | | | | | | | |
| ID | | | | | | | | | |
| NO: | | | | | | | | | |
| 163) | | | | | | | | | |

CHR6

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | chro- | | bind- | | | | chro- |
| | chro- | 13- | mo- | | ing | bind- | | | mo- |
| | mo- | all | some | | sites | ing | centro- | | some |
| | some | cen- | spe- | | (CHM- | sites | mere | | centro- |
| sg | centro- | tro- | ci- | | 13 | (hg38 | spe- | Ac- | mere |
| RNA | mere | mere | f- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| ATGGC | 1615 | 1615 | 1 | 50 | 1615 | 1615 | 1 | 0.28633287 | 0 |
| TGCAT | | | | | | | | | |
| TCCAC | | | | | | | | | |
| ACACA | | | | | | | | | |
| (SEQ | | | | | | | | | |
| ID | | | | | | | | | |
| NO: | | | | | | | | | |
| 24) | | | | | | | | | |
| GCTGC | 1611 | 1611 | 1 | 60 | 1611 | 1611 | 1 | 0.9049251 | 0 |
| ATTCC | | | | | | | | | |
| ACACA | | | | | | | | | |
| CACGG | | | | | | | | | |
| (SEQ | | | | | | | | | |
| ID | | | | | | | | | |
| NO: | | | | | | | | | |
| 164) | | | | | | | | | |
| TTTCC | 956 | 956 | 1 | 40 | 956 | 956 | 1 | 0.18244023 | 0 |
| AAAGA | | | | | | | | | |
| ATGCC | | | | | | | | | |
| TCCAA | | | | | | | | | |
| (SEQ | | | | | | | | | |
| ID | | | | | | | | | |
| NO: | | | | | | | | | |
| 165) | | | | | | | | | |
| CTTGG | 955 | 955 | 1 | 40 | 955 | 955 | 1 | 0.10608966 | 0 |
| AAATC | | | | | | | | | |
| CTACA | | | | | | | | | |
| AGAAC | | | | | | | | | |
| (SEQ | | | | | | | | | |
| ID | | | | | | | | | |
| NO: | | | | | | | | | |
| 166) | | | | | | | | | |
| GTTCT | 954 | 954 | 1 | 40 | 954 | 954 | 1 | 0.94269713 | 0 |
| TGTAG | | | | | | | | | |
| GATTT | | | | | | | | | |
| CCAAG | | | | | | | | | |
| (SEQ | | | | | | | | | |
| ID | | | | | | | | | |
| NO: | | | | | | | | | |
| 167) | | | | | | | | | |
| TCTCT | 952 | 952 | 1 | 40 | 952 | 952 | 1 | 0.18452955 | 0 |
| GAGAA | | | | | | | | | |
| TTCTT | | | | | | | | | |
| CTGTC | | | | | | | | | |
| (SEQ | | | | | | | | | |
| ID | | | | | | | | | |
| NO: | | | | | | | | | |
| 168) | | | | | | | | | |
| TGGAG | 952 | 952 | 1 | 40 | 952 | 952 | 1 | 0.1139595 | 0 |
| GCATT | | | | | | | | | |
| CTTTG | | | | | | | | | |
| GAAAA | | | | | | | | | |
| (SEQ | | | | | | | | | |
| ID | | | | | | | | | |
| NO: | | | | | | | | | |
| 169) | | | | | | | | | |
| TTGGA | 952 | 952 | 1 | 40 | 952 | 952 | 1 | 0.15648066 | 0 |
| GGCAT | | | | | | | | | |
| TCTTT | | | | | | | | | |
| GGAAA | | | | | | | | | |
| (SEQ | | | | | | | | | |
| ID | | | | | | | | | |
| NO: | | | | | | | | | |
| 170) | | | | | | | | | |
| TTTTC | 952 | 952 | 1 | 40 | 952 | 952 | 1 | 0.2370732 | 0 |
| CAAAG | | | | | | | | | |
| AATGC | | | | | | | | | |
| CTCCA | | | | | | | | | |
| (SEQ | | | | | | | | | |
| ID | | | | | | | | | |
| NO: | | | | | | | | | |
| 171) | | | | | | | | | |
| TGAAC | 950 | 950 | 1 | 45 | 950 | 950 | 1 | 0.37026507 | 0 |
| GCACA | | | | | | | | | |
| CATCA | | | | | | | | | |
| CAATG | | | | | | | | | |
| (SEQ | | | | | | | | | |
| ID | | | | | | | | | |
| NO: | | | | | | | | | |
| 172) | | | | | | | | | |
| GAGCC | 948 | 948 | 1 | 55 | 948 | 948 | 1 | 0.3039169 | 0 |
| CTTGG | | | | | | | | | |
| AGGCA | | | | | | | | | |
| TTCTT | | | | | | | | | |
| (SEQ | | | | | | | | | |
| ID | | | | | | | | | |
| NO: | | | | | | | | | |
| 173) | | | | | | | | | |
| TGTGA | 945 | 945 | 1 | 40 | 945 | 945 | 1 | 0.48406532 | 0 |
| ATTCA | | | | | | | | | |
| ACTCA | | | | | | | | | |
| CAGTG | | | | | | | | | |
| (SEQ | | | | | | | | | |
| ID | | | | | | | | | |
| NO: | | | | | | | | | |
| 174) | | | | | | | | | |
| TGGAT | 944 | 944 | 1 | 45 | 944 | 944 | 1 | 0.15656561 | 0 |
| ATTTT | | | | | | | | | |
| GAGCC | | | | | | | | | |
| CTTGG | | | | | | | | | |
| (SEQ | | | | | | | | | |
| ID | | | | | | | | | |
| NO: | | | | | | | | | |
| 175) | | | | | | | | | |
| ATGCG | 939 | 939 | 1 | 40 | 939 | 939 | 1 | 0.49594688 | 0 |
| CTATA | | | | | | | | | |
| AATAT | | | | | | | | | |
| CCCCT | | | | | | | | | |
| (SEQ | | | | | | | | | |
| ID | | | | | | | | | |
| NO: | | | | | | | | | |
| 176) | | | | | | | | | |
| ACTTG | 938 | 938 | 1 | 40 | 938 | 938 | 1 | 0.43765649 | 0 |
| CAGCT | | | | | | | | | |
| ACTAC | | | | | | | | | |
| AAGAA | | | | | | | | | |
| (SEQ | | | | | | | | | |
| ID | | | | | | | | | |
| NO: | | | | | | | | | |
| 177) | | | | | | | | | |
| TGTAC | 937 | 942 | 0.995 | 40 | 937 | 944 | 0.99258475 | 0.69280101 | 0 |
| ATTCA | | | | | | | | | |
| ACTCA | | | | | | | | | |
| CAGAG | | | | | | | | | |
| (SEQ | | | | | | | | | |
| ID | | | | | | | | | |
| NO: | | | | | | | | | |
| 178) | | | | | | | | | |
| CACAA | 936 | 936 | 1 | 40 | 936 | 936 | 1 | 0.15841395 | 0 |
| AGTCG | | | | | | | | | |
| TTTCT | | | | | | | | | |
| GAGAT | | | | | | | | | |
| (SEQ | | | | | | | | | |
| ID | | | | | | | | | |
| NO: | | | | | | | | | |
| 179) | | | | | | | | | |
| CTTCT | 936 | 936 | 1 | 45 | 936 | 936 | 1 | 0.62604576 | 0 |
| TGTAG | | | | | | | | | |
| TAGCT | | | | | | | | | |
| GCAAG | | | | | | | | | |
| (SEQ | | | | | | | | | |
| ID | | | | | | | | | |
| NO: | | | | | | | | | |
| 180) | | | | | | | | | |
| TTCCA | 936 | 936 | 1 | 45 | 936 | 936 | 1 | 0.50154664 | 0 |
| AAGAA | | | | | | | | | |
| GGCCT | | | | | | | | | |
| CCAAT | | | | | | | | | |
| (SEQ | | | | | | | | | |
| ID | | | | | | | | | |
| NO: | | | | | | | | | |
| 181) | | | | | | | | | |
| GACCT | 935 | 935 | 1 | 50 | 935 | 935 | 1 | 0.14894154 | 0 |
| ATTGG | | | | | | | | | |
| AGGCC | | | | | | | | | |
| TTCTT | | | | | | | | | |
| (SEQ | | | | | | | | | |
| ID | | | | | | | | | |
| NO: | | | | | | | | | |
| 182) | | | | | | | | | |
| TGGAT | 935 | 935 | 1 | 40 | 935 | 935 | 1 | 0.23089 | 0 |
| ATTTG | | | | | | | | | |
| GACCT | | | | | | | | | |
| ATTGG | | | | | | | | | |
| (SEQ | | | | | | | | | |
| ID | | | | | | | | | |
| NO: | | | | | | | | | |
| 183) | | | | | | | | | |
| TGAAA | 934 | 934 | 1 | 45 | 934 | 935 | 0.99893048 | 0.47041184 | 0 |
| ACCCG | | | | | | | | | |
| TTTCC | | | | | | | | | |
| AACGA | | | | | | | | | |
| (SEQ | | | | | | | | | |
| ID | | | | | | | | | |
| NO: | | | | | | | | | |
| 184) | | | | | | | | | |
| CACTT | 933 | 933 | 1 | 45 | 933 | 933 | 1 | 0.38227022 | 0 |
| GCAGC | | | | | | | | | |
| TACTA | | | | | | | | | |
| CAAGA | | | | | | | | | |
| (SEQ | | | | | | | | | |
| ID | | | | | | | | | |
| NO: | | | | | | | | | |
| 185) | | | | | | | | | |
| TCTGC | 932 | 932 | 1 | 50 | 932 | 932 | 1 | 0.91637015 | 0 |
| ATTCA | | | | | | | | | |
| ACTCA | | | | | | | | | |
| CCGAG | | | | | | | | | |
| (SEQ | | | | | | | | | |
| ID | | | | | | | | | |
| NO: | | | | | | | | | |
| 186) | | | | | | | | | |
| TTTGT | 932 | 932 | 1 | 40 | 932 | 932 | 1 | 0.37870049 | 0 |
| ATTTG | | | | | | | | | |
| GACCT | | | | | | | | | |
| CCTTG | | | | | | | | | |
| (SEQ | | | | | | | | | |
| ID | | | | | | | | | |
| NO: | | | | | | | | | |
| 187) | | | | | | | | | |
| GAAAT | 931 | 931 | 1 | 55 | 931 | 931 | 1 | 0.19107936 | 0 |
| GGTCC | | | | | | | | | |
| ACCGT | | | | | | | | | |
| GTGTG | | | | | | | | | |
| (SEQ | | | | | | | | | |
| ID | | | | | | | | | |
| NO: | | | | | | | | | |
| 188) | | | | | | | | | |
| TTGCA | 931 | 931 | 1 | 40 | 931 | 931 | 1 | 0.50236857 | 0 |
| GATCC | | | | | | | | | |
| TTCAG | | | | | | | | | |
| AAAGA | | | | | | | | | |
| (SEQ | | | | | | | | | |
| ID | | | | | | | | | |
| NO: | | | | | | | | | |
| 189) | | | | | | | | | |
| ACTCT | 930 | 930 | 1 | 40 | 930 | 930 | 1 | 0.18452955 | 0 |
| GAGAA | | | | | | | | | |
| TTCTT | | | | | | | | | |
| CTGTC | | | | | | | | | |
| (SEQ | | | | | | | | | |
| ID | | | | | | | | | |
| NO: | | | | | | | | | |
| 190) | | | | | | | | | |
| CTTGA | 930 | 930 | 1 | 40 | 930 | 930 | 1 | 0.17943416 | 0 |
| AGCGT | | | | | | | | | |
| ATGGT | | | | | | | | | |
| AGAAA | | | | | | | | | |
| (SEQ | | | | | | | | | |
| ID | | | | | | | | | |
| NO: | | | | | | | | | |
| 191) | | | | | | | | | |
| CTTGC | 930 | 930 | 1 | 45 | 930 | 930 | 1 | 0.30326172 | 0 |
| AGATC | | | | | | | | | |
| CTTCA | | | | | | | | | |
| GAAAG | | | | | | | | | |
| (SEQ | | | | | | | | | |
| ID | | | | | | | | | |
| NO: | | | | | | | | | |
| 192) | | | | | | | | | |
| CTTTC | 930 | 930 | 1 | 45 | 930 | 936 | 0.99358974 | 0.38118442 | 0 |
| TGAAG | | | | | | | | | |
| GATCT | | | | | | | | | |
| GCAAG | | | | | | | | | |
| (SEQ | | | | | | | | | |
| ID | | | | | | | | | |
| NO: | | | | | | | | | |
| 193) | | | | | | | | | |
| TTTCA | 929 | 929 | 1 | 40 | 930 | 938 | 0.99040512 | 0.21108746 | 0 |
| GGCCT | | | | | | | | | |
| ATGGT | | | | | | | | | |
| AGAAA | | | | | | | | | |
| (SEQ | | | | | | | | | |
| ID | | | | | | | | | |
| NO: | | | | | | | | | |
| 194) | | | | | | | | | |
| AAACT | 927 | 927 | 1 | 40 | 927 | 927 | 1 | 0.28347575 | 0 |
| GCTGT | | | | | | | | | |
| ATCCA | | | | | | | | | |
| AAGGA | | | | | | | | | |
| (SEQ | | | | | | | | | |
| ID | | | | | | | | | |
| NO: | | | | | | | | | |
| 195) | | | | | | | | | |
| GAACT | 924 | 924 | 1 | 50 | 924 | 924 | 1 | 0.15247547 | 0 |
| CCTTT | | | | | | | | | |
| GGGTC | | | | | | | | | |
| TTCGT | | | | | | | | | |
| (SEQ | | | | | | | | | |
| ID | | | | | | | | | |
| NO: | | | | | | | | | |
| 196) | | | | | | | | | |
| AGGCG | 921 | 921 | 1 | 50 | 921 | 921 | 1 | 0.75517146 | 0 |
| CTCTA | | | | | | | | | |
| AATAT | | | | | | | | | |
| CCGCT | | | | | | | | | |
| (SEQ | | | | | | | | | |
| ID | | | | | | | | | |
| NO: | | | | | | | | | |
| 197) | | | | | | | | | |
| ATTGA | 920 | 920 | 1 | 40 | 920 | 920 | 1 | 0.29349689 | 0 |
| AGCCC | | | | | | | | | |
| ACAGT | | | | | | | | | |
| AGAAA | | | | | | | | | |
| (SEQ | | | | | | | | | |
| ID | | | | | | | | | |
| NO: | | | | | | | | | |
| 198) | | | | | | | | | |
| TGTGC | 918 | 918 | 1 | 50 | 918 | 918 | 1 | 0.75587532 | 0 |
| ATTCA | | | | | | | | | |
| ACTCA | | | | | | | | | |
| GCGAG | | | | | | | | | |
| (SEQ | | | | | | | | | |
| ID | | | | | | | | | |
| NO: | | | | | | | | | |
| 199) | | | | | | | | | |
| TTCAG | 915 | 915 | 1 | 40 | 915 | 915 | 1 | 0.28564552 | 0 |
| GCCTA | | | | | | | | | |
| TGGTA | | | | | | | | | |
| GAAAA | | | | | | | | | |
| (SEQ | | | | | | | | | |
| ID | | | | | | | | | |
| NO: | | | | | | | | | |
| 200) | | | | | | | | | |
| TGTAT | 914 | 914 | 1 | 40 | 914 | 914 | 1 | 0.36205714 | 0 |
| ACTAA | | | | | | | | | |
| GAGCG | | | | | | | | | |
| CTTTG | | | | | | | | | |
| (SEQ | | | | | | | | | |
| ID | | | | | | | | | |
| NO: | | | | | | | | | |
| 201) | | | | | | | | | |
| CTTTG | 911 | 911 | 1 | 45 | 911 | 911 | 1 | 0.38248974 | 0 |
| GGTCT | | | | | | | | | |
| TCGTT | | | | | | | | | |
| GGAAA | | | | | | | | | |
| (SEQ | | | | | | | | | |
| ID | | | | | | | | | |
| NO: | | | | | | | | | |
| 202) | | | | | | | | | |
| CGTTT | 909 | 909 | 1 | 50 | 909 | 909 | 1 | 0.65761806 | 0 |
| CCAAC | | | | | | | | | |
| GAAGA | | | | | | | | | |
| CCCAA | | | | | | | | | |
| (SEQ | | | | | | | | | |
| ID | | | | | | | | | |
| NO: | | | | | | | | | |
| 203) | | | | | | | | | |
| CTTGA | 909 | 909 | 1 | 40 | 909 | 909 | 1 | 0.23363743 | 0 |
| AGCCT | | | | | | | | | |
| ATGCT | | | | | | | | | |
| AGAAA | | | | | | | | | |
| (SEQ | | | | | | | | | |
| ID | | | | | | | | | |
| NO: | | | | | | | | | |
| 204) | | | | | | | | | |
| TTTGG | 909 | 909 | 1 | 45 | 909 | 909 | 1 | 0.17090572 | 0 |
| GTCTT | | | | | | | | | |
| CGTTG | | | | | | | | | |
| GAAAC | | | | | | | | | |
| (SEQ | | | | | | | | | |
| ID | | | | | | | | | |
| NO: | | | | | | | | | |
| 205) | | | | | | | | | |
| TGAGA | 903 | 903 | 1 | 55 | 903 | 903 | 1 | 0.14700812 | 0 |
| GCGCT | | | | | | | | | |
| TTCAG | | | | | | | | | |
| GCCTA | | | | | | | | | |
| (SEQ | | | | | | | | | |
| ID | | | | | | | | | |
| NO: | | | | | | | | | |
| 206) | | | | | | | | | |
| GGTAC | 898 | 898 | 1 | 50 | 898 | 898 | 1 | 0.14434617 | 0 |
| ATTGA | | | | | | | | | |
| GAGCG | | | | | | | | | |
| CTTTC | | | | | | | | | |
| (SEQ | | | | | | | | | |
| ID | | | | | | | | | |
| NO: | | | | | | | | | |
| 207) | | | | | | | | | |
| TTCCA | 898 | 898 | 1 | 50 | 898 | 898 | 1 | 0.19250485 | 0 |
| AACTG | | | | | | | | | |
| CTCGG | | | | | | | | | |
| TCAAG | | | | | | | | | |
| (SEQ | | | | | | | | | |
| ID | | | | | | | | | |
| NO: | | | | | | | | | |
| 208) | | | | | | | | | |
| TTCCT | 897 | 897 | 1 | 50 | 897 | 897 | 1 | 0.15032714 | 0 |
| CTTGA | | | | | | | | | |
| CCGAG | | | | | | | | | |
| CAGTT | | | | | | | | | |
| (SEQ | | | | | | | | | |
| ID | | | | | | | | | |
| NO: | | | | | | | | | |
| 209) | | | | | | | | | |
| ATAGC | 896 | 896 | 1 | 55 | 896 | 896 | 1 | 0.69417341 | 0 |
| GCATT | | | | | | | | | |
| GAGCC | | | | | | | | | |
| TACGG | | | | | | | | | |
| (SEQ | | | | | | | | | |
| ID | | | | | | | | | |
| NO: | | | | | | | | | |
| 210) | | | | | | | | | |
| GATGT | 881 | 881 | 1 | 45 | 881 | 881 | 1 | 0.22709647 | 0 |
| TTCTT | | | | | | | | | |
| TTTCC | | | | | | | | | |
| GCCGT | | | | | | | | | |
| (SEQ | | | | | | | | | |
| ID | | | | | | | | | |
| NO: | | | | | | | | | |
| 211) | | | | | | | | | |
| GTCTT | 814 | 814 | 1 | 40 | 814 | 814 | 1 | 0.28260619 | 0 |
| CACAT | | | | | | | | | |
| AAAAG | | | | | | | | | |
| GCAGA | | | | | | | | | |
| (SEQ | | | | | | | | | |
| ID | | | | | | | | | |
| NO: | | | | | | | | | |
| 212) | | | | | | | | | |
| TGGAT | 804 | 804 | 1 | 45 | 804 | 804 | 1 | 0.54767999 | 0 |
| AATTG | | | | | | | | | |
| GACCT | | | | | | | | | |
| CCTAG | | | | | | | | | |
| (SEQ | | | | | | | | | |
| ID | | | | | | | | | |
| NO: | | | | | | | | | |
| 213) | | | | | | | | | |
| TGTGC | 730 | 730 | 1 | 50 | 730 | 730 | 1 | 0.82169685 | 0 |
| ATTCG | | | | | | | | | |
| ACGCA | | | | | | | | | |
| CAGAA | | | | | | | | | |
| (SEQ | | | | | | | | | |
| ID | | | | | | | | | |
| NO: | | | | | | | | | |
| 214) | | | | | | | | | |
| TTGGA | 729 | 729 | 1 | 50 | 729 | 729 | 1 | 0.18419878 | 0 |
| ACGCC | | | | | | | | | |
| TTGAA | | | | | | | | | |
| GCGTA | | | | | | | | | |
| (SEQ | | | | | | | | | |
| ID | | | | | | | | | |
| NO: | | | | | | | | | |
| 215) | | | | | | | | | |
| AGGCG | 722 | 722 | 1 | 50 | 722 | 722 | 1 | 0.75517146 | 0 |
| TTCCA | | | | | | | | | |
| AATAT | | | | | | | | | |
| CCGCT | | | | | | | | | |
| (SEQ | | | | | | | | | |
| ID | | | | | | | | | |
| NO: | | | | | | | | | |
| 216) | | | | | | | | | |
| AAACT | 718 | 718 | 1 | 45 | 718 | 718 | 1 | 0.53380525 | |
| GCTCT | | | | | | | | | |
| GTGAA | | | | | | | | | |
| AAGGG | | | | | | | | | |
| (SEQ | | | | | | | | | |
| ID | | | | | | | | | |
| NO: | | | | | | | | | |
| 217) | | | | | | | | | |
| TTCCA | 712 | 712 | 1 | 45 | 712 | 712 | 1 | 0.18753013 | 0 |
| AACTG | | | | | | | | | |
| CTCTC | | | | | | | | | |
| TCAAG | | | | | | | | | |
| (SEQ | | | | | | | | | |
| ID | | | | | | | | | |
| NO: | | | | | | | | | |
| 218) | | | | | | | | | |
| TTCCT | 712 | 712 | 1 | 45 | 712 | 712 | 1 | 0.17810984 | 0 |
| CTTGA | | | | | | | | | |
| GAGAG | | | | | | | | | |
| CAGTT | | | | | | | | | |
| (SEQ | | | | | | | | | |
| ID | | | | | | | | | |
| NO: | | | | | | | | | |
| 219) | | | | | | | | | |
| TGGAG | 702 | 702 | 1 | 45 | 702 | 704 | 0.99715909 | 0.14365031 | 0 |
| GCCTT | | | | | | | | | |
| CTTTG | | | | | | | | | |
| GAAAT | | | | | | | | | |
| (SEQ | | | | | | | | | |
| ID | | | | | | | | | |
| NO: | | | | | | | | | |
| 220) | | | | | | | | | |
| GGGAT | 667 | 667 | 1 | 40 | 668 | 668 | 0.99850299 | 0.24317244 | 0 |
| ATTTG | | | | | | | | | |
| GACTT | | | | | | | | | |
| CTTTG | | | | | | | | | |
| (SEQ | | | | | | | | | |
| ID | | | | | | | | | |
| NO: | | | | | | | | | |
| 221) | | | | | | | | | |
| TCTCA | 666 | 666 | 1 | 40 | 666 | 666 | 1 | 0.38344318 | 0 |
| GAAAC | | | | | | | | | |
| TACTG | | | | | | | | | |
| TGTGA | | | | | | | | | |
| (SEQ | | | | | | | | | |
| ID | | | | | | | | | |
| NO: | | | | | | | | | |
| 222) | | | | | | | | | |
| GAAAT | 658 | 658 | 1 | 50 | 658 | 658 | 1 | 0.21815953 | 0 |
| GTTCC | | | | | | | | | |
| ACCGT | | | | | | | | | |
| GTGTG | | | | | | | | | |
| (SEQ | | | | | | | | | |
| ID | | | | | | | | | |
| NO: | | | | | | | | | |
| 223) | | | | | | | | | |
| GTGGT | 652 | 652 | 1 | 40 | 652 | 652 | 1 | 0.87172674 | 0 |
| TGTAG | | | | | | | | | |
| TATTT | | | | | | | | | |
| CCAAG | | | | | | | | | |
| (SEQ | | | | | | | | | |
| ID | | | | | | | | | |
| NO: | | | | | | | | | |
| 224) | | | | | | | | | |
| TGGAT | 652 | 652 | 1 | 50 | 652 | 652 | 1 | 0.30232232 | 0 |
| AATTG | | | | | | | | | |
| GACCG | | | | | | | | | |
| CCTTG | | | | | | | | | |
| (SEQ | | | | | | | | | |
| ID | | | | | | | | | |
| NO: | | | | | | | | | |
| 225) | | | | | | | | | |
| GCACA | 649 | 649 | 1 | 40 | 649 | 649 | 1 | 0.25775484 | 0 |
| CAACC | | | | | | | | | |
| AAAGA | | | | | | | | | |
| AGTTT | | | | | | | | | |
| (SEQ | | | | | | | | | |
| ID | | | | | | | | | |
| NO: | | | | | | | | | |
| 226) | | | | | | | | | |
| CACAG | 640 | 640 | 1 | 50 | 640 | 640 | 1 | 0.24158431 | 0 |
| AGTGG | | | | | | | | | |
| AACCT | | | | | | | | | |
| TCCTT | | | | | | | | | |
| (SEQ | | | | | | | | | |
| ID | | | | | | | | | |
| NO: | | | | | | | | | |
| 227) | | | | | | | | | |
| TTCCT | 638 | 638 | 1 | 45 | 638 | 638 | 1 | 0.13426423 | 0 |
| CTTGG | | | | | | | | | |
| TAGAG | | | | | | | | | |
| CAGTT | | | | | | | | | |
| (SEQ | | | | | | | | | |
| ID | | | | | | | | | |
| NO: | | | | | | | | | |
| 228) | | | | | | | | | |
| ACAGA | 636 | 636 | 1 | 45 | 636 | 636 | 1 | 0.31688618 | 0 |
| GTGCA | | | | | | | | | |
| ACATT | | | | | | | | | |
| CCTCT | | | | | | | | | |
| (SEQ | | | | | | | | | |
| ID | | | | | | | | | |
| NO: | | | | | | | | | |
| 229) | | | | | | | | | |
| CCCAT | 635 | 635 | 1 | 40 | 635 | 639 | 0.99374022 | 0.39299603 | 0 |
| TGCAG | | | | | | | | | |
| ATTCT | | | | | | | | | |
| ACAAA | | | | | | | | | |
| (SEQ | | | | | | | | | |
| ID | | | | | | | | | |
| NO: | | | | | | | | | |
| 230) | | | | | | | | | |
| TTCCC | 635 | 635 | 1 | 50 | 635 | 635 | 1 | 0.56752219 | 0 |
| AACTG | | | | | | | | | |
| CTCTA | | | | | | | | | |
| CCAAG | | | | | | | | | |
| (SEQ | | | | | | | | | |
| ID | | | | | | | | | |
| NO: | | | | | | | | | |
| 231) | | | | | | | | | |
| ATTCC | 633 | 633 | 1 | 45 | 633 | 633 | 1 | 0.16059112 | 0 |
| TCTTG | | | | | | | | | |
| GTAGA | | | | | | | | | |
| GCAGT | | | | | | | | | |
| (SEQ | | | | | | | | | |
| ID | | | | | | | | | |
| NO: | | | | | | | | | |
| 232) | | | | | | | | | |
| AAGGC | 631 | 631 | 1 | 50 | 631 | 631 | 1 | 0.76283916 | 0 |
| GCTCT | | | | | | | | | |
| AATAT | | | | | | | | | |
| CCGCT | | | | | | | | | |
| (SEQ | | | | | | | | | |
| ID | | | | | | | | | |
| NO: | | | | | | | | | |
| 233) | | | | | | | | | |
| GCTTC | 627 | 627 | 1 | 40 | 627 | 627 | 1 | 0.60214312 | 0 |
| TGTCT | | | | | | | | | |
| TGGTT | | | | | | | | | |
| TTATG | | | | | | | | | |
| (SEQ | | | | | | | | | |
| ID | | | | | | | | | |
| NO: | | | | | | | | | |
| 234) | | | | | | | | | |
| GCGGA | 620 | 620 | 1 | 55 | 620 | 620 | 1 | 0.26408621 | 0 |
| TATTA | | | | | | | | | |
| GAGCG | | | | | | | | | |
| CCTTG | | | | | | | | | |
| (SEQ | | | | | | | | | |
| ID | | | | | | | | | |
| NO: | | | | | | | | | |
| 235) | | | | | | | | | |
| GCTTG | 563 | 563 | 1 | 40 | 563 | 563 | 1 | 0.61188789 | 0 |
| GAAAT | | | | | | | | | |
| ACTAC | | | | | | | | | |
| AACCA | | | | | | | | | |
| (SEQ | | | | | | | | | |
| ID | | | | | | | | | |
| NO: | | | | | | | | | |
| 236) | | | | | | | | | |
| TAGAG | 556 | 556 | 1 | 50 | 55€ | 556 | 1 | 0.89437142 | 0 |
| CAGTT | | | | | | | | | |
| TGAAA | | | | | | | | | |
| CGCCG | | | | | | | | | |
| (SEQ | | | | | | | | | |
| ID | | | | | | | | | |
| NO: | | | | | | | | | |
| 237) | | | | | | | | | |
| GTTGA | 405 | 405 | 1 | 45 | 405 | 405 | 1 | 0.897681 | 0 |
| ATGCA | | | | | | | | | |
| GACAT | | | | | | | | | |
| CACAG | | | | | | | | | |
| (SEQ | | | | | | | | | |
| ID | | | | | | | | | |
| NO: | | | | | | | | | |
| 238) | | | | | | | | | |

CHR7

| | bind- | | | | | | | | bind- | | | |
|---|---|---|---|---|---|---|---|---|---|---|---|---|
| | ing | | | | | | | | ing | | | |
| | sites | | | | | | | | sites | | | |
| | (CHM | | | | | | | | (CHM13- | | | |
| | 13- | bind- | | | | | | | spe- | | | |
| | spe- | ing | | | | | | | cif- | | | |
| | cif- | sites | | | | | | | ic | | | |
| | ic | (CHM | | | bind- | | | | chro- | | | |
| | chro- | 13- | chro- | | ing | bind- | | | mo- | | HOR_ | |
| | mo- | all | mo- | | sites | ing | centro- | | some | | L | HOR |
| | some | cen- | some | | (CHM- | sites | mere | | centro- | | in | in |
| sg | centro- | tro- | spe- | | 13 | (hg38 | spe- | Ac- | mere | bind- | this | this |
| RNA | mere | mere | cif- | GC_ | whole | whole | cif- | tiv- | Not | ing | chro- | chro- |
| _ | HOR_ | HOR_ | ic- | cont- | ge- | ge- | ic- | ity | HOR_ | HOR_ | mo- | mo- |
| seq | L) | L) | ity | ent | nome) | nome) | ity | score | L) | L | some | some |
| AATCT | 3042 | 3048 | 0.998 | 40 | 3042 | 3048 | 0.9980315 | 0.65711272 | 0 | hor_ | hor_ | hor_ |
| GCTCT | | | | | | | | | | 72 | 7_ | 7_ |
| CTCTA | | | | | | | | | | (S1C7 | 2 | 1 |
| AAGCA | | | | | | | | | | H1L) | (S1C7 | (S5C7 |
| (SEQ | | | | | | | | | | | H1L) | H2); |
| ID | | | | | | | | | | | | hor_ |
| NO: | | | | | | | | | | | | 7_ |
| 239) | | | | | | | | | | | | 2 |
| | | | | | | | | | | | | (S1C7 |
| | | | | | | | | | | | | H1L) |
| TTGCA | 3041 | 3064 | 0.992 | 50 | 3041 | 3065 | 0.99216966 | 0.62026561 | 0 | hor_ | hor_ | hor_ |
| ACGAA | | | | | | | | | | 7_ | 7_ | 7_ |
| GGCCT | | | | | | | | | | 2 | 2 | 1 |
| CAAAG | | | | | | | | | | (S1C7 | (S1C7 | (S5C7 |
| (SEQ | | | | | | | | | | H1L) | H1L) | H2); |
| ID | | | | | | | | | | | | hor_ |
| NO: | | | | | | | | | | | | 7_ |
| 240) | | | | | | | | | | | | 2 |
| | | | | | | | | | | | | (S1C7 |
| | | | | | | | | | | | | H1L) |
| TTTGA | 2981 | 3004 | 0.992 | 45 | 2981 | 3007 | 0.99135351 | 0.42142359 | 0 | hor_ | hor_ | hor_ |
| GGCCT | | | | | | | | | | 7_ | 7_ | 7_ |
| TCGTT | | | | | | | | | | 2 | 2 | 1 |
| GCAAA | | | | | | | | | | (S1C7 | (S1C7 | (S5C7 |
| (SEQ | | | | | | | | | | H1L) | H1L) | H2); |
| ID | | | | | | | | | | | | hor_ |
| NO: | | | | | | | | | | | | 7_ |
| 241) | | | | | | | | | | | | 2 |
| | | | | | | | | | | | | (S1C7 |
| | | | | | | | | | | | | H1L) |
| GACCG | 2914 | 2914 | 1 | 60 | 2914 | 2914 | 1 | 0.18978537 | 0 | hor_ | hor_ | hor_ |
| CATTG | | | | | | | | | | 7_ | 7_ | 7_ |
| AGGCC | | | | | | | | | | 2 | 2 | 1 |
| TTCGT | | | | | | | | | | (S1C7 | (S1C7 | (S5C7 |
| (SEQ | | | | | | | | | | H1L) | H1L) | H2); |
| ID | | | | | | | | | | | | hor_ |
| NO: | | | | | | | | | | | | 7_ |
| 242) | | | | | | | | | | | | 2 |
| | | | | | | | | | | | | (S1C7 |
| | | | | | | | | | | | | H1L) |
| TGGAT | 2904 | 2905 | 1 | 45 | 2904 | 2905 | 0.99965577 | 0.15893327 | 0 | hor_ | hor_ | hor_ |
| ATATG | | | | | | | | | | 7_ | 7_ | 7_ |
| GACCG | | | | | | | | | | 2 | 2 | 1 |
| CATTG | | | | | | | | | | (S1C7 | (S1C7 | (S5C7 |
| (SEQ | | | | | | | | | | H1L) | H1L) | H2); |
| ID | | | | | | | | | | | | hor_ |
| NO: | | | | | | | | | | | | 7_ |
| 25) | | | | | | | | | | | | 2 |
| | | | | | | | | | | | | (S1C7 |
| | | | | | | | | | | | | H1L) |
| TGAGG | 2883 | 2883 | 1 | 55 | 2883 | 2884 | 0.99965326 | 0.21573566 | 0 | hor_ | hor_ | hor_ |
| CCTTC | | | | | | | | | | 7_ | 7_ | 7_ |
| GTTGC | | | | | | | | | | 2 | 2 | 1 |
| AAACG | | | | | | | | | | (S1C7 | (S1C7 | (S5C7 |
| (SEQ | | | | | | | | | | H1L) | H1L) | H2); |
| ID | | | | | | | | | | | | hor_ |
| NO: | | | | | | | | | | | | 7_ |
| 243) | | | | | | | | | | | | 2 |
| | | | | | | | | | | | | (S1C7 |
| | | | | | | | | | | | | H1L) |
| AGAAA | 2844 | 2844 | 1 | 50 | 2844 | 2845 | 0.99964851 | 0.25993389 | 0 | hor_ | hor_ | hor_ |
| CCCCG | | | | | | | | | | 72 | 7_ | 7_ |
| TTTGC | | | | | | | | | | (S1C7 | 2 | 1 |
| AACGA | | | | | | | | | | H1L) | (S1C7 | (S5C7 |
| (SEQ | | | | | | | | | | | H1L) | H2); |
| ID | | | | | | | | | | | | hor_ |
| NO: | | | | | | | | | | | | 7_ |
| 244) | | | | | | | | | | | | 2 |
| | | | | | | | | | | | | (S1C7 |
| | | | | | | | | | | | | H1L) |
| CTTTT | 2738 | 2756 | 0.993 | 40 | 2738 | 2756 | 0.9934688 | 0.59068245 | 0 | hor_ | hor_ | hor_ |
| TGTGG | | | | | | | | | | 7_ | 7_ | 7_ |
| AGTTT | | | | | | | | | | 2 | 2 | 1 |
| GCAAG | | | | | | | | | | (S1C7 | (S1C7 | (S5C7 |
| (SEQ | | | | | | | | | | H1L) | H1L) | H2); |
| ID | | | | | | | | | | | | hor_ |
| NO: | | | | | | | | | | | | 7_ |
| 245) | | | | | | | | | | | | 2 |
| | | | | | | | | | | | | (S1C7 |
| | | | | | | | | | | | | H1L) |
| GATTT | 2728 | 2728 | 1 | 40 | 2728 | 2728 | 1 | 0.27834188 | 0 | hor_ | hor_ | hor_ |
| GAAAC | | | | | | | | | | 72 | 7_ | 7_ |
| ACTCT | | | | | | | | | | (S1C7 | 2 | 1 |
| TGCTG | | | | | | | | | | H1L) | (S1C7 | (S5C7 |
| (SEQ | | | | | | | | | | | H1L) | H2); |
| ID | | | | | | | | | | | | hor_ |
| NO: | | | | | | | | | | | | 7_ |
| 246) | | | | | | | | | | | | 2 |
| | | | | | | | | | | | | (S1C7 |
| | | | | | | | | | | | | H1L) |
| CTTGC | 2626 | 2626 | 1 | 50 | 2626 | 2626 | 1 | 0.50191502 | 0 | hor_ | hor_ | hor_ |
| TGTGG | | | | | | | | | | 72 | 7_ | 7_ |
| CATTT | | | | | | | | | | (S1C7 | 2 | 1 |
| TCAGG | | | | | | | | | | H1L) | (S1C7 | (S5C7 |
| (SEQ | | | | | | | | | | | H1L) | H2); |
| ID | | | | | | | | | | | | hor_ |
| NO: | | | | | | | | | | | | 7_ |
| 247) | | | | | | | | | | | | 2 |
| | | | | | | | | | | | | (S1C7 |
| | | | | | | | | | | | | H1L) |
| CTGCT | 2556 | 2556 | 1 | 45 | 2556 | 2556 | 1 | 0.21905467 | 0 | hor_ | hor_ | hor_ |
| TGTTA | | | | | | | | | | 72 | 7_ | 7_ |
| TGTCT | | | | | | | | | | (S1C7 | 2 | 1 |
| GCAAG | | | | | | | | | | H1L) | (S1C7 | (S5C7 |
| (SEQ | | | | | | | | | | | H1L) | H2); |
| ID | | | | | | | | | | | | hor_ |
| NO: | | | | | | | | | | | | 7_ |
| 26) | | | | | | | | | | | | 2 |
| | | | | | | | | | | | | (S1C7 |
| | | | | | | | | | | | | H1L) |
| TTCAA | 2341 | 2341 | 1 | 40 | 2341 | 2341 | 1 | 0.20271172 | 0 | hor_ | hor_ | hor_ |
| ATCTG | | | | | | | | | | 7_ | 7_ | 7_ |
| CTCTG | | | | | | | | | | 2 | 2 | 1 |
| TGCAA | | | | | | | | | | (S1C7 | (S1C7 | (S5C7 |
| (SEQ | | | | | | | | | | H1L) | H1L) | H2); |
| ID | | | | | | | | | | | | hor_ |
| NO: | | | | | | | | | | | | 7_ |
| 248) | | | | | | | | | | | | 2 |
| | | | | | | | | | | | | (S1C7 |
| | | | | | | | | | | | | H1L) |
| TCAAA | 2338 | 2338 | 1 | 40 | 2338 | 2338 | 1 | 0.15162949 | 0 | hor_ | hor_ | hor_ |
| TCTGC | | | | | | | | | | 7_ | 7_ | 7_ |
| TCTGT | | | | | | | | | | 2 | 2 | 1 |
| GCAAA | | | | | | | | | | (S1C7 | (S1C7 | (S5C7 |
| (SEQ | | | | | | | | | | H1L) | H1L) | H2); |
| ID | | | | | | | | | | | | hor_ |
| NO: | | | | | | | | | | | | 7_ |
| 249) | | | | | | | | | | | | 2 |
| | | | | | | | | | | | | (S1C7 |
| | | | | | | | | | | | | H1L) |
| TGGAT | 924 | 924 | 1 | 45 | 924 | 924 | 1 | 0.10208471 | 0 | hor_ | hor_ | hor_ |
| ACATG | | | | | | | | | | 7_ | 7_ | 7_ |
| GACCT | | | | | | | | | | 2 | 2 | 1 |
| GTTTG | | | | | | | | | | (S1C7 | (S1C7 | (S5C7 |
| (SEQ | | | | | | | | | | H1L) | H1L) | H2); |
| ID | | | | | | | | | | | | hor_ |
| NO: | | | | | | | | | | | | 7_ |
| 250) | | | | | | | | | | | | 2 |
| | | | | | | | | | | | | (S1C7 |
| | | | | | | | | | | | | H1L) |
| AAAGT | 893 | 893 | 1 | 40 | 893 | 893 | 1 | 0.11030888 | 0 | hor_ | hor_ | hor_ |
| CTGCA | | | | | | | | | | 7_ | 72 | 7_ |
| AGTGG | | | | | | | | | | 2 | (S1C7 | 1 |
| ATACA | | | | | | | | | | (S1C7 | H1L) | (S5C7 |
| (SEQ | | | | | | | | | | H1L) | | H2); |
| ID | | | | | | | | | | | | hor_ |
| NO: | | | | | | | | | | | | 7_ |
| 251) | | | | | | | | | | | | 2 |
| | | | | | | | | | | | | (S1C7 |
| | | | | | | | | | | | | H1L) |

CHR8

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| CTTCT | 1466 | 1466 | 1 | 40 | 1466 | 1466 | 1 | 0.57288025 | 0 |
| TTTGG | | | | | | | | | |
| AATCT | | | | | | | | | |
| GCAAG | | | | | | | | | |
| (SEQ | | | | | | | | | |
| ID | | | | | | | | | |
| NO: | | | | | | | | | |
| 252) | | | | | | | | | |
| AACCT | 1457 | 1457 | 1 | 45 | 1457 | 1457 | 1 | 0.1269202 | 0 |
| TCCGT | | | | | | | | | |
| TTCAT | | | | | | | | | |
| AGAGC | | | | | | | | | |
| (SEQ | | | | | | | | | |
| ID | | | | | | | | | |
| NO: | | | | | | | | | |
| 253) | | | | | | | | | |
| TTCCG | 1457 | 1458 | 0.999 | 45 | 1457 | 1458 | 0.99931413 | 0.46110367 | 0 |
| TTTCA | | | | | | | | | |
| TAGAG | | | | | | | | | |
| CAGGT | | | | | | | | | |
| (SEQ | | | | | | | | | |
| ID | | | | | | | | | |
| NO: | | | | | | | | | |
| 254) | | | | | | | | | |
| AACCT | 1454 | 1454 | 1 | 45 | 1454 | 1454 | 1 | 0.5901572 | 0 |
| GCTCT | | | | | | | | | |
| ATGAA | | | | | | | | | |
| ACGGA | | | | | | | | | |
| (SEQ | | | | | | | | | |
| ID | | | | | | | | | |
| NO: | | | | | | | | | |
| 28) | | | | | | | | | |
| CTTGA | 1417 | 1417 | 1 | 40 | 1417 | 1417 | 1 | 0.7904756 | 0 |
| TTGCA | | | | | | | | | |
| AACAT | | | | | | | | | |
| CACGA | | | | | | | | | |
| (SEQ | | | | | | | | | |
| ID | | | | | | | | | |
| NO: | | | | | | | | | |
| 255) | | | | | | | | | |
| GATAG | 1386 | 1395 | 0.994 | 45 | 1386 | 1398 | 0.99141631 | 0.7028255 | 0 |
| CTGTG | | | | | | | | | |
| AGGAT | | | | | | | | | |
| TTCGT | | | | | | | | | |
| (SEQ | | | | | | | | | |
| ID | | | | | | | | | |
| NO: | | | | | | | | | |
| 256) | | | | | | | | | |
| GAATG | 1380 | 1380 | 1 | 45 | 1380 | 1380 | 1 | 0.14307509 | 0 |
| TTCAA | | | | | | | | | |
| CTCTG | | | | | | | | | |
| AGAGC | | | | | | | | | |
| (SEQ | | | | | | | | | |
| ID | | | | | | | | | |
| NO: | | | | | | | | | |
| 29) | | | | | | | | | |
| TGAGA | 1343 | 1343 | 1 | 40 | 1343 | 1343 | 1 | 0.17224744 | 0 |
| ATCAC | | | | | | | | | |
| GTTTG | | | | | | | | | |
| TGATG | | | | | | | | | |
| (SEQ | | | | | | | | | |
| ID | | | | | | | | | |
| NO: | | | | | | | | | |
| 257) | | | | | | | | | |
| GAGAA | 833 | 833 | 1 | 40 | 833 | 833 | 1 | 0.10091445 | 0 |
| CACAC | | | | | | | | | |
| ATCAC | | | | | | | | | |
| AATCA | | | | | | | | | |
| (SEQ | | | | | | | | | |
| ID | | | | | | | | | |
| NO: | | | | | | | | | |
| 258) | | | | | | | | | |
| GAAAG | 826 | 827 | 0.999 | 40 | 827 | 831 | 0.99398315 | 0.26836476 | 0 |
| GTTCA | | | | | | | | | |
| ACTCT | | | | | | | | | |
| GTTAG | | | | | | | | | |
| (SEQ | | | | | | | | | |
| ID | | | | | | | | | |
| NO: | | | | | | | | | |
| 259) | | | | | | | | | |
| CAACT | 822 | 822 | 1 | 40 | 822 | 822 | 1 | 0.51843045 | 0 |
| GTCAG | | | | | | | | | |
| AATTG | | | | | | | | | |
| AACCT | | | | | | | | | |
| (SEQ | | | | | | | | | |
| ID | | | | | | | | | |
| NO: | | | | | | | | | |
| 260) | | | | | | | | | |
| TTGGA | 812 | 812 | 1 | 45 | 812 | 812 | 1 | 0.26386226 | 0 |
| GCGCT | | | | | | | | | |
| TTCTG | | | | | | | | | |
| AACTA | | | | | | | | | |
| (SEQ | | | | | | | | | |
| ID | | | | | | | | | |
| NO: | | | | | | | | | |
| 261) | | | | | | | | | |
| CTGCA | 809 | 809 | 1 | 50 | 809 | 809 | 1 | 0.16559802 | 0 |
| CAACT | | | | | | | | | |
| GCTCT | | | | | | | | | |
| ATGTG | | | | | | | | | |
| (SEQ | | | | | | | | | |
| ID | | | | | | | | | |
| NO: | | | | | | | | | |
| 262) | | | | | | | | | |
| CTTCT | 804 | 804 | 1 | 50 | 804 | 804 | 1 | 0.79103655 | 0 |
| CGTAC | | | | | | | | | |
| TATCT | | | | | | | | | |
| GGCAG | | | | | | | | | |
| (SEQ | | | | | | | | | |
| ID | | | | | | | | | |
| NO: | | | | | | | | | |
| 263) | | | | | | | | | |
| GCACA | 804 | 804 | 1 | 50 | 804 | 804 | 1 | 0.70967794 | 0 |
| ACTGC | | | | | | | | | |
| TCTAT | | | | | | | | | |
| GTGAG | | | | | | | | | |
| (SEQ | | | | | | | | | |
| ID | | | | | | | | | |
| NO: | | | | | | | | | |
| 264) | | | | | | | | | |
| TGCAC | 802 | 802 | 1 | 45 | 802 | 802 | 1 | 0.17797801 | 0 |
| AACTG | | | | | | | | | |
| CTCTA | | | | | | | | | |
| TGTGA | | | | | | | | | |
| (SEQ | | | | | | | | | |
| ID | | | | | | | | | |
| NO: | | | | | | | | | |
| 265) | | | | | | | | | |
| TTTCC | 798 | 798 | 1 | 40 | 798 | 798 | 1 | 0.71753553 | 0 |
| ATTCA | | | | | | | | | |
| AGTCA | | | | | | | | | |
| CAGAG | | | | | | | | | |
| (SEQ | | | | | | | | | |
| ID | | | | | | | | | |
| NO: | | | | | | | | | |
| 266) | | | | | | | | | |
| AGATT | 796 | 796 | 1 | 40 | 796 | 796 | 1 | 0.13510752 | 0 |
| CTGCA | | | | | | | | | |
| TGCGG | | | | | | | | | |
| ATATT | | | | | | | | | |
| (SEQ | | | | | | | | | |
| ID | | | | | | | | | |
| NO: | | | | | | | | | |
| 267) | | | | | | | | | |
| GAAGT | 792 | 792 | 1 | 40 | 792 | 792 | 1 | 0.45462331 | 0 |
| ACTGC | | | | | | | | | |
| ATGAA | | | | | | | | | |
| ACGAA | | | | | | | | | |
| (SEQ | | | | | | | | | |
| ID | | | | | | | | | |
| NO: | | | | | | | | | |
| 268) | | | | | | | | | |
| GTTCC | 792 | 792 | 1 | 45 | 792 | 792 | 1 | 0.5057401 | 0 |
| ACTCT | | | | | | | | | |
| GTGAC | | | | | | | | | |
| TTGAA | | | | | | | | | |
| (SEQ | | | | | | | | | |
| ID | | | | | | | | | |
| NO: | | | | | | | | | |
| 269) | | | | | | | | | |
| GATCG | 778 | 778 | 1 | 45 | 778 | 778 | 1 | 0.56805383 | 0 |
| CTTTG | | | | | | | | | |
| AGGAT | | | | | | | | | |
| TTCGT | | | | | | | | | |
| (SEQ | | | | | | | | | |
| ID | | | | | | | | | |
| NO: | | | | | | | | | |
| 270) | | | | | | | | | |
| CGGAT | 772 | 775 | 0.996 | 40 | 772 | 776 | 0.99484536 | 0.12605409 | 0 |
| ATTTG | | | | | | | | | |
| GATAG | | | | | | | | | |
| CTTTG | | | | | | | | | |
| (SEQ | | | | | | | | | |
| ID | | | | | | | | | |
| NO: | | | | | | | | | |
| 271) | | | | | | | | | |
| CTTTT | 754 | 756 | 0.997 | 40 | 754 | 757 | 0.99603699 | 0.33679531 | 0 |
| TGGAG | | | | | | | | | |
| TATCT | | | | | | | | | |
| GGAAG | | | | | | | | | |
| (SEQ | | | | | | | | | |
| ID | | | | | | | | | |
| NO: | | | | | | | | | |
| 272) | | | | | | | | | |
| AGTGG | 753 | 753 | 1 | 45 | 753 | 753 | 1 | 0.19282175 | 0 |
| ACATT | | | | | | | | | |
| TTGAG | | | | | | | | | |
| CTCCT | | | | | | | | | |
| (SEQ | | | | | | | | | |
| ID | | | | | | | | | |
| NO: | | | | | | | | | |
| 273) | | | | | | | | | |
| TGGAC | 750 | 750 | 1 | 45 | 750 | 751 | 0.99866844 | 0.29707776 | 0 |
| ATTTT | | | | | | | | | |
| GAGCT | | | | | | | | | |
| CCTTG | | | | | | | | | |
| (SEQ | | | | | | | | | |
| ID | | | | | | | | | |
| NO: | | | | | | | | | |
| 274) | | | | | | | | | |
| CTTTT | 739 | 739 | 1 | 50 | 739 | 740 | 0.99864865 | 0.48860712 | 0 |
| TCAGC | | | | | | | | | |
| ATAGG | | | | | | | | | |
| CCCCA | | | | | | | | | |
| (SEQ | | | | | | | | | |
| ID | | | | | | | | | |
| NO: | | | | | | | | | |
| 275) | | | | | | | | | |
| CTTGG | 737 | 737 | 1 | 50 | 737 | 738 | 0.99864499 | 0.32225034 | 0 |
| GGCCT | | | | | | | | | |
| ATGCT | | | | | | | | | |
| GAAAA | | | | | | | | | |
| (SEQ | | | | | | | | | |
| ID | | | | | | | | | |
| NO: | | | | | | | | | |
| 276) | | | | | | | | | |
| TGATG | 707 | 708 | 0.999 | 40 | 707 | 708 | 0.99858757 | 0.48471508 | 0 |
| TGTGT | | | | | | | | | |
| CCTCA | | | | | | | | | |
| ACAAA | | | | | | | | | |
| (SEQ | | | | | | | | | |
| ID | | | | | | | | | |
| NO: | | | | | | | | | |
| 277) | | | | | | | | | |
| AACGA | 705 | 705 | 1 | 40 | 705 | 705 | 1 | 0.27116448 | 0 |
| CATAG | | | | | | | | | |
| AAGCT | | | | | | | | | |
| ATCTC | | | | | | | | | |
| (SEQ | | | | | | | | | |
| ID | | | | | | | | | |
| NO: | | | | | | | | | |
| 278) | | | | | | | | | |
| AGGTT | 695 | 695 | 1 | 45 | 695 | 695 | 1 | 0.1253111 | 0 |
| CAAGT | | | | | | | | | |
| CCGTT | | | | | | | | | |
| TGTTG | | | | | | | | | |
| (SEQ | | | | | | | | | |
| ID | | | | | | | | | |
| NO: | | | | | | | | | |
| 279) | | | | | | | | | |
| AAAGT | 671 | 671 | 1 | 45 | 671 | 671 | 1 | 0.57744522 | 0 |
| GCTCT | | | | | | | | | |
| GTCCA | | | | | | | | | |
| AACCA | | | | | | | | | |
| (SEQ | | | | | | | | | |
| ID | | | | | | | | | |
| NO: | | | | | | | | | |
| 280) | | | | | | | | | |
| TGCTT | 613 | 613 | 1 | 40 | 613 | 613 | 1 | 0.19159194 | 0 |
| CTGTC | | | | | | | | | |
| TAGTT | | | | | | | | | |
| TCTGT | | | | | | | | | |
| (SEQ | | | | | | | | | |
| ID | | | | | | | | | |
| NO: | | | | | | | | | |
| 281) | | | | | | | | | |
| AAACT | 601 | 601 | 1 | 40 | 601 | 601 | 1 | 0.54390365 | 0 |
| GCTCT | | | | | | | | | |
| GTCAG | | | | | | | | | |
| TACAA | | | | | | | | | |
| (SEQ | | | | | | | | | |
| ID | | | | | | | | | |
| NO: | | | | | | | | | |
| 282) | | | | | | | | | |
| AGGAT | 557 | 560 | 0.995 | 40 | 557 | 560 | 0.99464286 | 0.23077555 | 0 |
| ATTTG | | | | | | | | | |
| GATAG | | | | | | | | | |
| CTGTG | | | | | | | | | |
| (SEQ | | | | | | | | | |
| ID | | | | | | | | | |
| NO: | | | | | | | | | |
| 283) | | | | | | | | | |
| TTCCC | 468 | 470 | 0.996 | 45 | 468 | 470 | 0.99574468 | 0.46110367 | 0 |
| ATTCA | | | | | | | | | |
| TAGAG | | | | | | | | | |
| CAGGT | | | | | | | | | |
| (SEQ | | | | | | | | | |
| ID | | | | | | | | | |
| NO: | | | | | | | | | |
| 284) | | | | | | | | | |

CHR9

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| GATAG | 2116 | 2116 | 1 | 40 | 2116 | 2116 | 1 | 0.41722232 | 0 |
| CTTTG | | | | | | | | | |
| AAGGT | | | | | | | | | |
| TTCGT | | | | | | | | | |
| (SEQ | | | | | | | | | |
| ID | | | | | | | | | |
| NO: | | | | | | | | | |
| 32) | | | | | | | | | |
| AACAC | 1390 | 1390 | 1 | 45 | 1390 | 1390 | 1 | 0.18665232 | 0 |
| TTCCC | | | | | | | | | |
| TTCAT | | | | | | | | | |
| ACAGC | | | | | | | | | |
| (SEQ | | | | | | | | | |
| ID | | | | | | | | | |
| NO: | | | | | | | | | |
| 31) | | | | | | | | | |
| GTTTC | 1384 | 1384 | 1 | 40 | 1384 | 1384 | 1 | 0.35145896 | 0 |
| AAACC | | | | | | | | | |
| TGCTG | | | | | | | | | |
| TATGA | | | | | | | | | |
| (SEQ | | | | | | | | | |
| ID | | | | | | | | | |
| NO: | | | | | | | | | |
| 33) | | | | | | | | | |
| GGATA | 1122 | 1122 | 1 | 40 | 1122 | 1122 | 1 | 0.2503094 | 0 |
| TTCGG | | | | | | | | | |
| ATAGC | | | | | | | | | |
| TTTGA | | | | | | | | | |
| (SEQ | | | | | | | | | |
| ID | | | | | | | | | |
| NO: | | | | | | | | | |
| 285) | | | | | | | | | |
| AACAG | 1083 | 1083 | 1 | 40 | 1083 | 1083 | 1 | 0.25610933 | 0 |
| AGTTG | | | | | | | | | |
| AACCT | | | | | | | | | |
| TTGTG | | | | | | | | | |
| (SEQ | | | | | | | | | |
| ID | | | | | | | | | |
| NO: | | | | | | | | | |
| 286) | | | | | | | | | |
| ACCTA | 1053 | 1054 | 0.999 | 45 | 1053 | 1054 | 0.99905123 | 0.22051994 | 0 |
| GAGAG | | | | | | | | | |
| AAGCA | | | | | | | | | |
| TTCTC | | | | | | | | | |
| (SEQ | | | | | | | | | |
| ID | | | | | | | | | |
| NO: | | | | | | | | | |
| 287) | | | | | | | | | |
| GATAG | 1021 | 1021 | 1 | 40 | 1021 | 1021 | 1 | 0.41748978 | 0 |
| CTAGG | | | | | | | | | |
| AAGAT | | | | | | | | | |
| TTCCT | | | | | | | | | |
| (SEQ | | | | | | | | | |
| ID | | | | | | | | | |
| NO: | | | | | | | | | |
| 288) | | | | | | | | | |
| ACTTG | 987 | 987 | 1 | 40 | 987 | 987 | 1 | 0.70864616 | 0 |
| AGTAC | | | | | | | | | |
| ACACA | | | | | | | | | |
| TCACA | | | | | | | | | |
| (SEQ | | | | | | | | | |
| ID | | | | | | | | | |
| NO: | | | | | | | | | |
| 34) | | | | | | | | | |
| GACAC | 944 | 944 | 1 | 50 | 944 | 944 | 1 | 0.11805473 | 0 |
| TCTTT | | | | | | | | | |
| CTGCA | | | | | | | | | |
| CTACC | | | | | | | | | |
| (SEQ | | | | | | | | | |
| ID | | | | | | | | | |
| NO: | | | | | | | | | |
| 289) | | | | | | | | | |
| CTGTT | 801 | 801 | 1 | 40 | 801 | 801 | 1 | 0.3657091 | 0 |
| AGTTG | | | | | | | | | |
| AGAAC | | | | | | | | | |
| ACACA | | | | | | | | | |
| (SEQ | | | | | | | | | |
| ID | | | | | | | | | |
| NO: | | | | | | | | | |
| 290) | | | | | | | | | |
| TTGAG | 788 | 794 | 0.992 | 45 | 788 | 795 | 0.99119497 | 0.12638271 | 0 |
| GATTT | | | | | | | | | |
| CGTTG | | | | | | | | | |
| GACAC | | | | | | | | | |
| (SEQ | | | | | | | | | |
| ID | | | | | | | | | |
| NO: | | | | | | | | | |
| 291) | | | | | | | | | |
| GTGTC | 787 | 787 | 1 | 45 | 787 | 787 | 1 | 0.61306686 | 0 |
| CAACG | | | | | | | | | |
| AAATC | | | | | | | | | |
| CTCAA | | | | | | | | | |
| (SEQ | | | | | | | | | |
| ID | | | | | | | | | |
| NO: | | | | | | | | | |
| 292) | | | | | | | | | |
| TTTGA | 736 | 742 | 0.992 | 40 | 736 | 743 | 0.99057873 | 0.19120266 | 0 |
| GGATT | | | | | | | | | |
| TCGTT | | | | | | | | | |
| GGACA | | | | | | | | | |
| (SEQ | | | | | | | | | |
| ID | | | | | | | | | |
| NO: | | | | | | | | | |
| 293) | | | | | | | | | |
| ATCTA | 682 | 684 | 0.997 | 45 | 682 | 684 | 0.99707602 | 0.20407526 | 0 |
| GGGAG | | | | | | | | | |
| AAGCA | | | | | | | | | |
| TTCTC | | | | | | | | | |
| (SEQ | | | | | | | | | |
| ID | | | | | | | | | |
| NO: | | | | | | | | | |
| 294) | | | | | | | | | |
| GATAG | 616 | 617 | 0.998 | 45 | 616 | 617 | 0.99837925 | 0.24487215 | 0 |
| CTTTG | | | | | | | | | |
| CGGAT | | | | | | | | | |
| TTCGT | | | | | | | | | |
| (SEQ | | | | | | | | | |
| ID | | | | | | | | | |
| NO: | | | | | | | | | |
| 295) | | | | | | | | | |
| TTTCA | 508 | 508 | 1 | 50 | 508 | 508 | 1 | 0.27701351 | 0 |
| GGCCT | | | | | | | | | |
| GTGGT | | | | | | | | | |
| GAGAA | | | | | | | | | |
| (SEQ | | | | | | | | | |
| ID | | | | | | | | | |
| NO: | | | | | | | | | |
| 296) | | | | | | | | | |
| TTTAA | 499 | 499 | 1 | 50 | 499 | 499 | 1 | 0.21202567 | 0 |
| GCGCT | | | | | | | | | |
| TTCAG | | | | | | | | | |
| GCCTG | | | | | | | | | |
| (SEQ | | | | | | | | | |
| ID | | | | | | | | | |
| NO: | | | | | | | | | |
| 297) | | | | | | | | | |
| TGTTC | 437 | 437 | 1 | 45 | 437 | 437 | 1 | 0.27266871 | 0 |
| AGTCC | | | | | | | | | |
| TGTGA | | | | | | | | | |
| CTTGA | | | | | | | | | |
| (SEQ | | | | | | | | | |
| ID | | | | | | | | | |
| NO: | | | | | | | | | |
| 298) | | | | | | | | | |
| GATGT | 418 | 418 | 1 | 45 | 418 | 418 | 1 | 0.32961057 | 0 |
| TTGCC | | | | | | | | | |
| TTCAA | | | | | | | | | |
| GTCAC | | | | | | | | | |
| (SEQ | | | | | | | | | |
| ID | | | | | | | | | |
| NO: | | | | | | | | | |
| 299) | | | | | | | | | |

CHR10

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| GACTT | 1787 | 1792 | 0.997 | 50 | 1787 | 1792 | 0.99720982 | 0.24633979 | 0 |
| CATTG | | | | | | | | | |
| AGGCC | | | | | | | | | |
| TTCGT | | | | | | | | | |
| (SEQ | | | | | | | | | |
| ID | | | | | | | | | |
| NO: | | | | | | | | | |
| 37) | | | | | | | | | |
| AAGTC | 1703 | 1703 | 1 | 40 | 1703 | 1703 | 1 | 0.1124147 | 0 |
| CAAAA | | | | | | | | | |
| AAGCA | | | | | | | | | |
| CTTGC | | | | | | | | | |
| (SEQ | | | | | | | | | |
| ID | | | | | | | | | |
| NO: | | | | | | | | | |
| 300) | | | | | | | | | |
| TTTGA | 1476 | 1476 | 1 | 40 | 1476 | 1476 | 1 | 0.38586189 | 0 |
| CGCCA | | | | | | | | | |
| ATCTT | | | | | | | | | |
| AGACA | | | | | | | | | |
| (SEQ | | | | | | | | | |
| ID | | | | | | | | | |
| NO: | | | | | | | | | |
| 301) | | | | | | | | | |
| TGATT | 1276 | 1276 | 1 | 40 | 1276 | 1276 | 1 | 0.10021575 | 0 |
| AGTTA | | | | | | | | | |
| GACCC | | | | | | | | | |
| CTTTG | | | | | | | | | |
| (SEQ | | | | | | | | | |
| ID | | | | | | | | | |
| NO: | | | | | | | | | |
| 302) | | | | | | | | | |
| ACTCT | 931 | 931 | 1 | 40 | 931 | 931 | 1 | 0.67092919 | 0 |
| GCTCT | | | | | | | | | |
| CTCTA | | | | | | | | | |
| AAGAA | | | | | | | | | |
| (SEQ | | | | | | | | | |
| ID | | | | | | | | | |
| NO: | | | | | | | | | |
| 303) | | | | | | | | | |
| ACTCT | 853 | 853 | 1 | 45 | 853 | 853 | 1 | 0.52361023 | 0 |
| TTGTA | | | | | | | | | |
| AGTCT | | | | | | | | | |
| GCAGG | | | | | | | | | |
| (SEQ | | | | | | | | | |
| ID | | | | | | | | | |
| NO: | | | | | | | | | |
| 304) | | | | | | | | | |
| CTTTC | 777 | 777 | 1 | 45 | 777 | 777 | 1 | 0.63862776 | 0 |
| TGTGG | | | | | | | | | |
| AGTTT | | | | | | | | | |
| GCAAG | | | | | | | | | |
| (SEQ | | | | | | | | | |
| ID | | | | | | | | | |
| NO: | | | | | | | | | |
| 305) | | | | | | | | | |
| CCTTT | 679 | 679 | 1 | 50 | 679 | 679 | 1 | 0.51073661 | 0 |
| CTTTA | | | | | | | | | |
| GAGGG | | | | | | | | | |
| AGCAG | | | | | | | | | |
| (SEQ | | | | | | | | | |
| ID | | | | | | | | | |
| NO: | | | | | | | | | |
| 306) | | | | | | | | | |
| CCTCT | 670 | 670 | 1 | 50 | 670 | 670 | 1 | 0.71419362 | 0 |
| GCTCC | | | | | | | | | |
| CTCTA | | | | | | | | | |
| AAGAA | | | | | | | | | |
| (SEQ | | | | | | | | | |
| ID | | | | | | | | | |
| NO: | | | | | | | | | |
| 307) | | | | | | | | | |

CHR11

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| GATAT | 3452 | 3452 | 1 | 45 | 3480 | 3480 | 0.99195402 | 0.51141482 | 28 |
| ACCCG | | | | | | | | | |
| TTTCG | | | | | | | | | |
| AAGGA | | | | | | | | | |
| (SEQ | | | | | | | | | |
| ID | | | | | | | | | |
| NO: | | | | | | | | | |
| 308) | | | | | | | | | |
| TTCCA | 3393 | 3408 | 0.996 | 40 | 3393 | 3408 | 0.99559859 | 0.62451756 | 0 |
| ACGAA | | | | | | | | | |
| ATCTT | | | | | | | | | |
| CACAG | | | | | | | | | |
| (SEQ | | | | | | | | | |
| ID | | | | | | | | | |
| NO: | | | | | | | | | |
| 39) | | | | | | | | | |
| TTTGT | 3390 | 3390 | 1 | 45 | 3417 | 3417 | 0.99209833 | 0.54304654 | 27 |
| GGCCT | | | | | | | | | |
| TCCTT | | | | | | | | | |
| CGAAA | | | | | | | | | |
| (SEQ | | | | | | | | | |
| ID | | | | | | | | | |
| NO: | | | | | | | | | |
| 309) | | | | | | | | | |
| TGAAG | 3385 | 3385 | 1 | 40 | 3413 | 3415 | 0.99121523 | 0.23652185 | 28 |
| ATATA | | | | | | | | | |
| CCCGT | | | | | | | | | |
| TTCGA | | | | | | | | | |
| (SEQ | | | | | | | | | |
| ID | | | | | | | | | |
| NO: | | | | | | | | | |
| 310) | | | | | | | | | |
| TTGTG | 3383 | 3383 | 1 | 50 | 3412 | 3412 | 0.99150059 | 0.15060442 | 29 |
| GCCTT | | | | | | | | | |
| CCTTC | | | | | | | | | |
| GAAAC | | | | | | | | | |
| (SEQ | | | | | | | | | |
| ID | | | | | | | | | |
| NO: | | | | | | | | | |
| 311) | | | | | | | | | |
| TGTCT | 3357 | 3357 | 1 | 45 | 3357 | 3357 | 1 | 0.63302776 | 0 |
| GTGAA | | | | | | | | | |
| TGCTT | | | | | | | | | |
| CCGTT | | | | | | | | | |
| (SEQ | | | | | | | | | |
| ID | | | | | | | | | |
| NO: | | | | | | | | | |
| 312) | | | | | | | | | |
| TTCGA | 3348 | 3348 | 1 | 50 | 3374 | 3374 | 0.99229401 | 0.50366609 | 26 |
| AGGAA | | | | | | | | | |
| GGCCA | | | | | | | | | |
| CAAAG | | | | | | | | | |
| (SEQ | | | | | | | | | |
| ID | | | | | | | | | |
| NO: | | | | | | | | | |
| 313) | | | | | | | | | |
| GAGTT | 3345 | 3347 | 0.999 | 45 | 3351 | 3353 | 0.99761408 | 0.11378584 | 0 |
| GAATG | | | | | | | | | |
| CAGTC | | | | | | | | | |
| ATCAC | | | | | | | | | |
| (SEQ | | | | | | | | | |
| ID | | | | | | | | | |
| NO: | | | | | | | | | |
| 314) | | | | | | | | | |
| GACCT | 3333 | 3348 | 0.996 | 45 | 3333 | 3348 | 0.99551971 | 0.59681901 | 0 |
| CTGTG | | | | | | | | | |
| AAGAT | | | | | | | | | |
| TTCGT | | | | | | | | | |
| (SEQ | | | | | | | | | |
| ID | | | | | | | | | |
| NO: | | | | | | | | | |
| 315) | | | | | | | | | |
| AGATT | 3327 | 3327 | 1 | 40 | 3328 | 3329 | 0.99939922 | 0.14609511 | 0 |
| TTCCT | | | | | | | | | |
| TTTCC | | | | | | | | | |
| ACCAC | | | | | | | | | |
| (SEQ | | | | | | | | | |
| ID | | | | | | | | | |
| NO: | | | | | | | | | |
| 316) | | | | | | | | | |
| TTTGA | 3327 | 3331 | 0.999 | 40 | 3327 | 3332 | 0.9984994 | 0.18722066 | 0 |
| GGCCT | | | | | | | | | |
| ACTGT | | | | | | | | | |
| AGTAA | | | | | | | | | |
| (SEQ | | | | | | | | | |
| ID | | | | | | | | | |
| NO: | | | | | | | | | |
| 317) | | | | | | | | | |
| TTCAG | 3308 | 3308 | 1 | 50 | 3308 | 3308 | 1 | 0.31957638 | 0 |
| AGCTG | | | | | | | | | |
| CTCTG | | | | | | | | | |
| TCAAG | | | | | | | | | |
| (SEQ | | | | | | | | | |
| ID | | | | | | | | | |
| NO: | | | | | | | | | |
| 38) | | | | | | | | | |
| TGACT | 3302 | 3303 | 1 | 45 | 3302 | 3303 | 0.99969724 | 0.76808132 | 0 |
| GCATT | | | | | | | | | |
| CAACT | | | | | | | | | |
| CACAG | | | | | | | | | |
| (SEQ | | | | | | | | | |
| ID | | | | | | | | | |
| NO: | | | | | | | | | |
| 318) | | | | | | | | | |
| GAGCT | 3202 | 3203 | 1 | 40 | 3231 | 3232 | 0.99071782 | 0.23842358 | 29 |
| GAACA | | | | | | | | | |
| TTCCT | | | | | | | | | |
| TTAGA | | | | | | | | | |
| (SEQ | | | | | | | | | |
| ID | | | | | | | | | |
| NO: | | | | | | | | | |
| 319) | | | | | | | | | |
| CTTTC | 2878 | 2878 | 1 | 40 | 2878 | 2878 | 1 | 0.43414669 | 0 |
| TTTGG | | | | | | | | | |
| ATTCT | | | | | | | | | |
| GCAAG | | | | | | | | | |
| (SEQ | | | | | | | | | |
| ID | | | | | | | | | |
| NO: | | | | | | | | | |
| 320) | | | | | | | | | |
| GGATT | 2776 | 2776 | 1 | 45 | 2776 | 2776 | 1 | 0.19174637 | 0 |
| CTGCA | | | | | | | | | |
| AGTGG | | | | | | | | | |
| ATATG | | | | | | | | | |
| (SEQ | | | | | | | | | |
| ID | | | | | | | | | |
| NO: | | | | | | | | | |
| 321) | | | | | | | | | |
| GAGGT | 2371 | 2371 | 1 | 50 | 2371 | 2371 | 1 | 0.12298616 | 0 |
| GAACA | | | | | | | | | |
| ATCCT | | | | | | | | | |
| GCTGA | | | | | | | | | |
| (SEQ | | | | | | | | | |
| ID | | | | | | | | | |
| NO: | | | | | | | | | |
| 322) | | | | | | | | | |
| GGAAA | 1822 | 1822 | 1 | 40 | 1822 | 1822 | 1 | 0.32767222 | 0 |
| GTTCA | | | | | | | | | |
| ATTCC | | | | | | | | | |
| TGAAG | | | | | | | | | |
| (SEQ | | | | | | | | | |
| ID | | | | | | | | | |
| NO: | | | | | | | | | |
| 323) | | | | | | | | | |
| TTGGA | 1350 | 1350 | 1 | 45 | 1350 | 1350 | 1 | 0.14861684 | 0 |
| AACTG | | | | | | | | | |
| CGCCA | | | | | | | | | |
| TCTAA | | | | | | | | | |
| (SEQ | | | | | | | | | |
| ID | | | | | | | | | |
| NO: | | | | | | | | | |
| 324) | | | | | | | | | |
| GATTC | 719 | 719 | 1 | 40 | 719 | 719 | 1 | 0.25278666 | 0 |
| TACAG | | | | | | | | | |
| AAAGT | | | | | | | | | |
| GGGTT | | | | | | | | | |
| (SEQ | | | | | | | | | |
| ID | | | | | | | | | |
| NO: | | | | | | | | | |
| 325) | | | | | | | | | |
| GGATT | 561 | 561 | 1 | 40 | 561 | 561 | 1 | 0.45882202 | 0 |
| CTGCA | | | | | | | | | |
| AGTTG | | | | | | | | | |
| ATATG | | | | | | | | | |
| (SEQ | | | | | | | | | |
| ID | | | | | | | | | |
| NO: | | | | | | | | | |
| 326) | | | | | | | | | |

CHR12

| | bind- | | | | | | | | bind- | | | | |
|---|---|---|---|---|---|---|---|---|---|---|---|---|---|
| | ing | | | | | | | | ing | | | | |
| | sites | | | | | | | | sites | | | | |
| | (CHM | | | | | | | | (CHM13- | | | | |
| | 13- | bind- | | | | | | | spe- | | | | |
| | spe- | ing | | | | | | | cif- | | | | |
| | cif- | sites | | | | | | | ic | | | | |
| | ic | (CHM | | | bind- | | | | chro- | | | | |
| | chro- | 13- | chro- | | ing | bind- | | | mo- | | | | |
| | mo- | all | mo- | | sites | ing | centro- | | some | | | | |
| | some | cen- | some | | (CHM- | sites | mere | | centro- | | | | |
| sg | centro- | tro- | spe- | | 13 | (hg38 | spe- | Ac- | mere | | | | |
| RNA | mere | mere | cif- | GC_ | whole | whole | cif- | tiv- | Not | | | | |
| _ | HOR_ | HOR_ | ic- | cont- | ge- | ge- | ic- | ity | HOR_ | | | | |
| seq | L) | L) | ity | ent | nome) | nome) | ity | score | L) | | | | |
| CAGAT | 2727 | 2727 | 1 | 40 | 2727 | 2728 | 0.99963343 | 0.13096201 | 0 | | | | |
| ATTTG | | | | | | | | | | | | | |
| GACCT | | | | | | | | | | | | | |
| CTTTG | | | | | | | | | | | | | |
| (SEQ | | | | | | | | | | | | | |
| ID | | | | | | | | | | | | | |
| NO: | | | | | | | | | | | | | |
| 327) | | | | | | | | | | | | | |
| TGCCT | 1741 | 1741 | 1 | 45 | 1742 | 1742 | 0.99942595 | 0.6121426 | 0 | | | | |
| CTATT | | | | | | | | | | | | | |
| CAACT | | | | | | | | | | | | | |
| CACAG | | | | | | | | | | | | | |
| (SEQ | | | | | | | | | | | | | |
| ID | | | | | | | | | | | | | |
| NO: | | | | | | | | | | | | | |
| 40) | | | | | | | | | | | | | |
| CACCT | 1727 | 1727 | 1 | 45 | 1728 | 1728 | 0.9994213 | 0.52149722 | 0 | | | | |
| CTGTG | | | | | | | | | | | | | |
| AGTTG | | | | | | | | | | | | | |
| AATAG | | | | | | | | | | | | | |
| (SEQ | | | | | | | | | | | | | |
| ID | | | | | | | | | | | | | |
| NO: | | | | | | | | | | | | | |
| 41) | | | | | | | | | | | | | |
| AATCT | 1651 | 1651 | 1 | 40 | 1651 | 1651 | 1 | 0.60806334 | 0 | | | | |
| GCTCT | | | | | | | | | | | | | |
| TTCTG | | | | | | | | | | | | | |
| AAGGA | | | | | | | | | | | | | |
| (SEQ | | | | | | | | | | | | | |
| ID | | | | | | | | | | | | | |
| NO: | | | | | | | | | | | | | |
| 328) | | | | | | | | | | | | | |
| GTCTT | 1648 | 1648 | 1 | 40 | 1648 | 1663 | 0.99098016 | 0.50188624 | 0 | | | | |
| TGTAA | | | | | | | | | | | | | |
| AGTCT | | | | | | | | | | | | | |
| GCAAG | | | | | | | | | | | | | |
| (SEQ | | | | | | | | | | | | | |
| ID | | | | | | | | | | | | | |
| NO: | | | | | | | | | | | | | |
| 329) | | | | | | | | | | | | | |
| TGATG | 1625 | 1626 | 0.999 | 40 | 1625 | 1629 | 0.99754451 | 0.1449803 | 0 | | | | |
| TGTGT | | | | | | | | | | | | | |
| GTTCA | | | | | | | | | | | | | |
| ACTCA | | | | | | | | | | | | | |
| (SEQ | | | | | | | | | | | | | |
| ID | | | | | | | | | | | | | |
| NO: | | | | | | | | | | | | | |
| 330) | | | | | | | | | | | | | |
| CTATT | 1609 | 1609 | 1 | 40 | 1609 | 1609 | 1 | 0.82269867 | 0 | | | | |
| TGTGC | | | | | | | | | | | | | |
| AGTTT | | | | | | | | | | | | | |
| CCAGT | | | | | | | | | | | | | |
| (SEQ | | | | | | | | | | | | | |
| ID | | | | | | | | | | | | | |
| NO: | | | | | | | | | | | | | |
| 331) | | | | | | | | | | | | | |
| CTTTT | 1608 | 1608 | 1 | 40 | 1609 | 1609 | 0.9993785 | 0.37384791 | 0 | | | | |
| TGTGG | | | | | | | | | | | | | |
| AATTT | | | | | | | | | | | | | |
| GCAGC | | | | | | | | | | | | | |
| (SEQ | | | | | | | | | | | | | |
| ID | | | | | | | | | | | | | |
| NO: | | | | | | | | | | | | | |
| 332) | | | | | | | | | | | | | |
| CTTTT | 1597 | 1597 | 1 | 40 | 1597 | 1597 | 1 | 0.73270864 | 0 | | | | |
| TGTGG | | | | | | | | | | | | | |
| AGTTT | | | | | | | | | | | | | |
| CCATG | | | | | | | | | | | | | |
| (SEQ | | | | | | | | | | | | | |
| ID | | | | | | | | | | | | | |
| NO: | | | | | | | | | | | | | |
| 333) | | | | | | | | | | | | | |
| CAGCT | 1547 | 1547 | 1 | 40 | 1548 | 1548 | 0.99935401 | 0.22461139 | 0 | | | | |
| GCAAA | | | | | | | | | | | | | |
| TTCCA | | | | | | | | | | | | | |
| CAAAA | | | | | | | | | | | | | |
| (SEQ | | | | | | | | | | | | | |
| ID | | | | | | | | | | | | | |
| NO: | | | | | | | | | | | | | |
| 334) | | | | | | | | | | | | | |
| AAGCG | 1538 | 1538 | 1 | 40 | 1538 | 1538 | 1 | 0.51818734 | 0 | | | | |
| ATTGA | | | | | | | | | | | | | |
| AATCT | | | | | | | | | | | | | |
| CCAAC | | | | | | | | | | | | | |
| (SEQ | | | | | | | | | | | | | |
| ID | | | | | | | | | | | | | |
| NO: | | | | | | | | | | | | | |
| 335) | | | | | | | | | | | | | |
| AAGCG | | | 1537 | 1537 | | 1 | 40 | | 1537 | 1537 | 1 | 0.7209175 | 0 |
| ATTGA | | | | | | | | | | | | | |
| AATCT | | | | | | | | | | | | | |
| CCACA | | | | | | | | | | | | | |
| (SEQ | | | | | | | | | | | | | |
| ID | | | | | | | | | | | | | |
| NO: | | | | | | | | | | | | | |
| 336) | | | | | | | | | | | | | |
| GATGT | | | 1481 | 1481 | | 1 | 40 | | 1481 | 1486 | 0.99663526 | 0.37701498 | 0 |
| TTCCT | | | | | | | | | | | | | |
| TTTCT | | | | | | | | | | | | | |
| ACCGT | | | | | | | | | | | | | |
| (SEQ | | | | | | | | | | | | | |
| ID | | | | | | | | | | | | | |
| NO: | | | | | | | | | | | | | |
| 337) | | | | | | | | | | | | | |
| TTCAA | | | 1455 | 1455 | | 1 | 40 | | 1455 | 1455 | 1 | 0.27838956 | 0 |
| TCGCT | | | | | | | | | | | | | |
| TTGAG | | | | | | | | | | | | | |
| ACCAA | | | | | | | | | | | | | |
| (SEQ | | | | | | | | | | | | | |
| ID | | | | | | | | | | | | | |
| NO: | | | | | | | | | | | | | |
| 338) | | | | | | | | | | | | | |

CHR13

| | bind- | | | | | | | | bind- | | | | | | |
|---|---|---|---|---|---|---|---|---|---|---|---|---|---|---|---|
| | ing | | | | | | | | ing | | | | | | |
| | sites | | | | | | | | sites | | | | | | |
| | (CHM | | | | | | | | (CHM13- | | | | | | |
| | 13- | bind- | | | | | | | spe- | | | | | | |
| | spe- | ing | | | | | | | cif- | | | | | | |
| | cif- | sites | | | | | | | ic | | | | | | |
| | ic | (CHM | | | bind- | | | | chro- | | | | | | |
| | chro- | 13- | chro- | | ing | bind- | | | mo- | | | | | | |
| | mo- | all | mo- | | sites | ing | centro- | | some | | | | | | |
| | some | cen- | some | | (CHM- | sites | mere | | centro- | | | | | | |
| sg | centro- | tro- | spe- | | 13 | (hg38 | spe- | Ac- | mere | | | | | | |
| RNA | mere | mere | cif- | GC_ | whole | whole | cif- | tiv- | Not | | | | | | |
| _ | HOR_ | HOR_ | ic- | cont- | ge- | ge- | ic- | ity | HOR_ | | | | | | |
| seq | L) | L) | ity | ent | nome) | nome) | ity | score | L) | | | | | | |
| TCGAC | 1257 | 1257 | 1 | | 40 | | | 1258 | | 1258 | 0.99920509 | 0.29133657 | 0 | hor_ | hor_ |
| TCATA | | | | | | | | | | | | | | 13_ | 13_ |
| GAGAT | | | | | | | | | | | | | | 3 | 3 |
| GAACA | | | | | | | | | | | | | | (S2C1 | (S2C1 |
| (SEQ | | | | | | | | | | | | | | 3/ | 3/ |
| ID | | | | | | | | | | | | | | 21H1L) | 21H1L) |
| NO: | | | | | | | | | | | | | | | |
| 49) | | | | | | | | | | | | | | | |
| TCCCA | 908 | 908 | 1 | | 45 | | | 908 | | 908 | 1 | 0.28432971 | 0 | hor_ | hor_ |
| GAAAA | | | | | | | | | | | | | | 13_ | 13_ |
| ACGAG | | | | | | | | | | | | | | 3 | 3 |
| ACAGA | | | | | | | | | | | | | | (S2C1 | (S2C1 |
| (SEQ | | | | | | | | | | | | | | 3/ | 3/ |
| ID | | | | | | | | | | | | | | 21H1L) | 21H1L) |
| NO: | | | | | | | | | | | | | | | |
| 339) | | | | | | | | | | | | | | | |

CHR14

| | | | | | | | | | bind- |
|---|---|---|---|---|---|---|---|---|---|
| | bind- | | | | | | | | ing |
| | ing | | | | | | | | sites |
| | sites | | | | | | | | (CHM13- |
| | (CHM13- | | | | | | | | spe- |
| | spe- | | | | | | | | cif- |
| | cif- | bind- | | | | | | | ic |
| | ic | ing | chro- | | bind- | | | | chro- |
| | chro- | sites | mo- | | ing | bind- | | | mo- |
| | mo- | (CHM13- | some | | sites | ing | centro- | | some |
| | some | all | spe- | | (CHM- | sites | mere | | centro- |
| | centro- | centro- | - | | 13 | (hg38 | spe- | Ac- | mere |
| sgRNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| ATGGA | 670 | 670 | 1 | 45 | 670 | 670 | 1 | 0.60023007 | 0 |
| AGTGG | | | | | | | | | |
| ACTTA | | | | | | | | | |
| TCGGA | | | | | | | | | |
| (SEQ | | | | | | | | | |
| ID | | | | | | | | | |
| NO: | | | | | | | | | |
| 340) | | | | | | | | | |

CHR15

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| TTTCA | 710 | 711 | 0.999 | 45 | 710 | 711 | 0.99859353 | 0.37331803 | 0 |
| GGCCT | | | | | | | | | |
| AAGGT | | | | | | | | | |
| GAGAA | | | | | | | | | |
| (SEQ | | | | | | | | | |
| ID | | | | | | | | | |
| NO: | | | | | | | | | |
| 341) | | | | | | | | | |
| TCTTA | 407 | 407 | 1 | 40 | 407 | 407 | 1 | 0.34926818 | 0 |
| GGCCT | | | | | | | | | |
| AAGGT | | | | | | | | | |
| GAAAA | | | | | | | | | |
| (SEQ | | | | | | | | | |
| ID | | | | | | | | | |
| NO: | | | | | | | | | |
| 51) | | | | | | | | | |
| CTCTT | 406 | 406 | 1 | 45 | 406 | 406 | 1 | 0.15006052 | 0 |
| AGGCC | | | | | | | | | |
| TAAGG | | | | | | | | | |
| TGAAA | | | | | | | | | |
| (SEQ | | | | | | | | | |
| ID | | | | | | | | | |
| NO: | | | | | | | | | |
| 342) | | | | | | | | | |
| CTGTT | 406 | 406 | 1 | 40 | 406 | 407 | 0.997543 | 0.3434124 | 0 |
| AGTTG | | | | | | | | | |
| AGTAC | | | | | | | | | |
| ACACA | | | | | | | | | |
| (SEQ | | | | | | | | | |
| ID | | | | | | | | | |
| NO: | | | | | | | | | |
| 343) | | | | | | | | | |
| TGGAC | 406 | 406 | 1 | 45 | 406 | 406 | 1 | 0.12766553 | 0 |
| ATTTC | | | | | | | | | |
| GAGCA | | | | | | | | | |
| CTCTT | | | | | | | | | |
| (SEQ | | | | | | | | | |
| ID | | | | | | | | | |
| NO: | | | | | | | | | |
| 344) | | | | | | | | | |
| AGGTT | 403 | 403 | 1 | 45 | 403 | 403 | 1 | 0.15598418 | 0 |
| GAACT | | | | | | | | | |
| CTGTG | | | | | | | | | |
| AGTTG | | | | | | | | | |
| (SEQ | | | | | | | | | |
| ID | | | | | | | | | |
| NO: | | | | | | | | | |
| 345) | | | | | | | | | |

CHR16

| | bind- | | | | | | | | bind- |
|---|---|---|---|---|---|---|---|---|---|
| | ing | | | | | | | | ing |
| | sites | | | | | | | | sites |
| | (CHM | | | | | | | | (CHM13- |
| | 13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| TGGAT | 1159 | 1159 | 1 | 45 | 1159 | 1159 | 1 | 0.41745491 | 0 |
| ATCTT | | | | | | | | | |
| GGCCT | | | | | | | | | |
| CTTAG | | | | | | | | | |
| (SEQ | | | | | | | | | |
| ID | | | | | | | | | |
| NO: | | | | | | | | | |
| 54) | | | | | | | | | |
| GGCCT | 1152 | 1152 | 1 | 60 | 1152 | 1152 | 1 | 0.45221867 | 0 |
| CTTAG | | | | | | | | | |
| AGGCC | | | | | | | | | |
| TTCGT | | | | | | | | | |
| (SEQ | | | | | | | | | |
| ID | | | | | | | | | |
| NO: | | | | | | | | | |
| 346) | | | | | | | | | |
| TTTGA | 1131 | 1134 | 0.997 | 40 | 1131 | 1134 | 0.9973545 | 0.27000218 | 0 |
| GGCCA | | | | | | | | | |
| AAAGC | | | | | | | | | |
| AGAAA | | | | | | | | | |
| (SEQ | | | | | | | | | |
| ID | | | | | | | | | |
| NO: | | | | | | | | | |
| 347) | | | | | | | | | |
| CTGTT | 1093 | 1093 | 1 | 55 | 1093 | 1093 | 1 | 0.37634959 | 0 |
| TGTGA | | | | | | | | | |
| AGCCT | | | | | | | | | |
| GCCAG | | | | | | | | | |
| (SEQ | | | | | | | | | |
| ID | | | | | | | | | |
| NO: | | | | | | | | | |
| 55) | | | | | | | | | |
| TGGAG | 1067 | 1072 | 0.995 | 50 | 1067 | 1074 | 0.99348231 | 0.1461381 | 0 |
| ACTTC | | | | | | | | | |
| AAGCG | | | | | | | | | |
| CTTTG | | | | | | | | | |
| (SEQ | | | | | | | | | |
| ID | | | | | | | | | |
| NO: | | | | | | | | | |
| 348) | | | | | | | | | |
| GCAGA | 1058 | 1058 | 1 | 40 | 1058 | 1058 | 1 | 0.22038978 | 0 |
| TTTGA | | | | | | | | | |
| GACAC | | | | | | | | | |
| TCTTT | | | | | | | | | |
| (SEQ | | | | | | | | | |
| ID | | | | | | | | | |
| NO: | | | | | | | | | |
| 349) | | | | | | | | | |
| TTCGA | 1050 | 1054 | 0.996 | 40 | 1050 | 1054 | 0.99620493 | 0.12234006 | 0 |
| ATCTG | | | | | | | | | |
| CTCTG | | | | | | | | | |
| TCTAA | | | | | | | | | |
| (SEQ | | | | | | | | | |
| ID | | | | | | | | | |
| NO: | | | | | | | | | |
| 350) | | | | | | | | | |
| AAAGA | 1033 | 1033 | 1 | 45 | 1033 | 1033 | 1 | 0.45397004 | 0 |
| GGTCC | | | | | | | | | |
| GAATA | | | | | | | | | |
| TCCAC | | | | | | | | | |
| (SEQ | | | | | | | | | |
| ID | | | | | | | | | |
| NO: | | | | | | | | | |
| 351) | | | | | | | | | |
| CTGTT | 1032 | 1034 | 0.998 | 50 | 1032 | 1037 | 0.9951784 | 0.61298827 | 0 |
| TGGAA | | | | | | | | | |
| AGTCT | | | | | | | | | |
| GCACG | | | | | | | | | |
| (SEQ | | | | | | | | | |
| ID | | | | | | | | | |
| NO: | | | | | | | | | |
| 352) | | | | | | | | | |
| TTAGA | 1023 | 1029 | 0.994 | 40 | 1023 | 1030 | 0.99320388 | 0.44633557 | 0 |
| TGCCT | | | | | | | | | |
| TCGTT | | | | | | | | | |
| GGAAA | | | | | | | | | |
| (SEQ | | | | | | | | | |
| ID | | | | | | | | | |
| NO: | | | | | | | | | |
| 353) | | | | | | | | | |
| TAGAT | 1009 | 1014 | 0.995 | 45 | 1009 | 1014 | 0.99506903 | 0.12608287 | 0 |
| GCCTT | | | | | | | | | |
| CGTTG | | | | | | | | | |
| GAAAC | | | | | | | | | |
| (SEQ | | | | | | | | | |
| ID | | | | | | | | | |
| NO: | | | | | | | | | |
| 354) | | | | | | | | | |
| GACCT | 1007 | 1008 | 0.999 | 50 | 1007 | 1008 | 0.99900794 | 0.47301652 | 0 |
| CTTAG | | | | | | | | | |
| ATGCC | | | | | | | | | |
| TTCGT | | | | | | | | | |
| (SEQ | | | | | | | | | |
| ID | | | | | | | | | |
| NO: | | | | | | | | | |
| 355) | | | | | | | | | |
| AGGTC | 1004 | 1004 | 1 | 55 | 1004 | 1004 | 1 | 0.15231774 | 0 |
| CGAAT | | | | | | | | | |
| ATCCA | | | | | | | | | |
| CTGGC | | | | | | | | | |
| (SEQ | | | | | | | | | |
| ID | | | | | | | | | |
| NO: | | | | | | | | | |
| 356) | | | | | | | | | |
| TTCCA | 1001 | 1006 | 0.995 | 45 | 1001 | 1007 | 0.99404171 | 0.54864956 | 0 |
| ACGAA | | | | | | | | | |
| GGCAT | | | | | | | | | |
| CTAAG | | | | | | | | | |
| (SEQ | | | | | | | | | |
| ID | | | | | | | | | |
| NO: | | | | | | | | | |
| 357) | | | | | | | | | |
| GATTT | 947 | 947 | 1 | 40 | 947 | 947 | 1 | 0.4252322 | 0 |
| GAGAC | | | | | | | | | |
| ACTCT | | | | | | | | | |
| TTTGG | | | | | | | | | |
| (SEQ | | | | | | | | | |
| ID | | | | | | | | | |
| NO: | | | | | | | | | |
| 358) | | | | | | | | | |
| TTCGA | 932 | 932 | 1 | 40 | 932 | 932 | 1 | 0.11308953 | 0 |
| TGCCA | | | | | | | | | |
| ATGGT | | | | | | | | | |
| AGAAA | | | | | | | | | |
| (SEQ | | | | | | | | | |
| ID | | | | | | | | | |
| NO: | | | | | | | | | |
| 359) | | | | | | | | | |
| TTCAA | 922 | 922 | 1 | 50 | 922 | 922 | 1 | 0.31052538 | 0 |
| GCGCT | | | | | | | | | |
| TCGAT | | | | | | | | | |
| GCCAA | | | | | | | | | |
| (SEQ | | | | | | | | | |
| ID | | | | | | | | | |
| NO: | | | | | | | | | |
| 360) | | | | | | | | | |
| TCTTT | 822 | 822 | 1 | 40 | 822 | 822 | 1 | 0.15713836 | 0 |
| CTCAG | | | | | | | | | |
| AAACT | | | | | | | | | |
| GCTCT | | | | | | | | | |
| (SEQ | | | | | | | | | |
| ID | | | | | | | | | |
| NO: | | | | | | | | | |
| 361) | | | | | | | | | |
| ATCTT | 821 | 821 | 1 | 40 | 821 | 822 | 0.99878345 | 0.12704844 | 0 |
| TCTCA | | | | | | | | | |
| GAAAC | | | | | | | | | |
| TGCTC | | | | | | | | | |
| (SEQ | | | | | | | | | |
| ID | | | | | | | | | |
| NO: | | | | | | | | | |
| 362) | | | | | | | | | |
| CTTGC | 449 | 449 | 1 | 40 | 449 | 449 | 1 | 0.26213196 | 0 |
| AGACT | | | | | | | | | |
| TTACA | | | | | | | | | |
| AACAC | | | | | | | | | |
| (SEQ | | | | | | | | | |
| ID | | | | | | | | | |
| NO: | | | | | | | | | |
| 363) | | | | | | | | | |

CHR17

| | | | | | | | | | bind- |
|---|---|---|---|---|---|---|---|---|---|
| | bind- | | | | | | | | ing |
| | ing | | | | | | | | sites |
| | sites | | | | | | | | (CHM13- |
| | (CHM13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| TTCCA | 1299 | 1310 | 0.992 | 40 | 1299 | 1312 | 0.99009146 | 0.19156279 | 0 |
| AACTG | | | | | | | | | |
| CTCTG | | | | | | | | | |
| TCAAA | | | | | | | | | |
| (SEQ | | | | | | | | | |
| ID | | | | | | | | | |
| NO: | | | | | | | | | |
| 364) | | | | | | | | | |
| TTTCC | 1297 | 1297 | 1 | 40 | 1297 | 1301 | 0.99692544 | 0.23347441 | 0 |
| AAACT | | | | | | | | | |
| GCTCT | | | | | | | | | |
| GTCAA | | | | | | | | | |
| (SEQ | | | | | | | | | |
| ID | | | | | | | | | |
| NO: | | | | | | | | | |
| 365) | | | | | | | | | |
| TTCCC | 1294 | 1294 | 1 | 45 | 1294 | 1294 | 1 | 0.17609664 | 0 |
| TTTGA | | | | | | | | | |
| CAGAG | | | | | | | | | |
| CAGTT | | | | | | | | | |
| (SEQ | | | | | | | | | |
| ID | | | | | | | | | |
| NO: | | | | | | | | | |
| 366) | | | | | | | | | |
| GAGGG | 1286 | 1286 | 1 | 55 | 1286 | 1286 | 1 | 0.22681535 | 0 |
| CTTTG | | | | | | | | | |
| TGGTT | | | | | | | | | |
| TGTGG | | | | | | | | | |
| (SEQ | | | | | | | | | |
| ID | | | | | | | | | |
| NO: | | | | | | | | | |
| 367) | | | | | | | | | |
| TTCAA | 1282 | 1282 | 1 | 40 | 1282 | 1282 | 1 | 0.25320634 | 0 |
| AGCTT | | | | | | | | | |
| CTCTC | | | | | | | | | |
| TCGAA | | | | | | | | | |
| (SEQ | | | | | | | | | |
| ID | | | | | | | | | |
| NO: | | | | | | | | | |
| 368) | | | | | | | | | |
| CATAA | 1264 | 1264 | 1 | 40 | 1264 | 1264 | 1 | 0.12664826 | 0 |
| TTCGT | | | | | | | | | |
| TTTCC | | | | | | | | | |
| ACCAC | | | | | | | | | |
| (SEQ | | | | | | | | | |
| ID | | | | | | | | | |
| NO: | | | | | | | | | |
| 369) | | | | | | | | | |
| GAATG | 1263 | 1263 | 1 | 45 | 1263 | 1263 | 1 | 0.21701208 | 0 |
| CAGAC | | | | | | | | | |
| ATCAC | | | | | | | | | |
| GAAGA | | | | | | | | | |
| (SEQ | | | | | | | | | |
| ID | | | | | | | | | |
| NO: | | | | | | | | | |
| 370) | | | | | | | | | |
| GCTAT | 1246 | 1246 | 1 | 40 | 1249 | 1249 | 0.99759808 | 0.48930092 | 0 |
| TCCCT | | | | | | | | | |
| TTACT | | | | | | | | | |
| ACCAT | | | | | | | | | |
| (SEQ | | | | | | | | | |
| ID | | | | | | | | | |
| NO: | | | | | | | | | |
| 371) | | | | | | | | | |
| CTCAA | 1242 | 1242 | 1 | 50 | 1242 | 1242 | 1 | 0.21427921 | 0 |
| ACCTG | | | | | | | | | |
| CTCCA | | | | | | | | | |
| TCCAA | | | | | | | | | |
| (SEQ | | | | | | | | | |
| ID | | | | | | | | | |
| NO: | | | | | | | | | |
| 372) | | | | | | | | | |
| GACCT | 1242 | 1242 | 1 | 50 | 1242 | 1242 | 1 | 0.34406756 | 0 |
| CTCCG | | | | | | | | | |
| AAGAT | | | | | | | | | |
| GTCTT | | | | | | | | | |
| (SEQ | | | | | | | | | |
| ID | | | | | | | | | |
| NO: | | | | | | | | | |
| 373) | | | | | | | | | |
| TCTGC | 1242 | 1242 | 1 | 45 | 1242 | 1242 | 1 | 0.55903398 | 0 |
| ATTCA | | | | | | | | | |
| ACTCA | | | | | | | | | |
| CAGTG | | | | | | | | | |
| (SEQ | | | | | | | | | |
| ID | | | | | | | | | |
| NO: | | | | | | | | | |
| 374) | | | | | | | | | |
| CTCTT | 1238 | 1238 | 1 | 50 | 1238 | 1238 | 1 | 0.4792925 | 0 |
| TCTGT | | | | | | | | | |
| GGCAT | | | | | | | | | |
| CTGCA | | | | | | | | | |
| (SEQ | | | | | | | | | |
| ID | | | | | | | | | |
| NO: | | | | | | | | | |
| 375) | | | | | | | | | |
| TCTTT | 1237 | 1237 | 1 | 45 | 1237 | 1237 | 1 | 0.49504363 | 0 |
| CTGTG | | | | | | | | | |
| GCATC | | | | | | | | | |
| TGCAA | | | | | | | | | |
| (SEQ | | | | | | | | | |
| ID | | | | | | | | | |
| NO: | | | | | | | | | |
| 376) | | | | | | | | | |
| CCCGT | 1225 | 1225 | 1 | 45 | 1225 | 1225 | 1 | 0.11027464 | 0 |
| TTCCA | | | | | | | | | |
| AAGAC | | | | | | | | | |
| ATCTT | | | | | | | | | |
| (SEQ | | | | | | | | | |
| ID | | | | | | | | | |
| NO: | | | | | | | | | |
| 377) | | | | | | | | | |
| TGAAT | 1222 | 1222 | 1 | 40 | 1222 | 1222 | 1 | 0.42232024 | 0 |
| GCAAA | | | | | | | | | |
| CATCA | | | | | | | | | |
| CGAAG | | | | | | | | | |
| (SEQ | | | | | | | | | |
| ID | | | | | | | | | |
| NO: | | | | | | | | | |
| 378) | | | | | | | | | |
| GCTTC | 1220 | 1220 | 1 | 40 | 1220 | 1220 | 1 | 0.50283523 | 0 |
| TGTTT | | | | | | | | | |
| TAGTT | | | | | | | | | |
| CTGTG | | | | | | | | | |
| (SEQ | | | | | | | | | |
| ID | | | | | | | | | |
| NO: | | | | | | | | | |
| 379) | | | | | | | | | |
| TCCGA | 1219 | 1219 | 1 | 40 | 1219 | 1219 | 1 | 0.1290401 | 0 |
| AGATG | | | | | | | | | |
| TCTTT | | | | | | | | | |
| GGAAA | | | | | | | | | |
| (SEQ | | | | | | | | | |
| ID | | | | | | | | | |
| NO: | | | | | | | | | |
| 380) | | | | | | | | | |
| CTTGT | 1217 | 1217 | 1 | 45 | 1217 | 1217 | 1 | 0.34655682 | 0 |
| TGTGG | | | | | | | | | |
| AATGT | | | | | | | | | |
| GCAAG | | | | | | | | | |
| (SEQ | | | | | | | | | |
| ID | | | | | | | | | |
| NO: | | | | | | | | | |
| 381) | | | | | | | | | |
| TTCCA | 1214 | 1214 | 1 | 45 | 1214 | 1214 | 1 | 0.42141996 | 0 |
| AAGAC | | | | | | | | | |
| ATCTT | | | | | | | | | |
| CGGAG | | | | | | | | | |
| (SEQ | | | | | | | | | |
| ID | | | | | | | | | |
| NO: | | | | | | | | | |
| 382) | | | | | | | | | |
| GTTTG | 1207 | 1207 | 1 | 40 | 1207 | 1207 | 1 | 0.17114529 | 0 |
| GAAAC | | | | | | | | | |
| ACTCT | | | | | | | | | |
| TGTTG | | | | | | | | | |
| (SEQ | | | | | | | | | |
| ID | | | | | | | | | |
| NO: | | | | | | | | | |
| 383) | | | | | | | | | |
| TTCTA | 1185 | 1185 | 1 | 40 | 1185 | 1185 | 1 | 0.2434113 | 0 |
| AACTG | | | | | | | | | |
| CTACA | | | | | | | | | |
| TCGCA | | | | | | | | | |
| (SEQ | | | | | | | | | |
| ID | | | | | | | | | |
| NO: | | | | | | | | | |
| 384) | | | | | | | | | |
| TCTGT | 1110 | 1110 | 1 | 45 | 1110 | 1110 | 1 | 0.52384293 | 0 |
| GTCCT | | | | | | | | | |
| TCGTT | | | | | | | | | |
| CGAAA | | | | | | | | | |
| (SEQ | | | | | | | | | |
| ID | | | | | | | | | |
| NO: | | | | | | | | | |
| 385) | | | | | | | | | |
| TTCGA | 1109 | 1109 | 1 | 50 | 1109 | 1109 | 1 | 0.52800048 | 0 |
| ACGAA | | | | | | | | | |
| GGACA | | | | | | | | | |
| CAGAG | | | | | | | | | |
| (SEQ | | | | | | | | | |
| ID | | | | | | | | | |
| NO: | | | | | | | | | |
| 386) | | | | | | | | | |
| CACAG | 1102 | 1102 | 1 | 50 | 1105 | 1105 | 0.99728507 | 0.18227416 | 0 |
| AGTTG | | | | | | | | | |
| AACCC | | | | | | | | | |
| TCCTA | | | | | | | | | |
| (SEQ | | | | | | | | | |
| ID | | | | | | | | | |
| NO: | | | | | | | | | |
| 387) | | | | | | | | | |
| CTGTG | 1093 | 1093 | 1 | 50 | 1093 | 1093 | 1 | 0.14031832 | 0 |
| TCCTT | | | | | | | | | |
| CGTTC | | | | | | | | | |
| GAAAC | | | | | | | | | |
| (SEQ | | | | | | | | | |
| ID | | | | | | | | | |
| NO: | | | | | | | | | |
| 388) | | | | | | | | | |
| TTCAA | 1089 | 1089 | 1 | 40 | 1089 | 1089 | 1 | 0.17363557 | 0 |
| CACTG | | | | | | | | | |
| CTCTA | | | | | | | | | |
| TCCAT | | | | | | | | | |
| (SEQ | | | | | | | | | |
| ID | | | | | | | | | |
| NO: | | | | | | | | | |
| 389) | | | | | | | | | |
| ACACT | 1077 | 1077 | 1 | 45 | 1077 | 1077 | 1 | 0.64887663 | 0 |
| GCTCT | | | | | | | | | |
| ATCCA | | | | | | | | | |
| TAGGA | | | | | | | | | |
| (SEQ | | | | | | | | | |
| ID | | | | | | | | | |
| NO: | | | | | | | | | |
| 390) | | | | | | | | | |
| AACAC | 1076 | 1076 | 1 | 45 | 1076 | 1076 | 1 | 0.33125897 | 0 |
| TGCTC | | | | | | | | | |
| TATCC | | | | | | | | | |
| ATAGG | | | | | | | | | |
| (SEQ | | | | | | | | | |
| ID | | | | | | | | | |
| NO: | | | | | | | | | |
| 391) | | | | | | | | | |
| TAGAT | 982 | 988 | 0.994 | 40 | 982 | 989 | 0.99292214 | 0.2493118 | 0 |
| ATTTG | | | | | | | | | |
| GACCT | | | | | | | | | |
| CTCTG | | | | | | | | | |
| (SEQ | | | | | | | | | |
| ID | | | | | | | | | |
| NO: | | | | | | | | | |
| 392) | | | | | | | | | |
| CTTTT | 831 | 831 | 1 | 40 | 831 | 831 | 1 | 0.6531611 | 0 |
| CGTAG | | | | | | | | | |
| TGTCT | | | | | | | | | |
| ACAAG | | | | | | | | | |
| (SEQ | | | | | | | | | |
| ID | | | | | | | | | |
| NO: | | | | | | | | | |
| 393) | | | | | | | | | |
| TTTGA | 818 | 818 | 1 | 40 | 818 | 818 | 1 | 0.24303259 | 0 |
| GGAGT | | | | | | | | | |
| ACCGT | | | | | | | | | |
| AGTAA | | | | | | | | | |
| (SEQ | | | | | | | | | |
| ID | | | | | | | | | |
| NO: | | | | | | | | | |
| 394) | | | | | | | | | |
| TGAAA | 793 | 793 | 1 | 40 | 793 | 793 | 1 | 0.24870862 | 0 |
| GGAAA | | | | | | | | | |
| GTTCA | | | | | | | | | |
| ACTCG | | | | | | | | | |
| (SEQ | | | | | | | | | |
| ID | | | | | | | | | |
| NO: | | | | | | | | | |
| 395) | | | | | | | | | |
| GACCT | 790 | 790 | 1 | 50 | 790 | 790 | 1 | 0.63351423 | 0 |
| CTGTG | | | | | | | | | |
| AGGAA | | | | | | | | | |
| TTCGT | | | | | | | | | |
| (SEQ | | | | | | | | | |
| ID | | | | | | | | | |
| NO: | | | | | | | | | |
| 396) | | | | | | | | | |
| GGAAA | 789 | 789 | 1 | 50 | 789 | 789 | 1 | 0.14498785 | 0 |
| CGGGA | | | | | | | | | |
| GAATC | | | | | | | | | |
| TTCAC | | | | | | | | | |
| (SEQ | | | | | | | | | |
| ID | | | | | | | | | |
| NO: | | | | | | | | | |
| 397) | | | | | | | | | |
| TTCCA | 789 | 789 | 1 | 45 | 789 | 789 | 1 | 0.56960046 | 0 |
| ACGAA | | | | | | | | | |
| TTCCT | | | | | | | | | |
| CACAG | | | | | | | | | |
| (SEQ | | | | | | | | | |
| ID | | | | | | | | | |
| NO: | | | | | | | | | |
| 398) | | | | | | | | | |
| GTGAG | 787 | 787 | 1 | 45 | 787 | 787 | 1 | 0.1004144 | 0 |
| GAATT | | | | | | | | | |
| CGTTG | | | | | | | | | |
| GAAAC | | | | | | | | | |
| (SEQ | | | | | | | | | |
| ID | | | | | | | | | |
| NO: | | | | | | | | | |
| 399) | | | | | | | | | |
| TGTGA | 786 | 786 | 1 | 40 | 786 | 786 | 1 | 0.19120266 | 0 |
| GGAAT | | | | | | | | | |
| TCGTT | | | | | | | | | |
| GGAAA | | | | | | | | | |
| (SEQ | | | | | | | | | |
| ID | | | | | | | | | |
| NO: | | | | | | | | | |
| 400) | | | | | | | | | |
| TGCAT | 785 | 785 | 1 | 45 | 785 | 785 | 1 | 0.20720756 | 0 |
| ATTTG | | | | | | | | | |
| GACCT | | | | | | | | | |
| CTGTG | | | | | | | | | |
| (SEQ | | | | | | | | | |
| ID | | | | | | | | | |
| NO: | | | | | | | | | |
| 401) | | | | | | | | | |
| TGGAT | 778 | 778 | 1 | 45 | 779 | 779 | 0.9987163 | 0.18322258 | 0 |
| ATTTG | | | | | | | | | |
| GTCCT | | | | | | | | | |
| CTCTG | | | | | | | | | |
| (SEQ | | | | | | | | | |
| ID | | | | | | | | | |
| NO: | | | | | | | | | |
| 402) | | | | | | | | | |
| TCATC | 762 | 762 | 1 | 45 | 762 | 762 | 1 | 0.18636349 | 0 |
| ACAGA | | | | | | | | | |
| GAAGC | | | | | | | | | |
| TTCTG | | | | | | | | | |
| (SEQ | | | | | | | | | |
| ID | | | | | | | | | |
| NO: | | | | | | | | | |
| 403) | | | | | | | | | |
| GGGGA | 731 | 731 | 1 | 45 | 731 | 731 | 1 | 0.12534446 | 0 |
| TAATT | | | | | | | | | |
| GCACT | | | | | | | | | |
| CTTTG | | | | | | | | | |
| (SEQ | | | | | | | | | |
| ID | | | | | | | | | |
| NO: | | | | | | | | | |
| 404) | | | | | | | | | |
| GTTTC | 692 | 692 | 1 | 40 | 692 | 692 | 1 | 0.25115033 | 0 |
| CAATC | | | | | | | | | |
| ACTCT | | | | | | | | | |
| TTCTG | | | | | | | | | |
| (SEQ | | | | | | | | | |
| ID | | | | | | | | | |
| NO: | | | | | | | | | |
| 405) | | | | | | | | | |
| GATTC | 669 | 669 | 1 | 40 | 669 | 669 | 1 | 0.58173194 | 0 |
| CACAG | | | | | | | | | |
| AAAGA | | | | | | | | | |
| GTGAT | | | | | | | | | |
| (SEQ | | | | | | | | | |
| ID | | | | | | | | | |
| NO: | | | | | | | | | |
| 406) | | | | | | | | | |
| TGGAT | 668 | 668 | 1 | 45 | 668 | 668 | 1 | 0.22992748 | 0 |
| ATTTA | | | | | | | | | |
| GGCCT | | | | | | | | | |
| CTCTG | | | | | | | | | |
| (SEQ | | | | | | | | | |
| ID | | | | | | | | | |
| NO: | | | | | | | | | |
| 407) | | | | | | | | | |
| TCTGA | 522 | 522 | 1 | 40 | 523 | 527 | 0.99051233 | 0.20687967 | 0 |
| GGAAT | | | | | | | | | |
| TCGTT | | | | | | | | | |
| GGAAA | | | | | | | | | |
| (SEQ | | | | | | | | | |
| ID | | | | | | | | | |
| NO: | | | | | | | | | |
| 408) | | | | | | | | | |
| TTCCA | 520 | 520 | 1 | 45 | 520 | 520 | 1 | 0.41439855 | 0 |
| ACGAA | | | | | | | | | |
| TTCCT | | | | | | | | | |
| CAGAG | | | | | | | | | |
| (SEQ | | | | | | | | | |
| ID | | | | | | | | | |
| NO: | | | | | | | | | |
| 409) | | | | | | | | | |
| TTTGA | 453 | 454 | 0.998 | 45 | 453 | 454 | 0.99779736 | 0.38523188 | 0 |
| GGCCT | | | | | | | | | |
| ACCGT | | | | | | | | | |
| AGTAA | | | | | | | | | |
| (SEQ | | | | | | | | | |
| ID | | | | | | | | | |
| NO: | | | | | | | | | |
| 410) | | | | | | | | | |
| GTCCT | 402 | 402 | 1 | 50 | 402 | 402 | 1 | 0.61333198 | 0 |
| CTCTG | | | | | | | | | |
| AGCAT | | | | | | | | | |
| TTCGT | | | | | | | | | |
| (SEQ | | | | | | | | | |
| ID | | | | | | | | | |
| NO: | | | | | | | | | |
| 411) | | | | | | | | | |

CHR18

| | | | | | | | | | bind- | | | |
|---|---|---|---|---|---|---|---|---|---|---|---|---|
| | bind- | | | | | | | | ing | | bind- | |
| | ing | | | | | | | | sites | | ing | |
| | sites | | | | | | | | (CHM13- | | sites | |
| | (CHM13- | bind- | | | | | | | spe- | | (CHM13- | |
| | spe- | ing | | | | | | | cif- | | spe- | |
| | cif- | sites | | | | | | | ic | | cif- | bind- |
| | ic | (CHM | | | bind- | | | | chro- | | ic | ing |
| | chro- | 13- | chro- | | ing | bind- | | | mo- | | chro- | sites |
| | mo- | all | mo- | | sites | ing | centro- | | some | | mo- | (CHM13- |
| | some | cen- | some | | (CHM- | sites | mere | | centro- | | some | all |
| sg | centro- | tro- | spe- | | 13 | (hg38 | spe- | Ac- | mere | | centro- | centro- |
| RNA | mere | mere | cif- | GC_ | whole | whole | cif- | tiv- | Not | sgRNA | mere | mere |
| _ | HOR_ | HOR_ | ic- | cont- | ge- | ge- | ic- | ity | HOR_ | _ | HOR_ | HOR_ |
| seq | L) | L) | ity | ent | nome) | nome) | ity | score | L) | seq | L) | L) |
| TTTCA | 4725 | 4725 | 1 | 40 | 4725 | 4725 | 1 | 0.44655237 | 0 | hor_ | hor_ | hor_ |
| AACCT | | | | | | | | | | 18_ | 18_ | 18_ |
| GCTCT | | | | | | | | | | 3 | 3 | 1 |
| ACCAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 412) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| ACAGA | 4701 | 4701 | 1 | 40 | 4701 | 4701 | 1 | 0.44212068 | 0 | hor_ | hor_ | hor_ |
| GTAGA | | | | | | | | | | 18_ | 18_ | 18_ |
| ACATT | | | | | | | | | | 3 | 3 | 1 |
| CCCTT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 62) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTCAA | 4643 | 4643 | 1 | 40 | 4643 | 4643 | 1 | 0.39833218 | 0 | hor_ | hor_ | hor_ |
| ACCTG | | | | | | | | | | 18_ | 18_ | 18_ |
| CTCTA | | | | | | | | | | 3 | 3 | 1 |
| CCAAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 413) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| AAACT | 4184 | 4188 | 0.999 | 45 | 4184 | 4188 | 0.99904489 | 0.71849454 | 0 | hor_ | hor_ | hor_ |
| GCTCC | | | | | | | | | | 18_ | 18_ | 18_ |
| TTCAA | | | | | | | | | | 3 | 3 | 1 |
| AACGG | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 414) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| GCTAG | 3531 | 3533 | 0.999 | 40 | 3531 | 3534 | 0.9991511 | 0.60665411 | 0 | hor_ | hor_ | hor_ |
| TTTTG | | | | | | | | | | 18_ | 18_ | 18_ |
| AGGAT | | | | | | | | | | 3 | 3 | 1 |
| TTCGT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 415) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTCCC | 2958 | 2958 | 1 | 45 | 2958 | 2958 | 1 | 0.46110367 | 0 | hor_ | hor_ | hor_ |
| TATCA | | | | | | | | | | 18_ | 18_ | 18_ |
| TAGAG | | | | | | | | | | 3 | 3 | 1 |
| CAGGT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 416) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| ATTCC | 2871 | 2871 | 1 | 40 | 2871 | 2871 | 1 | 0.31729118 | 0 | hor_ | hor_ | hor_ |
| AACCT | | | | | | | | | | 18_ | 18_ | 18_ |
| GCTCT | | | | | | | | | | 3 | 3 | 1 |
| ATGAT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 417) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTTCA | 2760 | 2760 | 1 | 40 | 2760 | 2766 | 0.9978308 | 0.25775686 | 0 | hor_ | hor_ | hor_ |
| GGCCT | | | | | | | | | | 18_ | 18_ | 18_ |
| ATGTT | | | | | | | | | | 3 | 3 | 1 |
| GGAAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 418) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TGCTT | 2472 | 2472 | 1 | 45 | 2472 | 2472 | 1 | 0.10303149 | 0 | hor_ | hor_ | hor_ |
| CTGCC | | | | | | | | | | 18_ | 18_ | 18_ |
| TAGTT | | | | | | | | | | 3 | 3 | 1 |
| GTTAC | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 419) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2- |
| | | | | | | | | | | | | A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTTCA | 2464 | 2464 | 1 | 45 | 2464 | 2464 | 1 | 0.33445306 | 0 | hor_ | hor_ | hor_ |
| GGCCT | | | | | | | | | | 18_ | 18_ | 18_ |
| ACGTT | | | | | | | | | | 3 | 3 | 1 |
| GGAAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 420) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| AGGTT | 2413 | 2413 | 1 | 40 | 2413 | 2413 | 1 | 0.17529222 | 0 | hor_ | hor_ | hor_ |
| CTACT | | | | | | | | | | 18_ | 18_ | 18_ |
| CCTTT | | | | | | | | | | 3 | 3 | 1 |
| AGTTG | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 421) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTTGA | 2360 | 2363 | 0.999 | 40 | 2360 | 2365 | 0.99788584 | 0.1082229 | 0 | hor_ | hor_ | hor_ |
| GGATT | | | | | | | | | | 18_ | 18_ | 18_ |
| TCGTG | | | | | | | | | | 3 | 3 | 1 |
| GGAAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 422) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| GCATA | 2338 | 2338 | 1 | 45 | 2338 | 2338 | 1 | 0.3837679 | 0 | hor_ | hor_ | hor_ |
| GCTTT | | | | | | | | | | 18_ | 18_ | 18_ |
| GAGGA | | | | | | | | | | 3 | 3 | 1 |
| TTTCG | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 423) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| GAGCG | 2234 | 2234 | 1 | 60 | 2234 | 2234 | 1 | 0.22998132 | 0 | hor_ | hor_ | hor_ |
| CTTTC | | | | | | | | | | 18_ | 18_ | 18_ |
| AGGCC | | | | | | | | | | 3 | 3 | 1 |
| TACGT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 424) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2- |
| | | | | | | | | | | | | D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| CCTAG | 2003 | 2003 | 1 | 50 | 2003 | 2003 | 1 | 0.52266406 | 0 | hor_ | hor_ | hor_ |
| CCTTG | | | | | | | | | | 18_ | 18_ | 18_ |
| AGGAT | | | | | | | | | | 3 | 3 | 1 |
| TTCGT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 425) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| CCAAC | 1995 | 1995 | 1 | 50 | 1995 | 1995 | 1 | 0.18331461 | 0 | hor_ | hor_ | hor_ |
| GAAAT | | | | | | | | | | 18_ | 18_ | 18_ |
| CCTCA | | | | | | | | | | 3 | 3 | 1 |
| AGGCT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 426) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTTCC | 1932 | 1932 | 1 | 40 | 1932 | 1936 | 0.99793388 | 0.19591543 | 0 | hor_ | hor_ | hor_ |
| TTTTT | | | | | | | | | | 18_ | 18_ | 18_ |
| CACCT | | | | | | | | | | 3 | 3 | 1 |
| TAGGC | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 427) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| GCTAG | 1832 | 1832 | 1 | 55 | 1832 | 1832 | 1 | 0.34289331 | 0 | hor_ | hor_ | hor_ |
| CTTTG | | | | | | | | | | 18_ | 18_ | 18_ |
| GGGAT | | | | | | | | | | 3 | 3 | 1 |
| TTCGC | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 428) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTTCA | 1829 | 1829 | 1 | 40 | 1829 | 1829 | 1 | 0.3272533 | 0 | hor_ | hor_ | hor_ |
| GGGCT | | | | | | | | | | 18_ | 18_ | 18_ |
| AAGGT | | | | | | | | | | 3 | 3 | 1 |
| GAAAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 429) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| AGTGG | 1810 | 1812 | 0.999 | 40 | 1810 | 1813 | 0.99834528 | 0.16281326 | 0 | hor_ | hor_ | hor_ |
| ATATT | | | | | | | | | | 18_ | 18_ | 18_ |
| TGGCT | | | | | | | | | | 3 | 3 | 1 |
| AGCTT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 430) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTTGG | 1717 | 1717 | 1 | 50 | 1717 | 1717 | 1 | 0.24902073 | 0 | hor_ | hor_ | hor_ |
| GGATT | | | | | | | | | | 18_ | 18_ | 18_ |
| TCGCT | | | | | | | | | | 3 | 3 | 1 |
| GGAAG | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 431) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| CCAGT | 1682 | 1682 | 1 | 40 | 1682 | 1682 | 1 | 0.59962433 | 0 | hor_ | hor_ | hor_ |
| TCCAG | | | | | | | | | | 18_ | 18_ | 18_ |
| ATACT | | | | | | | | | | 3 | 3 | 1 |
| ACAAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 432) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| CCTTT | 1681 | 1681 | 1 | 40 | 1681 | 1681 | 1 | 0.26403083 | 0 | hor_ | hor_ | hor_ |
| TGTAG | | | | | | | | | | 18_ | 18_ | 18_ |
| TATCT | | | | | | | | | | 3 | 3 | 1 |
| GGAAC | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 433) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| GGAAT | 1675 | 1678 | 0.998 | 45 | 1675 | 1679 | 0.99761763 | 0.10709911 | 0 | hor_ | hor_ | hor_ |
| CTGCA | | | | | | | | | | 18_ | 18_ | 18_ |
| AGTGG | | | | | | | | | | 3 | 3 | 1 |
| CTATT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 434) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TGGCT | 1658 | 1658 | 1 | 40 | 1658 | 1658 | 1 | 0.10658127 | 0 | hor_ | hor_ | hor_ |
| ATTTG | | | | | | | | | | 18_ | 18_ | 18_ |
| GCTAG | | | | | | | | | | 3 | 3 | 1 |
| ATTTG | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 435) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2- |
| | | | | | | | | | | | | A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTTCA | 1487 | 1487 | 1 | 45 | 1487 | 1487 | 1 | 0.29837788 | 0 | hor_ | hor_ | hor_ |
| GGCCC | | | | | | | | | | 18_ | 18_ | 18_ |
| ATGTT | | | | | | | | | | 3 | 3 | 1 |
| GGAAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 436) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| CTTTC | 1455 | 1455 | 1 | 50 | 1455 | 1455 | 1 | 0.16172013 | 0 | hor_ | hor_ | hor_ |
| AGGCC | | | | | | | | | | 18_ | 18_ | 18_ |
| CATGT | | | | | | | | | | 3 | 3 | 1 |
| TGGAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 437) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| AGTAT | 1343 | 1343 | 1 | 40 | 1343 | 1343 | 1 | 0.19078188 | 0 | hor_ | hor_ | hor_ |
| ATTTG | | | | | | | | | | 18_ | 18_ | 18_ |
| CCTAG | | | | | | | | | | 3 | 3 | 1 |
| CCTTG | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 438) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTGGA | 973 | 973 | 1 | 50 | 973 | 973 | 1 | 0.10950023 | 0 | hor_ | hor_ | hor_ |
| GCGAT | | | | | | | | | | 18_ | 18_ | 18_ |
| TTCAG | | | | | | | | | | 3 | 3 | 1 |
| GGCTA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 439) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| GGACT | 965 | 965 | 1 | 45 | 965 | 965 | 1 | 0.2684193 | 0 | hor_ | hor_ | hor_ |
| TTTGG | | | | | | | | | | 18_ | 18_ | 18_ |
| AGCGA | | | | | | | | | | 3 | 3 | 1 |
| TTTCA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 440) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2- |
| | | | | | | | | | | | | D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| CTTTC | 960 | 960 | 1 | 40 | 960 | 960 | 1 | 0.36339012 | 0 | hor_ | hor_ | hor_ |
| AGGCC | | | | | | | | | | 18_ | 18_ | 18_ |
| TATTT | | | | | | | | | | 3 | 3 | 1 |
| TGGAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 441) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTACC | 947 | 947 | 1 | 45 | 947 | 947 | 1 | 0.56035903 | 0 | hor_ | hor_ | hor_ |
| GGCCT | | | | | | | | | | 18_ | 18_ | 18_ |
| AAGGT | | | | | | | | | | 3 | 3 | 1 |
| GAAAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 442) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TGGAC | 930 | 930 | 1 | 45 | 930 | 930 | 1 | 0.10848833 | 0 | hor_ | hor_ | hor_ |
| ATTTG | | | | | | | | | | 18_ | 18_ | 18_ |
| GAGCA | | | | | | | | | | 3 | 3 | 1 |
| CTTAC | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 443) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| CTTTC | 909 | 909 | 1 | 45 | 909 | 909 | 1 | 0.13772721 | 0 | hor_ | hor_ | hor_ |
| AGGCC | | | | | | | | | | 18_ | 18_ | 18_ |
| TATGT | | | | | | | | | | 3 | 3 | 1 |
| TGGAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 444) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTCAG | 757 | 757 | 1 | 40 | 757 | 757 | 1 | 0.11520707 | 0 | hor_ | hor_ | hor_ |
| GACTG | | | | | | | | | | 18_ | 18_ | 18_ |
| CTCTA | | | | | | | | | | 3 | 3 | 1 |
| TGAAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 445) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TTTCA | 757 | 757 | 1 | 40 | 757 | 757 | 1 | 0.30380466 | 0 | hor_ | hor_ | hor_ |
| GGACT | | | | | | | | | | 18_ | 18_ | 18_ |
| GCTCT | | | | | | | | | | 3 | 3 | 1 |
| ATGAA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 446) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| AGGAT | 607 | 607 | 1 | 45 | 607 | 607 | 1 | 0.16306791 | 0 | hor_ | hor_ | hor_ |
| ATTTG | | | | | | | | | | 18_ | 18_ | 18_ |
| CCTAG | | | | | | | | | | 3 | 3 | 1 |
| CCTTG | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 60) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| TGGAT | 606 | 606 | 1 | 40 | 606 | 606 | 1 | 0.21358543 | 0 | hor_ | hor_ | hor_ |
| ATTTG | | | | | | | | | | 18_ | 18_ | 18_ |
| GCTAG | | | | | | | | | | 3 | 3 | 1 |
| TTTGG | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 447) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| GCTAG | 594 | 594 | 1 | 45 | 594 | 594 | 1 | 0.70851972 | 0 | hor_ | hor_ | hor_ |
| TTTGG | | | | | | | | | | 18_ | 18_ | 18_ |
| AGGAT | | | | | | | | | | 3 | 3 | 1 |
| TTCGT | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 448) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |
| GGACT | 590 | 590 | 1 | 50 | 590 | 590 | 1 | 0.17756767 | 0 | hor_ | hor_ | hor_ |
| TTTGG | | | | | | | | | | 18_ | 18_ | 18_ |
| AGCGC | | | | | | | | | | 3 | 3 | 1 |
| TTTCA | | | | | | | | | | (S2C1 | (S2C1 | (S2C1 |
| (SEQ | | | | | | | | | | 8H1L) | 8H1L) | 8pH2-A); |
| ID | | | | | | | | | | | | hor_ |
| NO: | | | | | | | | | | | | 18_ |
| 449) | | | | | | | | | | | | 2 |
| | | | | | | | | | | | | (S2C1 |
| | | | | | | | | | | | | 8qH2-B; |
| | | | | | | | | | | | | S2C1 |
| | | | | | | | | | | | | 8pH2-A); |
| | | | | | | | | | | | | hor_ |
| | | | | | | | | | | | | 18_ |
| | | | | | | | | | | | | 3 |
| | | | | | | | | | | | | (S2C1 |
| | | | | | | | | | | | | 8H1L); |
| | | | | | | | | | | | | hor_ |
| | | | | | | | | | | | | 18_ |
| | | | | | | | | | | | | 4 |
| | | | | | | | | | | | | (S2C1 |
| | | | | | | | | | | | | 8qH2-D); |
| | | | | | | | | | | | | hor_ |
| | | | | | | | | | | | | 18_ |
| | | | | | | | | | | | | 5 |
| | | | | | | | | | | | | (S2C1 |
| | | | | | | | | | | | | 8qH2-B, |
| | | | | | | | | | | | | S2C1 |
| | | | | | | | | | | | | 8qH2-E) |

CHR19

| | | | | | | | | | bind- |
|---|---|---|---|---|---|---|---|---|---|
| | bind- | | | | | | | | ing |
| | ing | | | | | | | | sites |
| | sites | | | | | | | | (CHM13- |
| | (CHM13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| CTTGA | 2527 | 2533 | 0.998 | 45 | 2527 | 2535 | 0.99684418 | 0.27382686 | 0 |
| GGCTT | | | | | | | | | |
| TCGTT | | | | | | | | | |
| GGAAA | | | | | | | | | |
| (SEQ | | | | | | | | | |
| ID | | | | | | | | | |
| NO: | | | | | | | | | |
| 450) | | | | | | | | | |
| TGGAT | 2466 | 2469 | 0.999 | 40 | 2466 | 2469 | 0.99878493 | 0.63196035 | 0 |
| ATTCA | | | | | | | | | |
| GACAT | | | | | | | | | |
| CCTTG | | | | | | | | | |
| (SEQ | | | | | | | | | |
| ID | | | | | | | | | |
| NO: | | | | | | | | | |
| 451) | | | | | | | | | |
| CGTTT | 2433 | 2439 | 0.998 | 50 | 2433 | 2441 | 0.99672265 | 0.37463603 | 0 |
| CCAAC | | | | | | | | | |
| GAAAG | | | | | | | | | |
| CCTCA | | | | | | | | | |
| (SEQ | | | | | | | | | |
| ID | | | | | | | | | |
| NO: | | | | | | | | | |
| 452) | | | | | | | | | |
| GACAT | 2383 | 2383 | 1 | 50 | 2383 | 2383 | 1 | 0.35195982 | 0 |
| CCTTG | | | | | | | | | |
| AGGCT | | | | | | | | | |
| TTCGT | | | | | | | | | |
| (SEQ | | | | | | | | | |
| ID | | | | | | | | | |
| NO: | | | | | | | | | |
| 63) | | | | | | | | | |
| TTCAG | 1733 | 1739 | 0.997 | 45 | 1733 | 1739 | 0.99654974 | 0.29010838 | 0 |
| CCGCT | | | | | | | | | |
| TTGAG | | | | | | | | | |
| TTCAA | | | | | | | | | |
| (SEQ | | | | | | | | | |
| ID | | | | | | | | | |
| NO: | | | | | | | | | |
| 453) | | | | | | | | | |
| GACAT | 440 | 440 | 1 | 45 | 440 | 443 | 0.99322799 | 0.30723848 | 0 |
| CTTTG | | | | | | | | | |
| AGGCT | | | | | | | | | |
| TTCGT | | | | | | | | | |
| (SEQ | | | | | | | | | |
| ID | | | | | | | | | |
| NO: | | | | | | | | | |
| 454) | | | | | | | | | |

CHR20

| | | | | | | | | | bind- |
|---|---|---|---|---|---|---|---|---|---|
| | bind- | | | | | | | | ing |
| | ing | | | | | | | | sites |
| | sites | | | | | | | | (CHM13- |
| | (CHM13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| AAACT | 1525 | 1525 | 1 | 40 | 1525 | 1525 | 1 | 0.61193758 | 0 |
| GCTCC | | | | | | | | | |
| TTCAA | | | | | | | | | |
| AACGA | | | | | | | | | |
| (SEQ | | | | | | | | | |
| ID | | | | | | | | | |
| NO: | | | | | | | | | |
| 64) | | | | | | | | | |
| TGGAT | \|791 | 791 | 1 | 45 | 791 | 791 | 1 | 0.37557712 | 0 |
| ATTAG | | | | | | | | | |
| GGCAG | | | | | | | | | |
| CTTTG | | | | | | | | | |
| (SEQ | | | | | | | | | |
| ID | | | | | | | | | |
| NO: | | | | | | | | | |
| 455) | | | | | | | | | |
| GGCAG | \|790 | 790 | 1 | 50 | 790 | 791 | 0.99873578 | 0.42529344 | 0 |
| CTTTG | | | | | | | | | |
| AGGAT | | | | | | | | | |
| TTCGT | | | | | | | | | |
| (SEQ | | | | | | | | | |
| ID | | | | | | | | | |
| NO: | | | | | | | | | |
| 66) | | | | | | | | | |
| GTTTT | 776 | 776 | 1 | 45 | 776 | 776 | 1 | 0.71990647 | 0 |
| CGTGG | | | | | | | | | |
| AATCT | | | | | | | | | |
| GCAAG | | | | | | | | | |
| (SEQ | | | | | | | | | |
| ID | | | | | | | | | |
| NO: | | | | | | | | | |
| 456) | | | | | | | | | |
| AGGTT | 772 | 772 | 1 | 45 | 772 | 772 | 1 | 0.10009128 | 0 |
| CAACA | | | | | | | | | |
| CTGTC | | | | | | | | | |
| AGTTG | | | | | | | | | |
| (SEQ | | | | | | | | | |
| ID | | | | | | | | | |
| NO: | | | | | | | | | |
| 457) | | | | | | | | | |
| GGTTC | \|772 | 772 | 1 | 45 | 772 | 772 | 1 | 0.37322806 | 0 |
| AACAC | | | | | | | | | |
| TGTCA | | | | | | | | | |
| GTTGA | | | | | | | | | |
| (SEQ | | | | | | | | | |
| ID | | | | | | | | | |
| NO: | | | | | | | | | |
| 67) | | | | | | | | | |
| TTTCA | 768 | 768 | 1 | 40 | 768 | 768 | 1 | 0.35069654 | 0 |
| GGTCT | | | | | | | | | |
| ACGGT | | | | | | | | | |
| GAAAA | | | | | | | | | |
| (SEQ | | | | | | | | | |
| ID | | | | | | | | | |
| NO: | | | | | | | | | |
| 458) | | | | | | | | | |
| TTGGA | 763 | 763 | 1 | 55 | 763 | 763 | 1 | 0.23198674 | 0 |
| GCGCT | | | | | | | | | |
| TTCAG | | | | | | | | | |
| GACGA | | | | | | | | | |
| (SEQ | | | | | | | | | |
| ID | | | | | | | | | |
| NO: | | | | | | | | | |
| 68) | | | | | | | | | |
| TTCAA | 760 | 760 | 1 | 40 | 760 | 761 | 0.99868594 | 0.12480639 | 0 |
| ACCTG | | | | | | | | | |
| CTCTC | | | | | | | | | |
| TCAAA | | | | | | | | | |
| (SEQ | | | | | | | | | |
| ID | | | | | | | | | |
| NO: | | | | | | | | | |
| 459) | | | | | | | | | |
| AACAT | 758 | 758 | 1 | 45 | 758 | 758 | 1 | 0.10870723 | 0 |
| TCCCT | | | | | | | | | |
| TTGAG | | | | | | | | | |
| AGAGC | | | | | | | | | |
| (SEQ | | | | | | | | | |
| ID | | | | | | | | | |
| NO: | | | | | | | | | |
| 577) | | | | | | | | | |
| TTTCA | 757 | 757 | 1 | 40 | 757 | 759 | 0.99736495 | 0.51196277 | 0 |
| AACCT | | | | | | | | | |
| GCTCT | | | | | | | | | |
| CTCAA | | | | | | | | | |
| (SEQ | | | | | | | | | |
| ID | | | | | | | | | |
| NO: | | | | | | | | | |
| 460) | | | | | | | | | |
| TTTCA | 755 | 755 | 1 | 45 | 755 | 755 | 1 | 0.35162575 | 0 |
| GGACG | | | | | | | | | |
| ACGGT | | | | | | | | | |
| GAAAA | | | | | | | | | |
| (SEQ | | | | | | | | | |
| ID | | | | | | | | | |
| NO: | | | | | | | | | |
| 461) | | | | | | | | | |
| AGCAT | 738 | 741 | 0.996 | 40 | 738 | 741 | 0.99595142 | 0.33614619 | 0 |
| TCTCA | | | | | | | | | |
| GAAAC | | | | | | | | | |
| TTCGT | | | | | | | | | |
| (SEQ | | | | | | | | | |
| ID | | | | | | | | | |
| NO: | | | | | | | | | |
| 462) | | | | | | | | | |
| GCATT | \|734 | 737 | 0.996 | 40 | 734 | 737 | 0.99592944 | 0.66927838 | 0 |
| CTCAG | | | | | | | | | |
| AAACT | | | | | | | | | |
| TCGTT | | | | | | | | | |
| (SEQ | | | | | | | | | |
| ID | | | | | | | | | |
| NO: | | | | | | | | | |
| 69) | | | | | | | | | |
| TGGGT | 731 | 731 | 1 | 55 | 731 | 731 | 1 | 0.4488897 | 0 |
| ATTAG | | | | | | | | | |
| GCCAG | | | | | | | | | |
| CTTGG | | | | | | | | | |
| (SEQ | | | | | | | | | |
| ID | | | | | | | | | |
| NO: | | | | | | | | | |
| 463) | | | | | | | | | |
| AAGTG | 728 | 728 | 1 | 50 | 728 | 728 | 1 | 0.26794908 | 0 |
| GGTAT | | | | | | | | | |
| TAGGC | | | | | | | | | |
| CAGCT | | | | | | | | | |
| (SEQ | | | | | | | | | |
| ID | | | | | | | | | |
| NO: | | | | | | | | | |
| 464) | | | | | | | | | |
| CTTTC | 713 | 713 | 1 | 50 | 713 | 713 | 1 | 0.33984173 | 0 |
| TGCAT | | | | | | | | | |
| TCCCT | | | | | | | | | |
| GGAAG | | | | | | | | | |
| (SEQ | | | | | | | | | |
| ID | | | | | | | | | |
| NO: | | | | | | | | | |
| 465) | | | | | | | | | |
| GATTT | 709 | 709 | 1 | 45 | 709 | 709 | 1 | 0.1950213 | 0 |
| CGTTG | | | | | | | | | |
| CAAAC | | | | | | | | | |
| GGGAA | | | | | | | | | |
| (SEQ | | | | | | | | | |
| ID | | | | | | | | | |
| NO: | | | | | | | | | |
| 466) | | | | | | | | | |
| AAGCT | 700 | 700 | 1 | 40 | 700 | 700 | 1 | 0.43774024 | 0 |
| GCTCT | | | | | | | | | |
| TTGCA | | | | | | | | | |
| AAGAA | | | | | | | | | |
| (SEQ | | | | | | | | | |
| ID | | | | | | | | | |
| NO: | | | | | | | | | |
| 467) | | | | | | | | | |
| TTCCC | 690 | 691 | 0.999 | 40 | 690 | 691 | 0.99855282 | 0.19128243 | 0 |
| TTTTA | | | | | | | | | |
| TAGAG | | | | | | | | | |
| CAGGT | | | | | | | | | |
| (SEQ | | | | | | | | | |
| ID | | | | | | | | | |
| NO: | | | | | | | | | |
| 468) | | | | | | | | | |
| AAGTG | 689 | 689 | 1 | 40 | 689 | 689 | 1 | 0.11679092 | 0 |
| GATAT | | | | | | | | | |
| TTGGC | | | | | | | | | |
| TAGCT | | | | | | | | | |
| (SEQ | | | | | | | | | |
| ID | | | | | | | | | |
| NO: | | | | | | | | | |
| 469) | | | | | | | | | |
| TTTCA | 685 | 686 | 0.999 | 40 | 686 | 687 | 0.99708879 | 0.36700474 | 0 |
| GGCCT | | | | | | | | | |
| AACGT | | | | | | | | | |
| GAAAA | | | | | | | | | |
| (SEQ | | | | | | | | | |
| ID | | | | | | | | | |
| NO: | | | | | | | | | |
| 470) | | | | | | | | | |
| TGGAT | 665 | 665 | 1 | 50 | 665 | 665 | 1 | 0.42230431 | 0 |
| ATTTG | | | | | | | | | |
| GCTAG | | | | | | | | | |
| CTGGG | | | | | | | | | |
| (SEQ | | | | | | | | | |
| ID | | | | | | | | | |
| NO: | | | | | | | | | |
| 71) | | | | | | | | | |
| TTTCA | 664 | 666 | 0.997 | 40 | 664 | 668 | 0.99401198 | 0.21768659 | 0 |
| GGCGT | | | | | | | | | |
| ATGGT | | | | | | | | | |
| GAAAA | | | | | | | | | |
| (SEQ | | | | | | | | | |
| ID | | | | | | | | | |
| NO: | | | | | | | | | |
| 471) | | | | | | | | | |
| GCTAG | 662 | 662 | 1 | 55 | 662 | 662 | 1 | 0.3560239 | 0 |
| CTGGG | | | | | | | | | |
| AGGAT | | | | | | | | | |
| TTCGT | | | | | | | | | |
| (SEQ | | | | | | | | | |
| ID | | | | | | | | | |
| NO: | | | | | | | | | |
| 472) | | | | | | | | | |
| GGGAG | 662 | 666 | 0.994 | 50 | 662 | 668 | 0.99101796 | 0.1080537 | 0 |
| GATTT | | | | | | | | | |
| CGTTG | | | | | | | | | |
| GAAAC | | | | | | | | | |
| (SEQ | | | | | | | | | |
| ID | | | | | | | | | |
| NO: | | | | | | | | | |
| 473) | | | | | | | | | |
| TGGGA | 647 | 651 | 0.994 | 45 | 647 | 651 | 0.99385561 | 0.10681745 | 0 |
| GGATT | | | | | | | | | |
| TCGTT | | | | | | | | | |
| GGAAA | | | | | | | | | |
| (SEQ | | | | | | | | | |
| ID | | | | | | | | | |
| NO: | | | | | | | | | |
| 474) | | | | | | | | | |
| GATGT | 549 | 549 | 1 | 40 | 549 | 549 | 1 | 0.15567912 | 0 |
| GTTTG | | | | | | | | | |
| CTCAA | | | | | | | | | |
| CTAAC | | | | | | | | | |
| (SEQ | | | | | | | | | |
| ID | | | | | | | | | |
| NO: | | | | | | | | | |
| 475) | | | | | | | | | |
| GGATT | 548 | 548 | 1 | 40 | 548 | 548 | 1 | 0.17051121 | 0 |
| GAACC | | | | | | | | | |
| ATCGT | | | | | | | | | |
| TTTGA | | | | | | | | | |
| (SEQ | | | | | | | | | |
| ID | | | | | | | | | |
| NO: | | | | | | | | | |
| 476) | | | | | | | | | |

CHR20

| | | | | | | | | | bind- |
|---|---|---|---|---|---|---|---|---|---|
| | bind- | | | | | | | | ing |
| | ing | | | | | | | | sites |
| | sites | | | | | | | | (CHM13- |
| | (CHM13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| GCAAT | 148 | 148 | 1 | 40 | 148 | 148 | 1 | 0.13095425 | 0 |
| TTGGA | | | | | | | | | |
| AACAC | | | | | | | | | |
| CCTTT | | | | | | | | | |
| (SEQ | | | | | | | | | |
| ID | | | | | | | | | |
| NO: | | | | | | | | | |
| 477) | | | | | | | | | |
| GACGT | 123 | 123 | 1 | 45 | 123 | 123 | 1 | 0.31009197 | 0 |
| TCCCT | | | | | | | | | |
| TTTTC | | | | | | | | | |
| ACCAA | | | | | | | | | |
| (SEQ | | | | | | | | | |
| ID | | | | | | | | | |
| NO: | | | | | | | | | |
| 73) | | | | | | | | | |
| CGTTC | 114 | 114 | 1 | 50 | 114 | 114 | 1 | 0.24060529 | 0 |
| TGGAG | | | | | | | | | |
| TATCT | | | | | | | | | |
| GGATG | | | | | | | | | |
| (SEQ | | | | | | | | | |
| ID | | | | | | | | | |
| NO: | | | | | | | | | |
| 478) | | | | | | | | | |
| ACTCT | 78 | 78 | 1 | 40 | 78 | 78 | 1 | 0.1606536 | 0 |
| TGTTG | | | | | | | | | |
| TGGAA | | | | | | | | | |
| AATGC | | | | | | | | | |
| (SEQ | | | | | | | | | |
| ID | | | | | | | | | |
| NO: | | | | | | | | | |
| 479) | | | | | | | | | |
| CTTGT | 78 | 78 | 1 | 45 | 78 | 78 | 1 | 0.71899628 | 0 |
| TGTGG | | | | | | | | | |
| AAAAT | | | | | | | | | |
| GCAGG | | | | | | | | | |
| (SEQ | | | | | | | | | |
| ID | | | | | | | | | |
| NO: | | | | | | | | | |
| 480) | | | | | | | | | |
| CTAGC | 42 | 42 | 1 | 45 | 42 | 42 | 1 | 0.16696786 | 0 |
| GATTT | | | | | | | | | |
| CGTTG | | | | | | | | | |
| GAAAC | | | | | | | | | |
| (SEQ | | | | | | | | | |
| ID | | | | | | | | | |
| NO: | | | | | | | | | |
| 481) | | | | | | | | | |
| GATAG | 42 | 42 | 1 | 45 | 42 | 42 | 1 | 0.47316073 | 0 |
| CTCTA | | | | | | | | | |
| GCGAT | | | | | | | | | |
| TTCGT | | | | | | | | | |
| (SEQ | | | | | | | | | |
| ID | | | | | | | | | |
| NO: | | | | | | | | | |
| 482) | | | | | | | | | |
| TCTAG | 42 | 42 | 1 | 40 | 42 | 42 | 1 | 0.20687967 | 0 |
| CGATT | | | | | | | | | |
| TCGTT | | | | | | | | | |
| GGAAA | | | | | | | | | |
| (SEQ | | | | | | | | | |
| ID | | | | | | | | | |
| NO: | | | | | | | | | |
| 483) | | | | | | | | | |
| TGTGT | 40 | 40 | 1 | 50 | 40 | 40 | 1 | 0.81440901 | 0 |
| ACTCG | | | | | | | | | |
| GCTAA | | | | | | | | | |
| CAGAG | | | | | | | | | |
| (SEQ | | | | | | | | | |
| ID | | | | | | | | | |
| NO: | | | | | | | | | |
| 484) | | | | | | | | | |

CHR22

| | | | | | | | | | bind- |
|---|---|---|---|---|---|---|---|---|---|
| | bind- | | | | | | | | ing |
| | ing | | | | | | | | sites |
| | sites | | | | | | | | (CHM13- |
| | (CHM13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| CGTTT | 235 | 235 | 1 | 45 | 235 | 235 | 1 | 0.34456061 | 0 |
| CAAAG | | | | | | | | | |
| AGCAG | | | | | | | | | |
| CTTTG | | | | | | | | | |
| (SEQ | | | | | | | | | |
| ID | | | | | | | | | |
| NO: | | | | | | | | | |
| 485) | | | | | | | | | |
| GCAGG | 177 | 177 | 1 | 45 | 177 | 177 | 1 | 0.16063784 | 0 |
| TTTGA | | | | | | | | | |
| AACGC | | | | | | | | | |
| TCTTT | | | | | | | | | |
| (SEQ | | | | | | | | | |
| ID | | | | | | | | | |
| NO: | | | | | | | | | |
| 486) | | | | | | | | | |
| AGAGT | 143 | 143 | 1 | 45 | 143 | 143 | 1 | 0.12349778 | 0 |
| GTATC | | | | | | | | | |
| CAAAC | | | | | | | | | |
| TGCTC | | | | | | | | | |
| (SEQ | | | | | | | | | |
| ID | | | | | | | | | |
| NO: | | | | | | | | | |
| 487) | | | | | | | | | |
| TTCCT | 143 | 143 | 1 | 45 | 143 | 143 | 1 | 0.17609664 | 0 |
| TTTGC | | | | | | | | | |
| CAGAG | | | | | | | | | |
| CAGTT | | | | | | | | | |
| (SEQ | | | | | | | | | |
| ID | | | | | | | | | |
| NO: | | | | | | | | | |
| 488) | | | | | | | | | |
| CGTTT | 108 | 108 | 1 | 55 | 108 | 108 | 1 | 0.61004275 | 0 |
| CAGAG | | | | | | | | | |
| AGCAG | | | | | | | | | |
| CTCTG | | | | | | | | | |
| (SEQ | | | | | | | | | |
| ID | | | | | | | | | |
| NO: | | | | | | | | | |
| 489) | | | | | | | | | |
| GGAGC | 63 | 63 | 1 | 65 | 63 | 63 | 1 | 0.83979326 | 0 |
| GCTCT | | | | | | | | | |
| GAGGT | | | | | | | | | |
| CTACG | | | | | | | | | |
| (SEQ | | | | | | | | | |
| ID | | | | | | | | | |
| NO: | | | | | | | | | |
| 490) | | | | | | | | | |
| TGGAG | 62 | 62 | 1 | 60 | 62 | 62 | 1 | 0.10242593 | 0 |
| CGCTC | | | | | | | | | |
| TGAGG | | | | | | | | | |
| TCTAC | | | | | | | | | |
| (SEQ | | | | | | | | | |
| ID | | | | | | | | | |
| NO: | | | | | | | | | |
| 491) | | | | | | | | | |
| GTGAA | 60 | 60 | 1 | 45 | 60 | 60 | 1 | 0.11364188 | 0 |
| CTCAG | | | | | | | | | |
| CTAAC | | | | | | | | | |
| AGATG | | | | | | | | | |
| (SEQ | | | | | | | | | |
| ID | | | | | | | | | |
| NO: | | | | | | | | | |
| 492) | | | | | | | | | |
| AAGTG | 59 | 59 | 1 | 50 | 59 | 59 | 1 | 0.15785619 | 0 |
| GACGT | | | | | | | | | |
| TTCGG | | | | | | | | | |
| ACGTT | | | | | | | | | |
| (SEQ | | | | | | | | | |
| ID | | | | | | | | | |
| NO: | | | | | | | | | |
| 493) | | | | | | | | | |
| TGGAC | 59 | 59 | 1 | 55 | 59 | 59 | 1 | 0.23252619 | 0 |
| GTTTC | | | | | | | | | |
| GGACG | | | | | | | | | |
| TTTGG | | | | | | | | | |
| (SEQ | | | | | | | | | |
| ID | | | | | | | | | |
| NO: | | | | | | | | | |
| 494) | | | | | | | | | |
| TTCGG | 59 | 59 | 1 | 60 | 59 | 59 | 1 | 0.1572775 | 0 |
| ACGTT | | | | | | | | | |
| TGGAG | | | | | | | | | |
| GCCCA | | | | | | | | | |
| (SEQ | | | | | | | | | |
| ID | | | | | | | | | |
| NO: | | | | | | | | | |
| 495) | | | | | | | | | |
| ATGGA | 58 | 58 | 1 | 45 | 58 | 58 | 1 | 0.74070067 | 0 |
| AGTAG | | | | | | | | | |
| ACGTT | | | | | | | | | |
| TCGGA | | | | | | | | | |
| (SEQ | | | | | | | | | |
| ID | | | | | | | | | |
| NO: | | | | | | | | | |
| 496) | | | | | | | | | |
| TTGGA | 58 | 58 | 1 | 50 | 58 | 58 | 1 | 0.11155911 | 0 |
| GAGCC | | | | | | | | | |
| TTGAC | | | | | | | | | |
| ACCTA | | | | | | | | | |
| (SEQ | | | | | | | | | |
| ID | | | | | | | | | |
| NO: | | | | | | | | | |
| 497) | | | | | | | | | |
| GGAAT | 55 | 55 | 1 | 40 | 55 | 55 | 1 | 0.24532106 | 0 |
| CTCAG | | | | | | | | | |
| AATCT | | | | | | | | | |
| TCTTC | | | | | | | | | |
| (SEQ | | | | | | | | | |
| ID | | | | | | | | | |
| NO: | | | | | | | | | |
| 498) | | | | | | | | | |
| AAGTG | 52 | 52 | 1 | 40 | 52 | 52 | 1 | 0.11924969 | 0 |
| GATGT | | | | | | | | | |
| TTGGA | | | | | | | | | |
| TAGCT | | | | | | | | | |
| (SEQ | | | | | | | | | |
| ID | | | | | | | | | |
| NO: | | | | | | | | | |
| 499) | | | | | | | | | |
| TGGAT | 52 | 52 | 1 | 45 | 52 | 52 | 1 | 0.34189267 | 0 |
| GTTTG | | | | | | | | | |
| GATAG | | | | | | | | | |
| CTTGG | | | | | | | | | |
| (SEQ | | | | | | | | | |
| ID | | | | | | | | | |
| NO: | | | | | | | | | |
| 500) | | | | | | | | | |
| AGTGA | 51 | 51 | 1 | 40 | 51 | 51 | 1 | 0.40360282 | 0 |
| GTGCA | | | | | | | | | |
| TACGT | | | | | | | | | |
| CATAA | | | | | | | | | |
| (SEQ | | | | | | | | | |
| ID | | | | | | | | | |
| NO: | | | | | | | | | |
| 501) | | | | | | | | | |
| TTTCA | 49 | 49 | 1 | 40 | 49 | 49 | 1 | 0.49750925 | 0 |
| AAGCT | | | | | | | | | |
| GCTCT | | | | | | | | | |
| CTGAA | | | | | | | | | |
| (SEQ | | | | | | | | | |
| ID | | | | | | | | | |
| NO: | | | | | | | | | |
| 502) | | | | | | | | | |
| GTGAA | 43 | 43 | 1 | 50 | 43 | 43 | 1 | 0.58378226 | 0 |
| CTCAG | | | | | | | | | |
| CTAAC | | | | | | | | | |
| ACACG | | | | | | | | | |
| (SEQ | | | | | | | | | |
| ID | | | | | | | | | |
| NO: | | | | | | | | | |
| 503) | | | | | | | | | |

CHRX

| | | | | | | | | | bind- |
|---|---|---|---|---|---|---|---|---|---|
| | bind- | | | | | | | | ing |
| | ing | | | | | | | | sites |
| | sites | | | | | | | | (CHM13- |
| | (CHM13- | bind- | | | | | | | spe- |
| | spe- | ing | | | | | | | cif- |
| | cif- | sites | | | | | | | ic |
| | ic | (CHM | | | bind- | | | | chro- |
| | chro- | 13- | chro- | | ing | bind- | | | mo- |
| | mo- | all | mo- | | sites | ing | centro- | | some |
| | some | cen- | some | | (CHM- | sites | mere | | centro- |
| sg | centro- | tro- | spe- | | 13 | (hg38 | spe- | Ac- | mere |
| RNA | mere | mere | cif- | GC_ | whole | whole | cif- | tiv- | Not |
| _ | HOR_ | HOR_ | ic- | cont- | ge- | ge- | ic- | ity | HOR_ |
| seq | L) | L) | ity | ent | nome) | nome) | ity | score | L) |
| GAGCT | 1428 | 1428 | 1 | 40 | 1428 | 1428 | 1 | 0.12636399 | 0 |
| GAACA | | | | | | | | | |
| TTCGT | | | | | | | | | |
| TATGA | | | | | | | | | |
| (SEQ | | | | | | | | | |
| ID | | | | | | | | | |
| NO: | | | | | | | | | |
| 504) | | | | | | | | | |
| CTTGC | 1414 | 1414 | 1 | 40 | 1414 | 1414 | 1 | 0.39549594 | 0 |
| AGATT | | | | | | | | | |
| CCAAA | | | | | | | | | |
| GAAAG | | | | | | | | | |
| (SEQ | | | | | | | | | |
| ID | | | | | | | | | |
| NO: | | | | | | | | | |
| 505) | | | | | | | | | |
| AGTTT | 1409 | 1409 | 1 | 40 | 1409 | 1409 | 1 | 0.12523394 | 0 |
| GCTTC | | | | | | | | | |
| CGTTC | | | | | | | | | |
| AGTTA | | | | | | | | | |
| (SEQ | | | | | | | | | |
| ID | | | | | | | | | |
| NO: | | | | | | | | | |
| 506) | | | | | | | | | |
| GTTTG | 1405 | 1405 | 1 | 40 | 1405 | 1405 | 1 | 0.28622966 | 0 |
| CTTCC | | | | | | | | | |
| GTTCA | | | | | | | | | |
| GTTAT | | | | | | | | | |
| (SEQ | | | | | | | | | |
| ID | | | | | | | | | |
| NO: | | | | | | | | | |
| 507) | | | | | | | | | |
| ACACT | 1403 | 1403 | 1 | 40 | 1403 | 1404 | 0.99928775 | 0.23455823 | 0 |
| TTTGG | | | | | | | | | |
| TAGAA | | | | | | | | | |
| TCTGC | | | | | | | | | |
| (SEQ | | | | | | | | | |
| ID | | | | | | | | | |
| NO: | | | | | | | | | |
| 508) | | | | | | | | | |
| GGAAT | 1402 | 1402 | 1 | 50 | 1402 | 1405 | 0.99786477 | 0.35126654 | 0 |
| CTGCA | | | | | | | | | |
| AGGGG | | | | | | | | | |
| ATATG | | | | | | | | | |
| (SEQ | | | | | | | | | |
| ID | | | | | | | | | |
| NO: | | | | | | | | | |
| 509) | | | | | | | | | |
| CTCTT | 1397 | 1397 | 1 | 40 | 1397 | 1397 | 1 | 0.27337848 | 0 |
| TCTTT | | | | | | | | | |
| GGAAT | | | | | | | | | |
| CTGCA | | | | | | | | | |
| (SEQ | | | | | | | | | |
| ID | | | | | | | | | |
| NO: | | | | | | | | | |
| 510) | | | | | | | | | |
| CTCTT | 1394 | 1394 | 1 | 55 | 1394 | 1394 | 1 | 0.67761774 | 0 |
| TCTGT | | | | | | | | | |
| GGGAT | | | | | | | | | |
| CCGCA | | | | | | | | | |
| (SEQ | | | | | | | | | |
| ID | | | | | | | | | |
| NO: | | | | | | | | | |
| 80) | | | | | | | | | |
| TCTTT | 1390 | 1390 | 1 | 50 | 1390 | 1390 | 1 | 0.67335444 | 0 |
| CTGTG | | | | | | | | | |
| GGATC | | | | | | | | | |
| CGCAA | | | | | | | | | |
| (SEQ | | | | | | | | | |
| ID | | | | | | | | | |
| NO: | | | | | | | | | |
| 511) | | | | | | | | | |
| CTTTC | 1386 | 1386 | 1 | 55 | 1387 | 1395 | 0.99354839 | 0.30475743 | 0 |
| TGTGG | | | | | | | | | |
| GATCC | | | | | | | | | |
| GCAAG | | | | | | | | | |
| (SEQ | | | | | | | | | |
| ID | | | | | | | | | |
| NO: | | | | | | | | | |
| 512) | | | | | | | | | |
| CACTT | 1366 | 1366 | 1 | 40 | 1366 | 1366 | 1 | 0.20885739 | 0 |
| GCAGA | | | | | | | | | |
| TTCTA | | | | | | | | | |
| CTACA | | | | | | | | | |
| (SEQ | | | | | | | | | |
| ID | | | | | | | | | |
| NO: | | | | | | | | | |
| 513) | | | | | | | | | |
| GACCT | 1365 | 1365 | 1 | 40 | 1366 | 1367 | 0.99853694 | 0.29517002 | 0 |
| CTTTG | | | | | | | | | |
| AAGAT | | | | | | | | | |
| TTCAC | | | | | | | | | |
| (SEQ | | | | | | | | | |
| ID | | | | | | | | | |
| NO: | | | | | | | | | |
| 514) | | | | | | | | | |
| GAGGT | 1358 | 1358 | 1 | 50 | 1358 | 1358 | 1 | 0.19283352 | 0 |
| CCAAA | | | | | | | | | |
| TATCC | | | | | | | | | |
| CCTTG | | | | | | | | | |
| (SEQ | | | | | | | | | |
| ID | | | | | | | | | |
| NO: | | | | | | | | | |
| 81) | | | | | | | | | |
| TTCAA | 1353 | 1353 | 1 | 40 | 1353 | 1357 | 0.99705232 | 0.48880396 | 0 |
| ACGAA | | | | | | | | | |
| GGCTA | | | | | | | | | |
| CAAAG | | | | | | | | | |
| (SEQ | | | | | | | | | |
| ID | | | | | | | | | |
| NO: | | | | | | | | | |
| 515) | | | | | | | | | |
| AGGGA | 1351 | 1351 | 1 | 45 | 1351 | 1353 | 0.9985218 | 0.30699075 | 0 |
| AAGTT | | | | | | | | | |
| CAACT | | | | | | | | | |
| CTGTG | | | | | | | | | |
| (SEQ | | | | | | | | | |
| ID | | | | | | | | | |
| NO: | | | | | | | | | |
| 516) | | | | | | | | | |
| GAACC | 1348 | 1348 | 1 | 50 | 1348 | 1348 | 1 | 0.17699115 | 0 |
| TGAAC | | | | | | | | | |
| TCTCA | | | | | | | | | |
| AAGGC | | | | | | | | | |
| (SEQ | | | | | | | | | |
| ID | | | | | | | | | |
| NO: | | | | | | | | | |
| 517) | | | | | | | | | |
| CTTTT | 1344 | 1344 | 1 | 40 | 1345 | 1356 | 0.99115044 | 0.65207576 | 0 |
| TCGAG | | | | | | | | | |
| AATCT | | | | | | | | | |
| GCAAG | | | | | | | | | |
| (SEQ | | | | | | | | | |
| ID | | | | | | | | | |
| NO: | | | | | | | | | |
| 518) | | | | | | | | | |
| TTTCG | 1323 | 1323 | 1 | 40 | 1323 | 1323 | 1 | 0.63110447 | 0 |
| AACCT | | | | | | | | | |
| GAACT | | | | | | | | | |
| CTCAA | | | | | | | | | |
| (SEQ | | | | | | | | | |
| ID | | | | | | | | | |
| NO: | | | | | | | | | |
| 519) | | | | | | | | | |
| CATAT | 1300 | 1301 | 0.999 | 45 | 1300 | 1301 | 0.99923136 | 0.55575224 | 0 |
| ACCCG | | | | | | | | | |
| TTTCG | | | | | | | | | |
| AACGA | | | | | | | | | |
| (SEQ | | | | | | | | | |
| ID | | | | | | | | | |
| NO: | | | | | | | | | |
| 520) | | | | | | | | | |
| GCGGG | 1273 | 1273 | 1 | 65 | 1273 | 1273 | 1 | 0.21655563 | 0 |
| CTTGG | | | | | | | | | |
| AGGAC | | | | | | | | | |
| TGTGT | | | | | | | | | |
| (SEQ | | | | | | | | | |
| ID | | | | | | | | | |
| NO: | | | | | | | | | |
| 521) | | | | | | | | | |
| AGAAT | 1172 | 1172 | 1 | 40 | 1172 | 1172 | 1 | 0.4615561 | 0 |
| CTGTA | | | | | | | | | |
| AGTGG | | | | | | | | | |
| ATACG | | | | | | | | | |
| (SEQ | | | | | | | | | |
| ID | | | | | | | | | |
| NO: | | | | | | | | | |
| 522) | | | | | | | | | |
| TTGGA | 1155 | 1155 | 1 | 40 | 1155 | 1155 | 1 | 0.15400953 | 0 |
| AACTG | | | | | | | | | |
| CTCCA | | | | | | | | | |
| TCAAA | | | | | | | | | |
| (SEQ | | | | | | | | | |
| ID | | | | | | | | | |
| NO: | | | | | | | | | |
| 523) | | | | | | | | | |
| TTTCA | 1137 | 1137 | 1 | 50 | 1137 | 1137 | 1 | 0.2041899 | 0 |
| GGCCT | | | | | | | | | |
| TTTCC | | | | | | | | | |
| ACCAC | | | | | | | | | |
| (SEQ | | | | | | | | | |
| ID | | | | | | | | | |
| NO: | | | | | | | | | |
| 524) | | | | | | | | | |
| GAGCT | 1099 | 1099 | 1 | 45 | 1099 | 1099 | 1 | 0.23088178 | 0 |
| GAACA | | | | | | | | | |
| TGCCT | | | | | | | | | |
| TTTGA | | | | | | | | | |
| (SEQ | | | | | | | | | |
| ID | | | | | | | | | |
| NO: | | | | | | | | | |
| 525) | | | | | | | | | |
| CACGT | 1090 | 1090 | 1 | 40 | 1090 | 1090 | 1 | 0.43318757 | 0 |
| TTTGT | | | | | | | | | |
| AGAAT | | | | | | | | | |
| CTGCA | | | | | | | | | |
| (SEQ | | | | | | | | | |
| ID | | | | | | | | | |
| NO: | | | | | | | | | |
| 526) | | | | | | | | | |
| AAGTG | 992 | 996 | 0.996 | 40 | 992 | 996 | 0.99598394 | 0.38038501 | 0 |
| GATAT | | | | | | | | | |
| TTGGA | | | | | | | | | |
| CCACT | | | | | | | | | |
| (SEQ | | | | | | | | | |
| ID | | | | | | | | | |
| NO: | | | | | | | | | |
| 527) | | | | | | | | | |
| TTTCT | 989 | 989 | 1 | 50 | 989 | 989 | 1 | 0.536591 | 0 |
| GAGAG | | | | | | | | | |
| TGCTA | | | | | | | | | |
| CCGTC | | | | | | | | | |
| (SEQ | | | | | | | | | |
| ID | | | | | | | | | |
| NO: | | | | | | | | | |
| 528) | | | | | | | | | |
| TGGAT | 976 | 976 | 1 | 50 | 976 | 976 | 1 | 0.48940547 | 0 |
| ATTTG | | | | | | | | | |
| GACCA | | | | | | | | | |
| CTGGG | | | | | | | | | |
| (SEQ | | | | | | | | | |
| ID | | | | | | | | | |
| NO: | | | | | | | | | |
| 529) | | | | | | | | | |
| TTCGA | 951 | 951 | 1 | 60 | 951 | 951 | 1 | 0.61949008 | 0 |
| ACGAA | | | | | | | | | |
| GGCCA | | | | | | | | | |
| CCCAG | | | | | | | | | |
| (SEQ | | | | | | | | | |
| ID | | | | | | | | | |
| NO: | | | | | | | | | |
| 530) | | | | | | | | | |
| GTGAC | 945 | 945 | 1 | 45 | 945 | 945 | 1 | 0.40978721 | 0 |
| GATGG | | | | | | | | | |
| AGTTT | | | | | | | | | |
| AACTC | | | | | | | | | |
| (SEQ | | | | | | | | | |
| ID | | | | | | | | | |
| NO: | | | | | | | | | |
| 531) | | | | | | | | | |
| TGGGT | 942 | 942 | 1 | 55 | 942 | 942 | 1 | 0.29922822 | 0 |
| GGCCT | | | | | | | | | |
| TCGTT | | | | | | | | | |
| CGAAA | | | | | | | | | |
| (SEQ | | | | | | | | | |
| ID | | | | | | | | | |
| NO: | | | | | | | | | |
| 532) | | | | | | | | | |
| GGGTG | 926 | 926 | 1 | 60 | 926 | 926 | 1 | 0.15471819 | 0 |
| GCCTT | | | | | | | | | |
| CGTTC | | | | | | | | | |
| GAAAC | | | | | | | | | |
| (SEQ | | | | | | | | | |
| ID | | | | | | | | | |
| NO: | | | | | | | | | |
| 533) | | | | | | | | | |
| GGATA | 915 | 920 | 0.995 | 40 | 915 | 921 | 0.99348534 | 0.41352806 | 0 |
| TTTGG | | | | | | | | | |
| ACCTC | | | | | | | | | |
| TTTGA | | | | | | | | | |
| (SEQ | | | | | | | | | |
| ID | | | | | | | | | |
| NO: | | | | | | | | | |
| 534) | | | | | | | | | |
| GTCAA | 903 | 903 | 1 | 45 | 903 | 903 | 1 | 0.382188 | 0 |
| AGCTG | | | | | | | | | |
| CGCTA | | | | | | | | | |
| TCAAA | | | | | | | | | |
| (SEQ | | | | | | | | | |
| ID | | | | | | | | | |
| NO: | | | | | | | | | |
| 535) | | | | | | | | | |
| TGTCA | 899 | 899 | 1 | 45 | 899 | 899 | 1 | 0.4308845 | 0 |
| AAGCT | | | | | | | | | |
| GCGCT | | | | | | | | | |
| ATCAA | | | | | | | | | |
| (SEQ | | | | | | | | | |
| ID | | | | | | | | | |
| NO: | | | | | | | | | |
| 536) | | | | | | | | | |
| AAAAC | 898 | 898 | 1 | 40 | 898 | 898 | 1 | 0.45289126 | 0 |
| TGCTC | | | | | | | | | |
| CATCA | | | | | | | | | |
| AAAGG | | | | | | | | | |
| (SEQ | | | | | | | | | |
| ID | | | | | | | | | |
| NO: | | | | | | | | | |
| 537) | | | | | | | | | |
| ATGTG | 897 | 897 | 1 | 40 | 897 | 897 | 1 | 0.30183801 | 0 |
| CAAGT | | | | | | | | | |
| GGCTA | | | | | | | | | |
| TTTAG | | | | | | | | | |
| (SEQ | | | | | | | | | |
| ID | | | | | | | | | |
| NO: | | | | | | | | | |
| 538) | | | | | | | | | |
| TGTGC | 866 | 866 | 1 | 45 | 866 | 866 | 1 | 0.32185331 | 0 |
| AAGTG | | | | | | | | | |
| GCTAT | | | | | | | | | |
| TTAGC | | | | | | | | | |
| (SEQ | | | | | | | | | |
| ID | | | | | | | | | |
| NO: | | | | | | | | | |
| 539) | | | | | | | | | |
| AAGTG | 861 | 861 | 1 | 50 | 861 | 861 | 1 | 0.10809985 | 0 |
| GCTAT | | | | | | | | | |
| TTAGC | | | | | | | | | |
| GGGCT | | | | | | | | | |
| (SEQ | | | | | | | | | |
| ID | | | | | | | | | |
| NO: | | | | | | | | | |
| 540) | | | | | | | | | |
| TGGCT | 842 | 842 | 1 | 55 | 842 | 842 | 1 | 0.1342237 | 0 |
| ATTTA | | | | | | | | | |
| GCGGG | | | | | | | | | |
| CTTGG | | | | | | | | | |
| (SEQ | | | | | | | | | |
| ID | | | | | | | | | |
| NO: | | | | | | | | | |
| 541) | | | | | | | | | |
| GAGTT | 818 | 818 | 1 | 40 | 818 | 818 | 1 | 0.18007795 | 0 |
| GAACA | | | | | | | | | |
| ATCCT | | | | | | | | | |
| TCTGA | | | | | | | | | |
| (SEQ | | | | | | | | | |
| ID | | | | | | | | | |
| NO: | | | | | | | | | |
| 542) | | | | | | | | | |
| CAGTT | 770 | 770 | 1 | 45 | 770 | 770 | 1 | 0.12445998 | 0 |
| GAACC | | | | | | | | | |
| CTCCT | | | | | | | | | |
| TTTGA | | | | | | | | | |
| (SEQ | | | | | | | | | |
| ID | | | | | | | | | |
| NO: | | | | | | | | | |
| 543) | | | | | | | | | |
| TTCTC | 756 | 756 | 1 | 40 | 756 | 758 | 0.99736148 | 0.11710228 | 0 |
| AGAAA | | | | | | | | | |
| CGACT | | | | | | | | | |
| TTGTG | | | | | | | | | |
| (SEQ | | | | | | | | | |
| ID | | | | | | | | | |
| NO: | | | | | | | | | |
| 544) | | | | | | | | | |
| TTTGA | 731 | 731 | 1 | 50 | 731 | 731 | 1 | 0.33345081 | 0 |
| GGCCT | | | | | | | | | |
| GTGGT | | | | | | | | | |
| AGTGA | | | | | | | | | |
| (SEQ | | | | | | | | | |
| ID | | | | | | | | | |
| NO: | | | | | | | | | |
| 545) | | | | | | | | | |
| CACTA | 730 | 730 | 1 | 55 | 730 | 730 | 1 | 0.34179826 | 0 |
| CCACA | | | | | | | | | |
| GGCCT | | | | | | | | | |
| CAAAG | | | | | | | | | |
| (SEQ | | | | | | | | | |
| ID | | | | | | | | | |
| NO: | | | | | | | | | |
| 546) | | | | | | | | | |
| TTTGA | 658 | 658 | 1 | 50 | 658 | 658 | 1 | 0.37983727 | 0 |
| GGCCT | | | | | | | | | |
| ACGGT | | | | | | | | | |
| CGTAT | | | | | | | | | |
| (SEQ | | | | | | | | | |
| ID | | | | | | | | | |
| NO: | | | | | | | | | |
| 547) | | | | | | | | | |
| AGGCC | 655 | 655 | 1 | 55 | 655 | 655 | 1 | 0.39638119 | 0 |
| TACGG | | | | | | | | | |
| TCGTA | | | | | | | | | |
| TAGGA | | | | | | | | | |
| (SEQ | | | | | | | | | |
| ID | | | | | | | | | |
| NO: | | | | | | | | | |
| 548) | | | | | | | | | |
| GTTCC | 643 | 643 | 1 | 50 | 643 | 643 | 1 | 0.51234892 | 0 |
| TTCCT | | | | | | | | | |
| ATACG | | | | | | | | | |
| ACCGT | | | | | | | | | |
| (SEQ | | | | | | | | | |
| ID | | | | | | | | | |
| NO: | | | | | | | | | |
| 549) | | | | | | | | | |
| GTTCT | 642 | 642 | 1 | 45 | 642 | 642 | 1 | 0.36706957 | 0 |
| TTCCT | | | | | | | | | |
| TCACT | | | | | | | | | |
| ACCAC | | | | | | | | | |
| (SEQ | | | | | | | | | |
| ID | | | | | | | | | |
| NO: | | | | | | | | | |
| 550) | | | | | | | | | |
| CAGAA | 547 | 547 | 1 | 40 | 547 | 547 | 1 | 0.13746078 | 0 |
| ACTAC | | | | | | | | | |
| TTTGT | | | | | | | | | |
| GAGGA | | | | | | | | | |
| (SEQ | | | | | | | | | |
| ID | | | | | | | | | |
| NO: | | | | | | | | | |
| 551) | | | | | | | | | |
| TTTGA | 504 | 505 | 0.998 | 45 | 504 | 507 | 0.99408284 | 0.29002874 | 0 |
| GGCCT | | | | | | | | | |
| GTGGT | | | | | | | | | |
| AGTAA | | | | | | | | | |
| (SEQ | | | | | | | | | |
| ID | | | | | | | | | |
| NO: | | | | | | | | | |
| 552) | | | | | | | | | |
| GTCGA | 502 | 502 | 1 | 50 | 502 | 502 | 1 | 0.43103106 | 0 |
| AGCTG | | | | | | | | | |
| CGCTA | | | | | | | | | |
| TCAAA | | | | | | | | | |
| (SEQ | | | | | | | | | |
| ID | | | | | | | | | |
| NO: | | | | | | | | | |
| 553) | | | | | | | | | |
| CGAAC | 501 | 501 | 1 | 40 | 501 | 501 | 1 | 0.48537886 | 0 |
| ACAAA | | | | | | | | | |
| CATCA | | | | | | | | | |
| CAAAG | | | | | | | | | |
| (SEQ | | | | | | | | | |
| ID | | | | | | | | | |
| NO: | | | | | | | | | |
| 554) | | | | | | | | | |
| TGTGC | 494 | 494 | 1 | 40 | 494 | 494 | 1 | 0.36441546 | 0 |
| AAGTG | | | | | | | | | |
| GATAT | | | | | | | | | |
| TTAGC | | | | | | | | | |
| (SEQ | | | | | | | | | |
| ID | | | | | | | | | |
| NO: | | | | | | | | | |
| 555) | | | | | | | | | |
| TGTCG | 493 | 493 | 1 | 50 | 493 | 493 | 1 | 0.48110465 | 0 |
| AAGCT | | | | | | | | | |
| GCGCT | | | | | | | | | |
| ATCAA | | | | | | | | | |
| (SEQ | | | | | | | | | |
| ID | | | | | | | | | |
| NO: | | | | | | | | | |
| 556) | | | | | | | | | |
| GTTCT | 490 | 490 | 1 | 40 | 490 | 490 | 1 | 0.28610917 | 0 |
| TTCCT | | | | | | | | | |
| TTACT | | | | | | | | | |
| ACCAC | | | | | | | | | |
| (SEQ | | | | | | | | | |
| ID | | | | | | | | | |
| NO: | | | | | | | | | |
| 557) | | | | | | | | | |
| TACTA | 490 | 491 | 0.998 | 50 | 490 | 491 | 0.99796334 | 0.42876294 | 0 |
| CCACA | | | | | | | | | |
| GGCCT | | | | | | | | | |
| CAAAG | | | | | | | | | |
| (SEQ | | | | | | | | | |
| ID | | | | | | | | | |
| NO: | | | | | | | | | |
| 558) | | | | | | | | | |
| TGGAT | 475 | 475 | 1 | 50 | 475 | 475 | 1 | 0.15478801 | 0 |
| ATTTA | | | | | | | | | |
| GCGGG | | | | | | | | | |
| CTTGG | | | | | | | | | |
| (SEQ | | | | | | | | | |
| ID | | | | | | | | | |
| NO: | | | | | | | | | |
| 559) | | | | | | | | | |
| AGGCC | 402 | 402 | 1 | 55 | 402 | 402 | 1 | 0.50634556 | 0 |
| TACGG | | | | | | | | | |
| TAGTA | | | | | | | | | |
| CAGGA | | | | | | | | | |
| (SEQ | | | | | | | | | |
| ID | | | | | | | | | |
| NO: | | | | | | | | | |
| 560) | | | | | | | | | |
| TTTGA | 402 | 402 | 1 | 50 | 402 | 402 | 1 | 0.21139188 | 0 |
| GGCCT | | | | | | | | | |
| ACGGT | | | | | | | | | |
| AGTAC | | | | | | | | | |
| (SEQ | | | | | | | | | |
| ID | | | | | | | | | |
| NO: | | | | | | | | | |
| 561) | | | | | | | | | |

CHRY(HG38)

| | bind- | | | | | | | |
|---|---|---|---|---|---|---|---|---|
| | ing | | | | | | | |
| | sites | | | | | | | |
| | (CHM13- | bind- | | | | | | |
| | spe- | ing | | | | | | |
| | cif- | sites | | | | | | |
| | ic | (CHM | chro- | | bind- | | | |
| | chro- | 13- | mo- | | ing | bind- | | |
| | mo- | all | some | | sites | ing | centro- | |
| | some | ce-n | spe- | | (CHM- | sites | mere | |
| sg | centro- | tro- | - | | 13 | (hg38 | spe- | Ac- |
| RNA | mere | mere | cif- | GC_ | whole | whole | cif- | tiv- |
| _ | HOR_ | HOR_ | ic- | cont- | ge- | ge- | ic- | ity |
| seq | L) | L) | ity | ent | nome) | nome) | ity | score |
| GAGCC | 43 | 47 | 0.915 | 60 | 43 | 66 | 0.65151515 | 0.114397 |
| CTTTG | | | | | | | | |
| CAGCC | | | | | | | | |
| TATGG | | | | | | | | |
| (SEQ | | | | | | | | |
| ID | | | | | | | | |
| NO: | | | | | | | | |
| 562) | | | | | | | | |
| TTGGA | 43 | 47 | 0.915 | 55 | 43 | 75 | 0.57333333 | 0.093487 |
| GCCCT | | | | | | | | |
| TTGCA | | | | | | | | |
| GCCTA | | | | | | | | |
| (SEQ | | | | | | | | |
| ID | | | | | | | | |
| NO: | | | | | | | | |
| 563) | | | | | | | | |
| TTTCC | 43 | 47 | 0.915 | 45 | 43 | 77 | 0.55844156 | 0.387383 |
| ACCAT | | | | | | | | |
| AGGCT | | | | | | | | |
| GCAAA | | | | | | | | |
| (SEQ | | | | | | | | |
| ID | | | | | | | | |
| NO: | | | | | | | | |
| 564) | | | | | | | | |
| TTTTC | 43 | 49 | 0.878 | 45 | 43 | 75 | 0.57333333 | 0.336784 |
| CACCA | | | | | | | | |
| TAGGC | | | | | | | | |
| TGCAA | | | | | | | | |
| (SEQ | | | | | | | | |
| ID | | | | | | | | |
| NO: | | | | | | | | |
| 565) | | | | | | | | |
| TTCCA | 41 | 42 | 0.976 | 40 | 41 | 50 | 0.82 | 0.281733 |
| AACTG | | | | | | | | |
| CTCAA | | | | | | | | |
| TCAAG | | | | | | | | |
| (SEQ | | | | | | | | |
| ID | | | | | | | | |
| NO: | | | | | | | | |
| 566) | | | | | | | | |
| TTCCT | 41 | 42 | 0.976 | 40 | 41 | 49 | 0.83673469 | 0.091921 |
| CTTGA | | | | | | | | |
| TTGAG | | | | | | | | |
| CAGTT | | | | | | | | |
| (SEQ | | | | | | | | |
| ID | | | | | | | | |
| NO: | | | | | | | | |
| 567) | | | | | | | | |
| CAGCG | 40 | 40 | 1 | 70 | 40 | 46 | 0.86956522 | 0.249916 |
| CTTTG | | | | | | | | |
| AGGCC | | | | | | | | |
| TGCGG | | | | | | | | |
| (SEQ | | | | | | | | |
| ID | | | | | | | | |
| NO: | | | | | | | | |
| 568) | | | | | | | | |
| GAGCA | 40 | 40 | 1 | 55 | 40 | 50 | 0.8 | 0.165628 |
| CTTTG | | | | | | | | |
| AGGCC | | | | | | | | |
| TGTTG | | | | | | | | |
| (SEQ | | | | | | | | |
| ID | | | | | | | | |
| NO: | | | | | | | | |
| 569) | | | | | | | | |
| GATAT | 40 | 40 | 1 | 40 | 40 | 47 | 0.85106383 | 0.201964 |
| TTCCT | | | | | | | | |
| TCTCC | | | | | | | | |
| ACAAC | | | | | | | | |
| (SEQ | | | | | | | | |
| ID | | | | | | | | |
| NO: | | | | | | | | |
| 570) | | | | | | | | |
| GGTTC | 40 | 40 | 1 | 40 | 40 | 47 | 0.85106383 | 0.233775 |
| AAATC | | | | | | | | |
| TGTCA | | | | | | | | |
| GTTGA | | | | | | | | |
| (SEQ | | | | | | | | |
| ID | | | | | | | | |
| NO: | | | | | | | | |
| 571) | | | | | | | | |
| TGATG | 40 | 40 | 1 | 45 | 40 | 51 | 0.78431373 | 0.201989 |
| TGTGC | | | | | | | | |
| ACTCA | | | | | | | | |
| TCTCA | | | | | | | | |
| (SEQ | | | | | | | | |
| ID | | | | | | | | |
| NO: | | | | | | | | |
| 572) | | | | | | | | |
| TGGAT | 40 | 43 | 0.93 | 45 | 40 | 74 | 0.54054054 | 0.068878 |
| ATTTG | | | | | | | | |
| CAGCG | | | | | | | | |
| CTTTG | | | | | | | | |
| (SEQ | | | | | | | | |
| ID | | | | | | | | |
| NO: | | | | | | | | |
| 573) | | | | | | | | |
| TTGCA | 40 | 42 | 0.952 | 60 | 40 | 51 | 0.78431373 | 0.098434 |
| GCGCT | | | | | | | | |
| TTGAG | | | | | | | | |
| GCCTG | | | | | | | | |
| (SEQ | | | | | | | | |
| ID | | | | | | | | |
| NO: | | | | | | | | |
| 574) | | | | | | | | |
| TTGGA | 40 | 41 | 0.976 | 45 | 40 | 62 | 0.64516129 | 0.051576 |
| GTGCT | | | | | | | | |
| TTGAG | | | | | | | | |
| GCATA | | | | | | | | |
| (SEQ | | | | | | | | |
| ID | | | | | | | | |
| NO: | | | | | | | | |
| 575) | | | | | | | | |
| TTTGA | 40 | 40 | 1 | 50 | 40 | 47 | 0.85106383 | 0.115221 |
| GGCCT | | | | | | | | |
| GTTGT | | | | | | | | |
| GGAGA | | | | | | | | |
| (SEQ | | | | | | | | |
| ID | | | | | | | | |
| NO: | | | | | | | | |
| 576) | | | | | | | | |

### Table S2

TABLE S2
Python-based quantification of FISH foci (see
also FIG. 3E for manual quantification)

| | | mean | mean | mean | mean |
|---|---|---|---|---|---|
| | | chr7 | chr7 | chr18 | chr 18 |
| | | gains | losses | gains | losses |
| GFP | sgNC | 2.8 | 3.1 | 2.9 | 3.5 |
| GFP | sgChr7-1 | 3.5 | 2.8 | 2.7 | 3 |
| GFP | sgChr18-4 | 3.4 | 3.4 | 3.4 | 2.9 |
| KNL1Mut-dCas9 | sgNC | 3.8 | 3.5 | 3.2 | 3.3 |
| KNL1Mut-dCas9 | sgChr7-1 | 11.7 | 14.5 | 3.4 | 3.8 |
| KNL1Mut-dCas9 | sgChr18-4 | 3.2 | 2.9 | 13.1 | 17.4 |

### Table S3

TABLE S3A
(1^{st} Part)

| Sample | change | chr1p | chr1q | chr2p | chr2q | chr3p | chr3q | chr4p | chr4q | chr5p | chr5q | chr6p | chr6q | chr7p |
|---|---|---|---|---|---|---|---|---|---|---|---|---|---|---|
| hCEC | gain | 1.5 | 1.4 | 1.5 | 1.2 | 0.5 | 1.8 | 0.6 | 1 | 1.1 | 1.8 | 1 | 1 | 1.5 |
| diploid | | | | | | | | | | | | | | |
| hCEC | loss | 1.1 | 1.6 | 1.6 | 1.5 | 1.3 | 1.5 | 1.1 | 2 | 0.5 | 1.9 | 1.3 | 1.9 | 1.3 |
| diploid | | | | | | | | | | | | | | |
| hCEC | gain | 1.8 | 1.6 | 2 | 1.6 | 1.2 | 1.9 | 2.1 | 1.7 | 1.8 | 2 | 0.7 | 0.2 | 72.3 |
| 47, +7, XY | | | | | | | | | | | | | | |
| hCEC | loss | 1.1 | 1.8 | 1.1 | 1.3 | 1.7 | 2 | 1.9 | 1.7 | 1.7 | 1.9 | 2 | 1.7 | 0.1 |
| 47, +7, XY | | | | | | | | | | | | | | |
| hCEC, | gain | 0.5 | 0.8 | 1.1 | 0.4 | 1.2 | 0.5 | 0.7 | 0.7 | 0.1 | 1.2 | 0 | 0.5 | 81.2 |
| complex | | | | | | | | | | | | | | |
| aneuploidy | | | | | | | | | | | | | | |
| hCEC, | loss | 1.4 | 2 | 2.3 | 1.5 | 0.7 | 1.1 | 0.7 | 1.6 | 0.7 | 1.7 | 1.7 | 1.5 | 0.3 |
| complex | | | | | | | | | | | | | | |
| aneuploidy | | | | | | | | | | | | | | |

TABLE S3A
(2nd Part)

| Sample | change | chr7q | chr8p | chr8q | chr9p | chr9q | chr10p | chr10q | chr11p | chr11q | chr12p | chr12q | chr13q | chr14q |
|---|---|---|---|---|---|---|---|---|---|---|---|---|---|---|
| hCEC | gain | 1.9 | 0.9 | 1 | 0.9 | 0.7 | 0.7 | 1.8 | 1.2 | 1.5 | 0.9 | 1.2 | 0.8 | 1.6 |
| diploid | | | | | | | | | | | | | | |
| hCEC | loss | 1 | 1.4 | 1 | 1.1 | 1.9 | 1.5 | 1.7 | 1.6 | 1.3 | 0.8 | 0.7 | 1.7 | 1.8 |
| diploid | | | | | | | | | | | | | | |
| hCEC | gain | 76.8 | 0.6 | 1.5 | 0.7 | 1.8 | 1.4 | 1.2 | 1.7 | 1.7 | 0.6 | 1.6 | 2.1 | 0.6 |
| 47, +7, XY | | | | | | | | | | | | | | |
| hCEC | loss | 0.9 | 2 | 1.9 | 2 | 1.9 | 1.4 | 1.7 | 2.2 | 0.6 | 1.9 | 1.6 | 1.8 | 1.5 |
| 47, +7, XY | | | | | | | | | | | | | | |
| hCEC, | gain | 77.3 | 0.7 | 0.4 | 1.2 | 0.3 | 0.7 | 1.5 | 1.1 | 1.1 | 1.1 | 0.7 | 0.9 | 0.3 |
| complex | | | | | | | | | | | | | | |
| aneuploidy | | | | | | | | | | | | | | |
| hCEC, | loss | 0.1 | 0.4 | 0.9 | 0.9 | 2.1 | 0.8 | 3.2 | 0.4 | 1.7 | 0.8 | 0.7 | 2.4 | 1.9 |
| complex | | | | | | | | | | | | | | |
| aneuploidy | | | | | | | | | | | | | | |

TABLE S3A
(3rd Part)

| Sample | change | chr15q | chr16p | chr16q | chr17p | chr17q | chr18 | chr19p |
|---|---|---|---|---|---|---|---|---|
| hCEC | gain | 1.7 | 1 | 0.6 | 1.7 | 1.4 | 0.6 | 1.6 |
| diploid | | | | | | | | |
| hCEC | loss | 1.8 | 1.6 | 1.4 | 1.5 | 1.8 | 0.7 | 2.2 |
| diploid | | | | | | | | |
| hCEC | gain | 1.4 | 1.2 | 0.9 | 0.6 | 2.6 | 0.2 | 2 |
| 47, +7, XY | | | | | | | | |
| hCEC | loss | 2 | 2.1 | 1.9 | 1.2 | 1.6 | 2.1 | 0.4 |
| 47, +7, XY | | | | | | | | |
| hCEC, | gain | 0.4 | 0.7 | 2.1 | 0.4 | 1.1 | 0.3 | 84.5 |
| complex | | | | | | | | |
| aneuploidy | | | | | | | | |
| hCEC, | loss | 1.5 | 1.6 | 1.7 | 1.9 | 1.7 | 76.8 | 0.1 |
| complex | | | | | | | | |
| aneuploidy | | | | | | | | |

| Sample | chr19q | chr20p | chr20q | chr21q | chr22q | chrXp | chrXq |
|---|---|---|---|---|---|---|---|
| hCEC | 1.5 | 1 | 1.6 | 1.1 | 0.9 | 1 | 1.7 |
| diploid | | | | | | | |
| hCEC | 1.7 | 1 | 0.5 | 0.5 | 1.7 | 1.1 | 0.8 |
| diploid | | | | | | | |
| hCEC | 0 | 1.2 | 0.3 | 1.7 | 2.3 | 0.9 | 0.3 |
| 47, +7, XY | | | | | | | |
| hCEC | 1.8 | 2 | 2.2 | 1.2 | 1.8 | 2.2 | 1.9 |
| 47, +7, XY | | | | | | | |
| hCEC, | 1.5 | 0.8 | 0.9 | 0.7 | 0.7 | 0.3 | 0.7 |
| complex | | | | | | | |
| aneuploidy | | | | | | | |
| hCEC, | 3.1 | 0.7 | 0.4 | 0.8 | 1.5 | 1.6 | 1.6 |
| complex | | | | | | | |
| aneuploidy | | | | | | | |

### Table S3B

TABLE S3B
(1^{st} Part)

| Sample | change | chr1p | chr1q | chr2p | chr2q | chr3p | chr3q | chr4p | chr4q | chr5p | chr5q | chr6p | chr6q |
|---|---|---|---|---|---|---|---|---|---|---|---|---|---|
| hCEC sgRNA NC | gain | 1.8 | 2 | 0.6 | 0.5 | 0.7 | 0.5 | 0.5 | 0.9 | 0.6 | 0.8 | 0.7 | 0.5 |
| hCEC sgRNA NC | loss | 1.4 | 1.9 | 1.5 | 0.6 | 1.2 | 1.5 | 1.2 | 1.4 | 0.8 | 1.5 | 1 | 1.5 |
| hCEC sgRNA 6-2 | gain | 1.9 | 1.8 | 1.6 | 0.5 | 1.6 | 1.9 | 1.5 | 1.8 | 1.8 | 1.8 | 10.2 | 12.5 |
| hCEC sgRNA 6-2 | loss | 1.8 | 1.6 | 0.7 | 1.4 | 1.6 | 1.8 | 1.8 | 1.5 | 0.9 | 1.7 | 15.4 | 16.6 |
| hCEC sgRNA 7-1 | gain | 1.8 | 1.4 | 1.3 | 0.7 | 0.6 | 1.2 | 1.2 | 0.8 | 1.1 | 1.4 | 0.5 | 0.9 |
| hCEC sgRNA 7-1 | loss | 1.8 | 1.9 | 1 | 1.5 | 1.4 | 1 | 0.8 | 1.4 | 0.6 | 1.3 | 0.7 | 1.3 |
| hCEC sgRNA 8-2 | gain | 1.4 | 2.3 | 1.4 | 1.3 | 0.8 | 1.2 | 1.2 | 0.9 | 1.2 | 1.6 | 0.3 | 0.8 |
| hCEC sgRNA 8-2 | loss | 1.6 | 1.8 | 1.1 | 1.5 | 1.3 | 1.4 | 1.2 | 1.4 | 0.7 | 2.5 | 2 | 1.7 |
| hCEC sgRNA 9-3 | gain | 2 | 1.9 | 0.8 | 1.7 | 1.1 | 1.8 | 1.3 | 1 | 1.1 | 1.9 | 0.7 | 1.1 |
| hCEC sgRNA 9-3 | loss | 1 | 1.6 | 1.1 | 1.6 | 1.2 | 1.1 | 1.6 | 1.6 | 0.5 | 1.8 | 1.9 | 1.3 |
| hCEC sgRNA 12-2 | gain | 1.5 | 2 | 1.3 | 2 | 0.9 | 1.2 | 0.7 | 0.9 | 0.9 | 2.7 | 0.7 | 1.2 |
| hCEC sgRNA 12-2 | loss | 1.5 | 2.5 | 0.9 | 1.3 | 0.6 | 1.3 | 1.1 | 0.8 | 0.8 | 1.4 | 0.7 | 1.9 |
| hCEC sgRNA 16-1 | gain | 0.4 | 0.4 | 0.3 | 0.5 | 0.1 | 0.1 | 0.8 | 0.1 | 1.8 | 1.1 | 0.4 | 0.2 |
| hCEC sgRNA 16-1 | loss | 0.9 | 2.7 | 1.1 | 0.9 | 0.5 | 0.9 | 1.3 | 1.6 | 1.8 | 0.7 | 1.3 | 1.6 |
| hCEC sgRNA 18-4 | gain | 1.7 | 1.5 | 1.2 | 1.9 | 2 | 2 | 1 | 1.4 | 0.4 | 1.6 | 1.1 | 1.8 |
| hCEC sgRNA 18-4 | loss | 1.6 | 2.1 | 2 | 0.8 | 1.3 | 1.1 | 2 | 1.6 | 1.2 | 2.1 | 2 | 2.1 |
| hCEC sgRNA X-1 | gain | 1.6 | 1.8 | 1.3 | 0.7 | 1.4 | 1.7 | 0.8 | 1.3 | 0.9 | 2 | 0.8 | 1.6 |
| hCEC sgRNA X-1 | loss | 1.3 | 1.7 | 0.9 | 1.3 | 1.3 | 1.6 | 2 | 1.7 | 0.6 | 2 | 1.2 | 1.4 |
| hCEC sgRNA 13-5 | gain | 1.2 | 1.3 | 1.4 | 0.9 | 1.3 | 1.4 | 1.2 | 0.8 | 1.7 | 2.2 | 1 | 0.7 |
| hCEC sgRNA 13-5 | loss | 1.8 | 1.7 | 1.3 | 1.8 | 1.1 | 1 | 1 | 1.9 | 1.3 | 1.5 | 1.5 | 2 |
| hCEC sgRNA 7-1 | gain | 1.5 | 1.8 | 1.8 | 1.3 | 0.7 | 1.8 | 0.4 | 0.7 | 1.2 | 0.6 | 0.2 | 0.7 |
| (high expression | | | | | | | | | | | | | |
| KNL1Mut-dCas9) | | | | | | | | | | | | | |
| hCEC sgRNA 7-1 | loss | 1.5 | 1.9 | 0.6 | 0.4 | 0.6 | 0.5 | 1.1 | 0.6 | 0.3 | 0.5 | 0.7 | 1 |
| (high expression | | | | | | | | | | | | | |
| KNL1Mut-dCas9) | | | | | | | | | | | | | |

TABLE S3B
(2^{nd} Part)

| Sample | change | chr7p | chr7q | chr8p | chr8q | chr9p | chr9q | chr10p | chr10q | chr11p | chr11q | chr12p | chr12q | chr13q |
|---|---|---|---|---|---|---|---|---|---|---|---|---|---|---|
| hCEC | gain | 0.8 | 1.3 | 0.6 | 1.3 | 0.3 | 0.9 | 1 | 1.4 | 0.9 | 1.4 | 1.2 | 0.8 | 0.7 |
| sgRNA NC | | | | | | | | | | | | | | |
| hCEC | loss | 0.6 | 1.2 | 1.5 | 1 | 0.7 | 1.3 | 1.3 | 1.7 | 1 | 1.2 | 1.2 | 0.9 | 2 |
| sgRNA NC | | | | | | | | | | | | | | |
| hCEC | gain | 1.8 | 1.9 | 0.6 | 2 | 1.4 | 1.4 | 1.1 | 3.4 | 1.9 | 0.9 | 2 | 2 | 1.8 |
| sgRNA 6-2 | | | | | | | | | | | | | | |
| hCEC | loss | 1.3 | 1.4 | 1.5 | 2 | 1.2 | 0.6 | 0.7 | 3.1 | 1.7 | 1.8 | 1.1 | 1.4 | 1.5 |
| sgRNA 6-2 | | | | | | | | | | | | | | |
| hCEC | gain | 9.2 | 8.6 | 1 | 1.9 | 0.8 | 1 | 1.4 | 1.7 | 1.1 | 1.9 | 0.8 | 1.6 | 1.1 |
| sgRNA 7-1 | | | | | | | | | | | | | | |
| hCEC | loss | 7.6 | 10.7 | 1.3 | 0.8 | 1.1 | 1.4 | 0.7 | 1.9 | 0.9 | 1 | 1 | 0.7 | 0.9 |
| sgRNA 7-1 | | | | | | | | | | | | | | |
| hCEC | gain | 1.3 | 1.7 | 6.1 | 8.2 | 1 | 0.8 | 1 | 3.8 | 0.8 | 2 | 1.5 | 1 | 1.4 |
| sgRNA 8-2 | | | | | | | | | | | | | | |
| hCEC | loss | 0.8 | 0.6 | 7.8 | 8.9 | 0.9 | 1.3 | 1.2 | 1.6 | 1.1 | 0.8 | 1.1 | 0.9 | 1.7 |
| sgRNA 8-2 | | | | | | | | | | | | | | |
| hCEC | gain | 1.1 | 1.4 | 0.8 | 1.4 | 4.5 | 5.6 | 0.8 | 3.8 | 1.9 | 1.5 | 1.4 | 1.6 | 1.4 |
| sgRNA 9-3 | | | | | | | | | | | | | | |
| hCEC | loss | 1.2 | 1.1 | 1.3 | 1.4 | 7.3 | 9.2 | 1.4 | 1.7 | 0.7 | 0.8 | 1.3 | 0.9 | 1.6 |
| sgRNA 9-3 | | | | | | | | | | | | | | |
| hCEC | gain | 0.8 | 1 | 1.2 | 1.4 | 0.8 | 0.8 | 1.1 | 3.2 | 1.4 | 2.2 | 5.6 | 5.1 | 1.4 |
| sgRNA 12-2 | | | | | | | | | | | | | | |
| hCEC | loss | 0.9 | 0.8 | 1 | 1.1 | 0.8 | 1.5 | 1.3 | 1.7 | 1.4 | 1.4 | 6.2 | 8.2 | 1.4 |
| sgRNA 12-2 | | | | | | | | | | | | | | |
| hCEC | gain | 0.6 | 0.3 | 0.7 | 0.3 | 0.1 | 0.2 | 2.4 | 1.4 | 0.8 | 1.6 | 0.2 | 0 | 0.4 |
| sgRNA 16-1 | | | | | | | | | | | | | | |
| hCEC | loss | 0.5 | 0.6 | 1.3 | 0.6 | 0.9 | 0.9 | 2.3 | 1.5 | 0.3 | 0.5 | 0.3 | 0.4 | 1.4 |
| sgRNA 16-1 | | | | | | | | | | | | | | |
| hCEC | gain | 1.6 | 2 | 1.1 | 1.9 | 0.7 | 0.5 | 1.1 | 3.6 | 2 | 1.9 | 2.2 | 1.2 | 0.9 |
| sgRNA 18-4 | | | | | | | | | | | | | | |
| hCEC | loss | 2 | 1.8 | 1.9 | 1.1 | 1.2 | 0.5 | 1 | 2.5 | 1.4 | 1.4 | 0.7 | 1.7 | 1.3 |
| sgRNA 18-4 | | | | | | | | | | | | | | |
| hCEC | gain | 1.5 | 1.6 | 0.9 | 1.1 | 1.4 | 0.9 | 1.2 | 2.2 | 1.8 | 1.5 | 1.5 | 1.5 | 1.3 |
| sgRNA X-1 | | | | | | | | | | | | | | |
| hCEC | loss | 1 | 1.3 | 1.1 | 1.4 | 0.7 | 1.7 | 1 | 2.5 | 1 | 2 | 0.9 | 1.5 | 1.3 |
| sgRNA X-1 | | | | | | | | | | | | | | |
| hCEC | gain | 1.6 | 1.2 | 0.7 | 2.2 | 0.3 | 1.5 | 1.3 | 3.1 | 1.7 | 1.2 | 1.3 | 1.1 | 11.5 |
| sgRNA 13-5 | | | | | | | | | | | | | | |
| hCEC | loss | 1 | 1.4 | 1.4 | 1.4 | 0.9 | 1.5 | 0.6 | 0.8 | 0.6 | 0.6 | 0.9 | 0.5 | 17.4 |
| sgRNA 13-5 | | | | | | | | | | | | | | |
| hCEC | gain | 15.1 | 16.4 | 1.9 | 2.1 | 0.6 | 0.7 | 1.6 | 2.1 | 1.3 | 1.1 | 1.1 | 1.4 | 0.7 |
| sgRNA 7-1 | | | | | | | | | | | | | | |
| (high | | | | | | | | | | | | | | |
| expression | | | | | | | | | | | | | | |
| KNL1Mut- | | | | | | | | | | | | | | |
| dCas9) | | | | | | | | | | | | | | |
| hCEC | loss | 16.6 | 22.4 | 0.5 | 2.1 | 0.4 | 0.4 | 0.8 | 1 | 0.7 | 0.4 | 0.8 | 1.4 | 0.8 |
| sgRNA 7-1 | | | | | | | | | | | | | | |
| (high | | | | | | | | | | | | | | |
| expression | | | | | | | | | | | | | | |
| KNL1Mut- | | | | | | | | | | | | | | |
| dCas9) | | | | | | | | | | | | | | |

TABLE S3B
(3^{rd} Part)

| Sample | change | chr14q | chr15q | chr16p | chr16q | chr17p | chr17q | chr18 | chr19p |
|---|---|---|---|---|---|---|---|---|---|
| hCEC | gain | 1.1 | 0.9 | 0.8 | 0.8 | 1 | 0.9 | 0.7 | 2.2 |
| sgRNA | | | | | | | | | |
| NC | | | | | | | | | |
| hCEC | loss | 1.3 | 1.6 | 0.9 | 1.8 | 1.9 | 1.4 | 0.7 | 1.2 |
| sgRNA | | | | | | | | | |
| NC | | | | | | | | | |
| hCEC | gain | 1.9 | 1.6 | 1.3 | 1.4 | 1.8 | 1.5 | 1.9 | 2.3 |
| sgRNA | | | | | | | | | |
| 6-2 | | | | | | | | | |
| hCEC | loss | 1.2 | 0.9 | 1.3 | 1.3 | 0.8 | 2 | 1.2 | 2 |
| sgRNA | | | | | | | | | |
| 6-2 | | | | | | | | | |
| hCEC | gain | 0.8 | 1.4 | 0.8 | 0.7 | 0.7 | 0.7 | 1.8 | 2.2 |
| sgRNA | | | | | | | | | |
| 7-1 | | | | | | | | | |
| hCEC | loss | 1.1 | 1.4 | 1.1 | 1.8 | 1.4 | 1 | 0.5 | 1.3 |
| sgRNA | | | | | | | | | |
| 7-1 | | | | | | | | | |
| hCEC | gain | 1 | 1 | 0.8 | 0.9 | 1.4 | 1.7 | 0.8 | 1.6 |
| sgRNA | | | | | | | | | |
| 8-2 | | | | | | | | | |
| hCEC | loss | 1.1 | 1.6 | 1.1 | 1.7 | 1.3 | 1.3 | 0.8 | 1.6 |
| sgRNA | | | | | | | | | |
| 8-2 | | | | | | | | | |
| hCEC | gain | 1.2 | 0.8 | 0.9 | 0.8 | 1.3 | 1.8 | 2.1 | 1.9 |
| sgRNA | | | | | | | | | |
| 9-3 | | | | | | | | | |
| hCEC | loss | 1.4 | 0.9 | 0.9 | 1.6 | 1.8 | 0.8 | 0.9 | 1.9 |
| sgRNA | | | | | | | | | |
| 9-3 | | | | | | | | | |
| hCEC | gain | 0.9 | 1 | 0.6 | 0.6 | 1.6 | 1.7 | 0.6 | 2.2 |
| sgRNA | | | | | | | | | |
| 12-2 | | | | | | | | | |
| hCEC | loss | 1.2 | 1 | 1 | 1.4 | 1.7 | 1.1 | 0.8 | 1.9 |
| sgRNA | | | | | | | | | |
| 12-2 | | | | | | | | | |
| hCEC | gain | 0.4 | 0.2 | 5.5 | 7.8 | 0.6 | 0.3 | 0.5 | 3.2 |
| sgRNA | | | | | | | | | |
| 16-1 | | | | | | | | | |
| hCEC | loss | 0.8 | 1.6 | 9.6 | 14.5 | 1.3 | 0.4 | 0.6 | 1.4 |
| sgRNA | | | | | | | | | |
| 16-1 | | | | | | | | | |
| hCEC | gain | 1.5 | 1.1 | 1.4 | 1.4 | 1.8 | 1 | 10.1 | 1.8 |
| sgRNA | | | | | | | | | |
| 18-4 | | | | | | | | | |
| hCEC | loss | 1.8 | 2 | 0.8 | 1.8 | 1.7 | 1.2 | 17.4 | 1.4 |
| sgRNA | | | | | | | | | |
| 18-4 | | | | | | | | | |
| hCEC | gain | 1.3 | 1 | 1.1 | 1.1 | 1 | 1.5 | 1.2 | 2 |
| sgRNA | | | | | | | | | |
| X-1 | | | | | | | | | |
| hCEC | loss | 0.9 | 0.8 | 0.9 | 1.9 | 1.6 | 1.3 | 0.8 | 1.8 |
| sgRNA | | | | | | | | | |
| X-1 | | | | | | | | | |
| hCEC | gain | 1.1 | 1.3 | 0.8 | 0.8 | 1.1 | 1.6 | 1.4 | 2.3 |
| sgRNA | | | | | | | | | |
| 13-5 | | | | | | | | | |
| hCEC | loss | 1 | 0.9 | 1.2 | 1.7 | 1.9 | 1.4 | 1.2 | 1.7 |
| sgRNA | | | | | | | | | |
| 13-5 | | | | | | | | | |
| hCEC | gain | 1.4 | 1.5 | 1.4 | 0.7 | 0.7 | 0.7 | 1.3 | 2.1 |
| sgRNA | | | | | | | | | |
| 7-1 (high | | | | | | | | | |
| expression | | | | | | | | | |
| KNL1Mut- | | | | | | | | | |
| dCas9) | | | | | | | | | |
| hCEC | loss | 0.8 | 0.5 | 0.8 | 0.5 | 1 | 0.7 | 0.5 | 1.8 |
| sgRNA | | | | | | | | | |
| 7-1 (high | | | | | | | | | |
| expression | | | | | | | | | |
| KNL1Mut- | | | | | | | | | |
| dCas9) | | | | | | | | | |

| | | | | | | | | Average |
|---|---|---|---|---|---|---|---|---|
| | | | | | | | | % for |
| | | | | | | | | Targeted |
| Sample | chr19q | chr20p | chr20q | chr21q | chr22q | chrXp | chrXq | chrom |
| hCEC | 2 | 0.5 | 1.2 | 0.9 | 1.1 | 1 | 1.4 | NA |
| sgRNA | | | | | | | | |
| NC | | | | | | | | |
| hCEC | 1.9 | 0.9 | 0.7 | 0.8 | 1.6 | 0.7 | 0.7 | NA |
| sgRNA | | | | | | | | |
| NC | | | | | | | | |
| hCEC | 1.9 | 0.9 | 1.8 | 1.3 | 0.9 | 1.3 | 1.5 | 11.35 |
| sgRNA | | | | | | | | |
| 6-2 | | | | | | | | |
| hCEC | 1.8 | 0.7 | 0.9 | 0.7 | 2 | 1.9 | 1.9 | 16 |
| sgRNA | | | | | | | | |
| 6-2 | | | | | | | | |
| hCEC | 1.3 | 1.3 | 1.1 | 0.9 | 0.8 | 1.4 | 1.4 | 8.9 |
| sgRNA | | | | | | | | |
| 7-1 | | | | | | | | |
| hCEC | 1.4 | 1.1 | 0.7 | 1.2 | 1.6 | 1.1 | 1.2 | 9.15 |
| sgRNA | | | | | | | | |
| 7-1 | | | | | | | | |
| hCEC | 1.4 | 0.8 | 1 | 1.4 | 1.5 | 1.3 | 1.7 | 7.15 |
| sgRNA | | | | | | | | |
| 8-2 | | | | | | | | |
| hCEC | 1.4 | 0.4 | 0.6 | 1.5 | 1.3 | 0.6 | 1.2 | 8.35 |
| sgRNA | | | | | | | | |
| 8-2 | | | | | | | | |
| hCEC | 1.6 | 0.6 | 1.3 | 0.8 | 1.2 | 1.5 | 1.1 | 5.05 |
| sgRNA | | | | | | | | |
| 9-3 | | | | | | | | |
| hCEC | 1.4 | 0.7 | 0.6 | 1.3 | 1.9 | 1.1 | 2 | 8.25 |
| sgRNA | | | | | | | | |
| 9-3 | | | | | | | | |
| hCEC | 1.2 | 0.9 | 0.9 | 1.1 | 1.2 | 1.4 | 1.5 | 5.35 |
| sgRNA | | | | | | | | |
| 12-2 | | | | | | | | |
| hCEC | 1.6 | 0.7 | 1.1 | 1 | 1.7 | 0.9 | 1.2 | 7.2 |
| sgRNA | | | | | | | | |
| 12-2 | | | | | | | | |
| hCEC | 0.3 | 0.7 | 0.6 | 1.4 | 0.7 | 1.2 | 0.4 | 6.7 |
| sgRNA | | | | | | | | |
| 16-1 | | | | | | | | |
| hCEC | 1.3 | 0.9 | 0.3 | 1.9 | 1.6 | 1.3 | 0.6 | 12.1 |
| sgRNA | | | | | | | | |
| 16-1 | | | | | | | | |
| hCEC | 1.9 | 0.6 | 1 | 0.6 | 1.2 | 1.3 | 2.1 | 10.1 |
| sgRNA | | | | | | | | |
| 18-4 | | | | | | | | |
| hCEC | 1.9 | 1.2 | 0.8 | 1.8 | 1.7 | 1 | 2.2 | 17.4 |
| sgRNA | | | | | | | | |
| 18-4 | | | | | | | | |
| hCEC | 1.8 | 1.5 | 1.9 | 0.9 | 1.4 | 12.3 | 11.5 | 11.9 |
| sgRNA | | | | | | | | |
| X-1 | | | | | | | | |
| hCEC | 1.7 | 1 | 0.5 | 1.8 | 1.8 | 10.6 | 15.8 | 13.2 |
| sgRNA | | | | | | | | |
| X-1 | | | | | | | | |
| hCEC | 1.5 | 0.2 | 1.1 | 9.1 | 1.3 | 0.8 | 0.3 | 10.3 |
| sgRNA | | | | | | | | |
| 13-5 | | | | | | | | |
| hCEC | 2 | 0.8 | 0.8 | 15.3 | 0.9 | 1.4 | 1 | 16.35 |
| sgRNA | | | | | | | | |
| 13-5 | | | | | | | | |
| hCEC | 1.5 | 1.8 | 1.4 | 2 | 1.3 | 1.2 | 1.5 | 15.75 |
| sgRNA | | | | | | | | |
| 7-1 (high | | | | | | | | |
| expression | | | | | | | | |
| KNL1Mut- | | | | | | | | |
| dCas9) | | | | | | | | |
| hCEC | 1.3 | 1.9 | 2.1 | 1.3 | 1.1 | 0.5 | 0.6 | 19.5 |
| sgRNA | | | | | | | | |
| 7-1 (high | | | | | | | | |
| expression | | | | | | | | |
| KNL1Mut- | | | | | | | | |
| dCas9) | | | | | | | | |
| | | | | | | | | 9.25 |
| | | | | | | | | 12.745 |

TABLE S3C

| Sample | change | chr1 | chr2 | chr3 | chr4 | chr5 | chr6 | chr7 | chr8 | chr9 | chr10 | chr11 | chr12 |
|---|---|---|---|---|---|---|---|---|---|---|---|---|---|
| hCEC | gain | 2 | 1.7 | 0.9 | 1 | 1.7 | 0.7 | 1.8 | 0.6 | 1 | 2.6 | 1.7 | 1 |
| sgRNA | | | | | | | | | | | | | |
| NC | | | | | | | | | | | | | |
| hCEC | loss | 2.4 | 1.4 | 1.3 | 1.1 | 1.5 | 2 | 1 | 1.4 | 1.3 | 1.7 | 1.2 | 1 |
| sgRNA | | | | | | | | | | | | | |
| NC | | | | | | | | | | | | | |
| hCEC | gain | 2.4 | 2.2 | 2.1 | 1.7 | 1.8 | 8.3 | 2.1 | 1.7 | 1.6 | 1.9 | 2.1 | 1.3 |
| sgRNA | | | | | | | | | | | | | |
| 6-2 | | | | | | | | | | | | | |
| hCEC | loss | 1.8 | 1.4 | 1.3 | 2.1 | 0.8 | 14.8 | 1.7 | 1.5 | 1 | 2.1 | 1.8 | 1.9 |
| sgRNA | | | | | | | | | | | | | |
| 6-2 | | | | | | | | | | | | | |
| hCEC | gain | 1.1 | 1.2 | 1 | 0.9 | 2.1 | 0.7 | 8.5 | 1.1 | 0.6 | 2.8 | 1.7 | 0.4 |
| sgRNA | | | | | | | | | | | | | |
| 7-1 | | | | | | | | | | | | | |
| hCEC | loss | 1.8 | 2.2 | 1.5 | 1.2 | 1.1 | 1.3 | 10.1 | 0.6 | 1.5 | 1.1 | 1.1 | 0.8 |
| sgRNA | | | | | | | | | | | | | |
| 7-1 | | | | | | | | | | | | | |
| hCEC | gain | 1.5 | 1.9 | 0.9 | 1 | 1.6 | 0.6 | 1.9 | 5.8 | 1.2 | 2.6 | 2.1 | 1.4 |
| sgRNA | | | | | | | | | | | | | |
| 8-2 | | | | | | | | | | | | | |
| hCEC | loss | 1.1 | 2.1 | 1.3 | 1.9 | 1.4 | 1.5 | 0.7 | 7.1 | 1.5 | 2.9 | 1 | 1 |
| sgRNA | | | | | | | | | | | | | |
| 8-2 | | | | | | | | | | | | | |
| hCEC | gain | 2.2 | 1.9 | 1.8 | 1.2 | 1.9 | 0.6 | 1.6 | 0.9 | 4.1 | 2.1 | 1.5 | 1.4 |
| sgRNA | | | | | | | | | | | | | |
| 9-3 | | | | | | | | | | | | | |
| hCEC | loss | 2.5 | 1.6 | 1.1 | 0.9 | 1.8 | 1.3 | 1.3 | 1.1 | 6.8 | 1.8 | 1.1 | 0.9 |
| sgRNA | | | | | | | | | | | | | |
| 9-3 | | | | | | | | | | | | | |
| hCEC | gain | 1.7 | 1.7 | 1.4 | 0.7 | 1.7 | 1.1 | 1.7 | 0.9 | 1 | 2.3 | 1.7 | 4.2 |
| sgRNA | | | | | | | | | | | | | |
| 12-2 | | | | | | | | | | | | | |
| hCEC | loss | 1.5 | 1.2 | 1.2 | 1.6 | 1.4 | 1.6 | 1.1 | 0.9 | 1.3 | 1.4 | 1.1 | 5.1 |
| sgRNA | | | | | | | | | | | | | |
| 12-2 | | | | | | | | | | | | | |
| hCEC | gain | 0.2 | 0.4 | 0.2 | 0.0 | 0.7 | 0.1 | 0.1 | 0.5 | 0.1 | 1.0 | 1.4 | 0.0 |
| sgRNA | | | | | | | | | | | | | |
| 16-1 | | | | | | | | | | | | | |
| hCEC | loss | 0.4 | 0.7 | 0.1 | 1.7 | 0.7 | 0.7 | 0.5 | 0.3 | 0.6 | 1.1 | 0.2 | 0.4 |
| sgRNA | | | | | | | | | | | | | |
| 16-1 | | | | | | | | | | | | | |
| hCEC | gain | 1.1 | 1.5 | 0.7 | 0.9 | 1.6 | 1.1 | 2.3 | 0.9 | 0.8 | 2.7 | 1.6 | 1.9 |
| sgRNA | | | | | | | | | | | | | |
| 18-4 | | | | | | | | | | | | | |
| hCEC | loss | 1 | 1.3 | 1.8 | 0.9 | 1.2 | 1.6 | 1.5 | 0.8 | 1.1 | 1.7 | 0.9 | 0.7 |
| sgRNA | | | | | | | | | | | | | |
| 18-4 | | | | | | | | | | | | | |
| hCEC | gain | 1.5 | 1.2 | 2.2 | 1.3 | 1.9 | 1.1 | 0.9 | 1.3 | 0.8 | 2.2 | 1.3 | 1.9 |
| sgRNA | | | | | | | | | | | | | |
| X-1 | | | | | | | | | | | | | |
| hCEC | loss | 1.3 | 0.9 | 1.2 | 2.1 | 1.2 | 2.2 | 1.6 | 1.7 | 1.7 | 1.9 | 2 | 1.3 |
| sgRNA | | | | | | | | | | | | | |
| X-1 | | | | | | | | | | | | | |
| hCEC | gain | 1.4 | 1.4 | 1.5 | 0.9 | 2 | 1.3 | 1.7 | 1.2 | 1.2 | 2.6 | 0.9 | 1.4 |
| sgRNA | | | | | | | | | | | | | |
| 13-5 | | | | | | | | | | | | | |
| hCEC | loss | 1.1 | 1.7 | 1 | 1.1 | 1.4 | 1.7 | 1.4 | 0.9 | 1.6 | 1.9 | 0.7 | 0.7 |
| sgRNA | | | | | | | | | | | | | |
| 13-5 | | | | | | | | | | | | | |
| hCEC | gair | 1.7 | 2.4 | 1.1 | 2.1 | 1.2 | 1.5 | 12.2 | 2.1 | 1.4 | 2.6 | 0.9 | 1.9 |
| sgRNA | | | | | | | | | | | | | |
| 7-1 | | | | | | | | | | | | | |
| (high | | | | | | | | | | | | | |
| expression | | | | | | | | | | | | | |
| KNL1Mut- | | | | | | | | | | | | | |
| dCas9) | | | | | | | | | | | | | |
| hCEC | loss | 2.1 | 1.7 | 0.8 | 1.5 | 0.9 | 1.1 | 15.3 | 1.2 | 0.9 | 1.1 | 1.9 | 1.5 |
| sgRNA | | | | | | | | | | | | | |
| 7-1 | | | | | | | | | | | | | |
| (high | | | | | | | | | | | | | |
| expression | | | | | | | | | | | | | |
| KNL1Mut- | | | | | | | | | | | | | |
| dCas9) | | | | | | | | | | | | | |

| | | | | | | | | | | | | Average |
|---|---|---|---|---|---|---|---|---|---|---|---|---|
| | | | | | | | | | | | | % for |
| | | | | | | | | | | | | Targeted |
| Sample | chr13 | chr14 | chr15 | chr16 | chr17 | chr18 | chr19 | chr20 | chr21 | chr22 | chr23 | chrom |
| hCEC | 0.8 | 1.2 | 1.5 | 0.9 | 1.3 | 1.2 | 1.8 | 1.6 | 1 | 1.3 | 1 | |
| sgRNA | | | | | | | | | | | | |
| NC | | | | | | | | | | | | |
| hCEC | 2.1 | 1.2 | 1.7 | 1.2 | 1.2 | 0.7 | 1 | 0.7 | 1 | 1.5 | 0.9 | |
| sgRNA | | | | | | | | | | | | |
| NC | | | | | | | | | | | | |
| hCEC | 2.1 | 1.9 | 1.8 | 1.5 | 1.3 | 2.1 | 3.1 | 2.2 | 1.2 | 1.9 | 1.7 | 8.3 |
| sgRNA | | | | | | | | | | | | |
| 6-2 | | | | | | | | | | | | |
| hCEC | 2.3 | 0.8 | 0.7 | 2.1 | 1.8 | 1.4 | 1.9 | 1.5 | 0.8 | 3 | 1.9 | 14.8 |
| sgRNA | | | | | | | | | | | | |
| 6-2 | | | | | | | | | | | | |
| hCEC | 1.1 | 1.9 | 0.3 | 0.6 | 1.6 | 0.2 | 1.2 | 1.1 | 0.3 | 0.5 | 2 | 8.5 |
| sgRNA | | | | | | | | | | | | |
| 7-1 | | | | | | | | | | | | |
| hCEC | 1 | 1.5 | 0.8 | 0.9 | 1.5 | 0.9 | 2.2 | 0.9 | 1.5 | 1.5 | 0.9 | 10.1 |
| sgRNA | | | | | | | | | | | | |
| 7-1 | | | | | | | | | | | | |
| hCEC | 1.7 | 1.1 | 1.4 | 0.8 | 1.7 | 1.2 | 1.9 | 1.5 | 1.4 | 1.4 | 1.4 | 5.8 |
| sgRNA | | | | | | | | | | | | |
| 8-2 | | | | | | | | | | | | |
| hCEC | 1.7 | 1 | 1.6 | 1.3 | 1.6 | 0.8 | 1.3 | 0.6 | 1.2 | 0.9 | 1.3 | 7.1 |
| sgRNA | | | | | | | | | | | | |
| 8-2 | | | | | | | | | | | | |
| hCEC | 1.6 | 1.2 | 1.1 | 0.9 | 1.5 | 1.9 | 2.3 | 1.3 | 0.8 | 1.2 | 1.1 | 4.1 |
| sgRNA | | | | | | | | | | | | |
| 9-3 | | | | | | | | | | | | |
| hCEC | 1.6 | 1.4 | 1.1 | 1.3 | 1.8 | 1.1 | 1.5 | 0.9 | 1.4 | 2.1 | 1.6 | 6.8 |
| sgRNA | | | | | | | | | | | | |
| 9-3 | | | | | | | | | | | | |
| hCEC | 1.5 | 1.1 | 0.9 | 0.9 | 1.9 | 1.2 | 1.1 | 1.5 | 1.2 | 1.4 | 1.3 | 4.2 |
| sgRNA | | | | | | | | | | | | |
| 12-2 | | | | | | | | | | | | |
| hCEC | 1.4 | 1.2 | 0.9 | 1.4 | 0.6 | 0.8 | 1.2 | 0.9 | 0.9 | 1.8 | 1.8 | 5.1 |
| sgRNA | | | | | | | | | | | | |
| 12-2 | | | | | | | | | | | | |
| hCEC | 0.4 | 0.4 | 0.2 | 4.8 | 0.2 | 0.5 | 0.6 | 0.6 | 1.8 | 0.6 | 0.4 | 7.2 |
| sgRNA | | | | | | | | | | | | |
| 16-1 | | | | | | | | | | | | |
| hCEC | 1.6 | 0.9 | 1.7 | 8.8 | 0.2 | 0.6 | 0.6 | 0.2 | 2.3 | 1.8 | 0.8 | 8.8 |
| sgRNA | | | | | | | | | | | | |
| 16-1 | | | | | | | | | | | | |
| hCEC | 0.9 | 1.4 | 0.7 | 0.7 | 1.2 | 10.1 | 1.1 | 1.4 | 0.2 | 0.7 | 1.5 | 10.1 |
| sgRNA | | | | | | | | | | | | |
| 18-4 | | | | | | | | | | | | |
| hCEC | 1.4 | 1.9 | 1.9 | 1.2 | 0.8 | 17.4 | 1.2 | 1.1 | 1.9 | 0.9 | 1.9 | 17.4 |
| sgRNA | | | | | | | | | | | | |
| 18-4 | | | | | | | | | | | | |
| hCEC | 1.9 | 2.6 | 1.2 | 1.7 | 1.5 | 1.7 | 1.2 | 1.8 | 1.3 | 1.2 | 10.4 | 10.4 |
| sgRNA | | | | | | | | | | | | |
| X-1 | | | | | | | | | | | | |
| hCEC | 1.2 | 1.8 | 2.3 | 1.7 | 2.2 | 0.6 | 2.6 | 1 | 1.2 | 2.2 | 14.6 | 14.6 |
| sgRNA | | | | | | | | | | | | |
| X-1 | | | | | | | | | | | | |
| hCEC | 11.5 | 1.1 | 1.5 | 0.8 | 1.6 | 1.2 | 1.9 | 1.2 | 9.1 | 1.4 | 0.9 | 10.3 |
| sgRNA | | | | | | | | | | | | |
| 13-5 | | | | | | | | | | | | |
| hCEC | 17.4 | 1 | 0.6 | 1.2 | 1.1 | 0.9 | 1.2 | 1.1 | 15.3 | 0.9 | 1.2 | 16.35 |
| sgRNA | | | | | | | | | | | | |
| 13-5 | | | | | | | | | | | | |
| hCEC | 1.8 | 0.7 | 1.2 | 0.8 | 2.2 | 1.6 | 0.9 | 0.7 | 1.4 | 1.1 | 0.7 | 12.2 |
| sgRNA | | | | | | | | | | | | |
| 7-1 | | | | | | | | | | | | |
| (high | | | | | | | | | | | | |
| expression | | | | | | | | | | | | |
| KNL1Mut- | | | | | | | | | | | | |
| dCas9) | | | | | | | | | | | | |
| hCEC | 1.6 | 1.1 | 1.9 | 1.2 | 1.7 | 0.8 | 0.8 | 1.1 | 1.5 | 1.2 | 0.9 | 15.3 |
| sgRNA | | | | | | | | | | | | |
| 7-1 | | | | | | | | | | | | |
| (high | | | | | | | | | | | | |
| expression | | | | | | | | | | | | |
| KNL1Mut- | | | | | | | | | | | | |
| dCas9) | | | | | | | | | | | | |
| | | | | | | | | | | | | 8.11 |
| | | | | | | | | | | | | 11.635 |

### REFERENCES

This reference listing is not an indication that any reference is material to patentability
1. Knouse, K. A., Wu, J., Whittaker, C. A., and Amon, A. (2014). Single cell sequencing reveals low levels of aneuploidy across mammalian tissues. Proc. Natl. Acad. Sci. U.S.A. 111, 13409-13414. 10.1073/pnas. 1415287111.
2. Knouse, K. A., Davoli, T., Elledge, S. J., and Amon, A. (2017). Aneuploidy in Cancer: Seq-ing Answers to Old Questions. Annu. Rev. Cancer Biol. 1, 335-354. 10.1146/annurev-cancerbio-042616-072231.
3. Beroukhim, R., Mermel, C. H., Porter, D., Wei, G., Raychaudhuri, S., Donovan, J., Barretina, J., Boehm, J. S., Dobson, J., Urashima, M., et al. (2010). The landscape of somatic copy-number alteration across human cancers. Nature 463, 899-905. 10.1038/nature08822.
4. Davoli, T., Xu, A. W., Mengwasser, K. E., Sack, L. M., Yoon, J. C., Park, P. J., and Elledge, S. J. (2013). Cumulative haploinsufficiency and triplosensitivity drive aneuploidy patterns and shape the cancer genome. Cell 155, 948-962. 10.1016/j.cell.2013.10.011.
5. Taylor, A. M., Shih, J., Ha, G., Gao, G. F., Zhang, X., Berger, A. C., Schumacher, S. E., Wang, C., Hu, H., Liu, J., et al. (2018). Genomic and Functional Approaches to Understanding Cancer Aneuploidy. Cancer Cell 33, 676-689.e3. 10.1016/j.ccell.2018.03.007.
6. William, W. N., Zhao, X., Bianchi, J. J., Lin, H. Y., Cheng, P., Lee, J. J., Carter, H., Alexandrov, L. B., Abraham, J. P., Spetzler, D. B., et al. (2021). Immune evasion in HPV-head and neck precancer-cancer transition is driven by an aneuploid switch involving chromosome 9p loss. Proc. Natl. Acad. Sci. U.S.A 118, e2022655118. 10.1073/pnas.2022655118.
7. Watkins, T. B. K., Lim, E. L., Petkovic, M., Elizalde, S., Birkbak, N. J., Wilson, G. A., Moore, D. A., Grönroos, E., Rowan, A., Dewhurst, S. M., et al. (2020). Pervasive chromosomal instability and karyotype order in tumour evolution. Nature 587, 126-132. 10.1038/s41586-020-2698-6.
8. Santaguida, S., Tighe, A., D'Alise, A. M., Taylor, S. S., and Musacchio, A. (2010). Dissecting the role of MPS1 in chromosome biorientation and the spindle checkpoint through the small molecule inhibitor reversine. J. Cell Biol. 190, 73-87. 10.1083/jcb.201001036.
9. Hewitt, L., Tighe, A., Santaguida, S., White, A. M., Jones, C. D., Musacchio, A., Green, S., and Taylor, S. S. (2010). Sustained Mps1 activity is required in mitosis to recruit O-Mad2 to the Mad1-C-Mad2 core complex. J. Cell Biol. 190, 25-34. 10.1083/jcb.201002133.
10. Fournier, R. E. (1981). A general high-efficiency procedure for production of microcell hybrids. Proc. Natl. Acad. Sci. U.S.A 78, 6349-6353. 10.1073/pnas. 78.10.6349.
11. Stingele, S., Stoehr, G., Peplowska, K., Cox, J., Mann, M., and Storchova, Z. (2012). Global analysis of genome, transcriptome and proteome reveals the response to aneuploidy in human cells. Mol. Syst. Biol. 8, 608. 10.1038/msb.2012.40.
12. Ly, P., Teitz, L. S., Kim, D. H., Shoshani, O., Skaletsky, H., Fachinetti, D., Page, D. C., and Cleveland, D. W. (2017). Selective Y centromere inactivation triggers chromosome shattering in micronuclei and repair by non-homologous end joining. Nat. Cell Biol. 19, 68-75. 10.1038/ncb3450.
13. Ly, P., Brunner, S. F., Shoshani, O., Kim, D. H., Lan, W., Pyntikova, T., Flanagan, A. M., Behjati, S., Page, D. C., Campbell, P. J., et al. (2019). Chromosome segregation errors generate a diverse spectrum of simple and complex genomic rearrangements. Nat. Genet. 51, 705-715. 10.1038/s41588-019-0360-8.
14. Rayner, E., Durin, M.-A., Thomas, R., Moralli, D., O'Cathail, S. M., Tomlinson, I., Green, C. M., and Lewis, A. (2019). CRISPR-Cas9 Causes Chromosomal Instability and Rearrangements in Cancer Cell Lines, Detectable by Cytogenetic Methods. CRISPR J. 2, 406-416. 10.1089/crispr.2019.0006.
15. Zuo, E., Huo, X., Yao, X., Hu, X., Sun, Y., Yin, J., He, B., Wang, X., Shi, L., Ping, J., et al. (2017). CRISPR/Cas9-mediated targeted chromosome elimination. Genome Biol. 18, 224. 10.1186/s13059-017-1354-4
16. Tovini, L., Johnson, S. C., Andersen, A. M., Spierings, D. C. J., Wardenaar, R., Foijer, F., and McClelland, S. E. (2022). Inducing Specific Chromosome Mis-Segregation in Human Cells. EMBO J 42: e111559. 10.15252/embj.2022111559
17. Truong, M. A., Cane-Gasull, P., Vries, S. G. de, Nijenhuis, W., Wardenaar, R., Kapitein, L. C., Foijer, F., and Lens, S. M. A. (2022). A motor-based approach to induce chromosome-specific mis-segregations in human cells. EMBO J 42: e111587. 10.15252/embj.2022111587
18. Barra, V., and Fachinetti, D. (2018). The dark side of centromeres: types, causes and consequences of structural abnormalities implicating centromeric DNA. Nat. Commun. 9, 4340. 10.1038/s41467-018-06545-y.
19. Hayden, K. E. (2012). Human centromere genomics: now it's personal. Chromosome Res. Int. J. Mol. Supramol. Evol. Asp. Chromosome Biol. 20, 621-633. 10.1007/s10577-012-9295-y.
20. Schueler, M. G., and Sullivan, B. A. (2006). Structural and functional dynamics of human centromeric chromatin. Annu. Rev. Genomics Hum. Genet. 7, 301-313. 10.1146/annurev.genom.7.080505.115613.
21. Altemose, N., Logsdon, G. A., Bzikadze, A. V., Sidhwani, P., Langley, S. A., Caldas, G. V., Hoyt, S. J., Uralsky, L., Ryabov, F. D., Shew, C. J., et al. (2022). Complete genomic and epigenetic maps of human centromeres. Science 376, eabl4178. 10.1126/science.abl4178.
22. Musacchio, A., and Desai, A. (2017). A Molecular View of Kinetochore Assembly and Function. Biology 6, E5. 10.3390/biology6010005.
23. Cheeseman, I. M. (2014). The kinetochore. Cold Spring Harb. Perspect. Biol. 6, a015826. 10.1101/cshperspect.a015826.
24. Musacchio, A. (2015). The Molecular Biology of Spindle Assembly Checkpoint Signaling Dynamics. Curr. Biol. CB 25, R1002-1018. 10.1016/j.cub.2015.08.051.
25. Stern, B. M., and Murray, A. W. (2001). Lack of tension at kinetochores activates the spindle checkpoint in budding yeast. Curr. Biol. CB 11, 1462-1467. 10.1016/s0960-9822 (01) 00451-1.
26. Liu, D., and Lampson, M. A. (2009). Regulation of kinetochore-microtubule attachments by Aurora B kinase. Biochem. Soc. Trans. 37.
27. Papini, D., Levasseur, M. D., and Higgins, J. M. G. (2021). The Aurora B gradient sustains kinetochore stability in anaphase. Cell Rep. 37, 109818. 10.1016/j.celrep.2021.109818.
28. Liu, D., Vleugel, M., Backer, C. B., Hori, T., Fukagawa, T., Cheeseman, I. M., and Lampson, M. A. (2010). Regulated targeting of protein phosphatase 1 to the outer kinetochore by KNL1 opposes Aurora B kinase. J. Cell Biol. 188, 809-820. 10.1083/jcb.201001006.
29. Nurk, S., Koren, S., Rhie, A., Rautiainen, M., Bzikadze, A. V., Mikheenko, A., Vollger, M. R., Altemose, N., Uralsky, L., Gershman, A., et al. (2022). The complete sequence of a human genome. Science 376, 44-53. 10.1126/science.abj6987.
30. Schneider, V. A., Graves-Lindsay, T., Howe, K., Bouk, N., Chen, H.-C., Kitts, P. A., Murphy, T. D., Pruitt, K. D., Thibaud-Nissen, F., Albracht, D., et al. (2017). Evaluation of GRCh38 and de novo haploid genome assemblies demonstrates the enduring quality of the reference assembly. Genome Res. 27, 849-864. 10.1101/gr.213611.116.
31. Sullivan, L. L., and Sullivan, B. A. (2020). Genomic and functional variation of human centromeres. Exp. Cell Res. 389, 111896. 10.1016/j.yexcr.2020.111896.
32. Willard, H. F. (1991). Evolution of alpha satellite. Curr. Opin. Genet. Dev. 1, 509-514. 10.1016/s0959-437× (05) 80200-x.
33. Uralsky, L. I., Shepelev, V. A., Alexandrov, A. A., Yurov, Y. B., Rogaev, E. I., and Alexandrov, I. A. (2019). Classification and monomer-by-monomer annotation dataset of suprachromosomal family 1 alpha satellite higher-order repeats in hg38 human genome assembly. Data Brief 24, 103708. 10.1016/j.dib.2019.103708.
34. Wang, T., Wei, J. J., Sabatini, D. M., and Lander, E. S. (2014). Genetic screens in human cells using the CRISPR-Cas9 system. Science 343, 80-84. 10.1126/science. 1246981.
35. Doench, J. G., Fusi, N., Sullender, M., Hegde, M., Vaimberg, E. W., Donovan, K. F., Smith, I., Tothova, Z., Wilen, C., Orchard, R., et al. (2016). Optimized sgRNA design to maximize activity and minimize off-target effects of CRISPR-Cas9. Nat. Biotechnol. 34, 184-191. 10.1038/nbt.3437.
36. Doench, J. G., Hartenian, E., Graham, D. B., Tothova, Z., Hegde, M., Smith, I., Sullender, M., Ebert, B. L., Xavier, R. J., and Root, D. E. (2014). Rational design of highly active sgRNAs for CRISPR-Cas9-mediated gene inactivation. Nat. Biotechnol. 32, 1262-1267. 10.1038/nbt.3026.
37. Meyers, R. M., Bryan, J. G., McFarland, J. M., Weir, B. A., Sizemore, A. E., Xu, H., Dharia, N. V., Montgomery, P. G., Cowley, G. S., Pantel, S., et al. (2017). Computational correction of copy number effect improves specificity of CRISPR-Cas9 essentiality screens in cancer cells. Nat. Genet. 49, 1779-1784. 10.1038/ng.3984.
38. Ly, P., Eskiocak, U., Kim, S. B., Roig, A. I., Hight, S. K., Lulla, D. R., Zou, Y. S., Batten, K., Wright, W. E., and Shay, J. W. (2011). Characterization of aneuploid populations with trisomy 7 and 20 derived from diploid human colonic epithelial cells. Neoplasia N. Y. N 13, 348-357. 10.1593/neo. 101580.
39. Maciejowski, J., Li, Y., Bosco, N., Campbell, P. J., and de Lange, T. (2015). Chromothripsis and Kataegis Induced by Telomere Crisis. Cell 163, 1641-1654. 10.1016/j.cell.2015.11.054.
40. Sanjana, N. E., Shalem, O., and Zhang, F. (2014). Improved vectors and genome-wide libraries for CRISPR screening. Nat. Methods 11, 783-784. 10.1038/nmeth.3047.
41. Bajaj, R., Bollen, M., Peti, W., and Page, R. (2018). KNL1 Binding to PP1 and Microtubules Is Mutually Exclusive. Structure 26, 1327-1336.e4. 10.1016/j.str.2018.06.013.
42. DeLuca, J. G., Gall, W. E., Ciferri, C., Cimini, D., Musacchio, A., and Salmon, E. D. (2006). Kinetochore microtubule dynamics and attachment stability are regulated by Hec1. Cell 127, 969-982. 10.1016/j.cell.2006.09.047.
43. Hatch, E. M., Fischer, A. H., Deerinck, T. J., and Hetzer, M. W. (2013). Catastrophic nuclear envelope collapse in cancer cell micronuclei. Cell 154, 47-60. 10.1016/j.cell.2013.06.007.
44. Meerbrey, K. L., Hu, G., Kessler, J. D., Roarty, K., Li, M. Z., Fang, J. E., Herschkowitz, J. I., Burrows, A. E., Ciccia, A., Sun, T., et al. (2011). The pINDUCER lentiviral toolkit for inducible RNA interference in vitro and in vivo. Proc. Natl. Acad. Sci. U.S.A 108, 3665-3670. 10.1073/pnas. 1019736108.
45. Banaszynski, L. A., Chen, L.-C., Maynard-Smith, L. A., Ooi, A. G. L., and Wandless, T. J. (2006). A rapid, reversible, and tunable method to regulate protein function in living cells using synthetic small molecules. Cell 126, 995-1004. 10.1016/j.cell.2006.07.025.
46. Gao, R., Bai, S., Henderson, Y. C., Lin, Y., Schalck, A., Yan, Y., Kumar, T., Hu, M., Sei, E., Davis, A., et al. (2021). Delineating copy number and clonal substructure in human tumors from single-cell transcriptomes. Nat. Biotechnol. 39, 599-608. 10.1038/s41587-020-00795-2.
47. Patel, A. P., Tirosh, I., Trombetta, J. J., Shalek, A. K., Gillespie, S. M., Wakimoto, H., Cahill, D. P., Nahed, B. V., Curry, W. T., Martuza, R. L., et al. (2014). Single-cell RNA-seq highlights intratumoral heterogeneity in primary glioblastoma. Science 344, 1396-1401. 10.1126/science.1254257.
48. Tirosh, I., Izar, B., Prakadan, S. M., Wadsworth, M. H., Treacy, D., Trombetta, J. J., Rotem, A., Rodman, C., Lian, C., Murphy, G., et al. (2016). Dissecting the multicellular ecosystem of metastatic melanoma by single-cell RNA-seq. Science 352, 189-196. 10.1126/science.aad0501.
49. The Cancer Genome Atlas Network (2012). Comprehensive molecular characterization of human colon and rectal cancer. Nature 487, 330-337. 10.1038/nature11252.
50. Love, M. I., Huber, W., and Anders, S. (2014). Moderated estimation of fold change and dispersion for RNA-seq data with DESeq2. Genome Biol. 15, 550. 10.1186/s13059-014-0550-8.
51. Massagué, J., Blain, S. W., and Lo, R. S. (2000). TGFbeta signaling in growth control, cancer, and heritable disorders. Cell 103, 295-309. 10.1016/s0092-8674 (00) 00121-5.
52. Drost, J., van Jaarsveld, R. H., Ponsioen, B., Zimberlin, C., van Boxtel, R., Buijs, A., Sachs, N., Overmeer, R. M., Offerhaus, G. J., Begthel, H., et al. (2015). Sequential cancer mutations in cultured human intestinal stem cells. Nature 521, 43-47. 10.1038/nature14415.
53. van de Wetering, M., Francies, H. E., Francis, J. M., Bounova, G., Iorio, F., Pronk, A., van Houdt, W., van Gorp, J., Taylor-Weiner, A., Kester, L., et al. (2015). Prospective derivation of a living organoid biobank of colorectal cancer patients. Cell 161, 933-945. 10.1016/j.cell.2015.03.053.
54. Woodford-Richens, K. L., Rowan, A. J., Gorman, P., Halford, S., Bicknell, D. C., Wasan, H. S., Roylance, R. R., Bodmer, W. F., and Tomlinson, I. P. M. (2001). SMAD4 mutations in colorectal cancer probably occur before chromosomal instability, but after divergence of the microsatellite instability pathway. Proc. Natl. Acad. Sci. 98, 9719-9723. 10.1073/pnas. 171321498.
55. Thiagalingam, S., Lengauer, C., Leach, F. S., Schutte, M., Hahn, S. A., Overhauser, J., Willson, J. K., Markowitz, S., Hamilton, S. R., Kern, S. E., et al. (1996). Evaluation of candidate tumour suppressor genes on chromosome 18 in colorectal cancers. Nat. Genet. 13, 343-346. 10.1038/ng0796-343.
56. Cheng, P., Zhao, X., Katsnelson, L., Camacho-Hernandez, E. M., Mermerian, A., Mays, J. C., Lippman, S. M., Rosales-Alvarez, R. E., Moya, R., Shwetar, J., et al. (2022). Proteogenomic analysis of cancer aneuploidy and normal tissues reveals divergent modes of gene regulation across cellular pathways. eLife 11, e75227. 10.7554/eLife.75227.
57. Eppert, K., Scherer, S. W., Ozcelik, H., Pirone, R., Hoodless, P., Kim, H., Tsui, L. C., Bapat, B., Gallinger, S., Andrulis, I. L., et al. (1996). MADR2 maps to 18q21 and encodes a TGFbeta-regulated MAD-related protein that is functionally mutated in colorectal carcinoma. Cell 86, 543-552. 10.1016/s0092-8674 (00) 80128-2.
58. Dumont, M., Gamba, R., Gestraud, P., Klaasen, S., Worrall, J. T., De Vries, S. G., Boudreau, V., Salinas-Luypaert, C., Maddox, P. S., Lens, S. M., et al. (2020). Human chromosome-specific aneuploidy is influenced by DNA-dependent centromeric features. EMBO J. 39. 10.15252/embj.2019102924.
59. Cimini, D., Howell, B., Maddox, P., Khodjakov, A., Degrassi, F., and Salmon, E. D. (2001). Merotelic kinetochore orientation is a major mechanism of aneuploidy in mitotic mammalian tissue cells. J. Cell Biol. 153, 517-527. 10.1083/jcb. 153.3.517.
60. Gregan, J., Polakova, S., Zhang, L., Tolić-Nørrelykke, I. M., and Cimini, D. (2011). Merotelic kinetochore attachment: causes and effects. Trends Cell Biol. 21, 374-381. 10.1016/j.tcb.2011.01.003.
61. Whinn, K. S., Kaur, G., Lewis, J. S., Schauer, G. D., Mueller, S. H., Jergic, S., Maynard, H., Gan, Z. Y., Naganbabu, M., Bruchez, M. P., et al. (2019). Nuclease dead Cas9 is a programmable roadblock for DNA replication. Sci. Rep. 9, 13292. 10.1038/s41598-019-49837-z.
62. Giunta, S., Hervé, S., White, R. R., Wilhelm, T., Dumont, M., Scelfo, A., Gamba, R., Wong, C. K., Rancati, G., Smogorzewska, A., et al. (2021). CENP-A chromatin prevents replication stress at centromeres to avoid structural aneuploidy. Proc. Natl. Acad. Sci. 118, e2015634118. 10.1073/pnas.2015634118.
63. Bury, L., Moodie, B., Ly, J., Mckay, L. S., Miga, K. H., and Cheeseman, I. M. (2020). Alpha-satellite RNA transcripts are repressed by centromere-nucleolus associations. eLife 9, e59770. 10.7554/eLife.59770.
64. McNulty, S. M., Sullivan, L. L., and Sullivan, B. A. (2017). Human Centromeres Produce Chromosome-Specific and Array-Specific Alpha Satellite Transcripts that Are Complexed with CENP-A and CENP-C. Dev. Cell 42, 226-240.e6. 10.1016/j.devcel.2017.07.001.
65. Chan, F. L., Marshall, O. J., Saffery, R., Won Kim, B., Earle, E., Choo, K. H. A., and Wong, L. H. (2012). Active transcription and essential role of RNA polymerase II at the centromere during mitosis. Proc. Natl. Acad. Sci. 109, 1979-1984. 10.1073/pnas. 1108705109.
66. Kabeche, L., Nguyen, H. D., Buisson, R., and Zou, L. (2018). A mitosis-specific and R loop-driven ATR pathway promotes faithful chromosome segregation. Science 359, 108-114. 10.1126/science.aan6490.
67. Sarli, L., Bottarelli, L., Bader, G., Iusco, D., Pizzi, S., Costi, R., Dâ€™ Adda, T., Bertolani, M., Roncoroni, L., and Bordi, C. (2004). Association Between Recurrence of Sporadic Colorectal Cancer, High Level of Microsatellite Instability, and Loss of Heterozygosity at Chromosome 18q. Dis. Colon Rectum 47, 1467-1482. 10.1007/s10350-004-0628-6.
68. Tanaka, T., Watanabe, T., Kazama, Y., Tanaka, J., Kanazawa, T., Kazama, S., and Nagawa, H. (2006). Chromosome 18q deletion and Smad4 protein inactivation correlate with liver metastasis: a study matched for T- and N-classification. Br. J. Cancer 95, 1562-1567. 10.1038/sⱼ.bjc.6603460.
69. McFadden, D. G., Papagiannakopoulos, T., Taylor-Weiner, A., Stewart, C., Carter, S. L., Cibulskis, K., Bhutkar, A., McKenna, A., Dooley, A., Vernon, A., et al. (2014). Genetic and clonal dissection of murine small cell lung carcinoma progression by genome sequencing. Cell 156, 1298-1311. 10.1016/j.cell.2014.02.031.
70. Trakala, M., Aggarwal, M., Sniffen, C., Zasadil, L., Carroll, A., Ma, D., Su, X. A., Wangsa, D., Meyer, A., Sieben, C. J., et al. (2021). Clonal selection of stable aneuploidies in progenitor cells drives high-prevalence tumorigenesis. Genes Dev. 35, 1079-1092. 10.1101/gad.348341.121.
71. Xue, W., Kitzing, T., Roessler, S., Zuber, J., Krasnitz, A., Schultz, N., Revill, K., Weissmueller, S., Rappaport, A. R., Simon, J., et al. (2012). A cluster of cooperating tumor-suppressor gene candidates in chromosomal deletions. Proc. Natl. Acad. Sci. U.S.A. 109, 8212-8217. 10.1073/pnas. 1206062109.
72. Chen, B., Gilbert, L. A., Cimini, B. A., Schnitzbauer, J., Zhang, W., Li, G.-W., Park, J., Blackburn, E. H., Weissman, J. S., Qi, L. S., et al. (2013). Dynamic Imaging of Genomic Loci in Living Human Cells by an Optimized CRISPR/Cas System. Cell 155, 1479-1491. 10.1016/j.cell.2013.12.001.
73. Ran, F. A., Hsu, P. D., Wright, J., Agarwala, V., Scott, D. A., and Zhang, F. (2013). Genome engineering using the CRISPR-Cas9 system. Nat. Protoc. 8, 2281-2308. 10.1038/nprot.2013.143.
74. Cong, L., Ran, F. A., Cox, D., Lin, S., Barretto, R., Habib, N., Hsu, P. D., Wu, X., Jiang, W., Marraffini, L. A., et al. (2013). Multiplex Genome Engineering Using CRISPR/Cas Systems. Science 339, 819-823. 10.1126/science. 1231143.
75. Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji—an Open Source platform for biological image analysis. Nat. Methods 9, 10.1038/nmeth.2019. 10.1038/nmeth.2019.
76. Gilbert, L. A., Horlbeck, M. A., Adamson, B., Villalta, J. E., Chen, Y., Whitehead, E. H., Guimaraes, C., Panning, B., Ploegh, H. L., Bassik, M. C., et al. (2014). Genome-Scale CRISPR-Mediated Control of Gene Repression and Activation. Cell 159, 647-661. 10.1016/j.cell.2014.09.029.
77. van der Walt, S., Schönberger, J. L., Nunez-Iglesias, J., Boulogne, F., Warner, J. D., Yager, N., Gouillart, E., Yu, T., and scikit-image contributors (2014). scikit-image: image processing in Python. PeerJ 2, e453. 10.7717/peerj.453.
78. Li, H., and Durbin, R. (2009). Fast and accurate short read alignment with Burrows-Wheeler transform. Bioinforma. Oxf. Engl. 25, 1754-1760. 10.1093/bioinformatics/btp324.
79. Van der Auwera, G. A. (2020). Genomics in the cloud: using Docker, GATK, and WDL in Terra First edition. (O'Reilly Media).
80. Kuilman, T., Velds, A., Kemper, K., Ranzani, M., Bombardelli, L., Hoogstraat, M., Nevedomskaya, E., Xu, G., de Ruiter, J., Lolkema, M. P., et al. (2015). CopywriteR: DNA copy number detection from off-target sequence data. Genome Biol. 16, 49. 10.1186/s13059-015-0617-1.
81. Dolgalev, Igor (2022). Seq-N-Slide. 10.5281/ZENODO.5550459.
82. Bolger, A. M., Lohse, M., and Usadel, B. (2014). Trimmomatic: a flexible trimmer for Illumina sequence data. Bioinforma. Oxf. Engl. 30, 2114-2120. 10.1093/bioinformatics/btu170.
83. Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinforma. Oxf. Engl. 29, 15-21. 10.1093/bioinformatics/bts635.
84. Liao, Y., Smyth, G. K., and Shi, W. (2014). featureCounts: an efficient general purpose program for assigning sequence reads to genomic features. Bioinforma. Oxf. Engl. 30, 923-930. 10.1093/bioinformatics/btt656.
85. Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc. Natl. Acad. Sci. U.S.A 102, 15545-15550. 10.1073/pnas.0506580102.
86. Hao, Y., Hao, S., Andersen-Nissen, E., Mauck, W. M., Zheng, S., Butler, A., Lee, M. J., Wilk, A. J., Darby, C., Zager, M., et al. (2021). Integrated analysis of multimodal single-cell data. Cell 184, 3573-3587.e29. 10.1016/j.cell.2021.04.048.
87. Gu, Z., Eils, R., and Schlesner, M. (2016). Complex heatmaps reveal patterns and correlations in multidimensional genomic data. Bioinformatics 32, 2847-2849. 10.1093/bioinformatics/btw313.
88. Liu, J., Lichtenberg, T., Hoadley, K. A., Poisson, L. M., Lazar, A. J., Cherniack, A. D., Kovatich, A. J., Benz, C. C., Levine, D. A., Lee, A. V., et al. (2018). An Integrated TCGA Pan-Cancer Clinical Data Resource to Drive High-Quality Survival Outcome Analytics. Cell 173, 400-416.e11. 10.1016/j.cell.2018.02.052.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1. A fusion protein comprising a mutated kinetochore protein and dCas9.

2. The fusion protein of claim 1, wherein the kinetochore protein comprises a segment of KNL1 protein, wherein the segment of the KNL1 protein comprises at least the first 86 N-terminal amino acids of the KNL1 protein, and wherein the first 86 N-terminal amino acids comprises a mutation of the sequence RVSF to AAAA, or S24A to S60A.

3. The fusion protein of claim 2, wherein the segment of KNL1 protein comprises the sequence
(SEQ ID NO: 1)
MDGVSSEANEENDNIERPVRRRHSSILKPPRSPLQDLRGGNETV
QESNALRNKKNSRAAAAADTIKVFQTESHMKIVRKSEMEETE
or
(SEQ ID NO: 2)
MDGVSSEANEENDNIERPVRRRHASILKPPRSPLQDLRGGNETV
QESNALRNKKNSRRVAFADTIKVFQTESHMKIVRKS

4. A composition comprising the fusion protein of claim 1.

5. The composition of claim 4, further comprising at least one guide RNA that targets the fusion protein to a location of kinetochore assembly on a centromere such that the fusion protein interferes with chromosome segregation.

6. A method comprising introducing into cells in vitro a fusion protein of claim 1 and at least one guide RNA that targets the fusion protein to a location of kinetochore assembly on a centromere of a specific chromosome such that the fusion protein interferes with segregation of the chromosome, and allowing cell division in the presence of the fusion protein and the guide RNA such that cell division results in divided cells that comprise an aneuploidy karyotype.

7. The method of claim 6, wherein the aneuploidy karyotype comprises a gain of a chromosome.

8. The method of claim 6, wherein the aneuploidy karyotype comprises a loss of a chromosome.

9. The method claim 6, wherein the aneuploidy karyotype is associated with a malignant cell phenotype.

10. An isolated population of cells which comprise an aneuploidy karyotype made by the method of claim 6.

11. The isolated population of cells of claim 10, wherein the aneuploidy karyotype comprises a loss of a chromosome.

12. The isolated population of cells of claim 10, wherein the aneuploidy karyotype comprises a gain of a chromosome.

13. The isolated population of cells of claim 10, wherein the aneuploidy karyotype is associated with a malignant cell phenotype.

14. A kit comprising a fusion protein of claim 1 or an expression vector encoding the fusion protein, and optionally one or more guide RNAs that target the fusion protein to a location of kinetochore assembly on a centromere, or one or more polynucleotides that encode the one or more guide RNAs.

15. A method comprising selecting a guide RNA that targets a location of kinetochore assembly on a centromere of a specific chromosome, and introducing into cells a combination of the selected guide RNA and a fusion protein comprising a mutated kinetochore protein and dCas9, and allowing cell divisional in the presence of the selected guide RNA and the fusion protein such that divided cells comprise an aneuploidy karyotype.

16. An expression vector encoding a fusion protein of claim 1.
